(19) United States
(12) Patent Application Publication
(10) Pub. No.: US 2025/0388892 A1
(43) Pub. Date: Dec. 25, 2025

(54) HLA PANELS FOR EPITOPE MAPPING AND METHODS FOR DESIGNING SUCH PANELS

(71) Applicant: One Lambda, Inc., West Hills, CA (US)

(72) Inventors: Zhifang Wang, Thousand Oaks, CA (US); Shi-Yuan Meng, Simi Valley, CA (US); David Philip Lowe, Alsager (GB)

(73) Assignee: One Lambda, Inc., West Hills, CA (US)

(21) Appl. No.: 19/316,393

(22) Filed: Sep. 2, 2025

Related U.S. Application Data
(63) Continuation of application No. PCT/US2024/051228, filed on Oct. 14, 2024.
(60) Provisional application No. 63/544,073, filed on Oct. 13, 2023.

(51) Int. Cl. C12N 15/10; C07K 14/74; G01N 33/68

(52) U.S. Cl. CPC C12N 15/1037; C07K 14/70539; G01N 33/6845

(57) ABSTRACT

Materials and methods for generating a panel including human leukocyte antigens (HLA) and engineered variants (EVs) thereof, as well as diagnostic and/or therapeutic methods of using the panel are provided.

### CROSS REFERENCE TO RELATED APPLICATIONS

This application is a continuation of International Application No. PCT/US2024/051228, filed Oct. 14, 2024, which claims the benefit of U.S. Provisional Application No. 63/544,073, filed Oct. 13, 2023, which is incorporated by reference herein in its entirety.

### SEQUENCE LISTING INCORPORATION

The Sequence Listing is submitted as an XML file in the form of the file named “9748-111081-03_Sequence_Listing” (10,800 bytes), which was created on Sep. 2, 2025, which is incorporated by reference herein.

### FIELD

The present disclosure relates generally to detection and characterization of antibodies specific for human leukocyte antigens (HLA), and more particularly to materials and methods for generating a panel (such as a single antigen bead (SAB) panel) that includes human leukocyte antigens (HLA) and engineered variants (EV) thereof, algorithms for panel design and reactivity analyses, as well as diagnostic and/or therapeutic methods of using the panel.

### BACKGROUND

Classical HLA are categorized based on sequence and structural homology into 11 loci, namely those containing the Class I HLA alleles: HLA-A, HLA-B and HLA-C, and those loci containing the Class II HLA alleles: HLA-DRB1, HLA-DRB3, HLA-DRB4, HLA-DRB5, HLA-DQA1, HLA-DQB1, HLA-DPA1 and HLA-DPB1. Within each locus, there are conserved and variant positions.

Exposure to foreign HLA, including from organ transplant, blood transfusion or pregnancy, generally initiates the host immune responses that lead to proliferation of foreign HLA-specific plasma cells and, in turn, production of antibodies (Ab) targeting foreign HLA, referred to as donor specific antibodies (DSA). In transplant recipients, such DSA are known to be associated with antibody-mediated rejection (AMR) which adversely impacts long-term survival of the transplant and recipient. Although a perfect or near-perfect match between donor and recipient HLA is ideal, such matches are rarely achieved due to the high degree of polymorphism in the HLA system. As a result, almost all recipients of organ transplants are exposed to HLA mismatches unless donor and recipient are identical twins.

HLA SAB assays are one method for detecting DSA present in organ transplant recipients for pre-transplant matching and post-transplant monitoring purposes. As is common in interactions between Ab and structurally complex antigens (Ag), DSA react with specific conformational epitopes that reside in one or more HLA molecules. Such conformational epitopes are not necessarily defined solely by a linear stretch of amino acid sequence, and they may be affected by noncontiguous residues that form part of the three-dimensional (3D) structure of the epitope. Further, HLA have both unique “private” epitopes and common “public” epitopes that are shared among multiple HLA molecules. Therefore, SAB assays are limited not only by the number but also the nature of HLA included on the SAB panel, making interpretation of reactivity patterns somewhat deficient and/or uncertain if critical donor alleles and specific epitopes are not included.

High resolution (2-field) typing at the HLA alleles of both the recipient and potential donor may be used to reveal exact amino acid mismatches, but high-resolution typing alone is not likely to replace SAB assays as the standard practice because it provides little information about immunogenicity. Depending upon the HLA molecules involved in a mismatch, different mismatches may have different immunological impact or outcome. Methods for distinguishing strongly immunogenic mismatches which are likely to result in development of DSA from acceptable mismatches which will not induce significant DSA production, would improve outcome and survival rates for transplant recipients particularly when two or more organ donor candidates are available and for transplant recipients who may require monitoring post-transplant or require additional future transplants.

General knowledge of the interface between an Ab's binding region (paratope) and its cognate Ag's binding region (epitope) is based on structural information. Although the interface covers a wide area of surface residues, for example up to 25 amino acids in a 15 Å radius, the so-called structural epitope, it is generally thought that contact point(s) of an Ag interacting with the complementarity-determining region 3 (CDR3) of the Ab heavy chain (HC) determines specificity, while additional contact points between Ag and Ab contribute to affinity. Such a specificity-defining epitope is termed functional epitope, and is thought to occupy an area of less than 3.5 Å radius. In the case of HLA molecules, due to the high overall sequence and structural homology, a single amino acid polymorphism could define a functional epitope. It is generally thought functional epitope determines the specificity of interaction with a paratope, and therefore the immunogenicity of a mismatch. Accordingly, Ab-verified functional epitopes are expected to be associated with higher risk of immunogenicity, although the verification methodology has not been standardized.

A functional epitope of HLA, by definition, is specific to a particular paratope of a monoclonal antibody (mAb). Such a functional epitope can be carried by a single allele or by multiple alleles. Because of their polymorphic nature, different HLA molecules carrying the same functional epitope could bind to the same mAb at a very different affinity depending on the overall composition of a complete structural epitope. On the other hand, a single mAb could recognize a single functional epitope on one allele and potentially a different functional epitope at a similar location on another allele. A functional epitope is necessary but insufficient by itself to support a measurable interaction between a specific Ag-Ab pair. A functional epitope combined with the structural epitope which covers the remainder of the interface with the mAb, defines a complete epitope which provides both specificity and affinity of a binding event under physiological conditions.

### SUMMARY

The prevailing concept of a functional epitope refers to the HLA eplet system defined by HLAMatchmaker™ where each potential functional epitope is theoretically predefined as an eplet that comprises the minimal amino acid configuration within a 3-3.5 Å radius needed to induce an Ab response. However, many eplets are traditionally defined based on sequence alignment of known alleles that do not provide sufficient resolution in excluding amino acids outside the 3.5 Å radius (epregistry.com.br/). However, there are deficiencies in adopting the eplet system for research purposes and clinical applications. That is why instead of imputing artificially predefined eplets without sufficient experimental data, this disclosure may utilize all individual amino acids at variant positions (eps), as well as eplet patterns present in a targeted population(s) regardless of whether they are registered eplets or not.

Considering the complexity of the binding signals of even one mAb to a panel of HLA molecules, additional complication due to the presence of polyclonal antibodies (pAb) in a test sample is inevitable. Most clinical samples from sensitized recipients contain pAb against single or multiple HLA molecules (HLA Ag(s)). To deconvolute, a portion of pAb present in a test sample may be adsorbed to a particular HLA molecule presented on cells or immobilized on a solid phase surface (e.g., solid support), such as bead surface, and then eluted out to separate from the remaining pAb that do not bind to the specific HLA molecule. By partitioning Ab binders to HLA molecules with distinct, functional epitopes, each round of adsorption-elution (Ads-Elu) can provide further clarity as to which functional epitopes are likely recognized by the pAb mix. Such Ads-Elu protocols are commonly practiced in the field of epitope mapping of complex antibody sample mixtures. In some cases, to avoid the potential artifacts that could be introduced during an elution, many researchers prefer measuring the remaining activities of the sample to decipher what Ab/HLA Ag binding signals have been reduced by adsorbing to the known HLA allele on cells or a solid phase surface.

mAb against various HLA alleles and corresponding HLA Ag amino acid sequences isolated from human subjects are known and several approaches have been used to define their cognate epitopes on such HLA molecules. More specifically, some functional epitopes, where a single amino acid change could lead to the loss of Ab recognition, have been identified through site-directed mutagenesis. Determination of antigenicity, and therefore the prediction of immunogenicity, at the individual amino acid level would significantly improve the field of HLA matching. However, currently available HLA panels, especially SAB panels, do not have sufficient coverage of certain populations and are unable to resolve the ambiguity of multiple residues unlikely to constitute the same functional epitope because of their distance and topology.

Conventionally, only known alleles have been included in SAB panels because of concerns that a change in the native wildtype (WT) sequence, even a single amino acid, could alter conformations of the remaining unchanged residues and potentially destroy the epitopes they present. However, it is disclosed herein that data from site-directed mutagenesis reveals that a residue swap at the same position known to harbor variants among alleles within the same locus, or replacement with a residue unknown to that position, generally has little detectable effect on the overall or local conformations of the remaining epitopes not involving such a position. Although the possibility of affecting the affinity of an overlapping epitope remains, a single or even multiple amino acid changes within a functional epitope is unlikely to affect another non-overlapping functional epitope. This realization lays out the concept for including engineered HLA variants (EV), that may or may not exist in global populations, in a panel to not only provide better coverage of HLA Ag but also confer higher resolution at the individual amino acid positions for more robust and efficacious clinical diagnostics. EV carrying an altered functional epitope from the corresponding WT HLA Ag are informative based on the impact of such an alteration to a WT-binding Ab. The alteration involving one or more than one amino acid residues within a radius that could be in contact with a CDR could lead to an altered specificity and/or affinity ranging from complete loss of binding/loss of function (LOF) for one mAb to gain of function (GOF) to another mAb.

Additionally, EV also enable functional epitope mapping of a sample including pAb. For example, epitopes of pAb of a sample may be directly mapped without resorting to Ads-Elu procedure if the pAb sample binds to the WT Ag but not its derived EV, which means at least one mAb in the pAb sample recognizes the functional epitope on the WT. On the other hand, no LOF to EV comparing to WT does not lead to the conclusion such a mAb does not exist in the sample because a 2^{nd} mAb that recognizes another functional epitope on the same Ag may continue to provide binding signals. In addition, a GOF observation confirms there is at least one mAb in the pAb sample recognizes the EV and its corresponding ep(s)-altered residue(s) at the position(s). Nonetheless, for a complex pAb sample, Ads-Elu may be used to partition the signals by alleles first before performing epitope mapping to reach resolution.

The term functional epitope was originally defined as the critical contact point(s) within a 3.5 Å radius interacting with Ab HC CDR3 (CDR-H3) that determines specificity, with the rest of the contact points between Ag and Ab contributing to affinity. All contacts points, including functional epitope(s), between Ab and Ag were considered the full structural epitope. In practice, for lack of a better term, structural epitope has also been used to describe contact points not implicated in a functional epitope as if the two terms are mutually exclusive.

However, based on emerging data from X-ray crystallography and cryogenic electron microscopy, those critical residues may not be limited to contacting CDR-H3 or just CDRs, and the EV vs WT HLA binding studies described herein have identified residues more than 3.5 Å apart which could both independently be considered essential in some cases. Therefore, there is a need to substitute the concepts of essential residue/region (“ER” as used herein) and ancillary residue/region (“AR” as used herein) for those of functional epitope and structural epitope respectively.

In certain aspects, the disclosure provides a method for designing a SAB panel, composed of HLA and EV thereof, that provides enhanced resolution in deciphering ER recognized by both mAb and pAb derived from sensitized individuals. Verification of ER under certain mismatch and physiological conditions is important for assessing relative immunogenicity risks critical to differentiating permissible/acceptable versus non-permissible/unacceptable mismatches between transplant recipients and donors. Some immunogenicity prediction methods have been developed and refined for this same purpose. However, in many cases, the clinical value of such in silico predictions is contingent upon unambiguous interpretations of experimental observations that are beyond the capabilities of current commercial SAB panels. In some examples, the panels of the present disclosure not only provide higher resolution in identifying functional epitopes but also cover a wider range of variants in targeted populations.

In various aspects, the disclosure provides HLA panels, methods for designing such HLA panels, and computer implemented algorithms for imputing ER based on the specificity profiles obtained from the panel assays. In some aspects, the HLA panel is a SAB panel that includes multiple HLA Class I alleles composed of HLA-A, HLA-B and HLA-C loci, and/or HLA Class II alleles including single or multiple HLA-DR, HLA-DQ and HLA-DP loci. The HLA SAB panel is capable of capturing substantially all variant positions and residues of common HLA alleles (with frequencies greater than 1 in 10,000), intermediate (with frequencies greater than 1 in 100,000), and/or well-documented alleles (with unclear frequencies but which are observed at least five times by DNA sequencing or three times in a shared haplotype) in a target population (CIWD 3.0.0, Hurley et al., CIWD version 3.0.0. HLA. 2020; 95: 516-531.) using a minimal number of HLA alleles to impute the position(s) and residue(s) that comprise, in part or in whole, an ER. Furthermore, 3D pattern recognition of more than one residue within a certain distance is also included for consideration.

HLA molecules are known to be translocated to cell surface plasma membrane through classical signal peptide directed secretory pathway. The mature HLA, post signal peptide cleavage, is anchored to the plasma membrane through a transmembrane domain with a cytoplasmic tail at the C-terminus. An epitope, by definition, must be accessible to a binding Ab. A binding Ab to an epitope has the potential to further initiate immune responses, alone or in combination with other binding Ab to different epitopes, leading to the destruction of the cells bearing the epitope or epitopes on the cell surface. Residues in transmembrane and cytoplasmic domains not exposed on the cell surface are less likely to be clinically significant.

The extracellular domain residues embedded under the molecule surface, deep in a narrow cleft, and/or close to the plasma membrane are generally considered inaccessible to a binding Ab circulating in the bloodstream and interstitial space. However, such notions are based on existing structural information primarily derived from molecular modeling without actual experimental data, and therefore the reliability of such modeling is subject to debate.

It has been reported that different amino acid residues at the same position of an HLA Ag likely demonstrate different degrees of surface or solvent exposure. In addition, inaccessible residues lining the peptide-binding pocket likely manifest their influence on epitope composition indirectly through the peptides presented. Furthermore, these Ab-inaccessible residues themselves likely contribute to the conformational change of a nearby solvent accessible residue(s). For these reasons, this disclosure does not differentiate whether the variant amino acid positions and different residues present at such positions are solvent exposed or not in the early selection process. All HLA variants may be included in the panel so their associated clinical impact can be captured. As more observations are collected, the variants associated with lower risk of DSA development may be considered to present less immunogenicity risk than those more frequently associated with DSA occurrences.

The panel design described herein can cover commonly occurring HLA epitopes in targeted populations with a minimal number of Ag on the panel, such as less than 5, less than 10, less than 15, less than 20, less than 25, less than 30, less than 35, less than 40, less than 45, less than 50, less than 60, less than 70, less than 80, less than 90, or less than 100, or at least 5, at least 10, at least 15, at least 20, at least 25, at least 30, at least 35, at least 40, at least 45, at least 50, or more. Therefore, alleles harboring unique and/or representative variants or patterns (a cluster of amino acid residues within a 3D space that could be in contact with an Ab) at certain positions are selected to provide the coverage while minimizing duplication. Following a methodological inclusion and exclusion criteria as described herein, a list of a minimal number of alleles covering all variant positions and/or patterns is obtained. Certain variants or patterns are represented by only a single allele while others may be represented by multiple alleles.

For HLA-DQ molecules with notable variations in both alpha and beta subunits, it is important to point out that conformations of the beta subunit may be influenced by the alpha subunit of the heterodimer and vice versa. Furthermore, alpha-beta junctions may create unique ER that are not present outside such a specific pair. It is known that certain alpha and beta subunits do not form detectable pairings while others tend to have a higher degree of association, as suggested by linkage disequilibrium. Alpha-beta pairing has also been observed in both cis (on the same haplotype) and trans association. Consequently, individual typing data, especially homozygous, are also relied upon to gauge the possibility of a successful pairing of each combination. However, the identities of many HLA-DQ and HLA-DP alpha subunits of individuals are unknown for the typed beta subunits.

An objective of aspects of this disclosure is to systematically increase alpha-beta pairing coverage without significantly increasing the number of different SAB required for the panel. One solution, using HLA-DQ Ag as an example, is to include at least all representative HLA-DQA1 subunits with at least one selected HLA-DQB1 subunit of the same 1^{st} field typing. HLA alleles of the same 1^{st} field typing share closer homology than alleles of a different 1^{st} field typing. For successful application of the present methods, the designed pairing is expressed. If unsuccessful, an alternative pairing is tested to ensure that at least one of each of the selected beta and alpha allele combinations is represented.

One application for the HLA SAB panel of the present disclosure is to map ER defined at single amino acid resolution as compared to the eplet level. In contrast to eplet, an “ep” is defined as a residue at a specific variant position of one or multiple HLA alleles. For example, 46E is an ep, glutamic acid at position 46, and a potential functional epitope of DQB1*02 alleles, but its ER status can be confirmed only if a LOF variant is defined by a DQ2 mAb. Because the variant positions are defined by which list of alleles are being compared, an ep may be identified on one but not another list of alleles. Therefore, an ep can also be a residue at a specific position, variant position or not, of an HLA molecule.

The scope of a panel is contingent upon the initial starting list of HLA alleles of interest. All variant positions, residues, and patterns within certain 3D distance of these alleles on the list are represented where the eps and their patterns serve as each other's positive and/or negative controls. This allows the imputation (inclusion and exclusion process) to proceed until a minimal number of potential eps, individual or in combination (ep patterns) are implicated for a binding specificity profile. However, in some cases, multiple eps and/or ep patterns cannot be further resolved because no other alleles on the panel or even any known alleles carry differentiating residues at these positions. One solution to address such a situation is to employ an artificially designed allele carrying one or more than one strategically positioned variant residue or ep, namely “engineered variant (EV),” that may or may not exist in a population. Because these positions are known to harbor various residues, substitution with a different amino acid is likely to be well tolerated to serve the purpose of ER mapping. If one or more than one amino acid residues within a certain 3D space change from the WT Ag result in a LOF phenotype to a mAb, then the implicated residue(s) on the WT Ag is qualified as a verified ER.

An engineered variant Ag can show evidence of expression and normal binding to at least one other mAb that recognizes a different epitope on the WT; if so, then the LOF to a specific mAb can be attributed to the loss of this specific ep(s). The implicated ep(s) is usually converted to the ep(s) in the non-binding alleles individually or in combination through recombinant DNA technology of site-directed mutagenesis or gene synthesis. However, amino acids not already known as eps of HLA molecules can also serve the purpose of creating a LOF variant providing that this EV expresses and binds to at least one other mAb that recognizes a different epitope on the WT. In some cases, a LOF ep to one mAb may create a GOF phenotype to another mAb if a cognate full epitope is created with the new ep. The LOF and GOF observations provide evidence of the modular nature of a structural epitope on HLA that can be exchanged to alter their serological specificity.

To facilitate amino acid sequence analysis and pattern recognition of HLA molecules for the design of an epitope panel and the imputation of implicated ER, computer programming is scripted and incorporated into HLA Fusion™ software (One Lambda) as the AA (AminoAcid) and AA3D (AminoAcid3D) modules. AA module recognizes ep patterns defined by consecutive linear sequences ranging from 1 amino acid up to the length of the user's choice. AA3D module recognizes ep patterns within a certain 3D radius (in A) of the user's choice. Theoretically, AA3D is more appropriate than AA module in analyzing highly conformational HLA molecules. However, because the 3D coordinates are derived from molecular modeling, more experimental data are needed to verify and enhance AA3D utility. On the other hand, AA module is useful up to certain lengths where linear distance no longer corresponds to spatial distance.

For panel design, the software can receive an input of the initial list of alleles with frequency information if available from a targeted population(s). If allele frequency is not available, the designer can specify selection priority. The ability to automate the selection process of a minimal number of alleles with maximal coverage of variants described herein allows the instant comparison of different population considerations to gauge the pros and cons of each selected list and the appropriate trade-offs. The software also provides the convenience in re-evaluating the panel based on updates such as the Immuno Polymorphism Database (IDP)-IMGT/HLA from WHO Nomenclature Committee for Factors of the HLA System (Barker et al., The IPD-IMGT/HLA Database. Nucleic Acids Res. 2023; 51: D1053-D1060.) and CIWD from the International HLA and Immunogenetics Workshop (Hurley et al., Common, intermediate and well-documented HLA alleles in world populations: CIWD version 3.0.0. HLA. 2020; 95: 516-531.), plus feedback from clinical observations.

Because of their genomic organization and mRNA splicing mechanism, some positive binding HLA antigens could always carry the same multiple variant residues at certain positions exceeding the footage of one Ab CDR, and therefore it could not be deconvoluted as to which ep(s) or ep cluster(s) is the ER in the absence of a suitable negative binding HLA antigen(s) without carrying the same set of variant residues. This kind of ambiguities often occur if the Ag panel is limited to WT Common (C, I and WD) alleles. To deconvolute and increase the resolution in identifying ER based on Ag-Ab specificity profiles, strategic inclusion of certain engineered variants (EV) can be advantageous.

In summary, this disclosure describes SAB panels that provide enhanced resolution and coverage not only in detecting Ab binding to specific alleles but also identifying specific ER and/or AR in these interactions. The understanding of which amino acid(s) at which position(s) can serve as an antigenic ER is a critical step toward predicting immunogenicity risk when considering transplant donor-recipient matching and monitoring.

### BRIEF DESCRIPTION OF THE DRAWINGS

For a fuller understanding of the nature and advantages of the present disclosure, reference should be made to the following detailed description taken in connection with the accompanying drawings. It is appreciated that these drawings depict only exemplary aspects and are therefore not to be considered limiting of its scope. The disclosure will be described and explained with additional specificity and detail through the use of the accompanying drawings.

FIGS. 1A-1Q depict the specificity profiles of human and mouse mAb based on an exemplary HLA-DQ Epitope Panel. FIGS. 1A-1L. Human mAb were tested at 10 μg/ml, 3.3 μg/ml, 1.1 μg/ml, and 0.37 μg/ml. The specificity profile from HLA Fusion™ of each sample is shown on the grid. Maximal MFI scale is standardized to 50,000. FIGS. 1M-1P depict mouse mAb derived from unpurified mouse ascites. Their specificity profiles were collected at a specific dilution previously determined using One Lambda LS2A04 HLA Class I SAB panel. Maximal MFI scale is standardized to 50,000. FIG. 1Q depicts the Ag identity of a HLA-DQ epitope panel to a specific mAb, LB_DQB0201_A or FS582 shown as an example sorted in the order of either beta or alpha subunit respectively. The asterisk following FS582 (FS582*) denotes this Ab recognizes alpha instead of beta subunit.

FIGS. 2A-2L are FACS histograms of sorted stable pool expression of WT and EV detected by human mAb. Mouse mAb FJ5109 (pan HLA-CII specific) and FM5148 (pan HLA-DQ specific) serve as the positive controls. The human and mouse IgG isotype-controlled negative mAb are displayed with a black outline. The binding pattern of mAb on each set of WT and its EV are shown with positive controls and test mAb.

FIGS. 2M-2O are FACS histograms of sorted stable pool expression of WT and EV detected by positive control mAb and test mouse mAb. The human and mouse IgG isotype-controlled negative mAb are displayed with a black outline.

FIG. 3 depicts the gain-of function EV derived from the non-binding WT DQB1*03:01/DQA1*03:03 to human mAb LB-DQB0402_A.

FIG. 4A depicts flow cytometry histograms of additional EV targeting residues directly neighboring the verified ER, 130R and 185T, on DQB1*02:02/DQA1*02:01 including conserved residue positions, marked with an “*”, to human mAb LB_DQB0501_C.

FIG. 4B depicts conserved locations of ER (132F*, 174H* and 183P*) next to the variant positions of ER (130R and 185T) based on the structure model of DQB1*02:01/DQA1*02:01

FIG. 5 is a bar graph showing experimental data generated using SAB coated with EV to further resolution in mapping ER. Three EV (L53Q, L53Q_P55R and P55R) derived from WT DQB1*03:01/DQA1*03:03 were coated on Luminex beads. FM5148 and FJ5109 are positive controls and LB_DQB0201_A is the negative control for all constructs.

FIGS. 6A-6C depict the specificity profiles of 8 human serum samples with a DQ epitope panel. An asterisk indicates sorting order is based on alpha subunit.

FIGS. 6D-6F depict the specificity profile of the 8 human serum samples from FIGS. 6A-6C based on LS2A01 HLA Class II SAB panel. Some samples also contain reactivities to DR and DP alleles. Individual allele specificities are not specified but grouped as DR, DQ or DP alleles.

FIG. 7 depicts efficient adsorption and elution of human serum S10848 with DQB1*03:13/DQA1*05:05 (DQ0313A0505) expressing cells. Because the specificity profile does not change between pre-adsorption and post-adsorption, S10848 behaves like a mAb.

FIG. 8 depicts adsorption and elution of human serum Y0026 with DQB1*02:01/DQA1*02:01 (DQ0201A0201) expressing cells. As shown in the elution, adsorption with DQ0201A0201 results in partitioning DQ2 specificities away from non-DQ2 signals shown in pre-adsorption.

FIGS. 9A-9C depict the adsorption and elution of pooled human sera CII_POOL_1 with DQ Ag coated magnetic Luminex beads (One Lambda MagSort). The pan-DQ reactivities observed for the pre-adsorbed CII_POOL_1 sample are partitioned into different specificity profiles with MagSort beads coated with DQB1*03:01/DQA1*02:01 (DQ0301A0201) (FIG. 9A), DQB1*05:01/DQA1*01:01 (DQ0501A00101) (FIG. 9B), and DQB1*06:03/DQA1*01:03 (DQ0603A0103) (FIG. 9C) respectively after elution.

### SEQUENCE LISTING

The nucleic and amino acid sequences listed in the accompanying sequence listing are shown using standard letter abbreviations for nucleotide bases, and single letter code for amino acids, as defined in 37 C.F.R. 1.822. Only one strand of each nucleic acid sequence is shown, but the complementary strand is understood as included by any reference to the displayed strand.

SEQ ID NO: 1 is the amino acid sequence of an exemplary HLA-A extracellular domain of
representative allele A*01:01:
GSHSMRYFFTSVSRPGRGEPRFIAVGYVDDTQFVRFDSDAASQKMEPRAPWIEQEGPEYWDQ
ETRNMKAHSQTDRANLGTLRGYYNQSEDGSHTIQIMYGCDVGPDGRFLRGYRQDAYDGKDY
IALNEDLRSWTAADMAAQITKRKWEAVHAAEQRRVYLEGRCVDGLRRYLENGKETLQRTDP
PKTHMTHHPISDHEATLRCWALGFYPAEITLTWQRDGEDQTQDTELVETRPAGDGTFQKWA
AVVVPSGEEQRYTCHVQHEGLPKPLTLRWELSSQPTIPIVG
SEQ ID NO: 2 is the amino acid sequence of an exemplary HLA-B extracellular domain of
representative allele B*07:02:
GSHSMRYFYTSVSRPGRGEPRFISVGYVDDTQFVRFDSDAASPREEPRAPWIEQEGPEYWDRN
TQIYKAQAQTDRESLRNLRGYYNQSEAGSHTLQSMYGCDVGPDGRLLRGHDQYAYDGKDYI
ALNEDLRSWTAADTAAQITQRKWEAAREAEQRRAYLEGECVEWLRRYLENGKDKLERADPP
KTHVTHHPISDHEATLRCWALGFYPAEITLTWQRDGEDQTQDTELVETRPAGDRTFQKWAAV
VVPSGEEQRYTCHVQHEGLPKPLTLRWEPSSQSTVPIVG
SEQ ID NO: 3 is the amino acid sequence of an exemplary HLA-C extracellular domain of
representative allele C*01:02:
CSHSMKYFFTSVSRPGRGEPRFISVGYVDDTQFVRFDSDAASPRGEPRAPWVEQEGPEYWDRE
TQKYKRQAQTDRVSLRNLRGYYNQSEAGSHTLQWMCGCDLGPDGRLLRGYDQYAYDGKD
YIALNEDLRSWTAADTAAQITQRKWEAAREAEQRRAYLEGTCVEWLRRYLENGKETLQRAE
HPKTHVTHHPVSDHEATLRCWALGFYPAEITLTWQWDGEDQTQDTELVETRPAGDGTFQKW
AAVMVPSGEEQRYTCHVQHEGLPEPLTLRWEPSSQPTIPIVG
SEQ ID NO: 4 is the amino acid sequence of an exemplary HLA-DPA1 extracellular
domain of representative allele DPA1*01:03:
IKADHVSTYAAFVQTHRPTGEFMFEFDEDEMFYVDLDKKETVWHLEEFGQAFSFEAQGGLAN
IAILNNNLNTLIQRSNHTQATNDPPEVTVFPKEPVELGQPNTLICHIDKFFPPVLNVTWLCNGEL
VTEGVAESLFLPRTDYSFHKFHYLTFVPSAEDFYDCRVEHWGLDQPLLKHWEAQEPIQMPETT
ET
SEQ ID NO: 5 is the amino acid sequence of an exemplary HLA-DPB1 extracellular
domain of representative allele DPB1*01:01:
RATPENYVYQGRQECYAFNGTQRFLERYIYNREEYARFDSDVGEFRAVTELGRPAAEYWNSQ
KDILEEKRAVPDRVCRHNYELDEAVTLQRRVQPKVNVSPSKKGPLQHHNLLVCHVTDFYPGS
IQVRWFLNGQEETAGVVSTNLIRNGDWTFQILVMLEMTPQQGDVYICQVEHTSLDSPVTVEW
KAQSDSAQSK
SEQ ID NO: 6 is the amino acid sequence of an exemplary HLA-DQA1 extracellular
domain of representative allele DQA1*01:01:
EDIVADHVASCGVNLYQFYGPSGQYTHEFDGDEEFYVDLERKETAWRWPEFSKFGGFDPQG
ALRNMAVAKHNLNIMIKRYNSTAATNEVPEVTVFSKSPVTLGQPNTLICLVDNIFPPVVNITW
LSNGQSVTEGVSETSFLSKSDHSFFKISYLTFLPSADEIYDCKVEHWGLDQPLLKHWEPEIPAP
MSELTET
SEQ ID NO: 7 is the amino acid sequence of an exemplary HLA-DQB1 extracellular
domain of representative allele DQB1*02:01:
RDSPEDFVYQFKGMCYFTNGTERVRLVSRSIYNREEIVRFDSDVGEFRAVTLLGLPAAEYWNS
QKDILERKRAAVDRVCRHNYQLELRTTLQRRVEPTVTISPSRTEALNHHNLLVCSVTDFYPAQ
IKVRWFRNDQEETAGVVSTPLIRNGDWTFQILVMLEMTPQRGDVYTCHVEHPSLQSPITVEW
RAQSESAQSK
SEQ ID NO: 8 is the amino acid sequence of an exemplary HLA-DRB1 extracellular
domain of representative allele DRB1*01:01:
GDTRPRFLWQLKFECHFFNGTERVRLLERCIYNQEESVRFDSDVGEYRAVTELGRPDAEYWN
SQKDLLEQRRAAVDTYCRHNYGVGESFTVQRRVEPKVTVYPSKTQPLQHHNLLVCSVSGFYP
GSIEVRWFRNGQEEKAGVVSTGLIQNGDWTFQTLVMLETVPRSGEVYTCQVEHPSVTSPLTV
EWRARSESAQSK
SEQ ID NO: 9 is the amino acid sequence of an exemplary HLA-DR3/4/5 extracellular
domain of representative allele DRB3*01:01:
GDTRPRFLWQLKFECHFFNGTERVRLLERCIYNQEESVRFDSDVGEYRAVTELGRPDAEYWN
SQKDLLEQRRAAVDTYCRHNYGVGESFTVQRRVEPKVTVYPSKTQPLQHHNLLVCSVSGFYP
GSIEVRWFRNGQEEKAGVVSTGLIQNGDWTFQTLVMLETVPRSGEVYTCQVEHPSVTSPLTV
EWRARSESAQSK

### DETAILED DESCRIPTION

In one aspect, the disclosure provides an HLA panel having HLA antigen(s) bound to a solid support, such as an SAB panel, a method of designing and developing such a panel, the use of ER imputation software, and the continued enhancement of such a panel based on clinical data collections and evolving HLA population genomics. Exemplary panels are provided wherein the addition of EV with altered residues from WT to the panel can confirm and resolve ambiguities at individual amino acid residue level allowing more robust assessment of immunologic compatibility between a potential or existing transplant donor and a transplant recipient, as well as use of EVs in other diagnostic methodology relevant to the field of transplant diagnostics.

Amino acid positions referred to throughout can be understood with reference to the corresponding representative extracellular domain sequences for each HLA type provided as SEQ ID NOs: 1-9. These sequences lack the signal peptide amino acid sequence.

Provided herein are engineered variants (EV) of an HLA. The EV includes an HLA extracellular domain including an altered essential residue or region (ER), wherein the altered ER is capable of contacting an Ab CDR and/or CDR adjacent framework region, and the altered ER includes at least 1, 2, 3, 4, 5, 6, or more amino acids. In some aspects, the ER includes a change (e.g., substitution) of one or more amino acids compared to at least one naturally occurring HLA. In some aspects, the EV consists of an HLA extracellular domain having an altered ER.

In some aspects, the altered ER includes 2 or 3 amino acids that are within about 1-15 Å of one another, for example, about 3-10, about 3.5-6, or about 4-5 Å of one another (such as within about 3, 3.5, 4, 4.5, 5, 5.5, 6, 7, 8, 9, 10, 11, 12, 13, 14, or 15 Å of one another) when measured in the context of a folded HLA protein or an assembled HLA heterodimer. In one example, the altered ER includes 2 or 3 amino acids that are within about 5 Å of one another. In another example, the altered ER includes 2 or 3 amino acids that are within about 3 Å of one another.

In other aspects, the altered ER includes 2 or 3 amino acids that are within about 1-15 amino acids of one another (such as within about 1-5, 3-10, or 8-15 amino acids) when measured in the context of an unfolded protein. In some examples, the altered ER includes 2 or 3 amino acids that are within 1, 2, 3, 4, 5, 6, 7, 8, 9, 10, 11, 12, 13, 14, or 15 amino acids of one another.

In some aspects, the altered ER includes at least one amino acid that is not a conserved residue in all alleles at an HLA locus. In some examples, a conserved residue is an amino acid that is present in at least 70% (such as at least 70%, at least 75%, at least 80%, at least 85%, at least 90%, at least 95%, at least 96%, at least 97%, at least 98%, at least 99%, or 100%) of alleles at the HLA locus (such as HLA-A, HLA-B, HLA-C, HLA-DRB1, HLA-DQA1, HLA-DQB1, HLA-DPA1, HLA-DPB1, HLA-DRB3, HLA-DRB4, or HLA-DRB5) of a population. In some examples, the altered ER includes at least one amino acid that is not conserved in an HLA extracellular domain. In one example, an amino acid present in a population at a frequency of ≤1 in 10,000 in a population is not conserved.

In additional aspects, the altered ER includes at least one amino acid that is predicted or confirmed to be surface (or solvent) exposed in an HLA allele. Methods of predicting surface or solvent exposure of amino acids in a protein are known to one of ordinary skill in the art and include molecular modeling and prediction software, such as pHLA3D (phla3d.com.br). In some examples, the at least one amino acid is predicted to be solvent exposed in at least one common allele (e.g., at least one allele present in a population at a frequency ≥1 in 10,000. Methods of confirming surface expression of a protein including the at least one amino acid are also known to one of ordinary skill in the art and include those described in the Examples below.

In further aspects, the altered ER includes at least one amino acid that is capable of or predicted to participate in peptide binding. In some examples, the at least one amino acid is predicted to participate in peptide binding in at least one common allele (e.g., at least one allele present in a population at a frequency ≥1 in 10,000.

In additional aspects, the EV may further include a second altered ER (or candidate ER) that is more than about 10 Å from the first altered ER, such as more than about 15 Å, more than about 20 Å, more than about 25 Å, or more than about 30 Å from the first altered ER when measured in the context of a folded HLA protein or extracellular domain or in the context of an assembled HLA heterodimer. In some examples the second altered ER (or candidate ER) is more than about 10-30 Å, about 15-25 Å, or about 18-22 Å from the first altered ER. In other examples the second altered ER (or candidate ER) is more than about 10 Å, about 11 Å, about 12 Å, about 13 Å, about 14 Å, about 15 Å, about 16 Å, about 17 Å, about 18 Å, about 19 Å, about 20 Å, about 21 Å, about 22 Å, about 23 Å, about 24 Å, about 25 Å, about 26 Å, about 27 Å, about 28 Å, about 29 Å, or about 30 Å from the first altered ER. In one specific example, the second altered ER is more than about 20 Å from the first altered ER.

The term “about” as used herein, represents an amount close to the specific stated amount that still performs a desired function or achieves a desired result. For example, the term “about” may refer to an amount that deviates by less than or equal to 10%, or by less than or equal to 5%, or by less than or equal to 1%, or by less than or equal to 0.1%, or by less than or equal to 0.01% from a specifically stated amount or condition. For example, the word “about” when immediately preceding a numerical value may mean a range of plus or minus 10% of that value, e.g., “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55,” “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

Also provided are compositions that include a substrate with an immobilized antigen that includes an EV of HLA or an extracellular domain thereof, such as those disclosed herein. In some examples, the immobilized antigen is selected from those described in Tables 7B-7D. In some aspects, the composition includes a plurality of substrates, wherein each substrate includes an immobilized antigen. In some aspects, each of the plurality of substrates includes a different immobilized antigen. In some examples, at least one of the immobilized antigens is a second EV of HLA or an extracellular domain thereof. In some examples, at least one of the immobilized antigen is a naturally occurring HLA or extracellular domain thereof. In some examples, the plurality of substrates includes 2-100 substrates (such as about 2-10, about 5-20, about 10-25, about 15-30, about 10-25, about 10-20, about 15-25, about 15-30, about 20-40, about 20-30, about 20-35, about 20-40, about 20-50, about 15-60, about 20-45, about 25-50, about 25-60, about 30-50, about 30-60, about 40-75, or about 60-100) or more. In some examples, the plurality of substrates includes at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more. In other aspects, the plurality of substrates includes 100-10,000 or more substrates (such as about 100, about 250, about 500, about 1000, about 2000, about 3000, about 4000, about 5000, about 6000, about 7000, about 8000, about 9000, about 10,000 or more). In some examples, the plurality of substrates includes less than 5, less than 10, less than 15, less than 20, less than 25, less than 30, less than 35, less than 40, less than 45, less than 50, less than 55, less than 60, less than 65, less than 70, less than 75, less than 80, less than 85, less than 90, less than 95, less than 100, or more. In other aspects, the plurality of substrates includes 100-10,000 or less substrates. In some aspects, the substrate is a bead or the plurality of substrates are beads; however, it will be appreciated that any solid support can be utilized.

In some examples, the composition includes less than 5 or fewer (such as less than 5, less than 10, less than 15, less than 20, less than 25, less than 30, less than 40, less than 50, less than 60, less than 70, less than 80, less than 90, or less than 100) immobilized antigens from naturally occurring HLA, or an extracellular domain thereof. In some examples, the composition includes at least 5 or more (such as at least 5, at least 10, at least 15, at least 20, at least 25, at least 30, at least 40, at least 50, at least 60, at least 70, at least 80, at least 90, at least 100, or more) immobilized antigens from naturally occurring HLA, or an extracellular domain thereof.

In some aspects, at least one of the immobilized antigens in the composition includes at least one EV selected from Tables 7B-7D or an extracellular domain thereof. In other aspects, at least one of the immobilized antigens in the composition is a naturally occurring HLA selected from those set forth in any one of Table 1A, Table 1B, Table 1C, Table 7B, Table 7C, Table 7D, Table 11A, Table 11B, Table 11C, Table 12B, Table 12C, Table 13, Table 14B, Table 14C, Table 15A, Table 15B, Table 15C, Table 16B, Table 16C, or Table 16D, or an extracellular domain thereof. In some examples, the composition includes at least 5 or more (such as at least 5, at least 10, at least 15, at least 20, at least 25, at least 30, at least 40, at least 50, or more) immobilized antigens from naturally occurring HLA, or an extracellular domain thereof. In some examples, the composition includes less than 5 or fewer (such as less than 5, less than 10, less than 15, less than 20, less than 25, less than 30, less than 40, less than 50, or more) immobilized antigens from naturally occurring HLA, or an extracellular domain thereof.

Also provided are HLA panels that include one or more antigens including an altered ER at a position selected from those set forth in Table 7A, Table 12A, Table 14A, Table 16A, Table 17A, or Table 17B. Further provided are HLA panels that include one or more of, or consist of the antigens set forth in one of Table 1A, Table 1B, Table 1C, Table 11A, Table 11B, Table 11C, Table 13, Table 15A, Table 15B, Table 15C, and/or combination thereof. Also provided are HLA panels that include or consist of a set of antigens including at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the antigens set forth in one of Table 1A, Table 1B, Table 1C, Table 11A, Table 11B, Table 11C, Table 13, Table 15A, Table 15B, or Table 15C. Also provided are HLA panels that include or consist of a set of antigens including less than 5%, less than 10%, less than 15%, less than 20%, less than 25%, less than 30%, less than 35%, less than 40%, less than 45%, less than 50%, less than 55%, less than 60%, less than 65%, less than 70%, less than 75%, less than 80%, less than 85%, less than 90%, less than 95%, less than 96%, less than 97%, less than 98%, or less than 99% of the antigens set forth in one of Table 1A, Table 1B, Table 1C, Table 11A, Table 11B, Table 11C, Table 13, Table 15A, Table 15B, or Table 15C. In some examples, the panel is a single antigen bead panel.

The current SAB assay is based on Luminex xMAP technology (DiaSorin Corporate) that allows multiplexing, such as of 500 targets in a single run using a single sample volume. This technology platform is particularly suitable for detecting HLA DSA in populations of diverse HLA typing. However, other existing or to-be-developed multiplex technology platforms could also be used to create a panel of selected HLA Ag as described in this disclosure which optionally utilize a minimal number of HLA Ag with increased diagnostic efficacy.

In certain aspects, the disclosure provides a method of generating a human leukocyte antigen (HLA) panel. The method may include:
a. selecting a population of HLA alleles;
b. identifying a putative essential residue or region (ER) contained in an extracellular domain of amino acid sequences of the population of HLA alleles;
c. selecting a minimal set of amino acid sequences from the population of HLA alleles capable of forming the putative ER;
d. determining the putative ER to be an HLA epitope; and
e. selecting antigens to include in an HLA panel.

In certain aspects, the disclosure provides a method of generating an engineered variant (EV) of a human leukocyte antigen (HLA). The method may include:
a. obtaining a compilation of HLA allele expressed amino acid sequences;
b. identifying an essential residue or region (ER) contained in an extracellular domain from the expressed amino acid sequences; and
c. generating an EV having an altered ER, wherein the altered ER has at least 1, 2, 3, 4, 5, 6 or more amino acid residues which are altered as compared to the identified ER, thereby generating the EV.

In one aspect, a method for designing a panel of the disclosure using AA3D module is described as follows which utilizes one or more computer implemented algorithms optionally performed using, or with the assistance of, artificial intelligence, e.g., machine learning, neural networks, and the like.

Compile an initial list of alleles within selected HLA classes or loci based on population frequencies as defined as common (C), intermediate (I), and well-documented (WD) (as defined in CIWD catalog, version 3.0.0, incorporated herein by reference in its entirety). Alleles designated as “common” in any of the world populations are of higher priority than “intermediate” alleles, and “intermediate” alleles are of higher priority than “well-documented” alleles. Examples disclosed herein often focus on common alleles, but the same algorithm can be applied to any starting list of HLA alleles.

Identify an ep including a variant position having different amino acid residues within the position and/or an ep pattern by aligning allele sequences in the categories of HLA Class I alleles (HLA-A, HLA-B and HLA-C), and Class II alleles including HLA-DR (HLA-DRB1, HLA-DRB3, HLA-DRB4 and HLA-DRB5), HLA-DQ (HLA-DQA1 and HLA-DQB1), or HLA-DP (HLA-DPA1 and HLA-DPB1). HLA molecules belonging to the same locus generally share higher sequence and structural homology than those from different loci. For HLA Class I alleles, although consisting of 3 loci, their sequences and structures are generally aligned well enough to be considered together. However, the designer may initially choose to align the sequences of each Class I locus separately and use AA3D algorithm to select a minimal number of alleles. These HLA-A, HLA-B and HLA-C lists can then be combined and subjected to AA3D algorithm again to remove unnecessary alleles with variant positions that have been covered by another allele in a different locus. Alternatively, the designer may apply AA3D algorithm on the combined HLA-A, HLA-B and HLA-C allele list from the start to select a minimal number of alleles with maximal coverage. If a variant position across multiple loci turns out to be a constant position within a locus, then that position is considered as “self” with the only exception concerning DRB3/DRB4/DRB5 loci, for which individuals may or may not carry such allele(s). The algorithm described herein allows users to start with different sets of allele sequences from targeted populations and to preview the lists of selected alleles before finalizing a list comprising a minimal number of HLA that provide the highest degree of ep coverage (variant and/or pattern).

AA3D panel design algorithm follows the stepwise inclusion and exclusion rationale described below to reach a minimal number of alleles that cover all variant position residues of the initial allele list and identify unique patterns in a 3D space, such as within about 15-0.5 Å (e.g., about 15, 14, 13, 12, 11, 10, 9, 8, 7, 6, 5.5, 5, 4.5, 4, 3.5, 3, 2.5, 2, 1.5, 1, or 0.5 Å), that could be in contact with Ab, such as when measured in the context of a folded HLA protein or when measured in the context of an assembled HLA heterodimer

Align the sequences of the extracellular domains of the initial allele list, excluding signal peptide, transmembrane, and cytoplasmic domains that are not available for Ab recognition on intact cells. Although an Ab targeting non-extracellular domain of an HLA molecule is theoretically possible, it is generally accepted that such an Ab is not clinically significant in initiating an immunological attack on healthy cells.

Instead of restricting to only “solvent exposed” residues which are predicted based on limited experimental structure information, the SAB panel of the present disclosure is designed to capture all variant positions, not limited to only “solvent exposed” residues. For example, buried residues lining the peptide-binding pocket could exert their impact on epitope indirectly through peptide presentation. In addition, these Ab-inaccessible residues themselves could contribute to the conformational change of a nearby solvent accessible residue(s) through an allosteric effect. Therefore, a SAB panel according to the present disclosure provides a way to detect clinically significant variants which might have been routinely overlooked.

Select alleles that possess unique ep(s) and/or ep pattern(s) not represented by other alleles on the list.

For ep or ep pattern that is carried by more than one allele, select the most representative allele based on population frequency if available such as CIWD 3.0, CWD 2.0, Allele Frequency Net Database (AFND), and International Histocompatibility Workshop Group (IHWG). If population frequency is unavailable, the designer can artificially prioritize the selection order.

For HLA-DQ and HLA-DP with substantial variations in both alpha and beta subunits, it is conceivable that variant positions, close to the alpha-beta pairing junctions, could create specific patterns only present in certain alpha-beta pairs but not in others. Methods disclosed herein, such as the AA3D panel design algorithm, can capture this type of patterns and recommends which alpha-beta pairs to include in the panel design.

In addition to AA3D-suggested pairing, pair alpha and beta subunits for HLA-DQ and HLA-DP molecules preferably including all representative alpha subunits with at least one selected beta subunit of the same 1^{st} field typing. The decision is hinged upon successful recombinant expression of such a pairing.

Produce a HLA SAB Panel and perform Ab specificity characterization.

Map potential ER recognized by available mAb, such as by using the AA3D imputation algorithm. In one example mapping can be accomplished by adding up all eps and ep 3D patterns present in each positive-binding Ag and subtracting all eps and ep 3D patterns present in the negative-binding Ag. For Class I alleles, HLA-A, HLA-B and HLA-C loci can be combined for analysis because their sequences align well and share a similar structural framework. For HLA-DQ and HLA-DP Ag, their alpha and beta loci can be combined for analysis especially if the potential ER are located close to the alpha-beta junctions. For HLA-DR Ag, including DRB1, DRB3, DRB4 and DRB5, they can be combined for analysis. Alternatively, the user can opt to do the analysis locus by locus with the understanding that certain candidate ER may not be detected or resolved. Eps and ep 3D patterns identified through this imputation process are likely involved in forming the ER recognized by a specific mAb.

Use EV of the implicated ep(s) and ep pattern(s) to confirm which ep(s) or ep pattern(s) is indeed the ER of a specific mAb based on LOF observations. The binding comparison between EV and its WT Ag can be performed on cells expressing the Ag on cell surface, or the EV and WT Ag can be compared after purification and immobilization on a solid surface such as the Luminex beads with multiplex capability. By definition, the interaction between an ER and its cognate Ab is highly specific and essential, a replacement with another amino acid residue would lead to complete LOF. Although a potential EV derived from a binding Ag is commonly designed to harbor an ep(s) or ep pattern(s) present in a non-binding Ag, other substitutions especially of a residue(s) with drastically different properties from the WT could lead to complete LOF if indeed the ER residue(s) is altered. If only partial LOF is observed, the WT ep or ep pattern is considered as AR.

A test is performed with HLA-sensitized human serum samples of a pAb nature. The ER recognized by such a complex sample may or may not be identified depending on the mAb constituents of the pAb sample. Conceptually, if one of the mAb in the serum sample recognizes a WT Ag but not its derived EV with an altered ER and there is no additional mAb that recognizes another epitope not affected by the ER change on the same WT and EV, the complete LOF observed can be interpreted that there is at least one mAb in the serum sample recognizing this epitope through the ER. On the other hand, if such a complete LOF is not observed, no conclusion can be drawn because the LOF for one mAb can be masked by another mAb recognizing a different epitope on the same allele not affected by the change in the EV.

For a pAb containing serum sample, after first round of testing with the innovative epitope panel(s) described here including both WT and EV Ag, a round of Ads-Elu method can be performed to deconvolute reactivity patterns based on key binding Ag carrying different epitopes from one another.

Optionally, perform on or more additional rounds of panel testing using eluted material from a specific Ag, complete or partial LOF may be observed to identify ER or AR.

With each round of data analysis, review the sequences of alleles on the panel list to identify the limitations that cannot be resolved. Addition of EV to the panel is expected to improve resolution of predicted but unverified ER. In some situations, the ambiguity may be deemed acceptable because further resolution does not appear to provide additional clinical advantage.

Because an ep or pattern replacement at a known variant position is generally well tolerated by the HLA molecule, EV is expected to express well and does not alter binding pattern by other mAb that recognize different ER on the same Ag so long as the respective AR are not significantly impacted either.

If an EV does not express well and/or changes its binding specificity to a known mAb whose ER and AR should not be affected by such a substitution, this EV can be excluded from the panel. In some cases, an EV may exist in nature but was not included in the initial list due to missing a priority inclusion criterium such as population frequency. In other cases, there has never been a reported allele or Ag of the same EV sequence, but this does not preclude the presence of such an allele or Ag in nature.

Continue to refine the Ag panel to address the remaining or future needs by starting with a different initial list to recreate a new design from scratch or targeting a specific deficiency such as adding additional EV to resolve the concurrent variant positions on the panel. Each round of optimization should go through the panel design algorithm described herein. By the same token, existing Ag can be removed from the panel if no measurable values are added after extensive testing or in-field surveillance.

Despite the continued growth of new HLA alleles and data from population genetics, the alleles defined by 2-field typing at amino acid level more-or-less are stabilized especially from developed countries with established transplant communities. Therefore, certain alleles and EV are likely to remain on the panel(s) regardless of what new alleles or frequency info are updated in the future. On the other hand, certain representative alleles of significant frequencies still emerging from developing countries are expected to be targeted for future panel enhancements.

This HLA panel design methodology addresses current and future needs in the field of transplant diagnostics. It is known that the designations of common, intermediate, and well documented alleles vary among different ethnic, ancestral, and/or geographic populations. Because of the sheer number of alleles identified in any given population, it is economically impractical or prohibitive to cover all variants, and therefore a strategy for maximizing such coverage while minimizing the number of HLA alleles is needed. This allows the identification of the ep(s) and ep cluster(s) associated with a binding pattern crucial for collecting scientific and clinical observations on a sound economic basis. Although this method can be applied to any initial list of alleles, the examples described here are based primarily on alleles designated as common in any one of the populations categorized by CIWD, so no population is excluded from or prioritized over another population: AFA (African/African American), API (Asian/Pacific Islands), EURO (European/European descent), MENA (Middle East/North Coast of Africa), HIS (South or Central America/Hispanic/Latino), NAM (Native American) and UNK (unknown/not asked/multiple ancestries/other). However, because allele frequency is a factor when selecting the most representative allele if multiple alleles carrying the same ep or ep pattern are eligible, populations with little or insufficient HLA typing information are disadvantaged, and therefore the SAB panel list should be periodically reviewed to reflect the current global HLA typing collection.

In addition to CIWD 3.0., in various aspects, other sources such as CWD 2.0, Allele Frequency Network Database (AFND), Histocompatibility Workshop Group (IHWG) collections at Fred Hutchinson Cancer Center and typing collections may be used in addition to CIWD 3.0., or alternatively to CIWD 3.0., to compile an initial list of alleles.

All individuals with a normal diploid set of chromosomes carry two HLA alleles of each Class I (A, B and C) and Class II (DRB1, DQA1, DQB1, DPA1 and DPB1) locus, in a homozygous or heterozygous state. Some individuals also carry one or two alleles of either DRB3, DRB4, DRB5, or in combination. The search for match (self) or mismatch (non-self) epitopes intuitively may be confined within the same locus because conserved residues within each locus are considered “self” even if they are different from another locus. However, since the conserved residues in each locus are represented by all alleles of the same locus, inclusion of one allele because of a “cross-locus variant residue” would cover all conserved residues within the same locus.

Therefore, it is intended for the methods disclosed herein to apply directly to all alleles of all loci from a targeted population(s). The latter approach is not practiced in the examples described below; however, the same method could be applied on an initial allele list consisting of more than one locus or all 11 loci. As to HLA Class I alleles, because they share a similar structural framework, it is deemed feasible to list all Class I alleles from a target population(s) to be considered together. Alternatively, even for HLA Class I alleles, the minimal number of alleles can be first selected for each locus as described herein and then combine all 3 lists together for another round of selection using the same logic described herein to further minimize the number of alleles needed for the same epitope coverage and ER prediction.

Because of the highly polymorphic nature of HLA alleles, it is impractical to cover all eps and ep patterns of known alleles in a single diagnostic HLA panel. Fortunately, there are relatively well characterized common alleles frequently encountered in patient and donor pools to prioritize over other less common alleles. In cases where a particular rare allele is not included in an HLA panel, but if the variant(s) is at a position where other eps and ep patterns have been identified as Ab-verified ER, then the risk of that mismatched variant can be acknowledged even without verification by a binding Ab and LOF EV. A goal of this disclosure is to generate panels capable of identifying as many ER as encountered from clinical samples to facilitate in silico prediction with confidence even in the absence of virtual crossmatching based on SAB assay.

Once a HLA panel is generated, it may be tested with available mAbs and the data analyzed by the companion AA3D module within HLA Fusion™ software to impute which ep(s) or ep pattern(s) is likely the ER. The number of available HLA specific human mAb is limited. However, many mouse mAb specific to HLA have been developed through hybridoma technology and their mapped epitopes are included in the HLA Eplet Registry (Duquesnoy et al., 16th IHIW: a website for antibody-defined HLA epitope Registry. Int J Immunogenet. 2013; 40: 54-9.) and corroborated with HLAMatchmaker™, a molecularly based algorithm for histocompatibility determination. Although mouse and human immune systems differ in perceiving self versus non-self, increased sensitivity of mouse immune system due to immunity boosting immunization strategy, and differences between mouse MHC and human HLA repertoires, mouse mAb against human HLA are suited for testing the utility of the SAB panel for ER mapping disclosed herein.

The HLA panel of the present disclosure is expected to capture more diverse binding signals due to the enhanced epitope coverage and, at the same time, more negative signals for use to determine clinical relevancy and increase the robustness and efficacy of determining clinical outcomes (e.g., prognostics and diagnostics). For each potential ER required for positive binding, there is at least one antigen that does not have the same ep(s). In other words, an HLA panel of the present disclosure provides better resolution in identifying ER. In addition, complete LOF of an EV with an altered ER provides confirmation that such an ER is recognized by a least one mAb in the pAb mix. On the other hand, an EV that results in GOF having an altered ep(s) also points out the critical nature of such a position(s).

It is known that serum samples from most transplant patients or multiparous females contain pAb of complex nature. As a result, positive signals from initial assaying may not provide sufficient resolution and the relevant EV may be missing from the panel and the LOF signals may be masked by another binder. To circumvent this, a subset of Ab is first isolated by adsorbing HLA Ag to cells or a solid phase, and then eluting for subsequent panel testing. It often takes more than one cycle of Ads-Elu to sufficiently deconvolute the signals and identify the likely ER by Ads-Elu protocols that are routinely practiced by researchers in the field.

Although a purpose of introducing EV with an altered ep(s) is to resolve the concurrency of multiple eps and/or ep patterns, implicated in a positive binding pattern by achieving a LOF effect, EV in some instances achieve a GOF effect to another mAb if sufficient affinity is provided by the surrounding AR. Just like an ER can be identified with a binding WT paired with its LOF EV derivative, an ER can also be implicated with a non-binding WT paired with its GOF EV derivative.

To illustrate the compositions and methodology of the disclosure, HLA Class II DQ panels are described in the examples herein. By extension, the same or similar methodology and algorithm(s) can be applied to generating an HLA-DP panel or other HLA and/or combination thereof. For HLA-DR antigens, the alpha subunit is considered practically invariable because there are very few variants identified. The general practice is to use DRA1*01:01 as the default alpha subunit for generating HLA-DR SA. Similarly, no alpha-beta pairing needs to be considered for HLA Class I (A, B and C) panel(s) where all alpha subunits are paired with the same invariable beta 2 microglobulin.

As used herein, “essential residue/region (ER)” means the critical contact point(s) with a CDR or other region of Ab variable domain which typically spans a radius of >3.5 Å. As such, the ER does not necessarily have to contact CDR-H3. More than one ER could be present for binding a mAb. For each epitope, there are always multiple ancillary residue/region (AR) referring to Ab-contacting residues where individual changes at those positions only affect affinity but not leading to complete LOF. Although individual AR changes only have partial or little observable effects, a combination of multiple AR changes could lead to complete LOF despite an intact ER.

As used herein, a “panel” means a collection of one or more EV, antigens, and/or compositions of the disclosure (e.g., a substrate having an immobilized HLA, EV or otherwise) which may be divided into specific categories or sub-panels (e.g., specific HLA classes, sub-classes (i.e., HLA-A, HLA-B, HLA-C, HLA-DQ and the like), or otherwise) and which may be assayed simultaneously or separately, optionally at different times.

As used herein, an “altered ER” means an ER from a WT antigen that has been altered by changing or deleting one or more amino acid residues of the WT antigen that constitutes the ER. In some aspects, the altered ER includes a substitution of an amino acid as compared to the WT ER. In some aspects, an altered ER includes one or more amino acid changes (e.g., substitution and/or deletion) of amino acids that occupy a space having a radius of less than about 3.5-6 Å or about 3-3.5 Å that form the WT ER. In some aspects, altering the WT ER to an altered ER changes antibody binding specificity and/or binding affinity conferring an activity ranging from complete loss of binding/loss of function (LOF) for one mAb to gain of function (GOF) to another mAb or pAB.

As used herein, “conservative amino acid substitution(s)” are the substitution of an amino acid for an amino acid with chemically similar properties. The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).
   An example of a conservative amino acid substitution would be a substitution of an Alanine for a Threonine residue. In some aspects, an EV includes 1, 2, 3, 4, 5, 6, 7, 8, 9, 10, or more conservative substitutions, compared to a reference sequence, such as a WT HLA amino acid sequence.

“Non-conservative amino acid substitution(s)” are substitution of amino acids for those with chemically dissimilar properties, for example those outside the list above, for example a substitution of an Alanine for a Leucine residue. An EV can include up to 1, 2, 3, 4, 5, 6, 7, 8, 9, 10, or more conservative substitutions compared to a reference sequence, such as a WT HLA sequence. In some aspects, an EV includes 1, 2, 3, 4, 5, 6, 7, 8, 9, 10, or more non-conservative substitutions, compared to a reference sequence, such as a WT HLA amino acid sequence.

As used herein the “extracellular domain” is the domain of a membrane protein that extends into the extracellular space. The extracellular domain can be identified by standard methods such as by excluding the transmembrane domains and/or cytosolic domains proteins disclosed herein. The transmembrane domain of a protein can be identified using standard methods, such as though the use of hydrophobicity scale tools such as DeepTMHHM™ (services.healthtech.dtu.dk/services/DeepTMHMM-1.0/). In some aspects, the extracellular domain includes only those amino acid residues of the protein that extend from a cellular membrane of a cell as is known by one of skill in the art with regard to transmembrane proteins.

Class I and II HLA alleles are disclosed herein, for example DQA1*01:03. Reference sequences for the HLA alleles disclosed herein can be obtained by standard methods, for example by querying an HLA allele database such as IMGT database (ebi.ac.uk/ipd/imgt/hla/alleles/).

In some aspects, compositions including a substrate having an immobilized antigen including an EV or extracellular domain thereof are provided. As used herein, a “substrate” is a solid support or surface. The configuration of the solid support can be flat (e.g., a plate or slide), spherical (e.g., a bead), or another configuration. Suitable substrate materials include, but are not limited to organic polymers such as polypropylene, polyethylene, polybutylene, polyisobutylene, polybutadiene, polyisoprene, polyvinylpyrrolidine, polytetrafluoroethylene, polyvinylidene difluoride, polyfluoroethylene-propylene, polyethylenevinyl alcohol, polymethylpentene, polycholorotrifluoroethylene, polysulfones, hydroxylated biaxially oriented polypropylene, aminated biaxially oriented polypropylene, thiolated biaxially oriented polypropylene, ethyleneacrylic acid, thylene methacrylic acid, and blends of copolymers thereof. In general, the material used for the substrate is amenable to surface activation such that upon activation, the surface of the substrate is capable of covalently attaching a biomolecule, such as an EV or extracellular domain thereof. Suitable methods for covalently coupling proteins to a substrate or solid support are known to those working in the field.

In particular examples, the substrate is a bead, such as a magnetic bead or a biotinylated bead. In some examples, an EV of an HLA or extracellular domain thereof is immobilized on (for example, linked or conjugated to) a Luminex® bead (Thermo Fisher Scientific®). In some examples, the beads are about 1 μm diameter, about 2.8 μm diameter, or about 4.5 μm diameter.

In certain aspects, the disclosure provides a computing device for performing a method of the disclosure. A computing device may include one or more processors in operable connection to one or more non-transitory computer readable storage media storing instructions executable by the one or more processors which when executed, cause the computing device to perform one or more processes of the methods described herein.

The computing device of the disclosure is illustrated as having a number of processors and storage media, but any one or more of these components may be omitted or duplicated, as suitable for the application and setting. In some aspects, some or all of the components included in the computing device can be attached to one or more motherboards and enclosed in a housing (e.g., including plastic, metal, and/or other materials). In some aspects, some these components may be fabricated onto a single system-on-a-chip (SoC) (e.g., an SoC may include one or more processing devices). Additionally, in various aspects, the computing device may include interface circuitry for coupling to the one or more components using any suitable interface (e.g., a Universal Serial Bus (USB) interface, a High-Definition Multimedia Interface (HDMI) interface, a Controller Area Network (CAN) interface, a Serial Peripheral Interface (SPI) interface, an Ethernet interface, a wireless interface, or any other appropriate interface). For example, the computing device may not include a display device, but may include display device interface circuitry (e.g., a connector and driver circuitry) to which a display device may be coupled.

The computing device can include a processing medium or device (e.g., one or more processing devices). As used herein, the term “processing device” refers to any device or portion of a device that processes electronic data from registers and/or memory to transform that electronic data into other electronic data that may be stored in registers and/or memory. The processing device can include one or more digital signal processors (DSPs), application-specific integrated circuits (ASICs), central computing devices (CPUs), graphics computing devices (GPUs), crypto processors (specialized processors that execute cryptographic algorithms within hardware), server processors, or any other suitable processing devices.

The computing device can also include a storage device (e.g., one or more storage devices). The storage device can include one or more memory devices such as random-access memory (RAM) (e.g., static RAM (SRAM) devices, magnetic RAM (MRAM) devices, dynamic RAM (DRAM) devices, resistive RAM (RRAM) devices, or conductive-bridging RAM (CBRAM) devices), hard drive-based memory devices, solid-state memory devices, networked drives, cloud drives, or any combination of memory devices. In some aspects, the storage device can include memory that shares a die with a processing device. In such an aspect, the memory can be used as cache memory and can include embedded dynamic random-access memory (eDRAM) or spin transfer torque magnetic random-access memory (STT-MRAM), for example. In some aspects, the storage device can include non-transitory computer-readable media having instructions thereon that, when executed by one or more processing devices (e.g., the processing device), cause the computing device to perform any appropriate one of or portions of the methods and operations disclosed herein.

The computing device can include an interface device (e.g., one or more interface devices). The interface device can include one or more communication chips, connectors, and/or other hardware and software to govern communications between the computing device and other computing devices. For example, the interface device can include circuitry for managing wireless communications for the transfer of data to and from the computing device. The term “wireless” and its derivatives are used to describe circuits, devices, systems, methods, techniques, communications channels, etc., that can communicate data using modulated electromagnetic radiation through a nonsolid medium. The term does not imply that the associated devices do not contain any wires, although in some aspects they might not. Circuitry included in the interface device for managing wireless communications can implement any of a number of wireless standards or protocols, including but not limited to Institute for Electrical and Electronic Engineers (IEEE) standards including Wi-Fi (IEEE 802.11 family), IEEE 802.16 standards (e.g., IEEE 802.16-2005 Amendment), Long-Term Evolution (LTE) project along with any amendments, updates, and/or revisions (e.g., advanced LTE project, ultra-mobile broadband (UMB) project (also referred to as “3GPP2”), etc.). In some aspects, circuitry included in the interface device for managing wireless communications may operate in accordance with a Global System for Mobile Communication (GSM), General Packet Radio Service (GPRS), Universal Mobile Telecommunications System (UMTS), High-Speed Packet Access (HSPA), Evolved HSPA (E-HSPA), or LTE network. In some aspects, circuitry included in the interface device for managing wireless communications can operate in accordance with Enhanced Data for GSM Evolution (EDGE), GSM EDGE Radio Access Network (GERAN), Universal Terrestrial Radio Access Network (UTRAN), or Evolved UTRAN (E-UTRAN). In some aspects, circuitry included in the interface device for managing wireless communications can operate in accordance with Code Division Multiple Access (CDMA), Time Division Multiple Access (TDMA), Digital Enhanced Cordless Telecommunications (DECT), Evolution-Data Optimized (EV-DO), and derivatives thereof, as well as any other wireless protocols that are designated as 3G, 4G, 5G, and beyond. In some aspects, the interface device can include one or more antennas (e.g., one or more antenna arrays) to receipt and/or transmission of wireless communications.

In some aspects, the interface device (e.g., computing device) includes one or more processors and/or memories, as well as circuitry for managing input/output communication via wired/wireless communications, such as electrical, optical, Wi-Fi, or any other suitable communication protocols. For example, the interface device can include circuitry to support communications in accordance with Ethernet technologies. In some aspects, the interface device can support both wireless and wired communication and/or may support multiple wired communication protocols and/or multiple wireless communication protocols. For example, a first set of circuitries of the interface device can be dedicated to shorter-range wireless communications such as Wi-Fi or Bluetooth, and a second set of circuitry of the interface device can be dedicated to longer-range wireless communications such as global positioning system (GPS), EDGE, GPRS, CDMA, WiMAX, LTE, EV-DO, or others. In some aspects, a first set of circuitries of the interface device can be dedicated to wireless communications, and a second set of circuitries of the interface device can be dedicated to wired communications.

The computing device can include battery/power circuitry. The battery/power circuitry can include one or more energy storage devices (e.g., batteries or capacitors) and/or circuitry for coupling components of the computing device to an energy source separate from the computing device (e.g., AC line power).

The computing device can include a display device (e.g., multiple display devices). The display device can include any visual indicators, such as a heads-up display, a computer monitor, a projector, a touchscreen display, a liquid crystal display (LCD), a light-emitting diode display, or a flat panel display.

The computing device can include other input/output (I/O) devices. The other I/O devices can include one or more audio output devices (e.g., speakers, headsets, earbuds, alarms, etc.), one or more audio input devices (e.g., microphones or microphone arrays), location devices (e.g., GPS devices in communication with a satellite-based system to receive a location of the computing device, as known in the art), audio codecs, video codecs, printers, sensors (e.g., thermocouples or other temperature sensors, humidity sensors, pressure sensors, vibration sensors, accelerometers, gyroscopes, etc.), image capture devices such as cameras, keyboards, cursor control devices such as a mouse, a stylus, a trackball, or a touchpad, bar code readers, Quick Response (QR) code readers, or radio frequency identification (RFID) readers, for example.

The computing device can have any suitable form factor for its application and setting, such as a handheld or mobile computing device (e.g., a cell phone, a smartphone, a mobile internet device, a tablet computer, a laptop computer, a netbook computer, an Ultrabook computer, a personal digital assistant (PDA), an ultra-mobile personal computer, etc.), a desktop computing device, or a server computing device or other networked computing components.

The following examples are provided to illustrate particular features of certain aspects of the disclosure, but the scope of the claims should not be limited to those features exemplified.

### EXAMPLES

### Example 1: Design of HLA-DQ Epitope Panel

A stepwise inclusion and exclusion considerations are illustrated below to select HLA-DQB1 alleles, HLA-DQA1 alleles, and their pairing in designing an exemplary SAB panel for mapping ER of HLA-DQ Ag is further illustrated.

HLA-DQB1 allele summary (HLA-DQB1_PrimaryData-IHWS-20200320.xlsx) was downloaded as part of CIWD 3.0.0 data from the 18th International HLA & Immunogenetics Workshop website (ihiw18.org/). All Common DQB1 alleles were included for panel consideration.

Because the total number of DQA1 alleles at 2 field is quite manageable, both Common and Well-Documented alleles based on CWD 2.0.0 catalog (Mack et al., Common and well-documented HLA alleles: 2012 update to the CWD catalogue. Tissue Antigens. 2013; 81: 194-203.) were included.

The extracellular domain amino acid sequences of the alleles selected were aligned to identify the variant residues at each variant position. The amino acid sequences were extracted from IPD-IMGT/HLA database (ebi.ac.uk/ipd/imgt/hla/alleles/).

Consensus residue at each position were considered “self” that is unlikely to initiate an immunogenic response unless it is part of a variant pattern consisting of multiple amino acids within 3.5 Å of each other.

The AA3D module was used for panel design that considers all unique and representative eps or ep patterns within a certain 3D distance that could form contacts with a mAb. Because the 3D coordinates of each residue are based on molecular modeling of a limited number of Ag, several distance units are considered to compensate for the potential margin of error. The longer the distance, the more permutations of unique patterns are encountered. For practical reasons, the distance was set to achieve a manageable list of Ag.

10 DQA1 and 19 DQB1 alleles were selected as having eps and/or ep patterns confined within each subunit, that likely form an ER. In addition, 38 DQA1-DQB1 pairs were identified for carrying unique or representative eps or ep patterns having both DQA1 and DQB1 subunits. For this experiment, the algorithm did not differentiate the pairing preference that exists in nature, as such, some of the pairs were excluded based on common knowledge and experimental observations. Where alpha and beta alleles are disclosed in pairs in the following tables, they are not separately listed individually. However, it should be understood that individual alpha and beta alleles from the pair were present in the panel. 7 DQA alleles, 12 DQB alleles, and 26 pairs were selected for panel considerations (Table 1 Å). All selected alleles are listed as DQB and DQA alleles without pairing (Table 1B).

TABLE 1A
The selected 45 minimal number of HLA-DQ alleles and pairs covering all unique or
representative ep(s) and ep patterns that could form an ER in contact with an Ab.

| Count | DQA | Count | DQB | Count | DQA/DQB Pair |
|---|---|---|---|---|---|
| 1 | DQA1*01:03 | 1 | DQB1*02:07 | 1 | DQA1*01:01/DQB1*05:01 |
| 2 | DQA1*01:04 | 2 | DQB1*03:05 | 2 | DQA1*01:01/DQB1*05:02 |
| 3 | DQA1*03:02 | 3 | DQB1*03:09 | 3 | DQA1*01:01/DQB1*05:23 |
| 4 | DQA1*05:02 | 4 | DQB1*03:11 | 4 | DQA1*01:01/DQB1*06:01 |
| 5 | DQA1*05:03 | 5 | DQB1*03:13 | 5 | DQA1*01:01/DQB1*06:02 |
| 6 | DQA1*05:08 | 6 | DQB1*03:16 | 6 | DQA1*01:01/DQB1*06:04 |
| 7 | DQA1*05:09 | 7 | DQB1*03:25 | 7 | DQA1*01:01/DQB1*06:49 |
| | | 8 | DQB1*03:42 | 8 | DQA1*01:02/DQB1*05:01 |
| | | 9 | DQB1*04:01 | 9 | DQA1*02:01/DQB1*02:01 |
| | | 10 | DQB1*05:04 | 10 | DQA1*02:01/DQB1*02:180 |
| | | 11 | DQB1*05:10 | 11 | DQA1*02:01/DQB1*03:01 |
| | | 12 | DQB1*06:10 | 12 | DQA1*02:01/DQB1*03:02 |
| | | | | 13 | DQA1*02:01/DQB1*06:01 |
| | | | | 14 | DQA1*02:01/DQB1*06:02 |
| | | | | 15 | DQA1*02:01/DQB1*06:04 |
| | | | | 16 | DQA1*02:01/DQB1*06:49 |
| | | | | 17 | DQA1*03:01/DQB1*02:01 |
| | | | | 18 | DQA1*03:01/DQB1*02:180 |
| | | | | 19 | DQA1*03:01/DQB1*03:01 |
| | | | | 20 | DQA1*03:01/DQB1*03:02 |
| | | | | 21 | DQA1*03:01/DQB1*06:01 |
| | | | | 22 | DQA1*03:01/DQB1*06:02 |
| | | | | 23 | DQA1*04:01/DQB1*02:01 |
| | | | | 24 | DQA1*04:01/DQB1*06:01 |
| | | | | 25 | DQA1*04:01/DQB1*06:02 |
| | | | | 26 | DQA1*04:01/DQB1*06:04 |

TABLE 1B
The selected 35 minimal number of HLA-DQ alleles covering
all unique or representative ep(s) and ep patterns
that could form an ER in contact with an Ab.

| Count | DQA | Count | DQB |
|---|---|---|---|
| 1 | DQA1*01:01 | 1 | DQB1*02:01 |
| 2 | DQA1*01:02 | 2 | DQB1*02:07 |
| 3 | DQA1*01:03 | 3 | DQB1*02:180 |
| 4 | DQA1*01:04 | 4 | DQB1*03:01 |
| 5 | DQA1*02:01 | 5 | DQB1*03:02 |
| 6 | DQA1*03:01 | 6 | DQB1*03:05 |
| 7 | DQA1*03:02 | 7 | DQB1*03:09 |
| 8 | DQA1*04:01 | 8 | DQB1*03:11 |
| 9 | DQA1*05:02 | 9 | DQB1*03:13 |
| 10 | DQA1*05:03 | 10 | DQB1*03:16 |
| 11 | DQA1*05:08 | 11 | DQB1*03:25 |
| 12 | DQA1*05:09 | 12 | DQB1*03:42 |
| | | 13 | DQB1*04:01 |
| | | 14 | DQB1*05:01 |
| | | 15 | DQB1*05:02 |
| | | 16 | DQB1*05:04 |
| | | 17 | DQB1*05:10 |
| | | 18 | DQB1*05:23 |
| | | 19 | DQB1*06:01 |
| | | 20 | DQB1*06:02 |
| | | 21 | DQB1*06:04 |
| | | 22 | DQB1*06:10 |
| | | 23 | DQB1*06:49 |

The AA3D module used for this example did not consider the potential conformational changes of the alpha subunits induced by pairing with different beta subunits, and vice versa. In some examples, epitope differences due to alpha-beta pairing could be detected, in order to broaden the pairing coverage.

### a Systematic Pairing Evaluation.

Step 1. A matrix was created encompassing all HLA-DQB1×HLA-DQA1 combinations based on the lists of HLA-DQB1 and HLA-DQA1 alleles selected above.

Step 2. It was anticipated that any one of the subunits needs to be presented only once on an Ag panel to minimize the number of Ag; however, when alpha and beta subunits come together to form a 3D structure, additional ER may be formed at the alpha-beta junctions and the conformation of one subunit even of the same amino acid sequence may be modified allosterically depending on what the other pairing subunit is. For that reason, special considerations below were needed to decide which alpha-beta pairs to include.

Step 3. Proved that the reliability of alpha-beta haplotype association collected in AFND depends on the quality of typing results from all over the world and whether such an association leads to a productive Ag expression. Therefore, a more comprehensive approach was taken to experimentally determine the success of pairing through recombinant expression technology. Poor or no expression points to incompatible pairing that is unlikely to exist in any population, and therefore was excluded from the panel.

Step 4. A representative subset of combinations was compiled where for each DQB1 serological typing group, representative DQA1 alleles covering at least one DQA1*01, DQA1*02, DQA1*03, DQA1*04, DQA1*05 and DQA1*06 allele were tested for expression.

Step 5. The list of successful pairing was further examined for DQ Ag selection for the panel. HLA-DQA and DQB alleles listed in Table 1 Å through prior variant selection were given priority so that they are represented at least once during the pairing selection. In addition, for each DQB1*02 (DQB1*02:01 or DQB1*02:02), DQB1*03 (DQB1*03:01, DQB1*03:02 or DQB1*03:03), DQB1*04 (DQB1*04:01 or DQB1*04:02), DQB1*05 (DQB1*05:01, DQB1*05:02 or DQB1*05:03) and DQB1*06 (DQB1*06:01, DQB1*06:02 or DQB1*06:03) alleles of highest population frequencies, pairing with multiple DQA1* alleles were selected to provide further investigation of whether the conformations of beta subunit were modified by its pairing alpha subunit, and if a new ep pattern is formed at the alpha-beta junction.

Step 6. Recombinant expression of various alpha-beta pairing was conducted to confirm successful cell surface expression. No or low detectable expression indicates incompatible or undesirable pairing that were excluded from the panel design.

A DQ panel listed in Table 1C was designed and produced for feasibility studies. Although this panel design is of a narrower scope from what is listed in Table 1B, it captures most of the design methodology, and was therefore suitable for serving as proof-of-concept. 36 Ag were selected. Nine DQ Ag are already included on One Lambda LABScreen™ CII SAB panels (in bold) and 4 Ag only in Werfen LSA™ NEXA2 SAB panel (underlined).

TABLE 1C
List of DQ antigens selected for feasibility
epitope panel production.

| Count | DQA | DQB |
|---|---|---|
| 1 | DQA1*02:01 | DQB1*02:01 |
| 2 | DQA1*03:01 | DQB1*02:01 |
| 3 | DQA1*04:01 | DQB1*02:01 |
| 4 | DQA1*05:01 | DQB1*02:01 |
| 5 | DQA1*02:01 | DQB1*02:07 |
| 6 | DQA1*03:03 | DQB1*02:180 |
| 7 | DQA1*05:01 | DQB1*02:180 |
| 8 | DQA1*02:01 | DQB1*03:02 |
| 9 | DQA1*03:01 | DQB1*03:02 |
| 10 | DQA1*03:02 | DQB1*03:02 |
| 11 | DQA1*04:01 | DQB1*03:02 |
| 12 | DQA1*05:02 | DQB1*03:02 |
| 13 | DQA1*05:03 | DQB1*03:02 |
| 14 | DQA1*05:09 | DQB1*03:02 |
| 15 | DQA1*02:01 | DQB1*03:09 |
| 16 | DQA1*05:05 | DQB1*03:13 |
| 17 | DQA1*02:01 | DQB1*03:16 |
| 18 | DQA1*03:02 | DQB1*03:42 |
| 19 | DQA1*02:01 | DQB1*04:01 |
| 20 | DQA1*03:01 | DQB1*04:01 |
| 21 | DQA1*03:02 | DQB1*04:01 |
| 22 | DQA1*04:01 | DQB1*04:01 |
| 23 | DQA1*01:01 | DQB1*05:01 |
| 24 | DQA1*01:03 | DQB1*05:02 |
| 25 | DQA1*01:04 | DQB1*05:02 |
| 26 | DQA1*01:02 | DQB1*05:10 |
| 27 | DQA1*01:01 | DQB1*05:23 |
| 28 | DQA1*01:03 | DQB1*06:01 |
| 29 | DQA1*01:04 | DQB1*06:01 |
| 30 | DQA1*02:01 | DQB1*06:01 |
| 31 | DQA1*03:01 | DQB1*06:01 |
| 32 | DQA1*04:01 | DQB1*06:01 |
| 33 | DQA1*05:03 | DQB1*06:01 |
| 34 | DQA1*01:03 | DQB1*06:04 |
| 35 | DQA1*01:03 | DQB1*06:110 |
| 36 | DQA1*01:02 | DQB1*06:49 |

### Example 2: Production of HLA-DQ Epitope Panel

This example illustrates expression and purification of HLA-DQ antigens, followed by single antigen bead production.

### Expression and Purification of HLA-DQ Antigens

Step 1. A synthetic DNA fragment including the coding sequence of the alpha or beta subunit was cloned into a bicistronic HLA Ag expression vector. If the complete amino acid sequence of a selected allele is not available, the remaining sequence was substituted with the sequence of its closest homolog to make a full-length construct.

Step 2. Mammalian expression vectors were used to generate expression cells. Many mammalian expression vectors including bicistronic, separate expression cassettes on the same vector, and co-transfection of 2 separate vectors each carrying an alpha or beta subunit can be used with the methods described herein to generate expression cells. Epitopes presented by the HLA extracellular domain are of important clinical relevance so expression constructs of only the extracellular domain (soluble HLA) of the Ag were used to detect DSA. However, it is appreciated that full-length HLA expression constructs may be used to express full-length membrane-bound HLA that closely mimic WT HLA structures and conformations.

Step 3. A plasmid was then transfected into a human host cell line without endogenous HLA Class II expression. Cell surface transient expression of the DQ Ag was detected 72 hours post transfection before initiating antibiotic selection to generate a stable expression pool.

Step 4. Once the expression of a stable pool was confirmed by flow cytometry, the top expressing cells were single cell sorted into a 96 well cell culture plate and/or bulk sorted into a 6 well plate. The surviving single cell lines were ranked for Ag expression and growth characteristics. The top ranked clones were banked for future use while the best performing one was expanded for Ag production.

Step 5. Ag expressing cells were expanded, harvested, and frozen at <−65° C. for long-term storage. Only cell pellets that have passed quality control to ensure identity and quality were used for Ag purification.

Step 6. Ag were released from lysed cells and cleared of debris before loading onto an affinity purification column. The captured Ag was washed and then eluted from the column before buffer exchange and concentration to the final storage solution and being frozen at <−65° C.

Step 7. The specificity and signal strength of each purified Ag was determined and confirmed by assay with a panel of reference Ab.

### LABScreen™ Single Antigen Bead (SAB) Production

Thirty-two (32) out of the 36 HLA DQ Ags listed in Table 1C were included for SAB panel production following LABScreen™ (One Lambda, Thermo Fisher Scientific) production protocol. Descriptions are provided in the following.

Step 1. The coating amount of each Ag to beads was predetermined at the small-scale Ag quality check described above.

Step 2. For bead production, a scaled-up protocol was applied to each Ag plus the negative and positive controls for coating onto a Luminex™ bead.

Step 3. A small sample from each bead production was pooled to check for quality metrics.

Step 4. After the small pool met its quality criteria, a large pool was made of all beads before dispensing into individual vials sufficient for 25 multiplex tests per vial. The final bead panel vials were kept at <65° C. for long-term storage.

Step 5. The quality of the post-dispense HLA-DQ Epitope Panel was checked by Luminex LABScreen™ assay with a panel of reference Ab to ensure the Ag performed consistently throughout the production stages.

### Example 3: Luminex Assay and Epitope Analysis

### Epitope Analysis for Monoclonal Antibodies

Human mAb were of the most relevant clinical significance for mapping functional epitopes. To demonstrate the utility of the epitope panel design described herein, mouse mAb were also included as test samples. mAb generated through other means were also utilized so long as so long they were also determined to be useful for mapping ER epitope(s).

Step 1. Following the instructions provided in One Lambda LABScreen™ Product Insert (thermofisher.com/onelambda/wo/en/home.html), a panel of mouse and human mAbs (Table 2) were characterized with the generated HLA-DQ epitope panel described herein using LABScan3D™ flow analyzer (Luminex® FLEXMAP 3D®).

TABLE 2
A list of mAb used as examples for epitope mapping.

| Human IgG mAb | Mouse IgM mAb |
|---|---|
| LB_DQB0201_A | FB1423 |
| LB_DQB0301_A | FB1922 |
| LB_DQB0303_A | FC1276 |
| LB_DQB0303_B | FD362 |
| LB_DQB0303_C | FS582 |
| LB_DQB0402_A | FS1023 |
| LB_DQB0501_A | FS1754 |
| LB_DQB0501_C | FT869 |
| LB_DQB0601_B | |
| LB_DQB0602_B | |
| LB_DQB0604_A | |
| LB_DQB0604_B | |

Step 2. The data acquired by Luminex LABScan™ 3D software xPONENT 4.2™ was exported as a csv file.

Step 3. Following the instructions provided in One Lambda HLA Fusion™ Software User Manual (thermofisher.com/onelambda/wo/en/home.html), the csv file was imported into HLA Fusion™ for data analysis.

Step 4. For each mAb sample, its DQ specificity profile was characterized by the DQ epitope panel (including 32 HLA Ag) at the 2-field molecular level. The total number of positive beads were antibody concentration dependent as shown due to affinity/avidity differences. The following phenomena were observed: 1) the mean fluorescence intensity signals (MFI) generally decreased as Ab concentration is reduced, 2) the MFI of some mAb decreased drastically when Ab concentration was diluted below certain level, and 3) the MFI of the same mAb binding to different Ag was drastically different at the same Ab concentration. This difference was further magnified at a lower Ab concentration where the weaker MFI signals totally disappeared. Therefore, the total number of positive beads included considerations of both experimental observations and theoretical possibility to identify which ep or ep pattern could match the observed or theoretical specificity pattern.

Step 5. Each specificity profile was analyzed using the amino acid analysis modules (AA and AA3D, based on linear or spatial pattern recognition, respectively) that impute the potential involvement of individual eps and ep patterns of the Ag in the DQ panel. The AA module used an algorithm that imputed linear consecutive amino acid patterns to predict the essential contact residue or region (ER) with the test Ab. As linear range increases and mismatches of spatial distance are observed, the algorithm no longer applies. As such, the AA3D module was used to considered spatial distance.

Step 6. The results of the AA and AA3D analyses in comparison with that of the HLAMatchmaker™ MM for both human and mouse mAb are summarized in Table 3 and Table 4. HLAMatchmaker™ (MM module) results are listed in comparison with AA and AA3D results. Only eps with occurrences matching the number of positive beads are shown. AA3D module also specifies whether the predicted residues are Ab accessible or inaccessible based on modeled structures; they are provided for consideration but not an absolute assignment.

TABLE 3
Epitope analysis of human mAb using HLA Fusion ™ AA and AA3D
modules based on specificity profile determined by HLA-DQ panel.

| | | Number of | AA3D Module | | | |
|---|---|---|---|---|---|---|
| mAb | Locus | Positive Beads | Ep | Occurrence | Accessible | Inaccessible |
| LB_DQB0201_A | DQB1 | 10 | 77 R | 10 | 77 | |
| | | 6 | 28S | 6 | | 28 |
| | | | 30S | 6 | | 30 |
| | | | 37I | 6 | | 37 |
| | | | 37I38V | 6 | | 37, 38 |
| | | | 45O46E | 6 | 45, 46 | |
| | | | 46E47F | 6 | 46 | 47 |
| | | | 47F | 6 | | 47 |
| | | | 51T52L | 6 | 51, 52 | |
| | | | 52L53L | 6 | 52, 53 | |
| | | | 55L | 6 | 55 | |
| | | | 55L56P | 6 | 55, 56 | |
| | | | 70R71K | 6 | 70, 71 | |
| | | | 71K | 6 | 71 | |
| | | | 74A | 6 | | 74 |
| | | | 74A75V | 6 | 75 | 74 |
| LB_DQB0301_A | DQB1 | 9 | 30Y9Y | 9 | | 30, 9 |
| | | 3 | 45E | 3 | 45 | |
| LB_DQB0303_A | DQB1 | 19 | 53L | | 53 | |
| | | | 84Q | | 84 | |
| | | | 84Q85L | 19 | 84 | 85 |
| | | | 85L86E | 19 | | 85, 86 |
| | | | 86E87L | 19 | 87 | 86 |
| | | | 87L | 19 | 87 | |
| | | | 89T | 19 | | 89 |
| | | | 88T90T | 19 | | 89, 90 |
| | | | 90T | 19 | | 90 |
| | | | 125A | 19 | | 125 |
| | | | 125A126Q | 19 | 126 | 125 |
| | | 18 | 81H84Q | 18 | 81, 84 | |
| LB_DQB0303_B | DQB1 | 13 | 52P53L | 13 | 52, 53 | |
| | | | 140T | 13 | 140 | |
| | | | 182N | 13 | 182 | |
| LB_DQB0303_C | DQB1 | 8 | 55P | 8 | 55 | |
| | | | 55P56P | 8 | 55, 56 | |
| LB_DQB0402_A | DQB1 | 31 | 81H | 31 | 81 | |
| | | 26 | 28T | 26 | | 28 |
| | | | 46V47Y | 26 | 46 | 47 |
| | | | 47Y | 26 | | 47 |
| | | 17 | 55R | 17 | 55 | |
| LB_DQB0501_A | DQB1 | 9 | 77R81H | 9 | 77, 81 | |
| | | 4 | 13G14L | 4 | | 13, 14 |
| | | | 14L | 4 | | 14 |
| | | | 37Y38V | 4 | | 37, 38 |
| | | | 70G71A | 4 | 70, 71 | |
| | | | 71A | 4 | 71 | |
| | | | 86A87Y | 4 | 87 | 88 |
| | | | 116I | 4 | | 116 |
| | | | 125S | 4 | | 125 |
| LB_DQB0501_C | DQB1 | 21 | 130R185T | 21 | 130, 185 | |
| | | 18 | | | | |
| LB_DQB0601_B | DQB1 | 17 | 55R | 17 | 55 | |
| | | 13 | 52P53Q | 13 | 52, 53 | |
| | | | 53Q | 13 | 53 | |
| | | | 55R56P | 13 | 55, 56 | |
| | | | 84E | 13 | 84 | |
| | | | 84E85V | 13 | 84 | 85 |
| | | | 89G | 13 | | 89 |
| | | | 89G90I | 13 | | 89, 90 |
| | | | 90I | 13 | | 90 |
| LB_DQB0602_B | DQB1 | 17 | 55R | 17 | 55 | |
| LB_DQB0604_A | DQB1 | 23 | 45G46V | 23 | 45, 46 | |
| LB_DQB0604_B | DQB1 | 17 | 55R | 17 | 55 | |
| | | 13 | 52P53Q | 13 | 52, 53 | |
| | | | 53Q | 13 | 53 | |
| | | | 55R56P | 13 | 55, 56 | |
| | | | 84E | 13 | 84 | |
| | | | 84E85V | 13 | 84 | 85 |
| | | | 89G | 13 | | 89 |
| | | | 89G90I | 13 | | 89, 90 |
| | | | 90I | 13 | | 90 |

| | AA Module | | MM Module | | |
|---|---|---|---|---|---|
| mAb | Ep | Occurrence | Eplet | Residue | Occurrence |
| LB_DQB0201_A | 77 R | 10 | 77R | 75V77R | 10 |
| | 28S | 6 | 52LL | 28S | 6 |
| | 30S | 6 | | 30S | 6 |
| | 37I | 6 | | 37I | 6 |
| | 47E | 6 | | | |
| | 47F | 6 | | | |
| | 52L | 6 | | 52L | 6 |
| | 55L | 6 | | 55L | 6 |
| | 71K | 6 | | | |
| | 74A | 6 | | | |
| LB_DQ80301_A | 75L | 9 | | | |
| | 77T | 9 | | | |
| | 81H | 9 | | | |
| | 84Q | 9 | | | |
| | 45E | 3 | 45EV | 45E46V47Y | 3 |
| | 182N185T | 3 | | | |
| LB_DQB0303_A | 53L | 19 | | | |
| | 84Q | 19 | | | |
| | 85L | 19 | | | |
| | 86E | 19 | | | |
| | 87L | 19 | | | |
| | 89T | 19 | | | |
| | 90T | 19 | | | |
| | 125A | 19 | | | |
| | 81H84Q | 18 | 84QL | 84Q86E87L89T90T125A | 18 |
| LB_DQB0303_B | 52P53L | 13 | | | |
| | 140T | 13 | | | |
| | 182N | 13 | 182N | 182N | 13 |
| LB_DQB0303_C | 55P | 8 | 55P | 55P56P | 8 |
| LB_DQB0402_A | 81H | 31 | | | |
| | 28T | 26 | 46VY | 46V52P28T | 26 |
| | 46V | 26 | | | |
| | 47Y | 26 | | | |
| | 55R | 17 | qb55R | 55R | 17 |
| LB_DQB0501_A | 77R81H | 9 | | | |
| | 13G14L | 4 | | | |
| | 14L | 4 | | | |
| | 37Y36V | 4 | q37YV | 37Y36V | 4 |
| | 70G71A | 4 | | | |
| | 71A | 4 | | | |
| | 66A67Y | 4 | | | |
| | 116I | 4 | 116I | 116I125S | 4 |
| | 125S | 4 | | | |
| LB_DQB0501_C | | | | | |
| LB_DQB0601_B | 55R | 17 | qb55R | 55R | 17 |
| | 53Q | 13 | 52PQ | 53Q89G90 | 13 |
| | 84E | 13 | | | |
| | 85V | 13 | | | |
| | 89G | 13 | | | |
| | 90I | 13 | | | |
| LB_DQB0602_B | 55R | 17 | qb55R | 55R | 17 |
| LB_DQB0604_A | 45G46V | 23 | 45GV | 45G46V | 23 |
| LB_DQB0604_B | 55R | 17 | qb55R | 55R | 17 |
| | 52P53Q | 13 | 52PQ | 53Q89G90 | 13 |
| | 53Q | 13 | | | |
| | 55R56P | 13 | | | |
| | 84E | 13 | | | |
| | 84E85V | 13 | | | |
| | 89G | 13 | | | |
| | 89G90I | 13 | | | |
| | 90I | 13 | | | |

TABLE 4
Epitope analysis of mouse mAb using HLA Fusion ™ AA and AA3D
modules based on specificity profile determined by HLA-DQ panel.

| | | Number of | AA3D Module | | | |
|---|---|---|---|---|---|---|
| mAb | Locus | Positive Beads | Ep | Occurrence | Accessible | Inaccessible |
| FB1423 | DQB1 | 13 | 52P53Q | 13 | 52, 53 | |
| | | | 53Q | 13 | 53 | |
| | | | 55R56P | 13 | 55, 56 | |
| | | | 84E | 13 | 84 | |
| | | | 84E85V | 13 | 84 | 85 |
| | | | 89G | 13 | | 89 |
| | | | 89G90I | 13 | | 89, 90 |
| | | | 90I | 13 | | 90 |
| FB1922/ | DQB1 | 6 | 28S | 6 | | 28 |
| FC1276 | | | 30S | 6 | | 30 |
| | | | 37I | 6 | | 37 |
| | | | 37I38V | 6 | | 37, 38 |
| | | | 45O46E | 6 | 45, 46 | |
| | | | 46E47F | 6 | 46 | 47 |
| | | | 47F | 6 | | 47 |
| | | | 51T52L | 6 | 51, 52 | |
| | | | 52L53L | 6 | 52, 53 | |
| | | | 55L | 6 | 55 | |
| | | | 55L56P | 6 | 55, 56 | |
| | | | 70R71K | 6 | 70, 71 | |
| | | | 71K | 6 | 71 | |
| | | | 74A | 6 | | 74 |
| | | | 74A75V | 6 | 75 | 74 |
| FD362 | DQB1 | 9 | 30Y9Y | 9 | | 30, 9 |
| | | | 75L81H84Q85L87L | 9 | 75, 81, 84, 87 | 85 |
| FS1754 | DQB1 | 4 | 23L | 4 | 23 | |
| | | | 55R56L | 4 | 55, 56 | |
| | | | 56L57D | 4 | 56 | 57 |
| | | | 70E | 4 | 70 | |
| | | | 70E71D | 4 | 70, 71 | |
| | | | 71D | 4 | 71 | |
| | | | 74S77T | 4 | 77 | 74 |
| FS582 | DQA1 | 9 | 11C | 9 | | 11 |
| | | | 18F | 9 | 18 | |
| | | | 45A | 9 | | 45 |
| | | | 47R | 9 | | 47 |
| | | | 47R48W | 9 | | 47, 48 |
| | | | 48W | 9 | | 48 |
| | | | 50E | 9 | 50 | |
| | | | 50E51F | 9 | 50 | 51 |
| | | | 51F52S | 9 | 52 | 51 |
| | | | 52S53K | 9 | 52, 53 | |
| | | | 53K54F | 9 | 53, 54 | |
| | | | 54F55G | 9 | 54, 55 | |
| | | | 55G56G | 9 | 55, 56 | |
| | | | 56G | 9 | 56 | |
| | | | 61G | 9 | 61 | |
| | | | 64R | 9 | 64 | |
| | | | 66M | 9 | | 66 |
| | | | 69A | 9 | | 69 |
| | | | 75I76M | 9 | 75 | 76 |
| | | | 76M | 9 | | 76 |
| | | | 80Y | 9 | | 80 |
| | | | 175Q | 9 | 175 | |
| FS1023 | DQB1 | 8 | 55P | 8 | 55 | |
| | | | 55P56P | 8 | 55, 56 | |
| FT869 | DQB1 | 3 | 45E | 3 | 45 | |
| | | | 45E46V | 3 | 45, 46 | |

| | AA Module | | MM Module | | |
|---|---|---|---|---|---|
| | | Occur- | | | Occur- |
| mAb | Ep | rence | Eplet | Residue | rence |
| FB1423 | 52P53Q | 13 | 52PQ | 53Q89G90I | 13 |
| | 53Q | 13 | | | |
| | 55R56P | 13 | | | |
| | 84E | 13 | | | |
| | 84E85V | 13 | | | |
| | 89G | 13 | | | |
| | 89G90I | 13 | | | |
| | 90I | 13 | | | |
| FB1922/ | 28S | 6 | | | |
| FC1276 | 30S | 6 | | | |
| | 37I | 6 | | | |
| | 46E | 6 | | | |
| | 47F | 6 | | | |
| | 52L | 6 | 52LL | 52L55L28S30S37I | 5 |
| | 55L | 6 | | | |
| | 71K | 6 | | | |
| | 74A | 6 | | | |
| FD362 | | | | | |
| | 75L77T81H84Q | 9 | | | |
| FS1754 | 23L | 4 | 23L | 23L | 4 |
| | 55R56L | 4 | 56L | 56L71D | 4 |
| | 56L57D | 4 | | | |
| | 70E | 4 | qp67IE | 67I70E | 4 |
| | 70E71D | 4 | | | |
| | 71D | 4 | | | |
| | 75V77T | 4 | rn76VT | 75v77T | 4 |
| FS582 | 11C | 9 | | | |
| | 18F | 9 | | | |
| | 45A | 9 | | | |
| | 47R | 9 | | | |
| | 48W | 9 | | | |
| | 50E | 9 | | | |
| | 52S | 9 | 52SK | 52S53K11C18F45A64R66M69A80Y | 9 |
| | 53K | 9 | | | |
| | 55O | 9 | | | |
| | 56G | 9 | | | |
| | 61G | 9 | | | |
| | 64R | 9 | | | |
| | 66M | 9 | | | |
| | 69A | 9 | | | |
| | 76M | 9 | | | |
| | 80Y | 9 | | | |
| | 175Q | 9 | | | |
| FS1023 | 55P | 8 | 55PP | 55P56P | 8 |
| FT869 | 45E | 3 | 45EV | 45E46V47Y | 3 |
| | 45E45V | 3 | | | |
| | 182N18 | 3 | | | |

Step 7. The probability of whether an ep or ep pattern was involved in an epitope was estimated by the number of its occurrences in the positive but not the negative beads versus the total number of observed and theoretical positive beads. If they match, then such an ep or ep pattern could be essential for binding to the test Ab.

Various situations are encountered when analyzing mAb specificity profiles using AA and AA3D algorithm:

A. In some cases, only a single ep or ep pattern is implicated in all positive Ag, such as 55P for mAb LB_DQB0303_C, 55R for mAb LB_DQB0602_B, and 45G46V for LB_DQB0604_A. They are likely the essential residue or region (ER) of the epitope with high confidence because they are accessible and can account for all positive and negative Ag.

B. In other cases, more than one ep or ep pattern are implicated, but they cannot be counted as the same ER due to their distance apart (>3.5 Å apart). For example, 52P53L, 140T or 182N could be accounted for the binding profile of LB_DQB0303_B but they cannot be resolved because they are simultaneously present in all positive Ag while absent from negative Ag.

C. In some cases, all the positive signals are strong (MFI >10,000) even at a diluted Ab concentration. In other cases, the mAb binds a subset of Ag on the panel appreciably much weaker than another set of Ag especially at a more diluted Ab concentration. This could be explained that although the mAb recognizes the same ER on all positive Ag, the different ancillary residues or regions (AR) on various Ag could account for the different affinities and MFI signals. For example, mAb LB_DQB0601_B at 1.1 μg/ml binds 13 Ag better than 4 others while at 10 μg/ml, it binds all 17 Ag strongly, but it only recognizes 13 Ag at 0.37 μg/ml. An ER of 55R is only predicted when all 17 Ag are considered positive. Similar situations are also observed for LB_DQB0604_B.

D. There were also situations where a subset of MFI signals could drop down to the negative range, and therefore a change to the specificity profile at a lower Ab concentration was needed, which in turn lead to a different prediction. For example, different eps or ep patterns are predicted due to different specificity profiles at different Ab concentrations. In fact, for 8 out of the 12 human mAb analyzed, the ep or ep pattern prediction outcome hinged upon the number of positive Ag called.

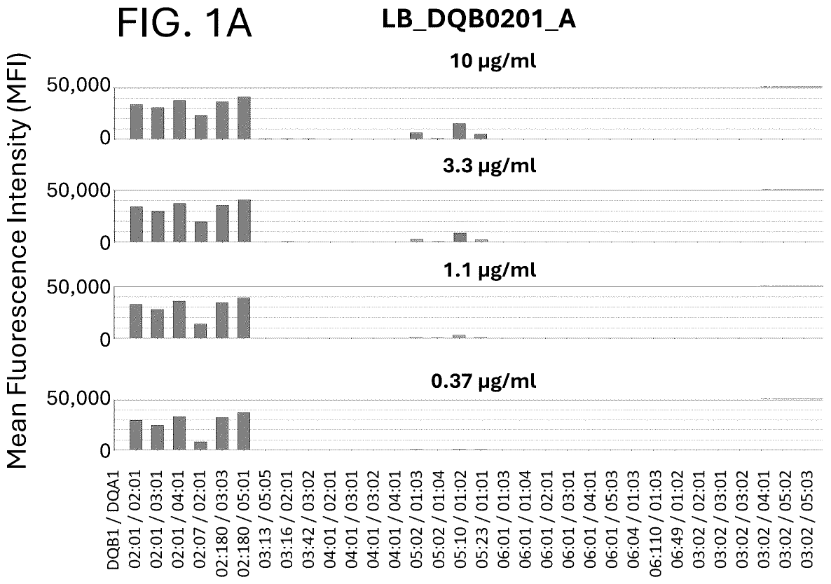

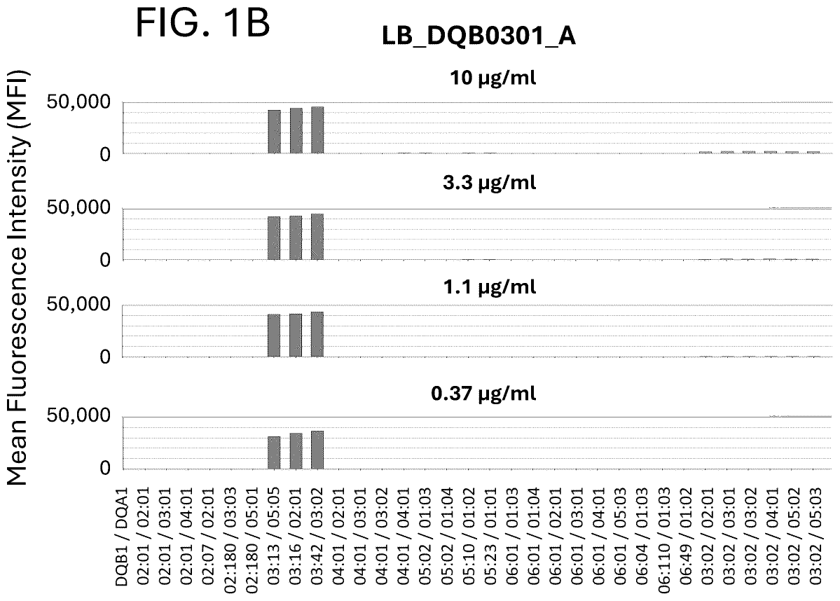

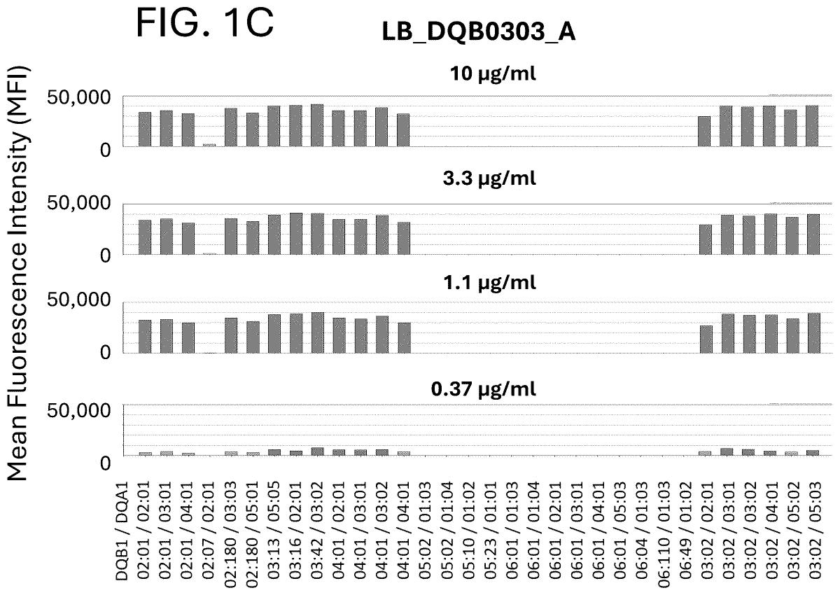

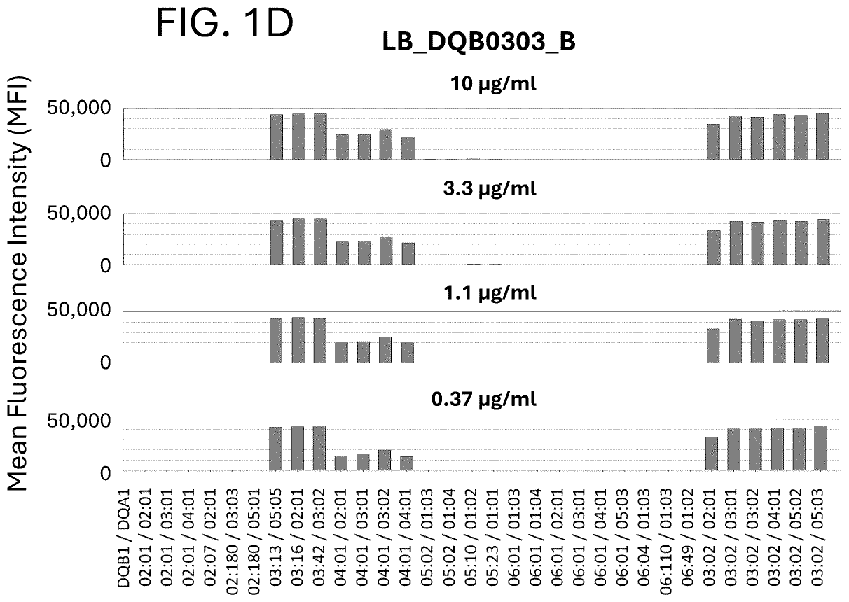

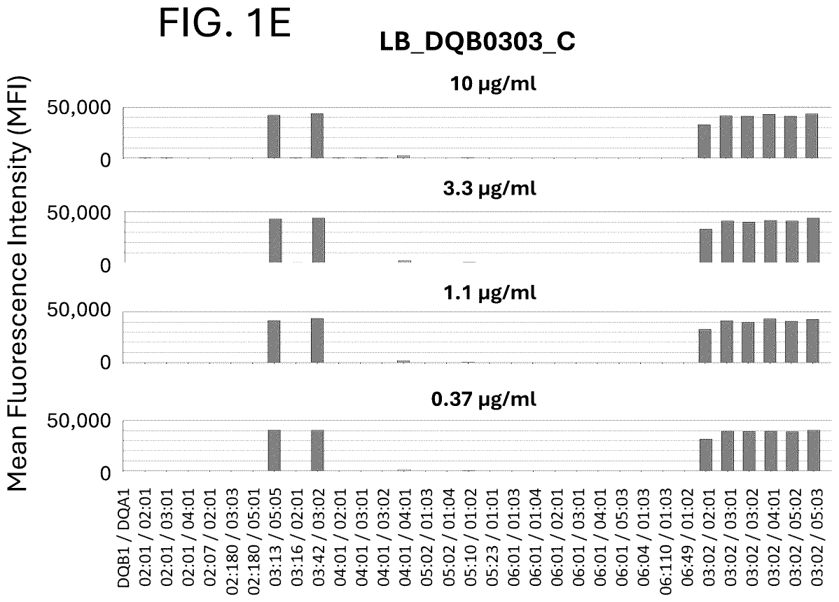

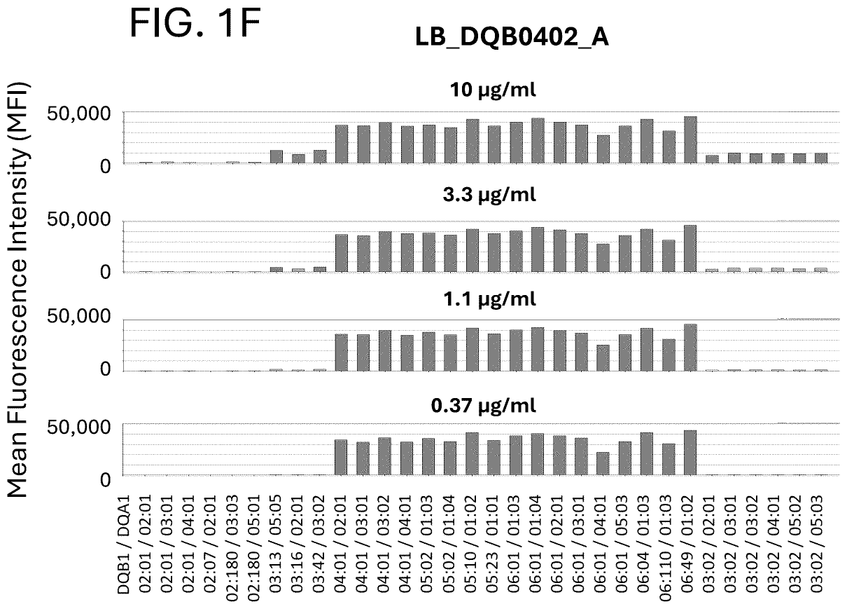

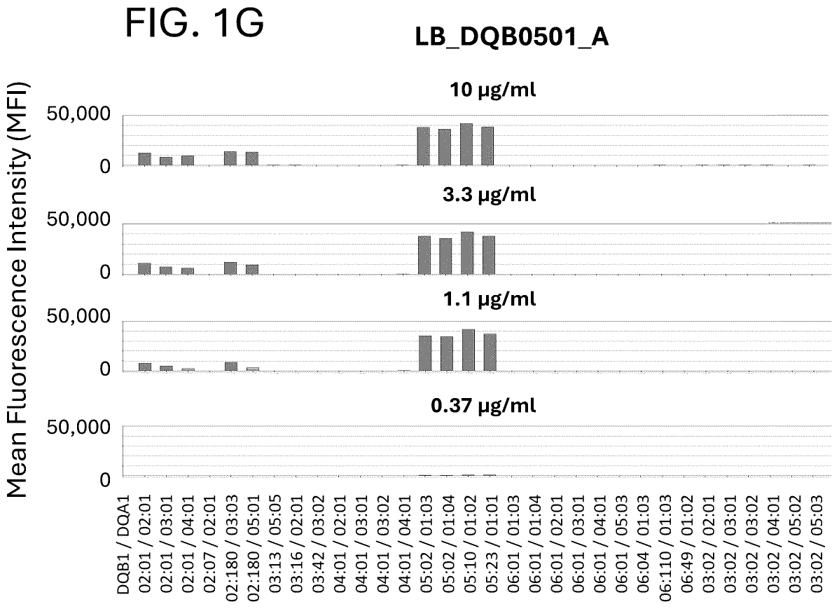

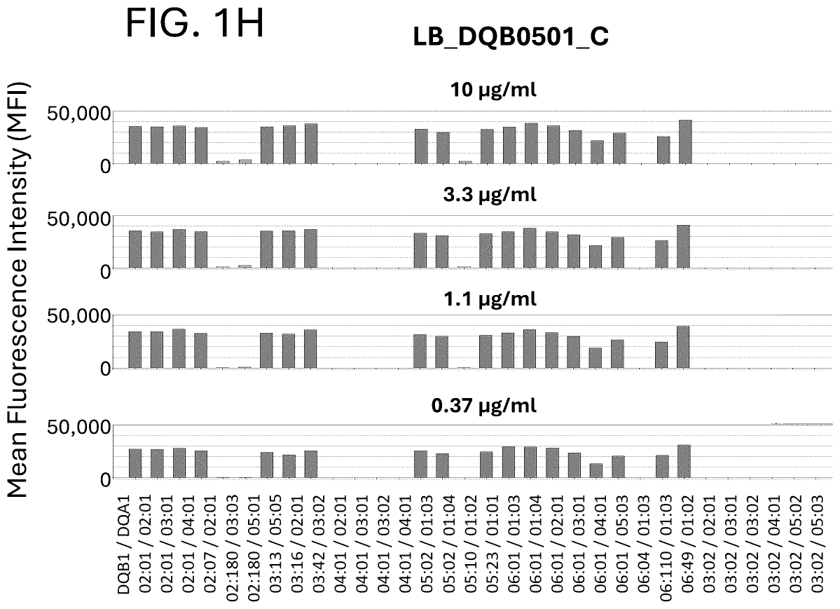

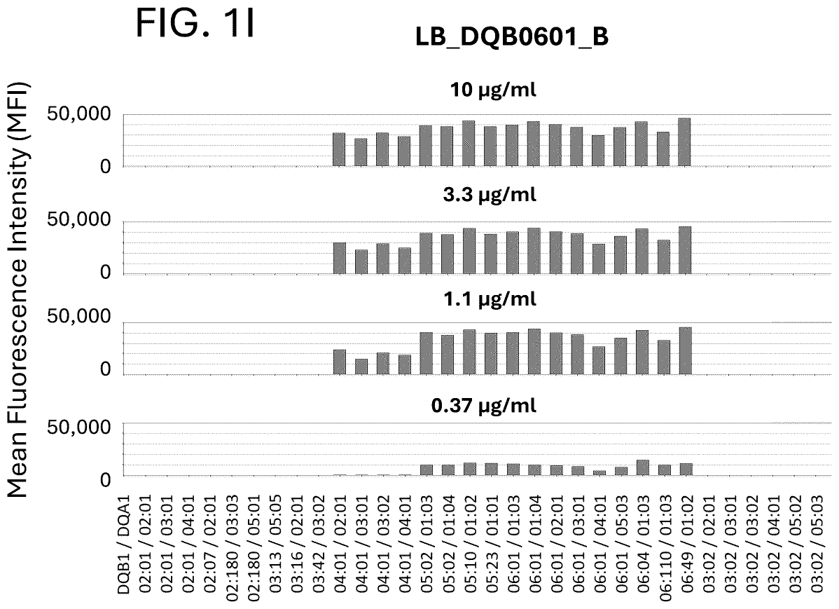

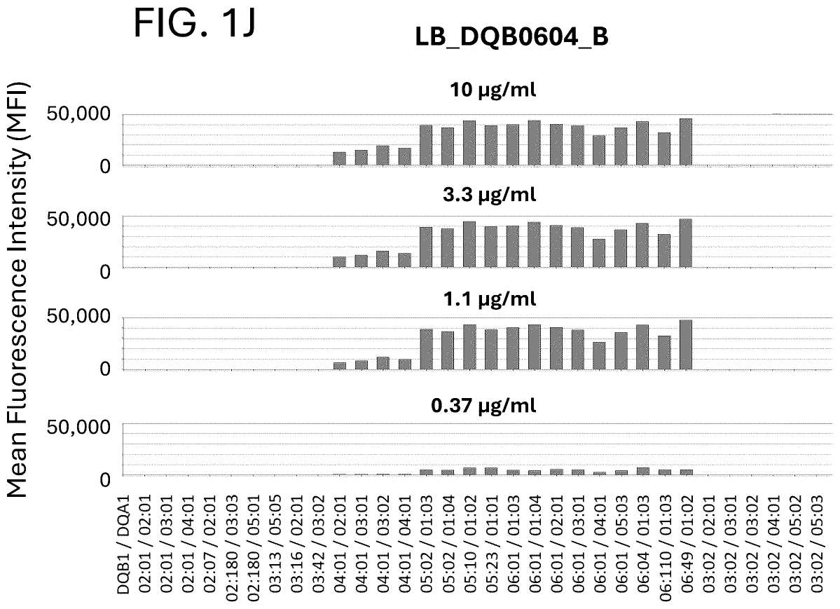

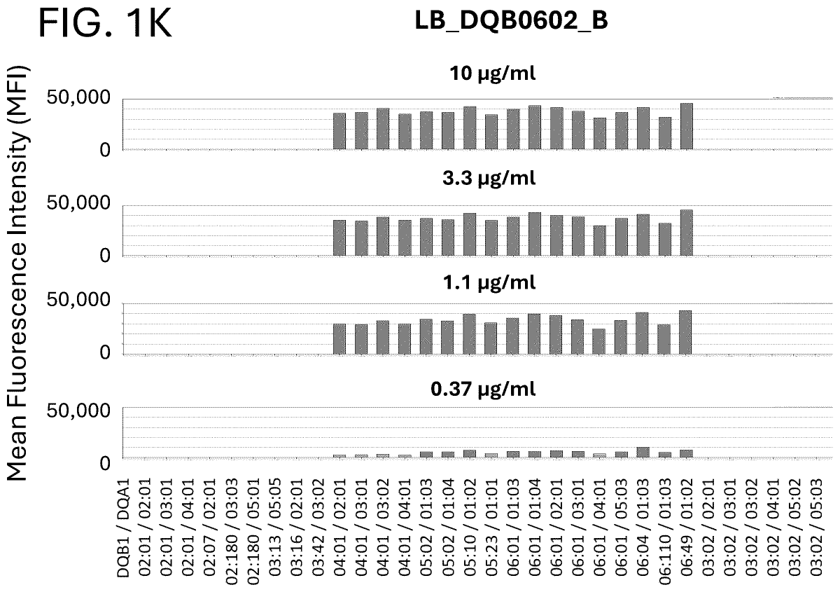

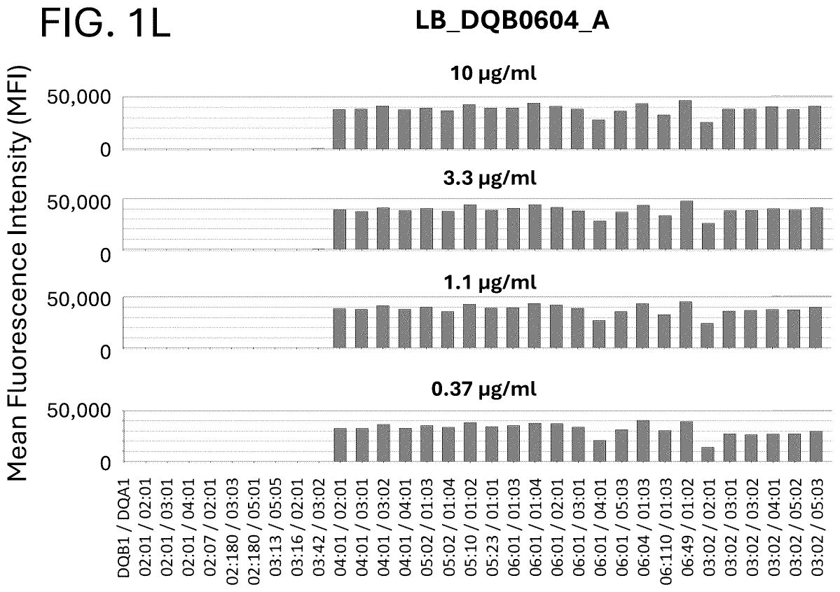

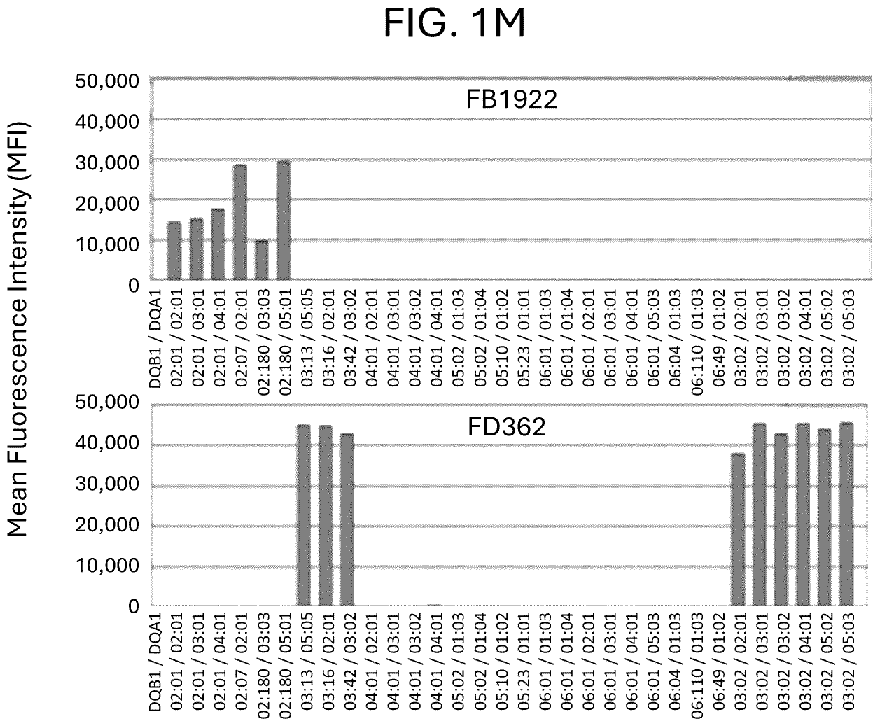

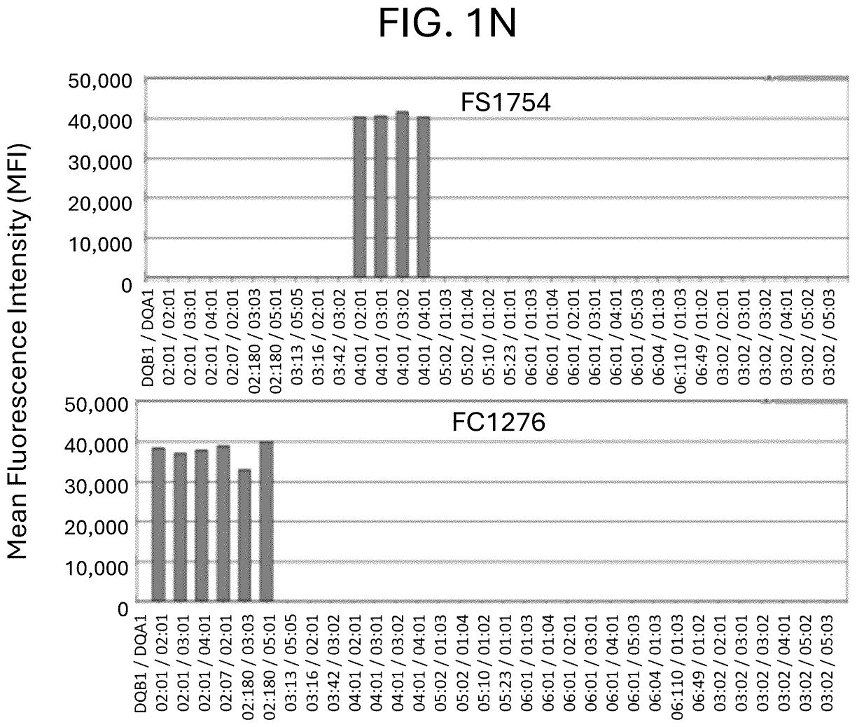

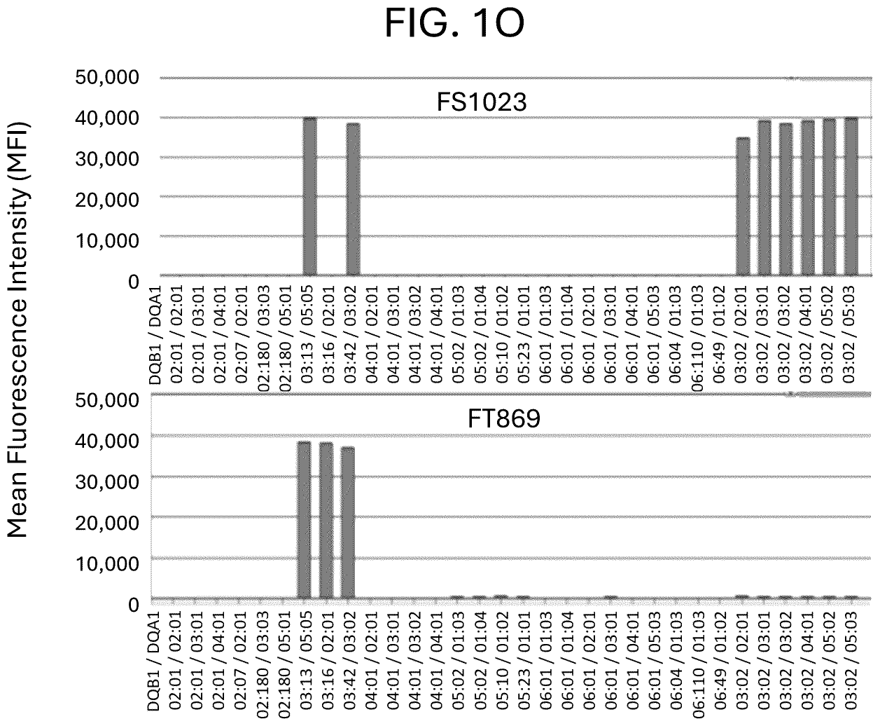

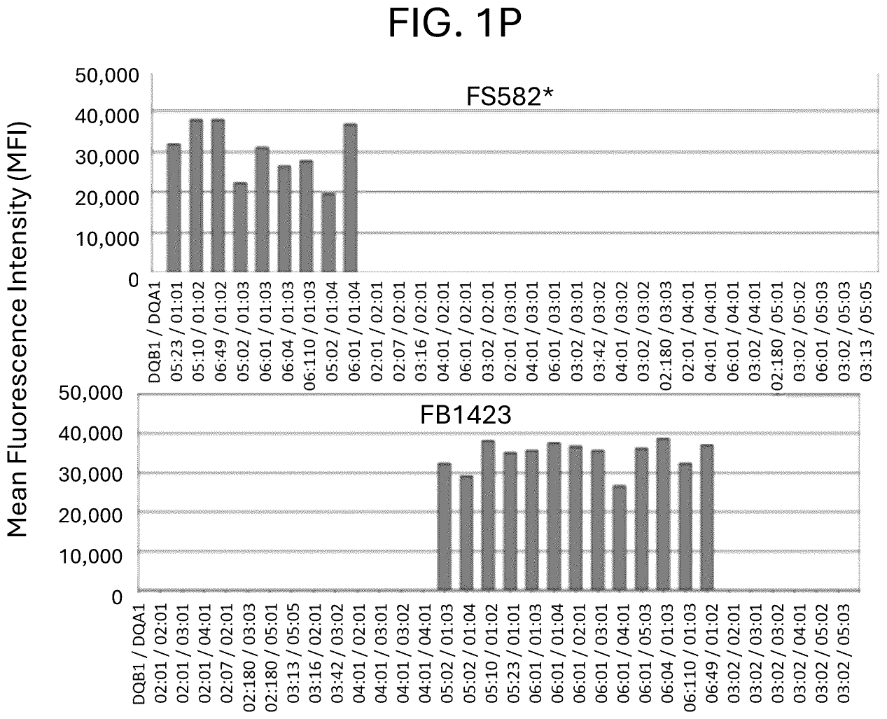

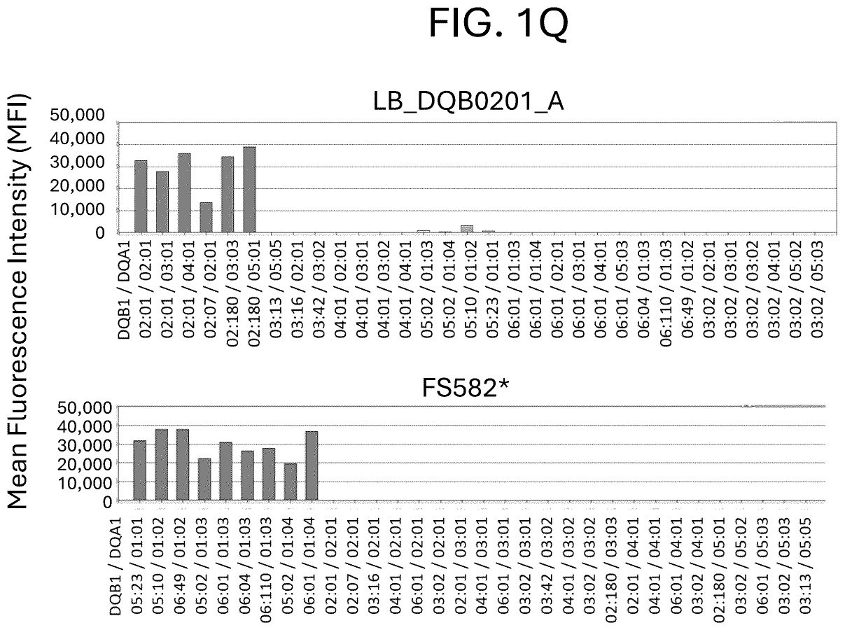

E. For mAb with a specificity profile of appreciably uneven binding signals—7 out of the 12 mAb analyzed here (FIGS. 1A-1L), there were also situations where different ER were predicted pending on whether the mAb recognized the same ER in all positive beads or different ER on the stronger binding Ag from the weaker ones. For example, mAb LB_DQB0501_A could recognize the same ep or ep pattern on all 9 Ag or only 4 stronger binders out of the 9 Ag. Another example is LB_DQB0601_B where different eps or ep patterns were predicted pending on whether the ER is present on all 17 Ag or just 13 of them.

F. The mouse mAb used for this study were not purified and their dilution factors were previously determined based on their clear signals to HLA-DQ Ag. No additional dilutions were tested on this HLA-DQ panel. Strong MFI were observed on all positive beads at the predetermined dilutions. Ab FB1922 and FC1276 share the same specificity profile and therefore the same set of potential ER or AR candidates, albeit FC1276 shows more homogeneous strength than FB1922 which could be due to concentration difference and/or the 2 mAb have different affinity preferences to Ag.

G. It is also common to have several eps or ep patterns simultaneously predicted even when the specificity profile is unequivocal. For example, for DQA1 binder mouse mAb FS582, it is not unexpected that so many eps and ep patterns are predicted because there are not enough naturally occurring C and WD DQA1 alleles to provide a better resolution, and mouse immune system may recognize an ER that is rarely recognized by human immune system.

H. As to mouse mAb FD362, 55P is predicted in only 8 out of 9 positive beads (not shown); on the other hand, after extending the cluster length or distance range, accessible patterns did match all 9 positive beads.

I. In the case of LB_DQ0501_C, 130R185T was only predicted with AA3D but not AA module, which clearly points out the power of 3D approach as positions 130 and 185 are 55 residues apart linearly. In addition, several distinct binding patterns were observed among Ag of the same 1^{st} field molecular typing. For example, strong for DQB1*02:01 and DQB1*02:07 but weak for DQB1*02:180, strong DQB1*05:02 and DQB1*05:23 but weak DQB1*05:10, and positive for DQB1*06:01, DQB1*06:49 and DQB1*06:110 but negative DQB1*06:04. These distinctly different binding patterns among Ag of highly homologous sequences provide information as to which ep or ep pattern is recognized by the Ab.

J. The eplet system adopted by HLAMatchmaker™ is based on the functional epitope hypothesis that is currently the prevailing approach in epitope mapping in the field. Based on the same specificity profile on the same HLA-DQ panel described herein, in some cases, the AA and AA3D predictions were consistent with MM predictions. However, surprisingly it was also apparent that many of the AA and AA3D predictions were not covered by MM. Eplets are mostly assigned artificially with limited few having experimental verification. It was observed that the implicated residues would not appear if not already assigned as an eplet. In contrast, the approach disclosed herein considers all variant positions for implicated residues (eps or ep patterns) within certain reasonable length or distance based on structural information and sequences.

K. Due to the all-encompassing nature of an AA3D approach, residues at positions not conventionally considered highly immunogenic such as regions outside the exon 2 (HLA-CII) or not solvent exposed were also predicted. Ab-accessible residues conceptually have direct impact on Ab binding and can be prioritized as area of interest; however, Ab-inaccessible residues if involved in peptide binding could exert their effect through the presented peptide if recognized by the Ab. In addition, the Ab-inaccessible residues could exert their allosteric impact on the conformations of the neighboring Ab-accessible residues. Therefore, the all-encompassing approach in panel design and epitope analysis disclosed herein is superior in identifying residue(s) (eps) and ep pattern(s) essential for Ab recognition without prematurely excluding informative observations.

L. The same human and mouse mAb were also tested on existing commercial LABScreen™ SAB products LS2A01 and LS2AEX01 which in combination carry 30 DQ Ag but only 9 overlaps with this HLA-DQ panel. Their resulting AA, AA3D and MM analyses are shown in Tables 5 and 6. In general, except for LB_DQB0303_B, LB_DQB0303_C, LB_DQB0602_B, LB_DQB0604_A, FB1922, FS1023, and FT869 (7 out of 19 mAb), specificity profiles based on LS2A01+LS2AEX01 panels resulted in different predictions and higher degrees of uncertainty, including an inability to differentiate between DQA1 and DQB1 epitopes from the analyses, as compared to the HLA-DQ panel described herein, highlighting the additional information provided by this panel in reducing ambiguities.

TABLE 5
Epitope analysis of human mAb using HLA Fusion ™ AA and AA3D modules
based on specificity profile determined by LABScreen ™ LS2A01 + LS2AEX01 panels.

| | | Number of | AA3D Module | | | |
|---|---|---|---|---|---|---|
| mAb | Locus | Positive Beads | Ep | Occurrence | Accessible | Inaccessible |
| LB_DQB0201_A | DQ81 | 5 | 28S | 5 | | 28 |
| | | | 30S | 5 | | 30 |
| | | | 37I | 5 | | 37 |
| | | | 37I38V | 5 | | 37, 38 |
| | | | 45O46E | 5 | 45, 46 | |
| | | | 46E47F | 5 | 46 | 47 |
| | | | 47F | 5 | | 47 |
| | | | 52L | 5 | 52 | |
| | | | 52L53L | 5 | 52, 53 | |
| | | | 55L | 5 | 55 | |
| | | | 55L56P | 5 | 55, 56 | |
| | | | 70R71K | 5 | 70, 71 | |
| | | | 71K | 5 | 71 | |
| | | | 74A | 5 | | 74 |
| | | | 74A75V | 5 | 75 | 74 |
| LB_DQB0303_A | DQA | 21 | 11Y | 21 | | 11 |
| | | | 18S | 21 | 18 | |
| | | | 45V | 21 | | 45 |
| | | | 48L | 21 | | 48 |
| | | | 61F | 21 | 61 | |
| | | | 64T | 21 | 64 | |
| | | | 66I | 21 | | 66 |
| | | | 80S | 21 | | 80 |
| | | | 129H130S | 21 | 129, 130 | |
| | DQB | | 53L | 21 | 53 | |
| | | | 84Q | 21 | 84 | |
| | | | 84Q85L | 21 | 84 | 85 |
| | | | 85L86E | 21 | | 85, 86 |
| | | | 86E87L | 21 | 87 | 86 |
| | | | 87L | 21 | 87 | |
| | | | 89T | 21 | | 89 |
| | | | 89T90T | 21 | | 89, 90 |
| | | | 90T | 21 | | 90 |
| | | | 125A | 21 | | 125 |
| | | | 125A126Q | 21 | 126 | 125 |
| LB_DQB0301_A | DQB1 | 12 | 55P | 12 | 55 | |
| | | | 55P56P | 12 | 55, 56 | |
| | | 6 | 45E | 6 | 45 | |
| | | | 45E46V | 6 | 45, 46 | |
| LB_DQB0303_B | DQB1 | 16 | 52P53L | 16 | 52, 53 | |
| | | | 140T | 16 | 140 | |
| | | | 182N | 16 | 182 | |
| LB_DQB0303_C | DQB1 | 12 | 55P | 12 | 55 | |
| | | | 55P56P | 12 | 55, 56 | |
| LB_DQB0402_A | DQB1 | 30 | | | | |
| | | 25 | 28T | 25 | | 28 |
| | | | 46V47Y | 25 | 46 | 47 |
| | | | 47Y | 25 | | 47 |
| | | | 52P | 25 | 52 | |
| | | 13 | 55R | 13 | 55 | |
| LB_DQB0501_A | DQB1 | 8 | 75V77R | 8 | 75,77 | |
| | | | 77R | 8 | 77 | |
| | | 3 | 13O14L | 3 | | 13, 14 |
| | | | 14L | 3 | | 14 |
| | | | 37Y38V | 3 | | 37, 38 |
| | | | 70O71A | 3 | 70, 71 | |
| | | | 71A | 3 | 71 | |
| | | | 74S77R | 3 | 77 | 74 |
| | | | 86A87Y | 3 | 87 | 86 |
| | | | 116I | 3 | | 116 |
| | | | 125S | 3 | | 125 |
| LB_DQB0501_C | | 15 | | | | |
| LB_DQB0502_B/ | DQB1 | 13 | 55R | 13 | 55 | |
| LB_DQB0504_B | | | | | | |
| LB_DQB0604_A | DQB1 | 19 | 45G45V | 19 | 45, 46 | |
| LB_DQB0601_B | DQB1 | 13 | 55R | 13 | 55 | |
| | DQA1 | 9 | 11C | 9 | | 11 |
| | | | 18F | 9 | 18 | |
| | | | 45A | 9 | | 45 |
| | | | 47R | 9 | | 47 |
| | | | 47R48W | 9 | | 47, 48 |
| | | | 48W | 9 | | 48 |
| | | | 50E | 9 | 50 | |
| | | | 50E51F | 9 | 50 | 51 |
| | | | 51F52S | 9 | 52 | 51 |
| | | | 52S53K | 9 | 52, 53 | |
| | | | 53K54F | 9 | 53, 54 | |
| | | | 54F55G | 9 | 54, 55 | |
| | | | 55G56G | 9 | 55, 56 | |
| | | | 56G | 9 | 56 | |
| | | | 61G | 9 | 61 | |
| | | | 64R | 9 | 64 | |
| | | | 66M | 9 | | 66 |
| | | | 69A | 9 | | 69 |
| | | | 75I76M | 9 | 75 | 76 |
| | | | 76M | 9 | | 76 |
| | | | 80Y | 9 | | 80 |
| | | | 175Q | 9 | 175 | |
| | DQB1 | | 52P53Q | 9 | 52, 53 | |
| | | | 53Q | 9 | 53 | |
| | | | 55R56P | 9 | 55, 55 | |
| | | | 84E | 9 | 84 | |
| | | | 84E85V | 9 | 84 | 85 |
| | | | 89O | 9 | | 89 |
| | | | 89O90I | 9 | | 89, 90 |
| | | | 90I | 9 | | 90 |

| | AA Module | | MM Module | | |
|---|---|---|---|---|---|
| | | Occur- | | | Occur- |
| mAb | Ep | rence | Eplet | Residue | rence |
| LB_DQB0201_A | 28S | 5 | | | |
| | 30S | 5 | | | |
| | 37I | 5 | | | |
| | 37I38V | 5 | | | |
| | 45O46E | 5 | | | |
| | 46E47F | 5 | | | |
| | 47F | 5 | | | |
| | 52L | 5 | 52LL | 52L55L28S30S37I | 5 |
| | 52L53L | 5 | | | |
| | 55L | 5 | | | |
| | 55L56P | 5 | | | |
| | 70R71K | 5 | | | |
| | 71K | 5 | | | |
| | 74A | 5 | | | |
| | 74A75V | 5 | | | |
| LB_DQB0303_A | 11Y | 21 | | | |
| | 18S | 21 | | | |
| | 45V | 21 | | | |
| | 48L | 21 | | | |
| | 61F | 21 | 61FT | 61F64T55R | 21 |
| | 64T | 21 | | | |
| | | | qa55R | 55R | 21 |
| | 66I | 21 | | | |
| | 80S | 21 | | | |
| | 129H130S | 21 | | | |
| | 53L | 21 | | | |
| | 84Q | 21 | 84QL | 84Q86E87L89T90T125A | 21 |
| | 84Q85L | 21 | | | |
| | 85L86E | 21 | | | |
| | 86E87L | 21 | | | |
| | 87L | 21 | | | |
| | 89T | 21 | | | |
| | 89T90T | 21 | | | |
| | 90T | 21 | | | |
| | 125A | 21 | | | |
| | 125A126Q | 21 | | | |
| LB_DQB0301_A | 55P | 12 | 55PP | 55P56P | 12 |
| | 55P56P | 12 | | | |
| | 45E | 6 | 45EV | 45E46V47Y | 6 |
| | 45E46V | 6 | | | |
| LB_DQB0303_B | 52P53L | 16 | | | |
| | 140T | 16 | | | |
| | 182N | 16 | 182N | 182N | 16 |
| LB_DQB0303_C | 55P | 12 | 55PP | 55P56P | 12 |
| | 55P56P | 12 | | | |
| LB_DQB0402_A | | | qa185I | 185I | 30 |
| | 28T | 25 | | | |
| | 46V | 25 | | | |
| | 47Y | 25 | | | |
| | 52P | 25 | | | |
| | 55R | 13 | qb55R | 55R | 13 |
| LB_DQB0501_A | 75V77R | 8 | 77R | 75V77R | 8 |
| | 77R | 8 | | | |
| | 14L | 3 | | | |
| | 37Y38V | 3 | q37YV | 37Y38V | 3 |
| | 71A | 3 | | | |
| | 71A74S | 3 | | | |
| | 74S75V77R | 3 | | | |
| | 86A87Y | 3 | | | |
| | 116I | 3 | 116I | 116I125S | 3 |
| | 125S | 3 | | | |
| LB_DQB0501_C | | | | | |
| LB_DQB0502_B/ | 55R | 13 | qb55R | 55R | 13 |
| LB_DQB0504_B | | | | | |
| LB_DQB0604_A | 45O46V | 19 | 45OV | 45O46V | 19 |
| LB_DQB0601_B | 55R | 13 | qb55R | 55R | 13 |
| | 11C | 9 | | | |
| | 18F | 9 | | | |
| | 45A | 9 | | | |
| | 47R | 9 | | | |
| | 47R48W | 9 | | | |
| | 48W | 9 | | | |
| | 50E | 9 | | | |
| | 50E51F | 9 | | | |
| | 51F52S | 9 | | | |
| | 52S53K | 9 | 52SK | 52S53K11C18F45A64R68M89A30V | 9 |
| | 53K54F | 9 | | | |
| | 54F55G | 9 | | | |
| | 55G56G | 9 | | | |
| | 56G | 9 | | | |
| | 61G | 9 | | | |
| | 64R | 9 | | | |
| | 66M | 9 | | | |
| | 69A | 9 | | | |
| | 75I76M | 9 | | | |
| | 76M | 9 | | | |
| | 80Y | 9 | | | |
| | 175Q | 9 | | | |
| | 52P53Q | 9 | 52PQ | 53Q89G90I | |
| | 53Q | 9 | | | |
| | 55R56P | 9 | | | |
| | 84E | 9 | | | |
| | 84E85V | 9 | | | |
| | 89G | 9 | | | |
| | 89G90I | 9 | | | |
| | 90I | 9 | | | |

TABLE 6
Epitope analysis of mouse mAb using HLA Fusion ™ AA3D modules
based on specificity profile determined by LABScreen ™ LS2A01 + LS2AEX01 panels.

| | | Number of | AA3D Module | | | |
|---|---|---|---|---|---|---|
| mAb | Locus | Positive Beads | Ep | Occurrence | Accessible | Inaccessible |
| FB1423 | DQA | 9 | 11C | 9 | | 11 |
| | | | 18F | 9 | 18 | |
| | | | 45A | 9 | | 45 |
| | | | 47R | 9 | | 47 |
| | | | 47R48W | 9 | | 47, 48 |
| | | | 48W | 9 | | 48 |
| | | | 50E | 9 | 50 | |
| | | | 50E51F | 9 | 50 | 51 |
| | | | 51F52S | 9 | 52 | 51 |
| | | | 52S53K | 9 | 52, 53 | |
| | | | 53K54F | 9 | 53, 54 | |
| | | | 54F55G | 9 | 54, 55 | |
| | | | 55G56G | 9 | 55, 56 | |
| | | | 56G | 9 | 56 | |
| | | | 61G | 9 | 61 | |
| | | | 64R | 9 | 64 | |
| | | | 66M | 9 | | 66 |
| | | | 69A | 9 | | 69 |
| | | | 75I76M | 9 | 75 | 76 |
| | | | 76M | 9 | | 76 |
| | | | 80Y | 9 | | 80 |
| | | | 175Q | 9 | 175 | |
| | DQB | | 52P53Q | 9 | 52, 53 | |
| | | | 53Q | 9 | 53 | |
| | | | 55R56P | 9 | 55, 56 | |
| | | | 84E | 9 | 84 | |
| | | | 84E85V | 9 | 84 | 85 |
| | | | 89G | 9 | | 89 |
| | | | 89G90I | 9 | | 89, 90 |
| | | | 90I | 9 | | 90 |
| FB1922 | DQB1 | 5 | 28S | 5 | | 28 |
| | | | 30S | 5 | | 30 |
| | | | 37I | 5 | | 37 |
| | | | 37I38V | 5 | | 37, 38 |
| | | | 45G46E | 5 | 45, 46 | |
| | | | 46E47F | 5 | 46 | 47 |
| | | | 47F | 5 | | 47 |
| | | | 52L | 5 | 52 | |
| | | | 52L53L | 5 | 52, 53 | |
| | | | 55L | 5 | 55 | |
| | | | 55L56P | 5 | 55, 56 | |
| | | | 70R71K | 5 | 70, 71 | |
| | | | 71K | 5 | 71 | |
| | | | 74A | 5 | | 74 |
| | | | 74A75V | 5 | 75 | 74 |
| FD362 | DQB1 | 12 | 55P | 12 | 55 | |
| | | | 55P56P | 12 | 55, 56 | |
| FS1754 | DQB1 | 4 | 55R56L | 4 | 55, 56 | |
| | | | 56L57D | 4 | 56 | 57 |
| | | | 70E | 4 | 70 | |
| | | | 70E71D | 4 | 70, 71 | |
| | | | 71D | 4 | 71 | |
| | | | 74S77T | 4 | 77 | 74 |
| FS582 | DQA1 | 9 | 11C | 9 | | 11 |
| | | | 18F | 9 | 18 | |
| | | | 45A | 9 | | 45 |
| | | | 47R | 9 | | 47 |
| | | | 47R48W | 9 | | 47, 48 |
| | | | 48W | 9 | | 48 |
| | | | 50E | 9 | 50 | |
| | | | 50E51F | 9 | 50 | 51 |
| | | | 51F52S | 9 | 52 | 51 |
| | | | 52S53K | 9 | 52, 53 | |
| | | | 53K54F | 9 | 53, 54 | |
| | | | 54F55G | 9 | 54, 55 | |
| | | | 55G56G | 9 | 55, 56 | |
| | | | 56G | 9 | 56 | |
| | | | 61G | 9 | 61 | |
| | | | 64R | 9 | 64 | |
| | | | 66M | 9 | | 66 |
| | | | 69A | 9 | | 69 |
| | | | 75I76M | 9 | 75 | 76 |
| | | | 76M | 9 | | 76 |
| | | | 80Y | 9 | | 80 |
| | | | 175Q | 9 | 175 | |
| | DQB1 | | 52P53Q | 9 | 52, 53 | |
| | | | 53Q | 9 | 53 | |
| | | | 55R56P | 9 | 55, 56 | |
| | | | 84E | 9 | 84 | |
| | | | 84E85V | 9 | 84 | 85 |
| | | | 89G | 9 | | 89 |
| | | | 89G90I | 9 | | 89, 90 |
| | | | 90I | 9 | | 90 |
| FS1023 | DQB1 | 12 | 55P | 12 | 55 | |
| | | | 55P56P | 12 | 55, 56 | |
| FTB69 | DQB1 | 6 | 45E | 6 | 45 | |
| | | | 45E46V | 6 | 45, 46 | |

It will be noted that AA and MM software modules are not listed in some examples above because Tables 3, 4, 5 and 6 already serve as examples for comparison.

Step 9. Based on the observations described in Step 8, it became apparent that the predicted ER frequently comes with multiple choices because WT Ag do not provide enough variations to deconvolute the various possibilities. Although WT Ag selected for the panel provide a greater coverage of all the potential ER in a targeted population, they cannot identify the ER unless there is another non-binding Ag that differs from the binding Ag by only a particular ER. Even when only a single ER is predicted with high confidence, experimental evidence was desired to confirm the prediction, especially if that ER has not been verified by another Ab.

Step 10. Commonly occurring HLA alleles do not always provide sufficient resolution to one another in identifying ER recognized by an Ab. By evolution, it is not uncommon to have blocks of genetic materials spliced together to form a gene, therefore certain alleles always carry certain eps and/or ep patterns simultaneously but too distant to be involved in the same ER. To pinpoint which ep or ep pattern is the ER, the most straightforward approach is to mutate these eps of the Ag one at a time or in combination to observe its effect on binding with Ab.

### Example 4: Use of Engineered Variants to Further Resolve Epitope Ambiguities

More often than desired, multiple eps or ep patterns implicated as ER by AA and AA3D analyses for many mAbs remained to be resolved. That is why the use of EV with altered ep or ep pattern was desirable to reach a satisfying assignment of ER. In some cases, even if the ER has been predicted to be a single ep with confidence, it is not with certainty unless a loss-of-function (LOF) is confirmed for an engineered variant (EV) with the altered WT ER.

Instead of looking for a native HLA molecule which may not exist for this purpose, strategically designed mutations derived from the binding HLA Ag were employed to provide clarity.

### Design of Engineered HLA-DQ Variants

Based on the test results of mAb samples described in the above examples and HLA sequence alignment, certain eps or ep patterns emerged as the most likely candidates. If multiple eps or ep patterns are simultaneously implicated by epitope analysis and present in sequence alignment, there may be a need to create EV with altered residue(s) at implicated position(s) to identify which ep(s) lead to LOF. Even if only one ep or one cluster is implicated, mutation of one ep at a time or in combination may be desirable to confirm which one(s) plays the essential role, especially when they are topologically close to one another. On the other hand, even without a test result, certain eps based on sequence and structural analyses theoretically could elicit immunogenic responses and serve as the antigenic determinant, but if they do not have a known counterpart allele that could point to the critical role of a specific ep or ep pattern, the epitope analysis cannot achieve a satisfactory resolution.

Because all variant positions, solvent exposed or not, are included in the Ag on the epitope panel, the implicated eps could exhibit different degrees of solvent exposure. The current knowledge of HLA structures is mostly based on molecular modeling which depicts the general structural framework well, but the exact orientation of an amino acid residue is less certain. Nonetheless, the eps predicted to be surface exposed are theoretically more likely to provide a direct contact with an Ab, and were therefore selected to be mutated at higher priority than the eps predicted to be less solvent exposed. This is not to rule out that the embedded ep could play an indirect role in contributing to binding through peptide presentation or an allosteric effect on the confirmations of neighboring residues. If none of the EV of the predicted surface ep(s) has any significant effect on binding to a specific mAb, the rest of the implicated eps could also be investigated. In some cases, the full impact of mAb binding may not be revealed unless multiple engineered eps are present in the same Ag. Stated alternatively, an ER could include >1 ep. These EV residues were introduced to the backbone of each subgroup of a locus such as DQB1*02:01, DQB1*03:01, DQB1*04:01, DQB1*05:01 and DQB1*06:01, but theoretically any binding Ag can be used to make an EV to identify ER recognized by a binding mAb. The ep of interest is usually engineered to a residue corresponding to a non-binding Ag known to be tolerated structurally, presuming this type of changes minimizes the potential disturbance of the global structures beyond the local conformations. However, according to experimental data described here, change of the ep in the WT Ag to another amino acid residue not known to be at this position is also mostly well tolerated.

The eps and ep patterns predicted by HLA Fusion™ AA and AA3D modules based on mAb specificity profiles were targeted for mutagenesis. However, the scope of these predictions was limited by the number of available mAb. To increase the scope, sequence alignment of the DQB1 or DQA1 alleles on the panel were performed to identify variant positions. These variants are prioritized for mutagenesis if they are predicted to be surface exposed and/or involved in peptide binding. EV with single or group mutations were generated and expressed to compare with WT in binding to mAb.

Because the number of EV, if each carrying only one altered ER from WT, needed to cover all the potential ER would be impractical, some variant positions were mutated simultaneously. EV that carry more than one altered ep or ep pattern may not provide resolution at a single ep level, but they can quickly narrow down where the ER could be with less cost and effort, and this level of resolution may be sufficient in reaching a clinical conclusion in some cases.

Tables 7A-D include a non-exhaustive list of EV examples for HLA-DQ. Some of them have been successfully used in mapping ER. The total and prioritized variant positions suitable for EV generation are listed in Table 7A. Exemplary designed single amino acid changes based on prioritized variant positions are listed in Tables 7B and 7C. Changes to multiple positions can be combined in 1 LV construct as demonstrated by the examples given in Table 7D that have been used for ER and/or AR verification described later.

TABLE 7A
List of exemplary HLA-DQ variant positions among common alleles
suitable for EV generation. Prioritized variant positions are
also shown that cover proposed Ab accessible positions and
positions likely impact peptide selection for presentation.
HLA-DQ Variant Positions

| | |
|---|---|
| DQA1 | DQB1 |

| Total | Prioritized | Total | Prioritized | Total | Prioritized |
|---|---|---|---|---|---|
| 1 | 1 | 3 | 3 | 130 | 130 |
| 2 | 2 | [symbol] | | 135 | 135 |
| 11 | 11 | 13 | 13 | 140 | 140 |
| 18 | 18 | 14 | | 163 | 163 |
| 25 | 25 | 23 | 23 | 167 | 167 |
| 26 | 26 | 26 | | 168 | 168 |
| 34 | 34 | 28 | 28 | 169 | 169 |
| 40 | 40 | 30 | 30 | 182 | 182 |
| 41 | 41 | 37 | | 185 | 185 |
| 45 | | 38 | | 197 | |
| 47 | 47 | 45 | 45 | | |
| 48 | | 46 | 46 | | |
| 50 | 50 | 47 | | | |
| 51 | | 51 | 51 | | |
| 52 | 52 | 52 | 52 | | |
| 53 | 53 | 53 | 53 | | |
| 54 | 54 | 55 | 55 | | |
| 55 | 55 | 56 | 56 | | |
| 56 | 56 | 57 | 57 | | |
| [symbol] | [symbol] | [symbol] | [symbol] | | |
| 61 | 61 | 67 | 67 | | |
| 64 | 64 | 70 | 70 | | |
| 68 | | 71 | 71 | | |
| 69 | 69 | 74 | 74 | | |
| 75 | 75 | 75 | | | |
| 76 | 76 | 77 | 77 | | |
| 80 | 80 | 81 | 81 | | |
| 107 | | 84 | 84 | | |
| 129 | 129 | 85 | 65 | | |
| 130 | 130 | 86 | 67 | | |
| 156 | | 87 | | | |
| 160 | 160 | 89 | | | |
| 161 | 161 | 90 | | | |
| 163 | 163 | 11 [symbol] | | | |
| 175 | 175 | 125 | | | |
| 187 | | 126 | 126 | | |

[symbol]

indicates data missing or illegible when filed

Tables 7B and 7C. List of exemplary HLA-DQ engineered variants suitable for ER and/or AR verification. Representative DQA1 (Table 7B) and DQB1 (Table 7C) alleles are included as examples. DQA1*06:01 only differs from DQA1*04:01 in position 25, therefore exemplary DQA1*06:01 EV can be understood by referencing those listed under DQA1*04:01 EV. Some EV designs are underlined because they are changes among small residues (amino acids A, G and S) that may not result in a conspicuous impact.

TABLE 7B
Exemplary HLA-DQA1 EVs.
DQA1 Prioritized EV Design

| Change to existing e p | Change to alanine |
|---|---|
| DQA1*01:01_E1K | DQA1*01:01_E1A |
| DQA1*01:01_D2G | DQA1*01:01_D2A |
| DQA1*01:01_C11Y | DQA1*01:01_C11A |
| DQA1*01:01_F18S | DQA1*01:01_F18A |
| DQA1*01:01_Y25F | DQA1*01:01_Y25A |
| DQA1*01:01_T26S | DQA1*01:01_T26A |
| DQA1*01:01_E34Q | DQA1*01:01_E34A |
| DQA1*01:01_E40G | DQA1*01:01_E40A |
| DQA1*01:01_R41K | DQA1*01:01_R41A |
| DQA1*01:01_R47Q | DQA1*01:01_R47A |
| DQA1*01:01_E50V | DQA1*01:01_E50A |
| DQA1*01:01_S52R | DQA1*01:01_S52A |
| DQA1*01:01_K53Q | DQA1*01:01_K53A |
| DQA1*01:01_F54L | DQA1*01:01_F54A |
| DQA1*01:01_G55R | DQA1*01:01_G55A |
| DQA1*01:01_G56R | DQA1*01:01_G56A |
| DQA1*01:01_P59R | DQA1*01:01_P59A |
| DQA1*01:01_G61F | DQA1*01:01_G61A |
| DQA1*01:01_R64T | DQA1*01:01_R64A |
| DQA1*01:01_A69L | |
| DQA1*01:01_I75S | DQA1*01:01_I75A |
| DQA1*01:01_M76L | DQA1*01:01_M76A |
| DQA1*01:01_Y80S | DQA1*01:01_Y80A |
| DQA1*01:01_Q129H | DQA1*01:01_Q129A |
| DQA1*01:01_S130A | |
| DQA1*01:01_A160D | |
| DQA1*01:01_D161E | DQA1*01:01_D161A |
| DQA1*01:01_I163S | DQA1*01:01_I163A |
| DQA1*01:01_Q175K | DQA1*01:01_Q175A |
| DQA1*02:01_E1K | DQA1*02:01_E1A |
| DQA1*02:01_D2G | DQA1*02:01_D2A |
| DQA1*02:01_Y11C | DQA1*02:01_Y11A |
| DQA1*02:01_S18F | DQA1*02:01_S18A |
| DQA1*02:01_F25Y | DQA1*02:01_F25A |
| DQA1*02:01_T26S | DQA1*02:01_T26A |
| DQA1*02:01_E34Q | DQA1*02:01_E34A |
| DQA1*02:01_E40G | DQA1*02:01_E40A |
| DQA1*02:01_R41K | DQA1*02:01_R41A |
| DQA1*02:01_K47Q | DQA1*02:01_K47A |
| DQA1*02:01_L50E | DQA1*02:01_L50A |
| DQA1*02:01_H52S | DQA1*02:01_H52A |
| DQA1*02:01_R53Q | DQA1*02:01_R53A |
| DQA1*02:01_L54F | DQA1*02:01_L54A |
| DQA1*02:01_R55G | DQA1*02:01_R55A |
| DQA1*02:01_.56R | DQA1*02:01_.56A |
| DQA1*02:01_P59R | DQA1*02:01_P59A |
| DQA1*02:01_F61G | DQA1*02:01_F61A |
| DQA1*02:01_T64R | DQA1*02:01_T64A |
| DQA1*02:01_L69T | DQA1*02:01_L69A |
| DQA1*02:01_I75S | DQA1*02:01_I75A |
| DQA1*02:01_L76M | DQA1*02:01_L76A |
| DQA1*02:01_S80Y | DQA1*02:01_S80A |
| DQA1*02:01_H129Q | DQA1*02:01_H129A |
| DQA1*02:01_S130A | |
| DQA1*02:01_A160D | |
| DQA1*02:01_D161E | DQA1*02:01_D161A |
| DQA1*02:01_I163S | DQA1*02:01_I163A |
| DQA1*02:01_E175K | DQA1*02:01_E175A |
| DQA1*03:01_E1K | DQA1*03:01_E1A |
| DQA1*03:01_D2G | DQA1*03:01_D2A |
| DQA1*03:01_Y11C | DQA1*03:01_Y11A |
| DQA1*03:01_S18F | DQA1*03:01_S18A |
| DQA1*03:01_Y25F | DQA1*03:01_Y25A |
| DQA1*03:01_S26T | DQA1*03:01_S26A |
| DQA1*03:01_E34Q | DQA1*03:01_E34A |
| DQA1*03:01_E40G | DQA1*03:01_E40A |
| DQA1*03:01_R41K | DQA1*03:01_R41A |
| DQA1*03:01_Q47R | DQA1*03:01_Q47A |
| DQA1*03:01_L50E | DQA1*03:01_L50A |
| DQA1*03:01_R52S | DQA1*03:01_R52A |
| DQA1*03:01_R53Q | DQA1*03:01_R53A |
| DQA1*03:01_F54L | DQA1*03:01_F54A |
| DQA1*03:01_R55G | DQA1*03:01_R55A |
| DQA1*03:01_R56R | DQA1*03:01_R56A |
| DQA1*03:01_P59R | DQA1*03:01_P59A |
| DQA1*03:01_F61G | DQA1*03:01_F61A |
| DQA1*03:01_T64R | DQA1*03:01_T64A |
| DQA1*03:01_L69T | DQA1*03:01_L69A |
| DQA1*03:01_I75S | DQA1*03:01_I75A |
| DQA1*03:01_V76M | DQA1*03:01_V76A |
| DQA1*03:01_S80Y | DQA1*03:01_S80A |
| DQA1*03:01_H129Q | DQA1*03:01_H129A |
| DQA1*03:01_S130A | |
| DQA1*03:01_A160D | |
| DQA1*03:01_D161E | DQA1*03:01_D161A |
| DQA1*03:01_I163S | DQA1*03:01_I163A |
| DQA1*03:01_E175Q | DQA1*03:01_E175A |
| DQA1*04:01_E1K | DQA1*04:01_E1A |
| DQA1*04:01_D2G | DQA1*04:01_D2A |
| DQA1*04:01_Y11C | DQA1*04:01_Y11A |
| DQA1*04:01_S18F | DQA1*04:01_S18A |
| DQA1*04:01_Y25F | DQA1*04:01_Y25A |
| DQA1*04:01_T26S | DQA1*04:01_T26A |
| DQA1*04:01_Q34E | DQA1*04:01_Q34A |
| DQA1*04:01_G40E | DQA1*04:01_G40A |
| DQA1*04:01_R41K | DQA1*04:01_R41A |
| DQA1*04:01_C47Q | DQA1*04:01_C47A |
| DQA1*04:01_V50E | DQA1*04:01_V50A |
| DQA1*04:01_R52S | DQA1*04:01_R52A |
| DQA1*04:01_R53Q | DQA1*04:01_R53A |
| DQA1*04:01_F54L | DQA1*04:01_F54A |
| DQA1*04:01_R55G | DQA1*04:01_R55A |
| DQA1*04:01_.56R | DQA1*04:01_.56A |
| DQA1*04:01_P59R | DQA1*04:01_P59A |
| DQA1*04:01_F61G | DQA1*04:01_F61A |
| DQA1*04:01_T64R | DQA1*04:01_T64A |
| DQA1*04:01_T69L | DQA1*04:01_T69A |
| DQA1*04:01_I75S | DQA1*04:01_I75A |
| DQA1*04:01_L76V | DQA1*04:01_L76A |
| DQA1*04:01_S80Y | DQA1*04:01_S80A |
| DQA1*04:01_H129Q | DQA1*04:01_H129A |
| DQA1*04:01_S130A | |
| DQA1*04:01_A160D | |
| DQA1*04:01_D161E | DQA1*04:01_D161A |
| DQA1*04:01_I163S | DQA1*04:01_I163A |
| DQA1*04:01_E175Q | DQA1*04:01_E175A |
| DQA1*05:01_E1K | DQA1*05:01_E1A |
| DQA1*05:01_D2G | DQA1*05:01_D2A |
| DQA1*05:01_Y11C | DQA1*05:01_Y11A |
| DQA1*05:01_S18F | DQA1*05:01_S18A |
| DQA1*05:01_Y25F | DQA1*05:01_Y25A |
| DQA1*05:01_T26S | DQA1*05:01_T26A |
| DQA1*05:01_Q34E | DQA1*05:01_Q34A |
| DQA1*05:01_G40E | DQA1*05:01_G40A |
| DQA1*05:01_R41K | DQA1*05:01_R41A |
| DQA1*05:01_C47Q | DQA1*05:01_C47A |
| DQA1*05:01_V50E | DQA1*05:01_V50A |
| DQA1*05:01_R52S | DQA1*05:01_R52A |
| DQA1*05:01_R53Q | DQA1*05:01_R53A |
| DQA1*05:01_F54L | DQA1*05:01_F54A |
| DQA1*05:01_R55G | DQA1*05:01_R55A |
| DQA1*05:01_.56R | DQA1*05:01_.56A |
| DQA1*05:01_P59R | DQA1*05:01_P59A |
| DQA1*05:01_F61G | DQA1*05:01_F61A |
| DQA1*05:01_T64R | DQA1*05:01_T64A |
| DQA1*05:01_L69T | DQA1*05:01_L69A |
| DQA1*05:01_S75I | DQA1*05:01_S75A |
| DQA1*05:01_L76V | DQA1*05:01_L76A |
| DQA1*05:01_S80Y | DQA1*05:01_S80A |
| DQA1*05:01_H129Q | DQA1*05:01_H129A |
| DQA1*05:01_S130A | |
| DQA1*05:01_A160D | |
| DQA1*05:01_E161D | DQA1*05:01_E161A |
| DQA1*05:01_S163I | DQA1*05:01_S163A |
| DQA1*05:01_K175E | DQA1*05:01_K175A |
| DQA1*06:01_F25Y | DQA1*06:01_F25A |

TABLE 7C
Exemplary HLA-DQB1 EVs.
DQB1 Prioritized EV Design

| Change to existing e p | Change to alanine |
|---|---|
| DQB1*02:01_S3P | DQB1*02:01_S3A |
| DQB1*02:01_G13V | DQB1*02:01_G13A |
| DQB1*02:01_R23L | DQB1*02:01_R23A |
| DQB1*02:01_S28T | DQB1*02:01_S28A |
| DQB1*02:01_S30Y | DQB1*02:01_S30A |
| DQB1*02:01_G45E | DQB1*02:01_G45A |
| DQB1*02:01_E46V | DQB1*02:01_E46A |
| DQB1*02:01_T51K | DQB1*02:01_T51A |
| DQB1*02:01_L52P | DQB1*02:01_L52A |
| DQB1*02:01_L53Q | DQB1*02:01_L53A |
| DQB1*02:01_L55R | DQB1*02:01_L55A |
| DQB1*02:01_P56L | DQB1*02:01_P56A |
| DQB1*02:01_A57D | |
| DQB1*02:01_D66E | DQB1*02:01_D66A |
| DQB1*02:01_I67V | DQB1*02:01_I67A |
| DQB1*02:01_R70E | DQB1*02:01_R70A |
| DQB1*02:01_K71T | DQB1*02:01_K71A |
| DQB1*02:01_A74E | |
| DQB1*02:01_R77T | DQB1*02:01_R77A |
| DQB1*02:01_H81P | DQB1*02:01_H81A |
| DQB1*02:01_Q84E | DQB1*02:01_Q84A |
| DQB1*02:01_L85V | DQB1*02:01_L85A |
| DQB1*02:01_L87Y | DQB1*02:01_L87A |
| DQB1*02:01_Q126H | DQB1*02:01_Q126A |
| DQB1*02:01_R130Q | DQB1*02:01_R130A |
| DQB1*02:01_D135G | DQB1*02:01_D135A |
| DQB1*02:01_A140T | |
| DQB1*02:01_M163V | DQB1*02:01_M163A |
| DQB1*02:01_R167H | DQB1*02:01_R167A |
| DQB1*02:01_G168E | DQB1*02:01_G168A |
| DQB1*02:01_D169A | |
| DQB1*02:01_S182N | DQB1*02:01_S182A |
| DQB1*02:01_T185I | DQB1*02:01_T185A |
| DQB1*03:01_S3P | DQB1*03:01_S3A |
| DQB1*03:01_A13V | |
| DQB1*03:01_R23L | DQB1*03:01_R23A |
| DQB1*03:01_T28S | DQB1*03:01_T28A |
| DQB1*03:01_Y30H | DQB1*03:01_Y30A |
| DQB1*03:01_E45G | DQB1*03:01_E45A |
| DQB1*03:01_V46E | DQB1*03:01_V46A |
| DQB1*03:01_T51K | DQB1*03:01_T51A |
| DQB1*03:01_P52L | DQB1*03:01_P52A |
| DQB1*03:01_L53Q | DQB1*03:01_L53A |
| DQB1*03:01_P55R | DQB1*03:01_P55A |
| DQB1*03:01_P56L | DQB1*03:01_P56A |
| DQB1*03:01_D57S | DQB1*03:01_D57A |
| DQB1*03:01_E66D | DQB1*03:01_E66A |
| DQB1*03:01_V67I | DQB1*03:01_V67A |
| DQB1*03:01_R70G | DQB1*03:01_R70A |
| DQB1*03:01_T71K | DQB1*03:01_T71A |
| DQB1*03:01_E74S | DQB1*03:01_E74A |
| DQB1*03:01_T77R | DQB1*03:01_T77A |
| DQB1*03:01_H81P | DQB1*03:01_H81A |
| DQB1*03:01_Q84E | DQB1*03:01_Q84A |
| DQB1*03:01_L85V | DQB1*03:01_L85A |
| DQB1*03:01_L87F | DQB1*03:01_L87A |
| DQB1*03:01_Q126H | DQB1*03:01_Q126A |
| DQB1*03:01_R130Q | DQB1*03:01_R130A |
| DQB1*03:01_D135G | DQB1*03:01_D135A |
| DQB1*03:01_T140A | |
| DQB1*03:01_M163V | DQB1*03:01_M163A |
| DQB1*03:01_H167R | DQB1*03:01_H167A |
| DQB1*03:01_G168E | DQB1*03:01_G168A |
| DQB1*03:01_D169A | |
| DQB1*03:01_N182S | DQB1*03:01_D182A |
| DQB1*03:01_T185I | DQB1*03:01_T185A |
| DQB1*04:02_S3P | DQB1*04:02_S3A |
| DQB1*04:02_G13V | DQB1*04:02_G13A |
| DQB1*04:02_R23L | DQB1*04:02_R23A |
| DQB1*04:02_T28S | DQB1*04:02_T28A |
| DQB1*04:02_Y30S | DQB1*04:02_Y30A |
| DQB1*04:02_G45E | DQB1*04:02_G45A |
| DQB1*04:02_V46E | DQB1*04:02_V46A |
| DQB1*04:02_T51K | DQB1*04:02_T51A |
| DQB1*04:02_P52L | DQB1*04:02_P52A |
| DQB1*04:02_L53Q | DQB1*04:02_L53A |
| DQB1*04:02_R55P | DQB1*04:02_R55A |
| DQB1*04:02_L56P | DQB1*04:02_L56A |
| DQB1*04:02_D57V | DQB1*04:02_D57A |
| DQB1*04:02_D66E | DQB1*04:02_D66A |
| DQB1*04:02_I67V | DQB1*04:02_I67A |
| DQB1*04:02_E70R | DQB1*04:02_E70A |
| DQB1*04:02_D71K | DQB1*04:02_D71A |
| DQB1*04:02_S74E | DQB1*04:02_S74A |
| DQB1*04:02_T77R | DQB1*04:02_T77A |
| DQB1*04:02_H81P | DQB1*04:02_H81A |
| DQB1*04:02_Q84E | DQB1*04:02_Q84A |
| DQB1*04:02_L85V | DQB1*04:02_L85A |
| DQB1*04:02_L87Y | DQB1*04:02_L87A |
| DQB1*04:02_Q126H | DQB1*04:02_Q126A |
| DQB1*04:02_R130Q | DQB1*04:02_R130A |
| DQB1*04:02_D135G | DQB1*04:02_D135A |
| DQB1*04:02_T140A | |
| DQB1*04:02_M163V | DQB1*04:02_M163A |
| DQB1*04:02_R167H | DQB1*04:02_R167A |
| DQB1*04:02_G168E | DQB1*04:02_G168A |
| DQB1*04:02_D169A | |
| DQB1*04:02_N182R | DQB1*04:02_N182A |
| DQB1*04:02_I185T | DQB1*04:02_I185A |
| DQB1*05:01_S3P | DQB1*05:01_S3A |
| DQB1*05:01_G13V | DQB1*05:01_G13A |
| DQB1*05:01_R23L | DQB1*05:01_R23A |
| DQB1*05:01_T28S | DQB1*05:01_T28A |
| DQB1*05:01_H30Y | DQB1*05:01_H30A |
| DQB1*05:01_G45E | DQB1*05:01_G45A |
| DQB1*05:01_V46E | DQB1*05:01_V46A |
| DQB1*05:01_T51K | DQB1*05:01_T51A |
| DQB1*05:01_P52L | DQB1*05:01_P52A |
| DQB1*05:01_Q53L | DQB1*05:01_Q53A |
| DQB1*05:01_R55L | DQB1*05:01_R55A |
| DQB1*05:01_P56L | DQB1*05:01_P56A |
| DQB1*05:01_V57D | DQB1*05:01_V57A |
| DQB1*05:01_E66D | DQB1*05:01_E66A |
| DQB1*05:01_V67I | DQB1*05:01_V67A |
| DQB1*05:01_G70R | DQB1*05:01_G70A |
| DQB1*05:01_A71D | |
| DQB1*05:01_S74E | DQB1*05:01_S74A |
| DQB1*05:01_R77T | DQB1*05:01_R77A |
| DQB1*05:01_H81P | DQB1*05:01_H81A |
| DQB1*05:01_E84Q | DQB1*05:01_E84A |
| DQB1*05:01_V85L | DQB1*05:01_V85A |
| DQB1*05:01_Y87L | DQB1*05:01_Y87A |
| DQB1*05:01_Q126H | DQB1*05:01_Q126A |
| DQB1*05:01_R130Q | DQB1*05:01_R130A |
| DQB1*05:01_D135G | DQB1*05:01_D135A |
| DQB1*05:01_A140T | |
| DQB1*05:01_M163V | DQB1*05:01_M163A |
| DQB1*05:01_R167H | DQB1*05:01_R167A |
| DQB1*05:01_G168E | DQB1*05:01_G168A |
| DQB1*05:01_D169A | |
| DQB1*05:01_S182N | DQB1*05:01_S182A |
| DQB1*05:01_T185I | DQB1*05:01_T185A |
| DQB1*06:02_S3P | DQB1*06:02_S3A |
| DQB1*06:02_G13V | DQB1*06:02_G13A |
| DQB1*06:02_R23L | DQB1*06:02_R23A |
| DQB1*06:02_T28S | DQB1*06:02_T28A |
| DQB1*06:02_Y30H | DQB1*06:02_Y30A |
| DQB1*06:02_G45E | DQB1*06:02_G45A |
| DQB1*06:02_V46E | DQB1*06:02_V46A |
| DQB1*06:02_T51K | DQB1*06:02_T51A |
| DQB1*06:02_P52L | DQB1*06:02_P52A |
| DQB1*06:02_Q53L | DQB1*06:02_Q53A |
| DQB1*06:02_R55L | DQB1*06:02_R55A |
| DQB1*06:02_P56L | DQB1*06:02_P56A |
| DQB1*06:02_D57V | DQB1*06:02_D57A |
| DQB1*06:02_E66D | DQB1*06:02_E66A |
| DQB1*06:02_V67I | DQB1*06:02_V67A |
| DQB1*06:02_G70R | DQB1*06:02_G70A |
| DQB1*06:02_T71D | DQB1*06:02_T71A |
| DQB1*06:02_E74S | DQB1*06:02_E74A |
| DQB1*06:02_T77R | DQB1*06:02_T77A |
| DQB1*06:02_H81P | DQB1*06:02_H81A |
| DQB1*06:02_E84Q | DQB1*06:02_E84A |
| DQB1*06:02_V85L | DQB1*06:02_V85A |
| DQB1*06:02_F87L | DQB1*06:02_F87A |
| DQB1*06:02_Q126H | DQB1*06:02_Q126A |
| DQB1*06:02_R130Q | DQB1*06:02_R130A |
| DQB1*06:02_D135G | DQB1*06:02_D135A |
| DQB1*06:02_A140T | |
| DQB1*06:02_M163V | DQB1*06:02_M163A |
| DQB1*06:02_R167H | DQB1*06:02_R167A |
| DQB1*06:02_G168E | DQB1*06:02_G168A |
| DQB1*06:02_D169A | |
| DQB1*06:02_S182N | DQB1*06:02_S182A |
| DQB1*06:02_T185I | DQB1*06:02_T185A |

TABLE 7D
List of exemplary EV derived from HLA-DQ used for ER and/
or AR verification. Positions changed to alanine marked
with “*” are conserved positions among Common DQB1 alleles.
Antigen

| DQB1 | DQAI |
|---|---|
| DQB1*02:01_R70E | DQA1*02:01 |
| DQB1*02:01_R70E_K71D | DQA1*02:01 |
| DQB1*02:01_R70E_R77T | DQA1*02:01 |
| DQB1*02:01_K71T | DQA1*02:01 |
| DQB1*02:01_K71T_A74E | DQA1*02:01 |
| DQB1*02:01_K71T_R77T | DQA1*02:01 |
| DQB1*02:01_A74E | DQA1*02:01 |
| DQB1*02:01_A74S | DQA1*02:01 |
| DQB1*02:01_R77T | DQA1*02:01 |
| DQB1*02:01_K128A* | DQA1*02:01 |
| DQB1*02:01_R130Q | DQA1*02:01 |
| DQB1*02:01_R130Q_T185I | DQA1*02:01 |
| DQB1*02:01_F132A* | DQA1*02:01 |
| DQB1*02:01_E137A* | DQA1*02:01 |
| DQB1*02:01_H174A* | DQA1*02:01 |
| DQB1*02:01_E176A* | DQA1*02:01 |
| DQB1*02:01_P183A* | DQA1*02:01 |
| DQB1*02:01_T185I | DQA1*02:01 |
| DOB1*02:01_E187A* | DQA1*02:01 |
| DQB1*03:01_F45G | DQA1*03:03 |
| DQB1*03:01_P52L | DQA1*03:03 |
| DQB1*03:01_P52L_P53Q | DQA1*03:03 |
| DQB1*03:01_P52L_P55L | DQA1*03:03 |
| DQB1*03:01_L53Q | DQA1*03:03 |
| DQB1*03:01_L53Q_P55R | DQA1*03:03 |
| DQB1*03:01_P55A | DQA1*03:03 |
| DOB1*03:01_P55R | DQA1*03:03 |
| DQB1*03:01_H81A | DQA1*03:03 |
| DQB1*03:01_H81P | DQA1*03:03 |
| DQB1*03:01_H81P_Q84E | DQA1*03:03 |
| DQB1*03:01_Q84A | DQA1*03:03 |
| DQB1*03:01_Q84E | DQA1*03:03 |
| DQB1*03:01_Q84E_L85V | DQA1*03:03 |
| DQB1*03:01_Q84E_L87F | DQA1*03:03 |
| DQB1*03:01_L87F | DQA1*03:03 |
| DQB1*03:01_T140A | DQA1*03:03 |
| DQB1*03:01_N182S | DQA1*03:03 |
| DQB1*05:01_E66D_V67I_G70E_A71D_R77T | DQA1*01:02 |
| DQB1*05:01_G70E | DQA1*01:02 |
| DQB1*05:01_G70R | DQA1*01:02 |
| DQB1*05:01_G70E_A71D | DQA1*01:02 |
| DQB1*05:01_G70E_R77T | DQA1*01:02 |
| DQB1*05:01_R77T | DQA1*01:02 |
| DQB1*05:01_877T_H81P | DQA1*01:02 |
| DQB1*05:01_H81P | DQA1*01:02 |
| DQB1*06:01_G45E | DQA1*02:01 |
| DQB1*06:01_G45E_V46E | DQA1*02:01 |
| DQB1*06:01_V46E | DQA1*02:01 |
| DQB1*06:01_P52L | DQA1*02:01 |
| DQB1*06:01_Q53L | DQA1*02:01 |
| DQB1*06:01_Q53L_R55P | DQA1*02:01 |
| DQB1*06:01_R55P | DQA1*02:01 |
| DQB1*06:01_V85L | DQA1*02:01 |
| DQB1*06:01_G89T | DQA1*02:01 |

Some positions of conserved residues may provide critical structural support of the molecule and therefore may not be good candidates for mutagenesis. If such a situation occurs, positive control Ab such as pan-CII Ab FJ5109 and pan-DQ Ab FM5148 are contemplated to detect a gross structural change. Although conserved residues are considered self and should not initiate an immunogenic response by themselves, if close to a variant residue and particularly surface exposed, they can be recognized by an Ab through the affinity maturation process. In such cases, LV of these conserved positions can still provide valuable information for epitope mapping. That is why EVs are not limited to variant positions.

### Use of EV Expressed on Cell Surface to Detect LOF Ep or Ep Pattern

Step 1. The expression construct and recombinant cell line used in this example have been described in Example 2.

Step 2. Each transfection and selection lead to a pool of cells with heterogeneous expression levels including non-expressers that complicated the interpretation of the binding signals. Therefore, only sorted pools of the highest expression population by a high affinity pan-HLA CII mAb were used to evaluate whether the test mAb binds to cell surface Ag.

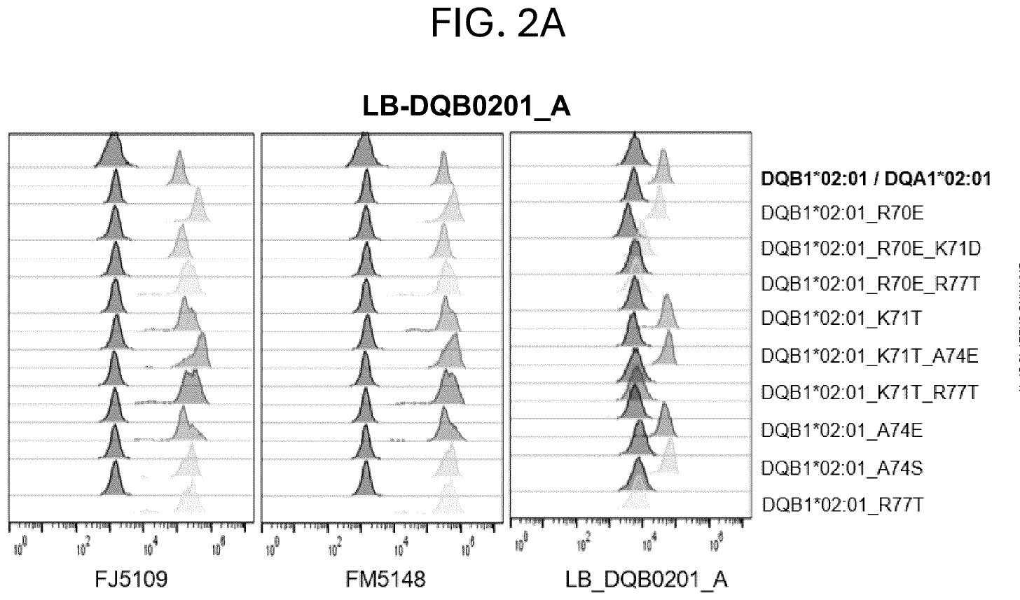

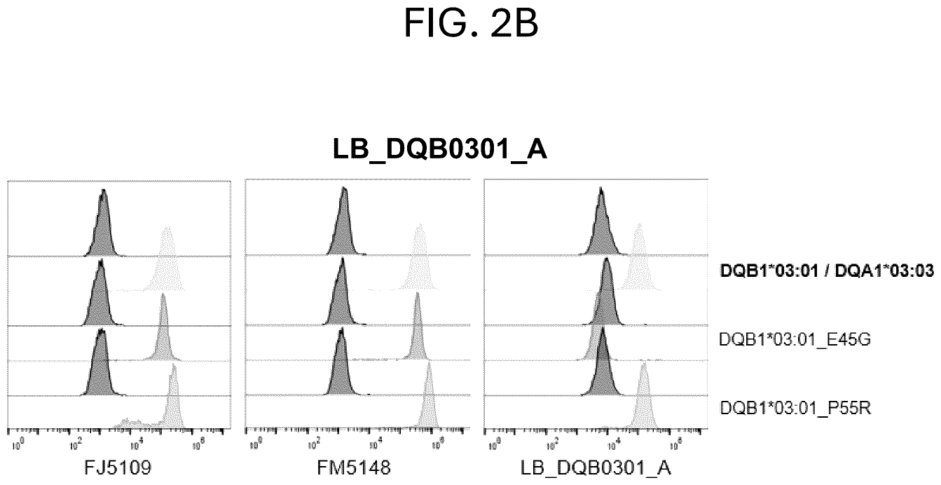

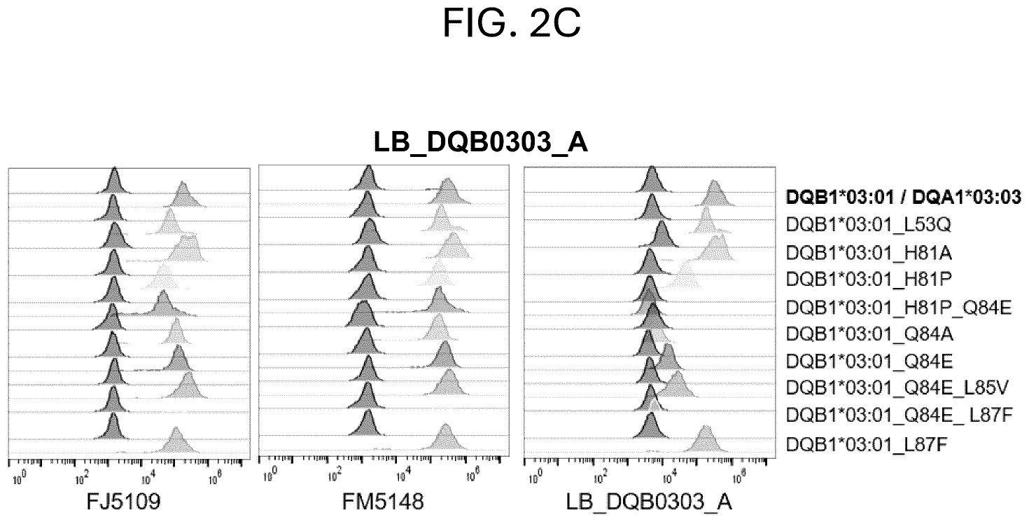

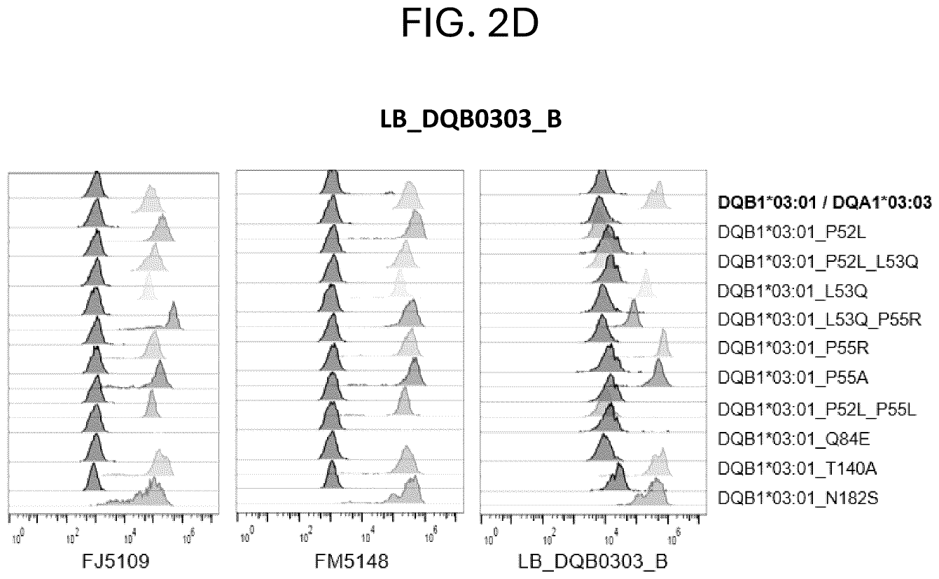

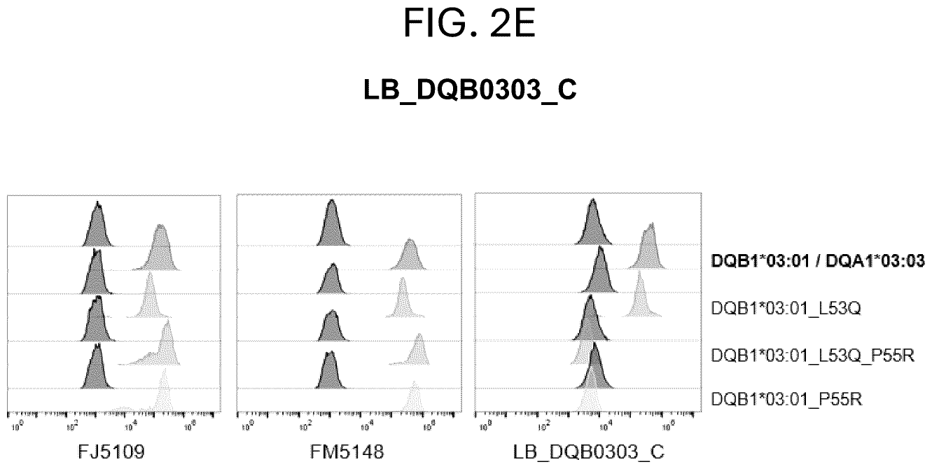

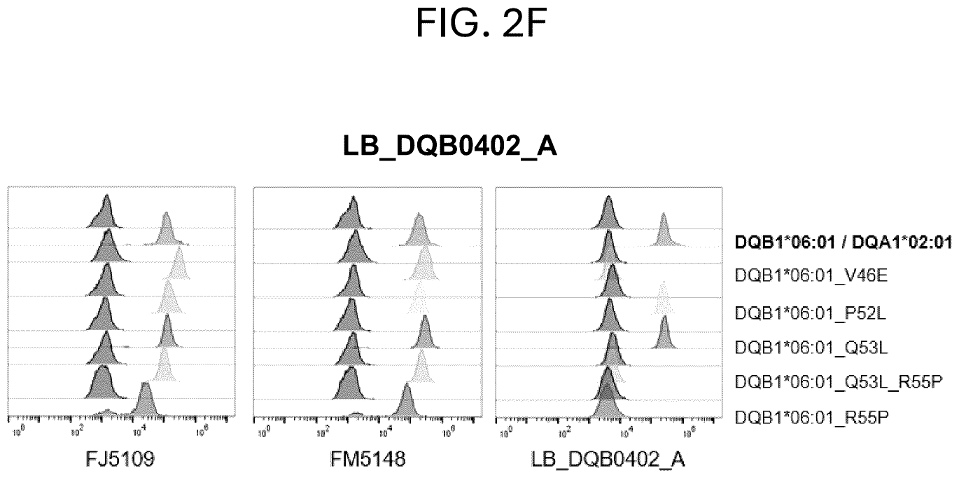

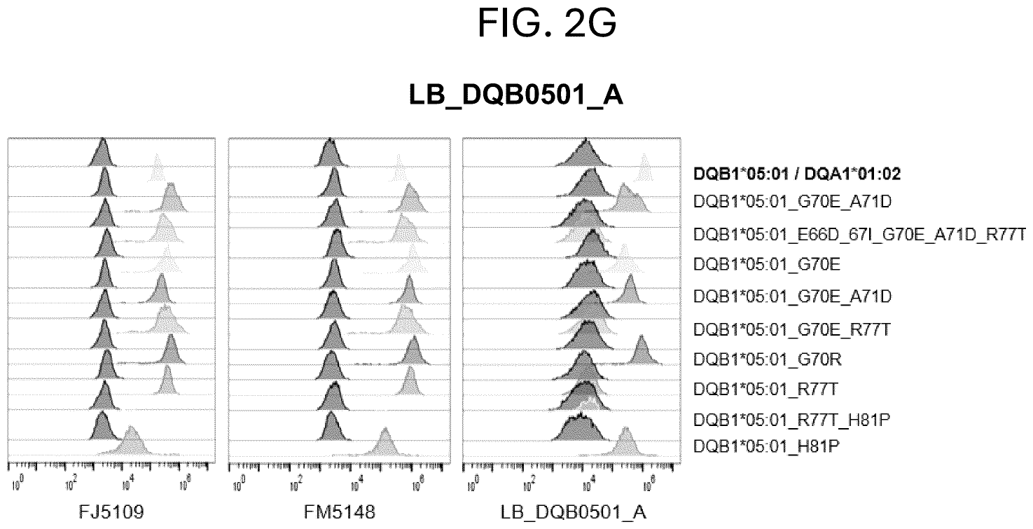

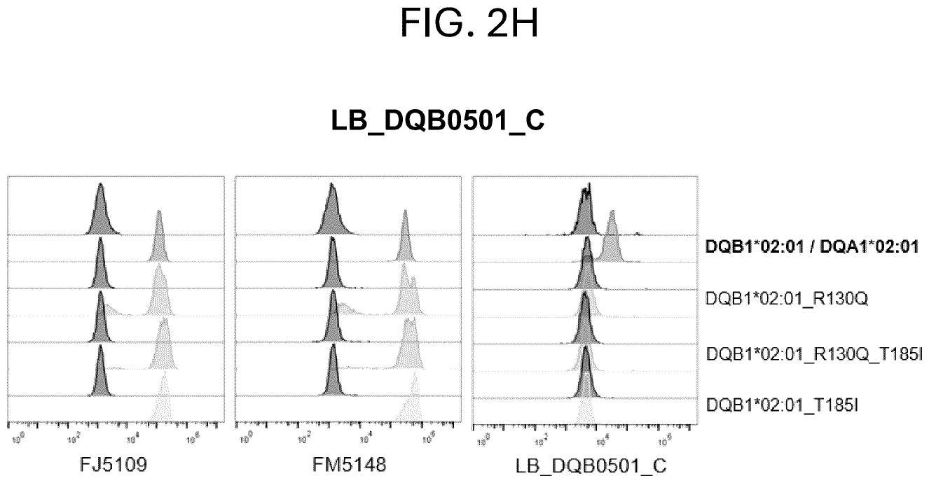

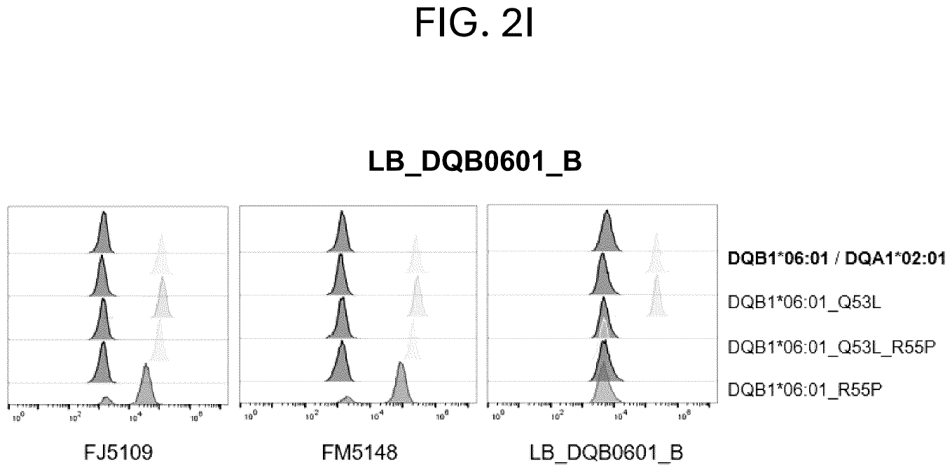

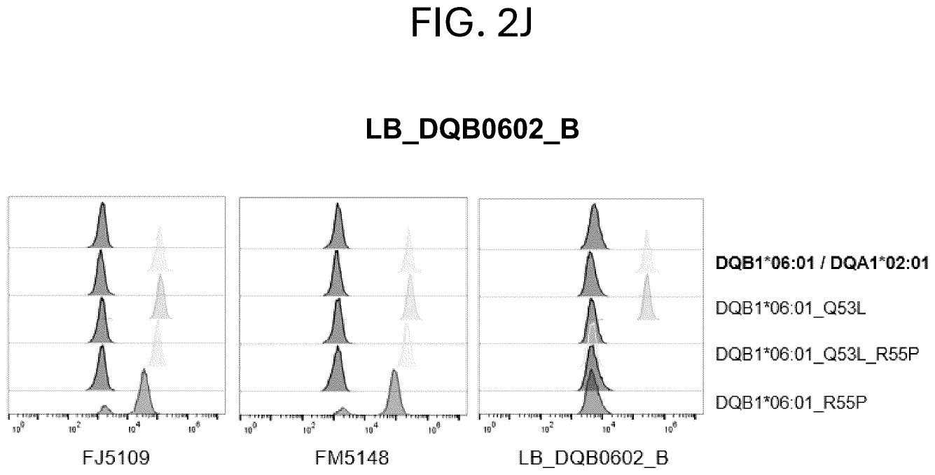

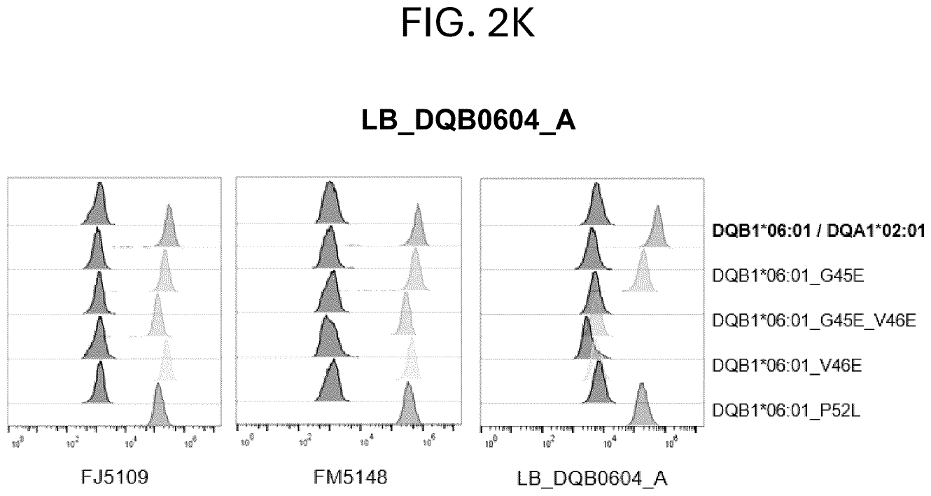

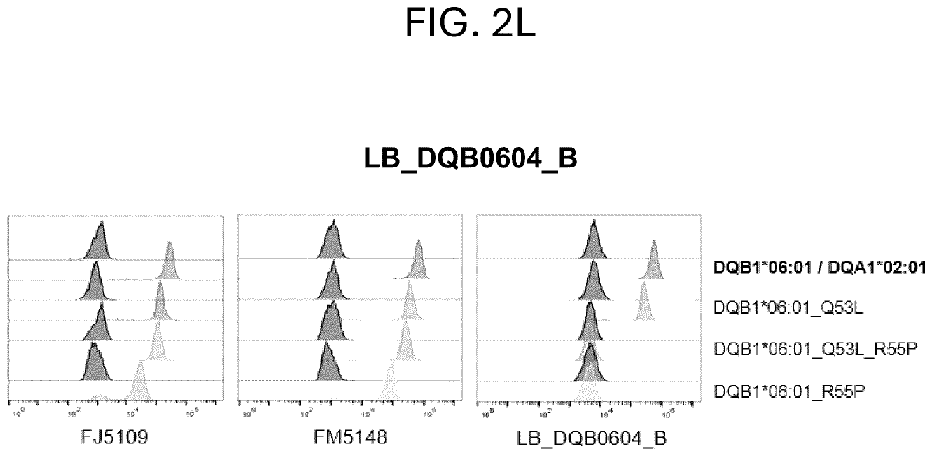

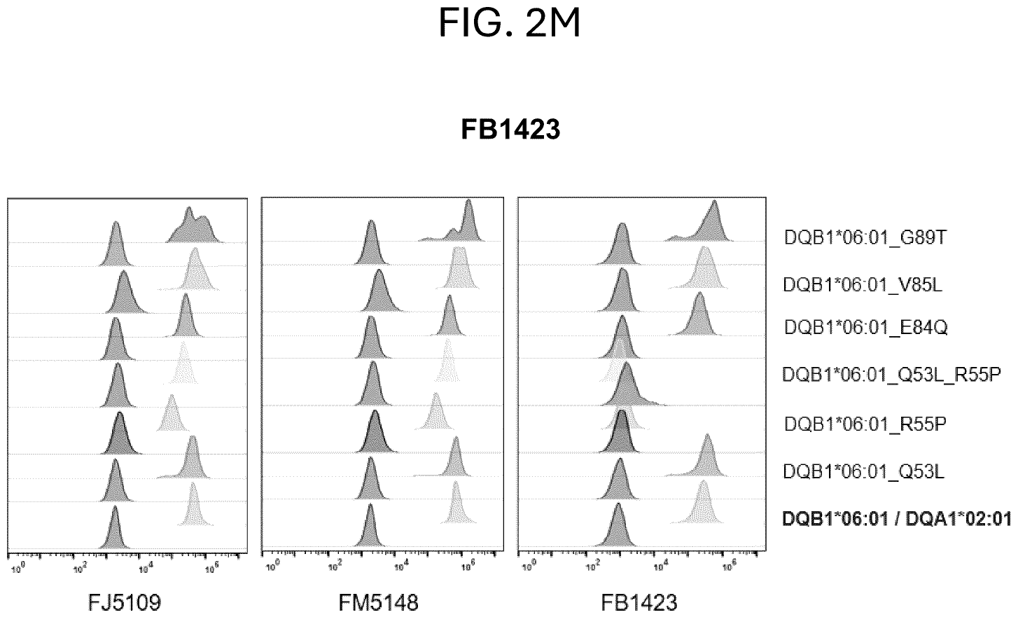

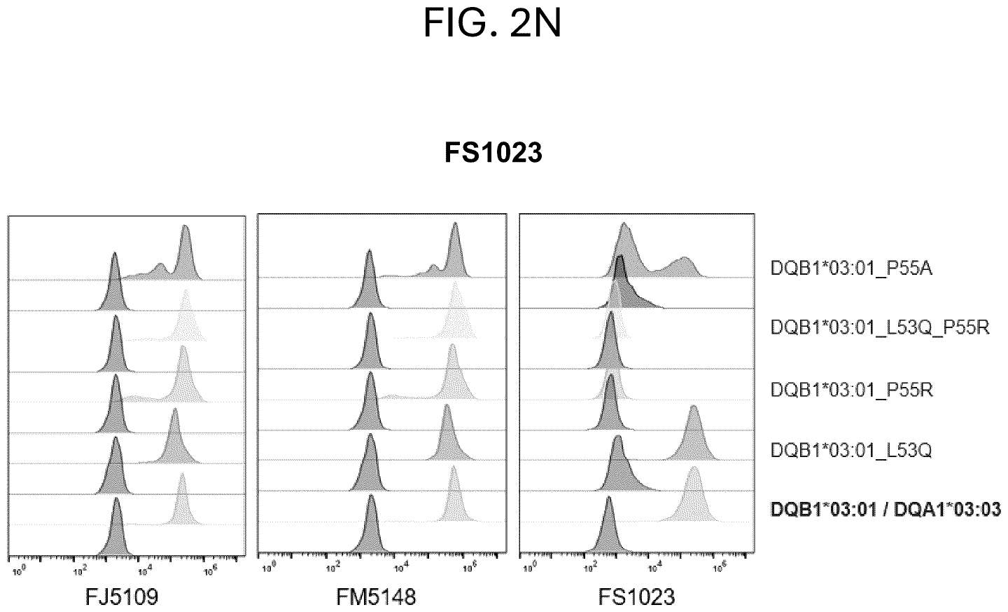

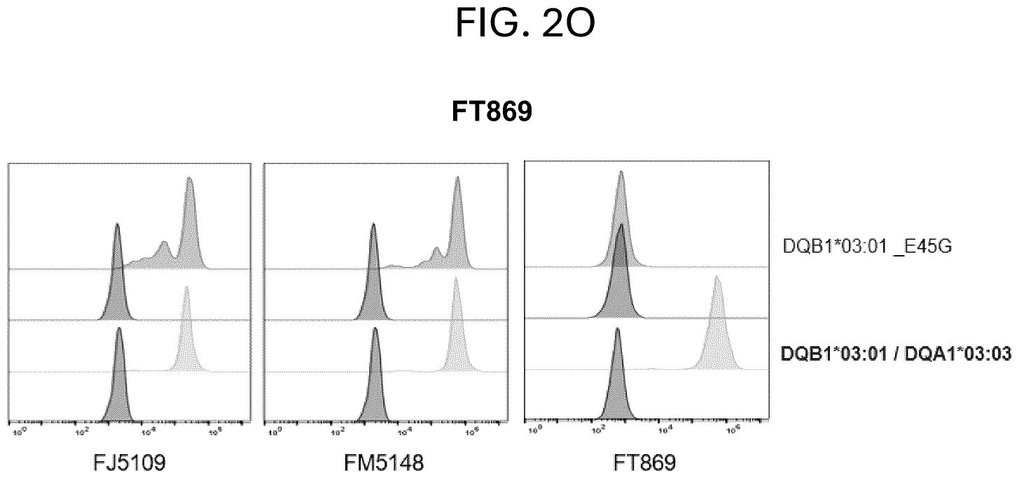

Step 3. The binding patterns of the sorted pools were compared between WT Ag and its EV (FIG. 2) to determine if particular ep(s) were indeed the ER as predicted (Table 3 and Table 4) or an AR contributing to affinity.

The term “complete LOF” on cells could be difficult to quantify due to the variable nature of cell assay: different expression levels among pools, heterogeneous expression levels within pool, variable non-HLA cell backgrounds, etc. Therefore, complete LOF generally refers to no appreciable positive population when compared to isotype negative Ab control. Partial LOF generally refers to that there are appreciable signals when compared to isotype negative control, but the signals are weaker than the WT. Because the WT and EV pools may have different expression levels to start with, an assignment of pLOF should take that into account and be conservative.

In some cases, change of individual eps may result in only pLOF, but in combination they may lead to a complete LOF. If they are neighboring residues, they can be collectively defined as the same ER.

In other cases, changes in multiple positions even in combination only lead to pLOF, then they are assigned as ancillary residue (AR).

ER is the essential but insufficient contact point(s) for a stable Ag-Ab interaction. If multiple ep changes lead to a complete LOF only in combination, but they are not neighboring residues, the LOF is probably due to the accumulative effect of the changes of multiple AR.

Step 4. Based on FIGS. 2A-2K and Table 5, the following summary can be drawn:

A. 77R is an ER and 70R an AR to LB_DQB0201_A.

B. 45E is an ER to LB_DQB0301_A. However, if the weak signals to DQ8 are considered, alternative ER is predicted.

C. For LB_DQB0303_A, 84Q is an ER when EV_Q84A is observed but an AR based on EV_Q84E. This residue-dependent effect manifests the complex nature of Ab-Ag interactions. Interestingly, when in combination, H81P_Q84E or Q84E_L87F, complete LOF is observed. Although H81 Å and L87F individually do not have appreciable effect, 81H and 87L are considered AR due to their contributions when combined with Q84E.

D. For LB_DQB0303_B, 52P is an ER based on cell assay. However, 52P53L together is predicted by AA3D and P52L may still have residual signals but too weak to be observed on cells due to lower Ag density on cells versus beads. The conclusion is best based on bead assay. 53L and 55P appear to be AR although individual contribution is not clearly demonstrated, in combination 53L55P certainly lead to pLOF.

E. 55P is an ER to LB_DQB0303_C.

F. For LB_DQB0402_A, 46V and 55R independently meets the definition of an ER based on cell assay. However, the residual signals on DQ2 beads if considered positive, 81H could be an ER as predicted. The status of 81H will be determined using EV on beads.

G. 77R is an ER and 70G an AR to LB_DQB0501_A.

H. 130R and 185T individually is an ER to LB_DQB0501_C based on cell assay. Whether 130R185T together is an ER as predicted awaits bead assay results.

I. 55R is an ER to LB_DQB0601_B.

J. 55R is an ER to LB_DQB0602_B.

K. 46V appears to be an ER to LB_DQ0604_A based on cell assay. However, 45G46V together is predicted by AA3D. So, whether V46E alone or G45E_V46E combined is an ER is pending on bead assay. Nonetheless, 45G at least meet the definition of an AR.

L. 55R is an ER to LB_DQB0604_B.

M. 55R is an ER to mouse mAb FB1423.

N. 55P is an ER to mouse mAb FS1023.

O. 45E is an ER to mouse mAb FT869.

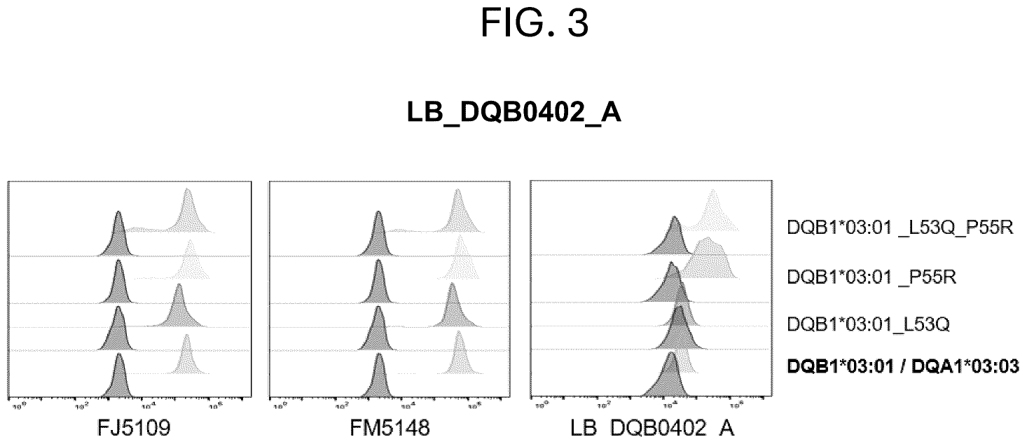

There are also situations where EV improve binding. If WT does not have appreciable binding but EV does, it is considered gain-of-function (GOF); if WT already has appreciable binding, the increased binding is considered partial GOF (pGOF). A complete GOF indicates where the ER is while a pGOF indicates a favorable change in AR. Because it is difficult to quantify signals from cells, GOF and pGOF are assigned herein only when obvious differences are observed from flow cytometry histograms. For example, in FIG. 3, DQB1*03:01_P55R and DQB1*03:01_L53Q_P55R lead to GOF to LB_DQB0402_A, indicating 55R is an ER. DQB1*03:01_L53Q alone does not lead to appreciable GOF. Whether L53Q_P55R binds better than P55R alone to LB_DQB0402_A remains to be confirmed on beads. If so, 53L can be considered an AR.

Interest is focused on variant positions herein in part because this is how patient and donor are matched by sequence. It is theorized that these mismatched eps on Ag could determine specificity and contribute to affinity of Ab. Because there are multiple contact points between epitope and paratope, conserved residues neighboring an ER at variant position(s) could affect Ag-Ab interactions. A set of designed EV was made at positions immediately neighboring an ER, conserved or not, to investigate their contribution to binding. If conserved, the residue is replaced with an alanine; if variant, the residue is switched to the one in a non-binding allele.

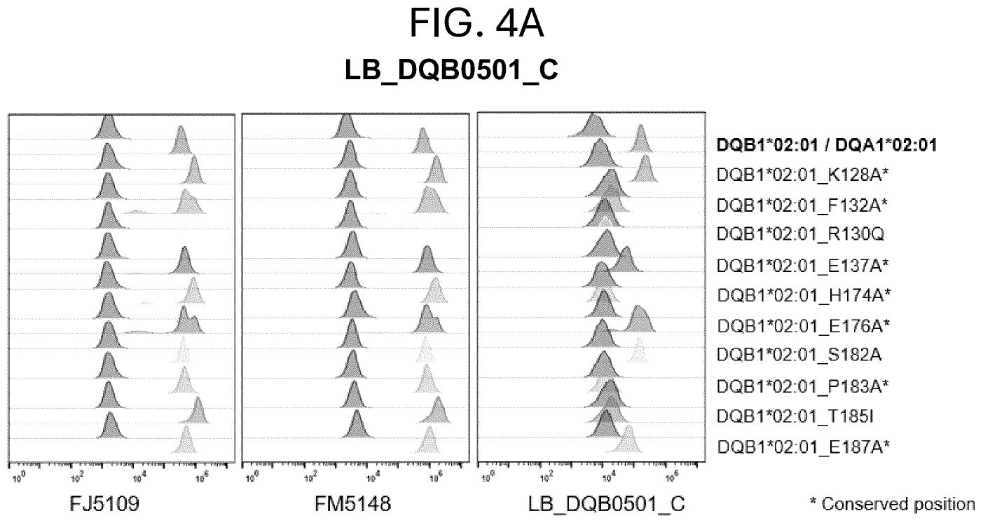

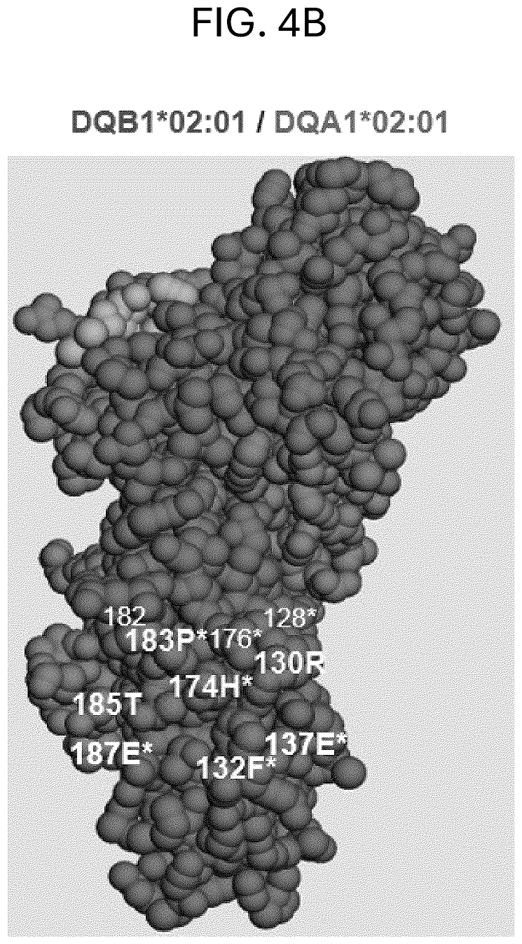

Using LB_DQB0501_C as an example (FIGS. 4A and 4B) where R130Q or T185I leads to complete LOF affirming their ER status (FIG. 2H). Interestingly, a single change at several neighboring conserved residues also lead to complete LOF (F132A, H174A or P183A) and pLOF (E137A or E187A) while other positions that might have subtle effect or no effect (K128A or E176A). The effect of a change in variant position S182A is also uncertain. These observations were further evaluated on beads. Apparently, ER definition can extend to a conserved residue also, suggesting the cluster nature of an ER despite there may be only 1 variant position in the cluster.

In FIG. 2C, 84Q is mapped to be a residue-dependent ER (Q84A vs Q84E) and 81H as a residue-dependent AR (H81A vs H81P). The synergistic effect of combining 2 individual changes is also shown (e.g. H81P_Q84E and Q84E_L87F), again suggesting the cluster nature of an ER. This also demonstrates the value to adding DQB1*02:07, the only common DQB allele carrying 81P, to the DQ Epitope panel.

Multiple ep/ep patterns are predicted to be ER for LB_DQ0303_B, but 140T and 182N are ruled out by EV T140A and N182S respectively which retains binding (FIG. 2D). Only EV containing P52L show complete LOF. However, because 52P53L was predicted by AA3D and AA, whether P52L alone can lead to cLOF remained to be seen on beads. The observation was that eps 53Land 55P were implicated as AR individually or in combination.

As described for LB_DQB0402_A (FIG. 3), a change at an ER position could have a GOF effect on a mAb and LOF to another. Although GOF is much harder to achieve than LOF because all surrounding ER and AR need to work together to secure an appreciable affinity.

### Use of EV Coated on SAB for Epitope Mapping

HLA Ag expressed on cell surface have passed through multiple cellular checkpoints during its translocation to plasma membrane and therefore are considered to possess its native (WT) conformations. Early detection on cell surface by flow cytometry skips the lengthy and costly production process including Ag purification and bead coating that may introduce some undesirable artifacts in SAB assay. However, a cell-based assay has its own inherent uncertainties: heterogeneous expression within pool, various expression among pools, other cell surface proteins interacting with fluorescence-labeled secondary Ab and/or other non-HLA Ab in a sample, etc. Therefore, relevant positive and negative controls weigh in when interpreting binding of test Ab. Further, Ag density on cell surface generally is much less than what is coated on bead, if the test Ab affinity is below a certain threshold, it may still be observed on bead due to avidity but not on cell.

The ER predictions for the 12 human mAb by AA3D and AA based on bead assay have been verified with EV in cell assay as described herein. However, there were discrepancies as to whether a single or a combined change leading to cLOF need to be determined using beads because of their higher sensitivities.

The cell culture expansion, cell pellet harvest, Ag purification, bead coating, Ag and bead quality control have been described in Example 2.

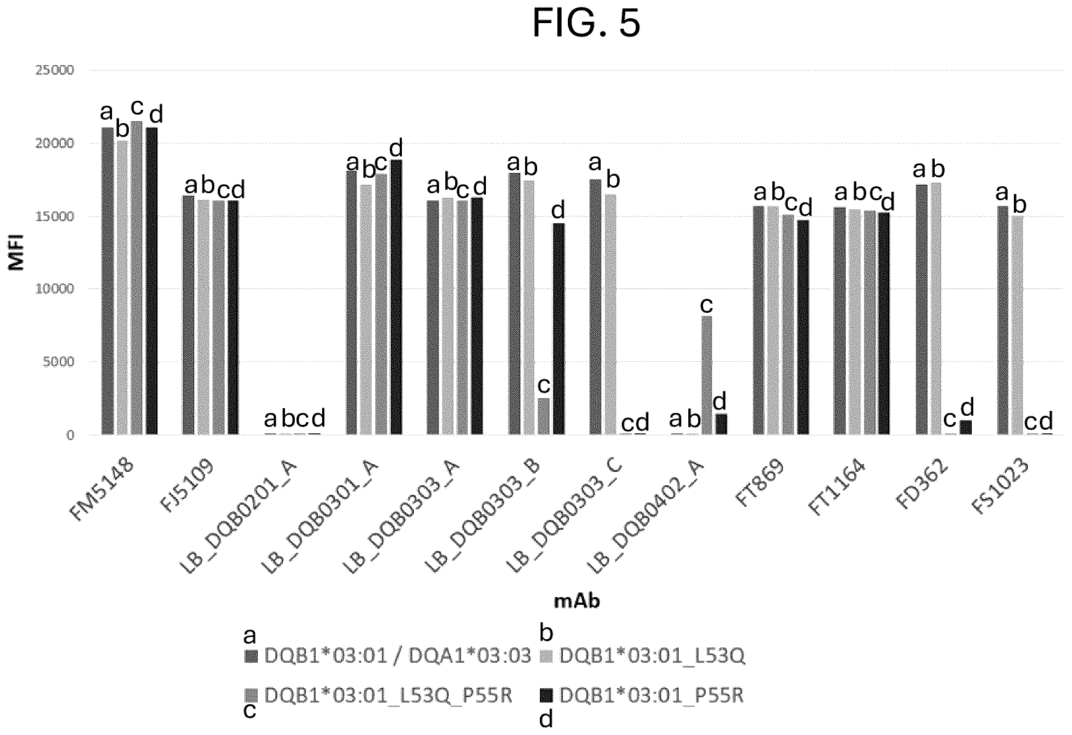

Single Ag beads (SAB) of DQB1*03:01/DQA1*03:03, DQB1*03:01_L53Q/DQA1*03:03, DQB1*03:01_L53Q_P55R/DQA1*03:03 and DQB1*03:01_P55R/DQA1*03:03 were generated and tested in Luminex assay with a set of HLA specific mAb (FIG. 5). The following were observed:

A. The positive controls pan HLA-DQ mAb FM5148 and pan HLA Class II mAb FJ5109 showed no binding affinity to any of the EV. The negative control LB_DQB0201_A has no binding to DQB1*03:01/DQA1*03:03 and its EV.

B. The EV also have no effect on the binding of LB_DQB0301_A, LB_DQB0303_A, FT869 and FT1164. The ER of LB_DQB0301_A, LB_DQB0303_A and FT869 has been mapped to 45E, 84Q and 45E respectively with cells, apparently not affected by L53Q and P55R changes.

C. L53Q_P55R leads to drastic reduction of binding to LB_DQB0303_B while L53Q or P55R alone has no or only slight effect on binding. Although the effect of P55R in cell assay is unclear, it is fair to say there is a difference on beads. Ep 52P is an ER for LB_DQB0303_B based on cell assay; therefore, it is not surprising its neighboring residues have AR effect.

D. Both L53Q_P55R and P55R but not L53Q alone lead to cLOF binding to LB_DQB0303_C indicating 55P is an ER consistent with cell assay.

E. FS1023 has the same specificity profile as LB_DQB0303_C for which 55P is the verified ER, so it is consistent that both to L53Q_P55R and P55R variants lead to cLOF.

F. The specificity profile of FD362 differs from FS1023 and LB_DQB0303_C in that FD362 also recognizes DQB1*03:16 which has 55Q instead of 55P indicating some tolerance at this position. Additionally, based on AA3D prediction, 55P56P could be an ER, explaining why there is still some residual binding of FD362 to P55R.

G. Although LB_DQB0402_A does not bind to DQB1*03:01/DQA1*03:03 WT and L53Q EV, it selectively binds to L53Q_P55R and P55R EV indicating a GOF switch. Higher degree of GOF is obtained for L53Q_P55R than for P55R on beads but not as clearcut on cells (FIG. 3), indicating the higher sensitivity of use of a SAB bead assay (e.g., panel).

These results made using a cell assay and further differentiate the degree of binding, in turn demonstrating the utility of the approach of using EV for ER verification.

### Example 5: Enhancement of Epitope Panel Design and Prediction with HLA Fusion™ AA and AA3D Software Modules

There are several reasons as to why an alternative epitope analysis software is needed instead of using the existing HLAMatchmaker™ (MM) module in HLA Fusion™. Although HLAMatchmaker™ is well established in the field of transplant diagnostics, the definition of each eplet, which relies on human curation, has deficiencies. Several reasons are set out below.

A. Many HLA structures are based on molecular modeling that may not be precise enough to predict local conformations at a radius of <3.5 Å that define an eplet or functional epitope.

B. The number of eplets continues to increase as more and more alleles are identified and some eplets may not have been named and/or assigned to certain alleles.

C. Certain variant positions distant from the alpha helical structures are not considered for eplet assignment.

D. The definitions of eplets are further complicated not only by intra-locus but also inter-locus cross-reactivities.

E. For some eplets, the constituent amino acids are too far apart to fit the definition of a functional epitope.

F. In multiple instances (Example 3), AA and AA3D modules provide more complete coverage that accounts for all positive and negative signals than MM module does.

G. Although eplets are further tiered as Ab verified vs unverified, the verification methods and criteria are not standardized especially for pAb, and the number and types of mAb are limited for eplet verification.

For any of these reasons, instead of pre-assigning potential participating amino acids to an eplet based on perceived sequence and structural association derived from molecular modeling, the methods of this disclosure bypass the eplet system and directly consider all residues (eps) individually and as ep patterns. The disclosed methodology utilizes the concept that an Ab can directly identify which ep(s) or ep pattern(s) could be the ER based on SAB specificity profile, instead of looking for a predefined eplet in the registry.

The AA module as disclosed herein identifies all potential contact points whether single or in combination within a user-defined consecutive linear amino acid range. The linear approach is suitable for amino acids in close vicinity linearly and spatially, and indeed, as such, many ER may be predicted by the methods described herein.

As described in Example 4, EV provides valuable information in identifying both ER and AR describing the interactions between Ag and Ab. Without EV, the ER may not be resolved to satisfaction for many mAb or pAb. As more ER are being predicted with increasing number of available human mAb and pAB, more EV is expected to be needed to confirm the predictions.

Some ep or ep patterns, if far apart from each other, or on the opposite side of the Ag, may be engineered on the same Ag because the presence of one altered ep or ep pattern is not expected to significantly interfere with the effect caused by another altered ep or ep pattern if they cannot constitute the same epitope of an Ab. In this manner, the number of EV on the panel can be reduced.

### Example 6: Epitope Analysis of Polyclonal Antibodies Using HLA-DQ Panel

Because of the polyclonal Ab (pAb) nature of human serum samples, the binding signals to each Ag could come from more than 1 epitope/paratope interactions, so the imputation logic used for predicting ER does not completely apply to pAb. Nonetheless, depending on the complexity of the pAb mix, an epitope panel described herein provides direction as to where to look for ER. For example, even if the pAb sample contains both DR and DQ mAb, its DR or DQ specificity can be analyzed separately using HLA Fusion™ AA and AA3D modules to partition DR vs DQ specific mAb. If there are multiple DQ mAb targeting different sets of DQ Ag and/or the same DQ Ag, it is difficult to deconvolute without using an adsorption-elution (Ads-Elu) protocol. On the other hand, the ER that are recognized, allow for signals between WT and its EV to provide binding data for a mAb and pAb in a mixed sample.

Because multiple mAb can bind to the same Ag but recognize distinctly different or overlapping epitopes of similar or different affinities, it was realized that it is better to compare WT Ag and EV at a sub-saturating Ab concentration by a serial dilution to differentiate Ab of different affinities where lower binding affinity occurs. The concept is different from epitope mapping for mAb where the general approach is to increase Ab concentration to confirm weaker Ab/HLA binding affinity signals so they can be taken into considerations.

Another difference between epitope mapping mAb and pAb is that the ER epitope is supposed to be present in all binders for mAb while the ER of one mAb may be present only in part of the overall binders in the pAb mix. To segregate pAb signals for individual ER imputation, AA3D module also allows manual assignment of positive and negative Ag based on experimental observations and known mAb specificity profiles. Potential ER can be predicted using this segregated approach if the individual ER predictions are compatible with the overall signals.

Although there are ways described above to gain insights to where the ER might be, for highly sensitized patients, it is recommended to apply Ads-Elu protocol with known Ag on cells or solid phase to deconvolute the signals.

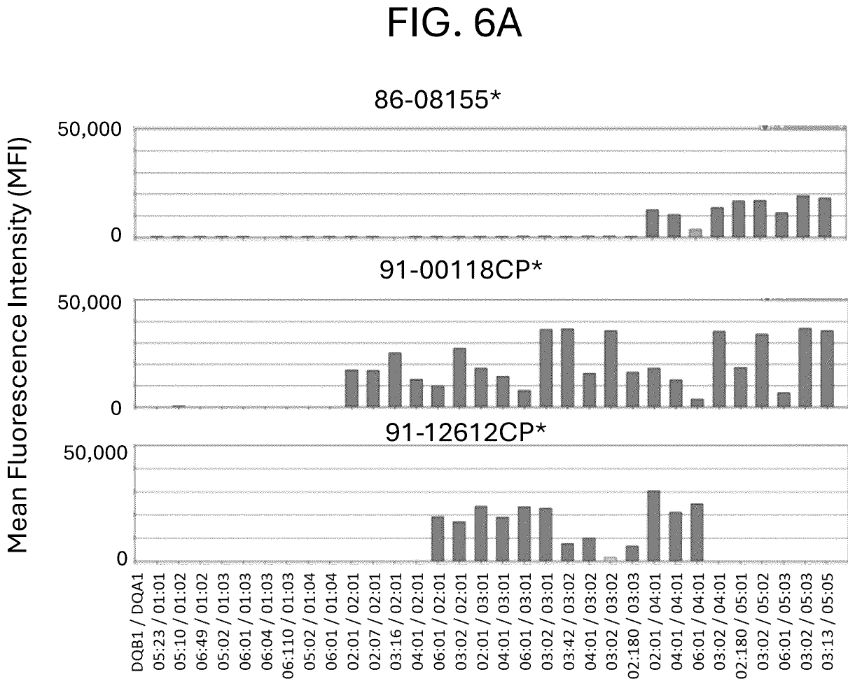

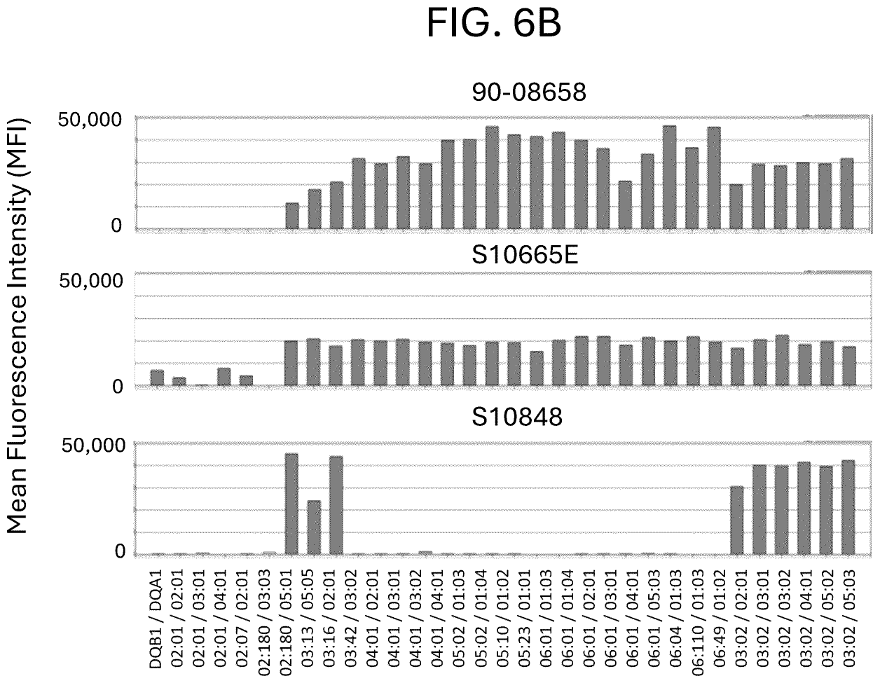

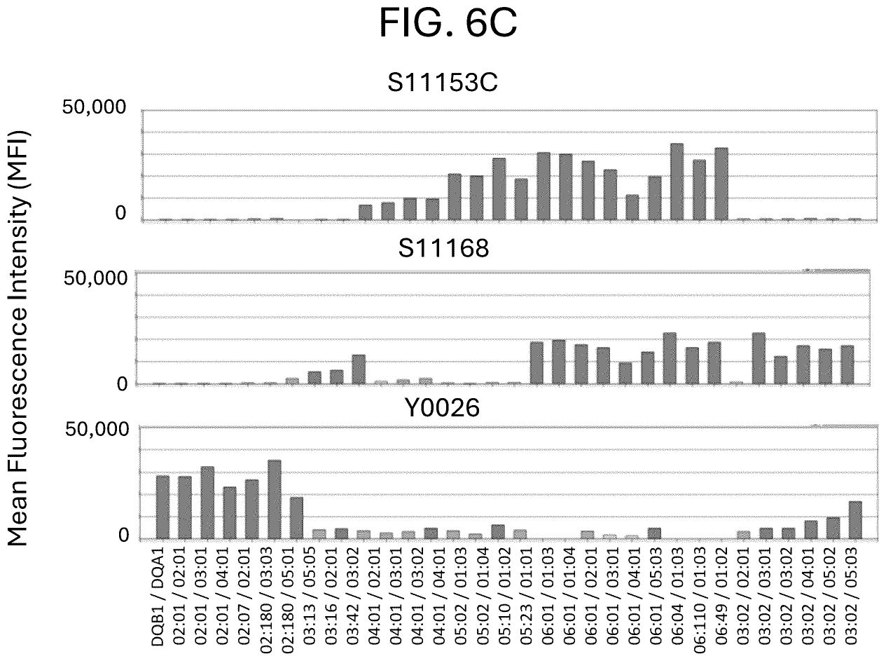

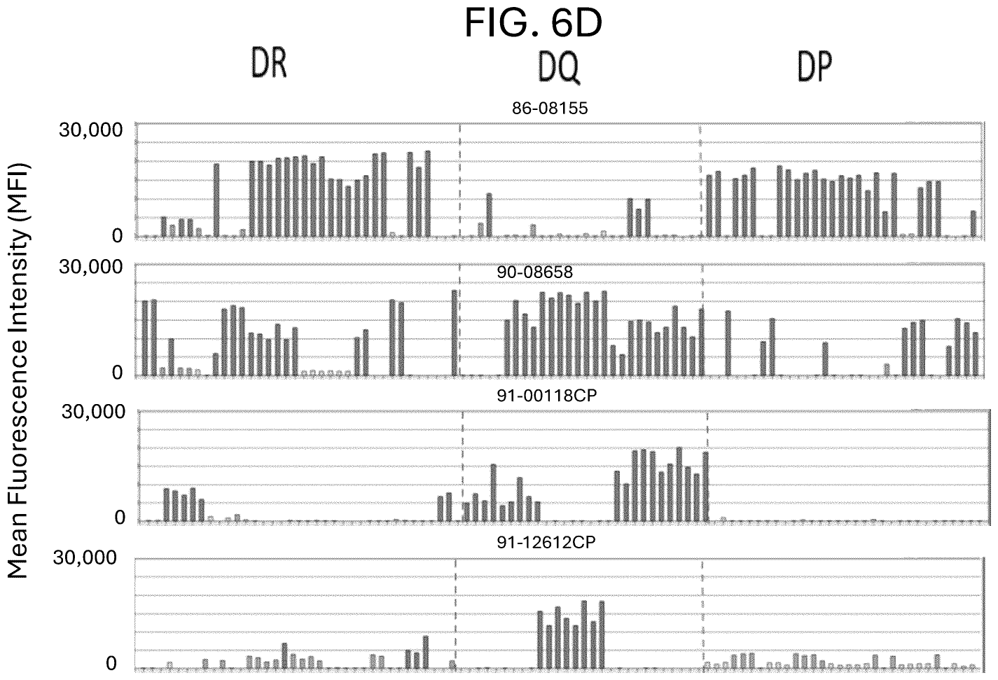

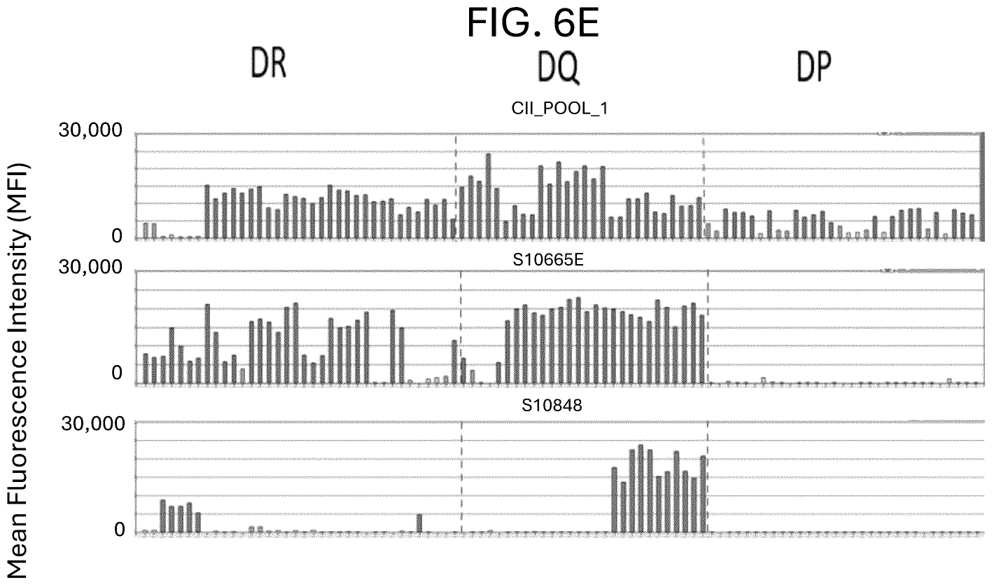

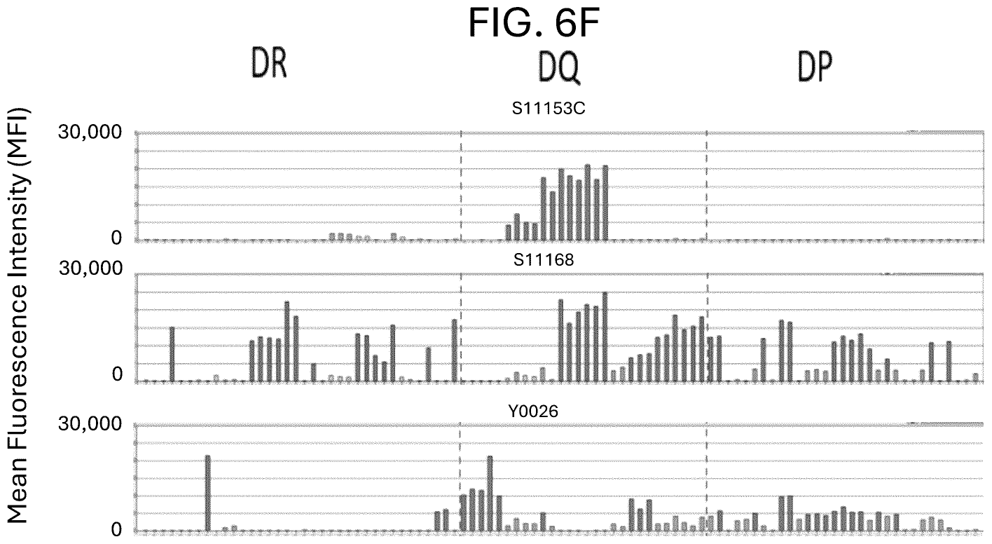

Human pAb serum samples (Table 8) were run on the DQ epitope panel, their specificity profiles with DQ epitope panel are shown in FIGS. 6A-6C, and ER predictions are listed in Table 9.

TABLE 8
Human serum samples tested on HLA-DQ panel for epitope analysis
Human pAb

86-08155
90-08658
91-00118CP
91-12612CP
S10848
S11153C
Y0026

TABLE 9
Epitope analysis and ER prediction of pAb using HLA Fusion ™ AA and
AA3D modules based on specificity profile determined by HLA-DQ panel.

| | | Number of | AA3D Module | | | | AA Module |
|---|---|---|---|---|---|---|---|
| pAb | Locus | Positive Beads | Ep | Occurrence | Accessible | Inaccessible | Ep |
| 86-08155 | DQA1 | 9 | 40G | 9 | 40 | | 40G |
| | | | 40G41R | 9 | 40, 41 | | 40G41R |
| | | | 47C | 9 | | 47 | 47C |
| | | | 47C48L | 9 | | 47, 48 | 47C48L |
| | | | 50V | 9 | 50 | | 50V |
| | | | 50V51L | 9 | 50 | 51 | 50V51L |
| | | | 51L52R | 9 | 52 | 51 | 51L52R |
| | | | 52R53Q | 9 | 52, 53 | | 52R53Q |
| | | | 53Q54F | 9 | 53, 54 | | 53Q54F |
| 90-08658 | DQB1 | 26 | 28T | 26 | | 28 | 28T |
| | | | 46V47Y | 26 | 46 | 47 | 46V |
| | | | 47Y | 26 | | 47 | 47Y |
| 91-00118CP | DQA1 | 23 | 11Y | 23 | | 11 | 11Y |
| | | | 18S | 23 | 18 | | 18S |
| | | | 45V | 23 | | 45 | 45V |
| | | | 48L | 23 | | 48 | 48L |
| | | | | | | | 55R |
| | | | 61F | 23 | 61 | | 61F |
| | | | 64T | 23 | 64 | | 64T |
| | | | 66I | 23 | | 66 | 66I |
| | | | 80S | 23 | | 80 | 80S |
| | | | 129H130S | 23 | 129, 130 | | |
| 91-12612CP | DQB1 | 13 | 52P53Q | 13 | 52, 53 | | 52P53Q |
| | | | 53Q | 13 | 53 | | 53Q |
| | | | 55R56P | 13 | 55, 56 | | 55R56P |
| | | | 54E | 13 | 84 | | 84E |
| | | | 54E85V | 13 | 84 | 85 | 84E85V |
| | | | 59G | 13 | | 89 | 89G |
| | | | 59G90I | 13 | | 89, 90 | 89G90I |
| | | | 90I | 13 | | 90 | 90I |
| | DQA1 | 9 | 11C | 9 | | 11 | 11C |
| | | | 18F | 9 | 18 | | 18F |
| | | | 45A | 9 | | 45 | 45A |
| | | | 47R | 9 | | 47 | 47R |
| | | | 47R48W | 9 | | 47, 48 | 47R48W |
| | | | 48W | 9 | | 48 | 48W |
| | | | 50E | 9 | 50 | | 50E |
| | | | 50E51F | 9 | 50 | 51 | 50E51F |
| | | | 51F52S | 9 | 52 | 51 | 51F52S |
| | | | 52S53K | 9 | 52, 53 | | 52S53K |
| | | | 53K54F | 9 | 53, 54 | | 53K54F |
| | | | 54F55G | 9 | 54, 55 | | 54F55G |
| | | | 55G56G | 9 | 55, 56 | | 55G56G |
| | | | 56G | 9 | 56 | | 56G |
| | | | 61G | 9 | 61 | | 61G |
| | | | 64R | 9 | 64 | | 64R |
| | | | 66M | 9 | | 66 | 66M |
| | | | 69A | 9 | | 69 | 69A |
| | | | 75I76M | 9 | 75 | 76 | 75I76M |
| | | | 76M | 9 | | 76 | 76M |
| | | | 80Y | 9 | | 80 | 80Y |
| | | | | | | | 175Q |
| S10665E | | 30 | | | | | |
| | DQB | 26 | 28T | 26 | | 28 | 28T |
| | | | 46V | 26 | 46 | | 46V |
| | | | 47Y | 26 | | 47 | 47Y |
| | | | 52P | 26 | 52 | | 52P |
| S10848 | DQB1 | 9 | 30Y9Y | 9 | | 39, 9 | |
| | | | 38A53L56P | 9 | 53, 56 | 38 | |
| | | | 52P53L56P | 9 | 52, 53, 56 | | 75L77T81H84Q |
| | | | 75L84Q | 9 | 75 ,84 | | |
| | | 8 | 55P | 8 | 55 | | 55P |
| | | | 55P56P | 8 | 55, 56 | | 55P56P |
| S11153C | DQB1 | 17 | 55R | 17 | 55 | | 55R |
| | | 13 | 52P53Q | 13 | | | 52P53Q |
| | | | 53Q | 13 | | | 53Q |
| | | | 55R56P | 13 | | | 55R56P |
| | | | 81H84E | 13 | | | 81H84E |
| | | | 84E | 13 | | | 84E |
| | | | 84E85V | 13 | | | 84E85V |
| | | | 89G | 13 | | | 89G |
| | | | 89G90I | 13 | | | 89G90I |
| | | | 90I | 13 | | | 90I |
| S11168 | | 17+ | | | | | |
| Y0026 | | 16+ | | | | | |

| | AA Module | MM Module | | |
|---|---|---|---|---|
| pAb | Occurrence | Eplet | Residue | Occurrence |
| 86-08155 | 9 | 40GR | 40G47C50V51L | 9 |
| | 9 | | | |
| | 9 | | | |
| | 9 | | | |
| | 9 | | | |
| | 9 | | | |
| | 9 | | | |
| | 9 | | | |
| | 9 | | | |
| 90-08658 | 26 | | | |
| | 26 | 46VY | 46V52P28T | 28 |
| | 26 | | | |
| 91-00118CP | 23 | | | |
| | 23 | | | |
| | 23 | | | |
| | 23 | | | |
| | 23 | qa55R | 55R | 23 |
| | 23 | 61FT | 61F64T55R | 23 |
| | 23 | | | |
| | 23 | | | |
| | 23 | | | |
| 91-12612CP | 13 | 52PQ | 53Q89G90I | 13 |
| | 13 | | | |
| | 13 | | | |
| | 13 | | | |
| | 13 | | | |
| | 13 | | | |
| | 13 | | | |
| | 13 | | | |
| | 9 | | | |
| | 9 | | | |
| | 9 | | | |
| | 9 | | | |
| | 9 | | | |
| | 9 | | | |
| | 9 | | | |
| | 9 | | | |
| | 9 | 52SK | 52S53K11C18F45A54R66M69A80Y | 9 |
| | 9 | | | |
| | 9 | | | |
| | 9 | | | |
| | 9 | | | |
| | 9 | | | |
| | 9 | | | |
| | 9 | | | |
| | 9 | | | |
| | 9 | | | |
| | 9 | | | |
| | 9 | | | |
| | 9 | | | |
| | 9 | | | |
| S10665E | | | | |
| | 26 | 46VY | 46V52P28T | 26 |
| | 26 | | | |
| | 26 | | | |
| | 26 | | | |
| S10848 | | | | |
| | 8 | 55PP | 55P56P | 8 |
| | 8 | | | |
| S11153C | 17 | qb55R | 55R | 17 |
| | 13 | 52PQ | 53Q89G90I | 13 |
| | 13 | | | |
| | 13 | | | |
| | 13 | | | |
| | 13 | | | |
| | 13 | | | |
| | 13 | | | |
| | 13 | | | |
| | 13 | | | |
| S11168 | | | | |
| Y0026 | | | | |

Some pAb samples (86-08155, 90-08658, 91-00118CP, and S10848) resemble mAb profiles where potential ER are predicted with all positive and negative beads accounted for, but for others (91-12612CP, S10665E and S11153C), the ER candidates are predicted only after artificial reassignments of the positive and negative beads, and for S11168 and Y0026, no ER can be predicted even with artificial assignments. This points out the challenging nature of pAb samples and the advantages of fractionating the signals using Ads-Elu protocol.

A. For 86-08155, it is DQA4/5/6 positive but DQA1/2/3 negative, behaving like a mAb. Two clusters are predicted to be ER that need EV to deconvolute. Both are in close enough range to be recognized by the same mAb.

B. For 90-08658, it is DQ3/4/5/6/8 positive but DQ2 negative, behaving like a mAb. 46V could account for an ER that needs EV to confirm.

C. For 91-00118CP, it is DQA2/3/4/5 positive but DQA1 negative. Although ER predictions can be derived if it is treated like a mAb, the significant signal variations within each DQA subgroup cast doubts about its clonality, or the affinities appear to be influenced by beta subunits. Or it could be DQ2/3/4/8 positive but DQ5 negative with DQ6 signals alpha dependent. Ads-Elu may help to deconvolute.

D. For 91-12612CP, it is DQ5/6 positive but DQ2/3/4/8 negative. However, the signals are stronger if paired with DQA1 than DQA2/3/4/5, particularly much weaker when DQA4 is paired. It remains to be seen if this is a mAb behavior. Ads-Elu may help to deconvolute.

E. For S10665E, there are apparently 30 positives, but ER can be only predicted for 26 positives suggesting more than 1 mAb is in the mix.

For S10848, its specificity profile is same as FD362 which is a mAb with DQ3/8 specificity. If it is a mAb, there are 9 positives and multiple potential ER are predicted.

For S11153C, it appears to fit a mAb with DQ4/5/6 specificities. If so, single ER prediction is made for 17 positives. If not a mAb and DQ4 is separated from DQ5/6 specificities, multiple ER are predicted for 13 positives.

For S11168, there are more than 17 positives, but no ER predictions can be made suggesting a pAb nature.

Same with Y0026, no ER predictions can be made.

The HLA Class II specificity profiles of these pAb are also determined by the existing One Lambda SAB product LS2A01 (FIGS. 6D-6F). Many of them have specificities to DR and DP Ag as well. Despite the complex reactivity profiles, the DQ epitope panel draws the focus to DQ Ag only, in a way filtering out non-DQ specific signals. Most human mAb against HLA Class II Ag is usually locus specific and its cross-locus reactivity is likely fortuitous due to some level of conformational similarity. If a mAb has inter-locus cross-specificity, multi-loci specificities can be combined for ER imputation. However, if uncertain whether the signals are from one or more than one mAb, it is best to start with locus specific analysis. The DQ epitope panel designs described here should be sufficient in capturing the potential ER even without knowing the sample's reactivities to other loci.

Such locus specific and/or combined loci analyses can also be achieved through software design; that is, all or part of SAB data can be collected simultaneously or separately, and then analyzed in part or in whole to cover all likely scenarios for the best predictions.

The HLA-DQ ER epitopes predicted for these pAb by HLA Fusion™ AA and AA3D modules are located at similar positions as the ones predicted for mAb, so the same EV for mapping ER for mAb can be tried for mapping pAb too. However, the lack of LOF observations does not rule out the possibility because the LOF binding could be masked by another binder that recognizes another ER on the same Ag. It is advisable to include Ads-Elu protocol when working with a pAb serum sample.

### Use of Ads-Elu Protocols to Deconvolute pAb Specificities and Map ER Epitopes

If the specificity profiles of a pAb sample to WT and its corresponding EV are difficult to discern, several Elu-Ads strategies can be applied: 1) using native cells expressing Ag of interest, 2) using recombinant cells expressing single Ag of interest, and 3) using purified Ag immobilized on a solid phase. Each approach has its own scientific validity and limitations. Purified Ag immobilized on magnetic beads such as One Lambda MagSort is often the choice due to the following advantages: 1) ease of us, 2) superior sensitivity and 3) less cellular background. On the other hand, Ag expressed on cells, especially native cells, present native conformations with a higher degree of confidence and reflect better the Ag-Ab recognition in vivo. To minimize complexity, only single Ag are used for MagSort beads. The cell and bead numbers, serum dilution factors, and binding and elution conditions are optimized to achieve the detection threshold of Ads-Elu that provides sufficiently differentiating Luminex SAB signals from the post-adsorption and post-elution fractions.

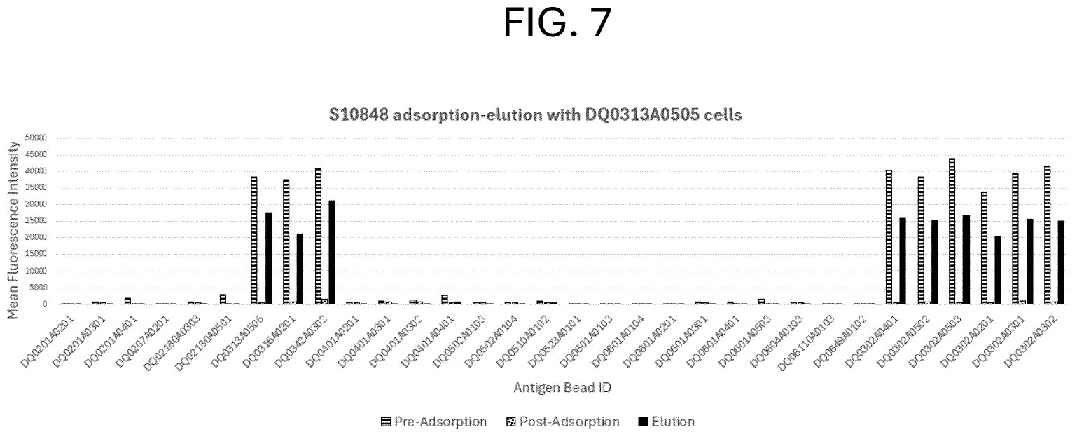

For serum sample S10848, it could be a mAb with 9 positives, but it could also be another mAb with 8 positives, so the sample was adsorbed onto recombinant cells expressing single antigen DQB1*03:13/DQA1*05:05. Its pre-adsorption, post-adsorption and elution specificity profiles are compared. S10848 adsorbs to and elutes from DQB1*03:13/DQA1*05:05 well. Because the specificity profile of the eluent does not change, suggesting it is likely a mAb (FIG. 7).

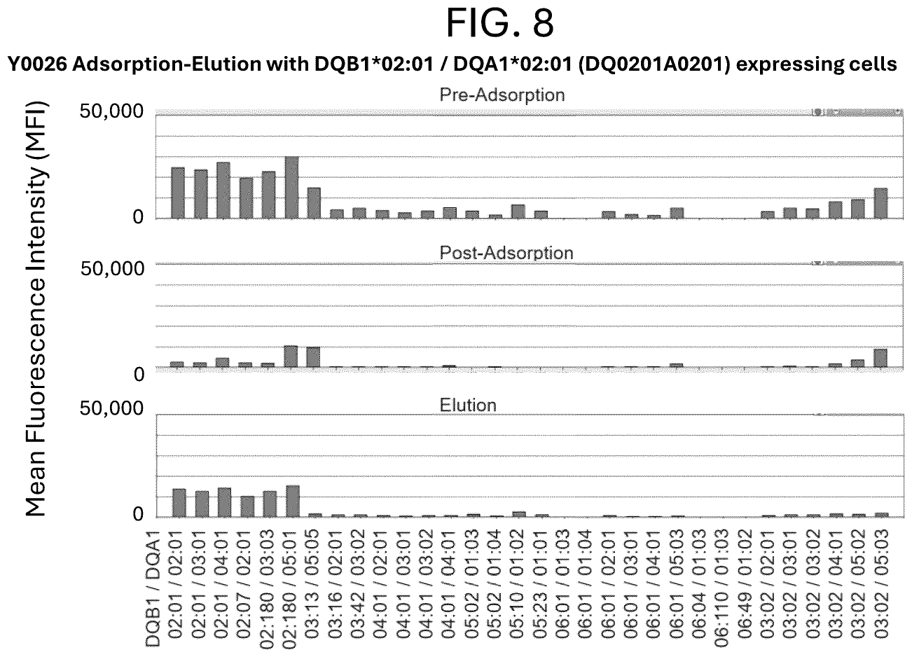

On the other hand, Y0026 only selectively binds to DQB1*02:01/DQA1*02:01. Its specificities to DQB1*03:13/DQA1*05:05 and other non-DQ2 Ag appear to be lost (FIG. 8). By analyzing its DQ2 specificities in the eluant, multiple ER are predicted (Table 10) suggesting Y0026 contains at least one DQ2 specific mAb.

TABLE 10
ER predictions of human serum samples post Ads-Elu with
single antigen expressed on cells or coated on beads.

| | Adsorption- | | | Number of | AA3D Module | | | |
|---|---|---|---|---|---|---|---|---|
| pAb | Elution | Surface | Locus | Positive | Ep | Occurrence | Accessible | Inaccessible |
| Y0026 | DQ0201A0201 | Cell | DQE | 6 | 28S | 6 | | 28 |
| | | | | | 30S | 6 | | 30 |
| | | | | | 37I | 6 | | 37 |
| | | | | | 37I38V | 6 | | 37, 38 |
| | | | | | 45G46E | 6 | 45, 46 | |
| | | | | | 46E47F | 6 | 46 | 47 |
| | | | | | 47F | 6 | | 47 |
| | | | | | 51T52L | 6 | 51, 52 | |
| | | | | | 52L53L | 6 | 52, 53 | |
| | | | | | 55L | 6 | 55 | |
| | | | | | 55L56P | 6 | 55, 56 | |
| | | | | | 70R71K | 6 | 70, 71 | |
| | | | | | 71K | 6 | 71 | |
| | | | | | 74A | 6 | | 74 |
| | | | | | 74A75V | 6 | 75 | 74 |
| CII_POOL_1 | DQ0301A0201 | Bead | DQA | 23 | 11Y | 23 | | 11 |
| | | | | | 18S | 23 | 18 | |
| | | | | | 45V | 23 | | 45 |
| | | | | | 48L | 23 | | 48 |
| | | | | | 61F | 23 | 61 | |
| | | | | | 64T | 23 | 64 | |
| | | | | | 66I | 23 | | 66 |
| | | | | | 80S | 23 | | 80 |
| | | | | | 129H130S | 23 | 129, 130 | |
| | DQ0501A0101 | Bead | DQB | 29 | 45G | 29 | 45 | |
| | | | | 19 | 140A | 19 | | 140 |
| | DQ0603A0103 | Bead | DQB | 23 | 45G46V | 23 | 45, 46 | |
| | | | | 13 | 52P53Q | 13 | 52, 53 | |
| | | | | | 53Q | 13 | 53 | |
| | | | | | 55R56F | 13 | 55, 56 | |
| | | | | | 84E | 13 | 84 | |
| | | | | | 84E85V | 13 | 84 | 85 |
| | | | | | 89O | 13 | | 89 |
| | | | | | 89O90I | 13 | | 89, 90 |
| | | | | | 90I | 13 | | 90 |

| | AA Module | | MM Module | | |
|---|---|---|---|---|---|
| pAb | Ep | Occurrence | Eplet | Residue | Occurrence |
| Y0026 | 28S | 6 | 52LL | 62L55L28S30S37I | 6 |
| | 30S | 6 | | | |
| | 37I | 6 | | | |
| | 46E | 6 | | | |
| | 47F | | | | |
| | 52L | 6 | | | |
| | 55L | 6 | | | |
| | 71K | 6 | | | |
| | 74A | 6 | | | |
| CII_POOL_1 | 11Y | 23 | 61FT | 61F54T55R | 23 |
| | 18S | 23 | qa55R | 55R | 23 |
| | 45V | 23 | | | |
| | 48L | 23 | | | |
| | 61F | 23 | | | |
| | 64T | 23 | | | |
| | 66I | 23 | | | |
| | 80S | 23 | | | |
| | 129H130S | 23 | | | |
| | 45G | 29 | 45G | 45G | 29 |
| | 140A | 19 | | | |
| | 45G46V | 23 | 45GV | 45G46V | 23 |
| | 53Q | 13 | 62PQ | 53Q89O90I | 13 |
| | 84E | 13 | | | |
| | 85V | 13 | | | |
| | 89O | 13 | | | |
| | 90I | 13 | | | |

Besides using single antigen expressing cells, magnetic beads coated with single antigens, such as One Lambda MagSort (Thermo Fisher Scientific, Inc.), can also be used to deconvolute pAb signals.

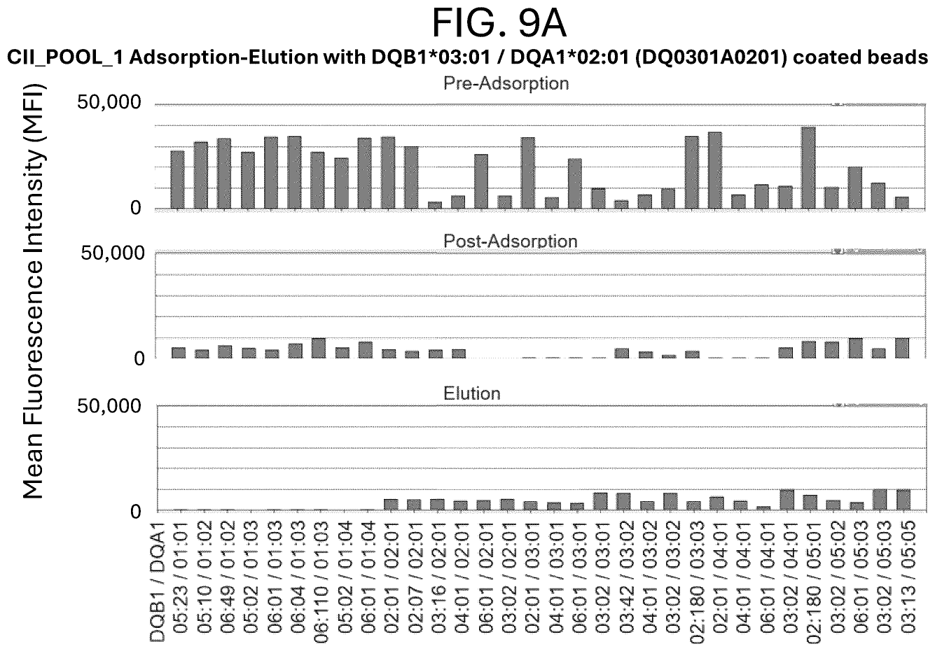

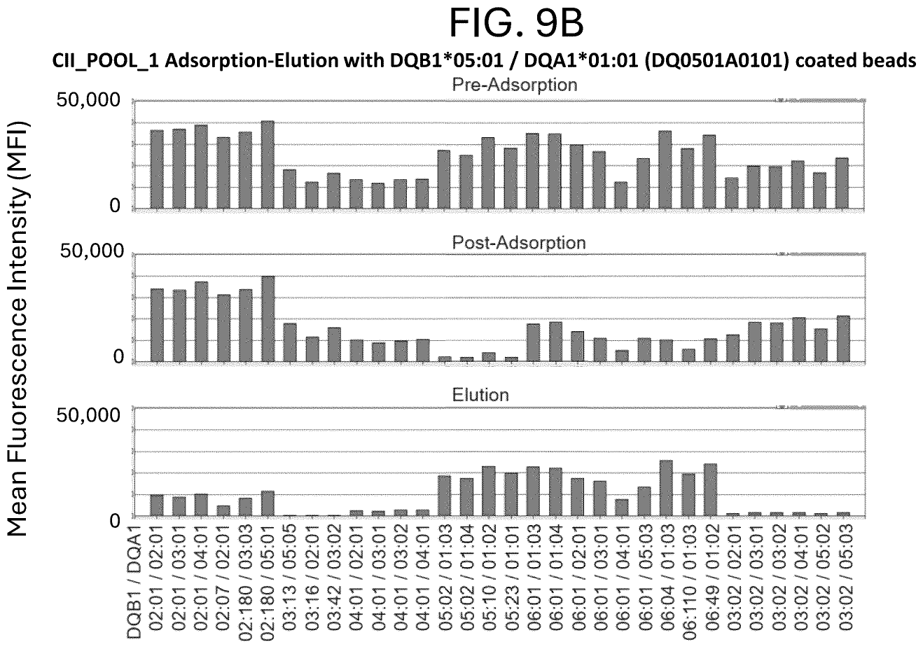

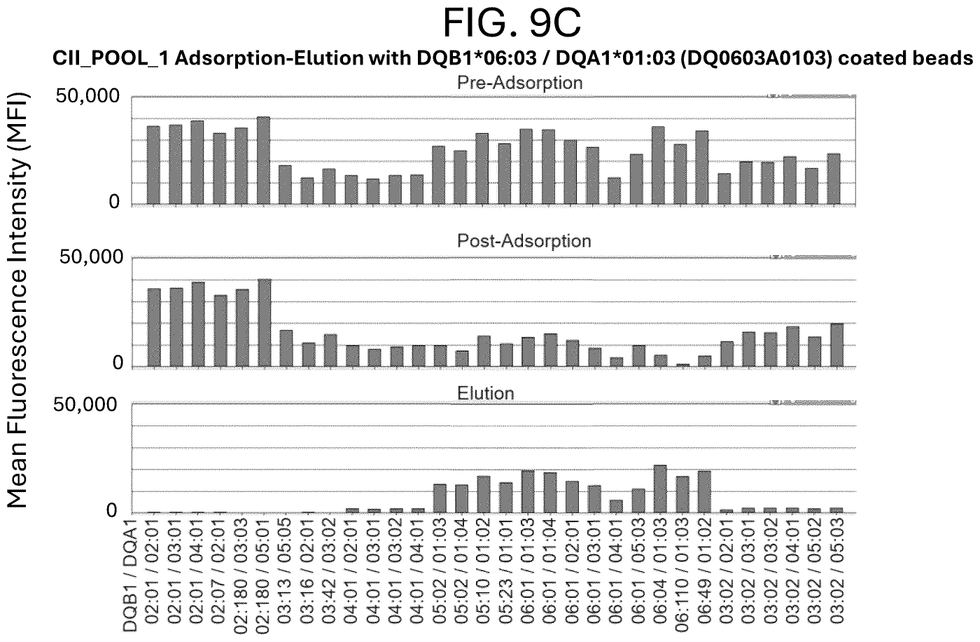

For CII_POOL_1, a pooled human serum sample with pan-HLA class II reactivities, is used for adsorption-elution with MagSort beads. Three different specificity profiles are observed in the eluants with DQB1*03:01/DQA1*02:01 (DQ0301A0201), DQB1*05:01/DQA1*01:01 (DQ0501A0101), and DQB1*06:01/DQA1*01:03 (DQ0603A0103) MagSort beads (FIGS. 9A, 9B, and 9C respectively). MagSort DQ0301A0201 can partition DQA02/03/04/05 signals from DQA01 signals in CII_POOL_1. Similarly, MagSort DQ0501A0101 eluted fraction does not recognize DQ3 signals anymore; instead, a new profile emerges with DQ2/5/6 signals stronger than DQ4/8. Again, MagSort DQ0602A0103 eluted fraction no longer binds to DQ2/3 but binds to DQ5/6 stronger than DQ4/8. All the above fractionation of signals either with single Ag expressed on cells or coated on beads leads to matching ER predictions (Table 10).

The examples provided above for designing HLA-DQ epitope panel, the use of such a panel to predict ER of epitopes, and the final confirmation of the ER by EV with altered ER for both mAb and pAb can be applied to designing additional panels not only for HLA-DP Ag which also consist of variant alpha and beta subunits but also HLA-DR (with constant alpha subunit) and HLA-CI Ag (with constant beta 2 microglobulin subunit). For HLA-DR and HLA-CI panels, there is no need to consider alpha-beta pairing in the design. Similar logic and thought process for designing additional HLA epitope panels are described in the following examples.

### Example 7: Design of HLA-DP Epitope Panel and Engineered Variants

### Design of HLA-DP Epitope Panel

Similar as described in Example 1, Common DPB1 alleles listed in CIWD 3.0 are included in the initial coverage. As to DPA1, alleles with significant population representation such as in CWD 2.0 and/or AFND are also included for AA3D panel design. The resulting minimal number of HLA-DP alleles and pairs are listed in Table 11A and Table 11B.

TABLE 11A
Selected 45 minimal number of HLA-DP alleles and pairs covering all unique or
representative eps and ep patterns that could form an ER in contact with an Ab.

| Count | DPA | Count | DPB | Count | DPA/DPB Pair |
|---|---|---|---|---|---|
| 1 | DPA1*02:06 | 1 | DPB1*04:02 | 1 | DPA1*01:12/DPB1*01:01 |
| 2 | DPA1*02:07 | 2 | DPB1*06:01 | 2 | DPA1*01:03/DPB1*01:01 |
| | | 3 | DPB1*11:01 | 3 | DPA1*01:03/DPB1*02:01 |
| | | 4 | DPB1*28:01 | 4 | DPA1*01:03/DPB1*03:01 |
| | | 5 | DPB1*34:01 | 5 | DPA1*01:03/DPB1*04:01 |
| | | 6 | DPB1*41:01 | 6 | DPA1*01:03/DPB1*05:01 |
| | | 7 | DPB1*55:01 | 7 | DPA1*01:03/DPB1*13:01 |
| | | 8 | DPB1*281:01 | 8 | DPA1*01:03/DPB1*15:01 |
| | | 9 | DPB1*415:01 | 9 | DPA1*01:03/DPB1*17:01 |
| | | | | 10 | DPA1*01:03/DPB1*21:01 |
| | | | | 11 | DPA1*01:03/DPB1*38:01 |
| | | | | 12 | DPA1*01:03/DPB1*40:01 |
| | | | | 13 | DPA1*01:03/DPB1*85:01 |
| | | | | 14 | DPA1*01:12/DPB1*03:01 |
| | | | | 15 | DPA1*01:12/DPB1*04:01 |
| | | | | 16 | DPA1*01:12/DPB1*17:01 |
| | | | | 17 | DPA1*02:01/DPB1*03:01 |
| | | | | 18 | DPA1*02:01/DPB1*04:01 |
| | | | | 19 | DPA1*02:01/DPB1*15:01 |
| | | | | 20 | DPA1*02:02/DPB1*01:01 |
| | | | | 21 | DPA1*02:02/DPB1*03:01 |
| | | | | 22 | DPA1*02:02/DPB1*04:01 |
| | | | | 23 | DPA1*02:02/DPB1*17:01 |
| | | | | 24 | DPA1*03:01/DPB1*01:01 |
| | | | | 25 | DPA1*03:01/DPB1*03:01 |
| | | | | 26 | DPA1*03:01/DPB1*04:01 |
| | | | | 27 | DPA1*03:01/DPB1*17:01 |
| | | | | 28 | DPA1*04:01/DPB1*01:01 |
| | | | | 29 | DPA1*04:01/DPB1*03:01 |
| | | | | 30 | DPA1*04:01/DPB1*04:01 |
| | | | | 31 | DPA1*04:01/DPB1*05:01 |
| | | | | 32 | DPA1*04:01/DPB1*17:01 |
| | | | | 33 | DPA1*04:01/DPB1*21:01 |
| | | | | 34 | DPA1*04:01/DPB1*40:01 |

TABLE 11B
Selected 30 minimal number of HLA-DP alleles covering
all unique or representative eps and ep patterns
that could form an ER in contact with an Ab.

| Count | DPA | Count | DPB |
|---|---|---|---|
| 1 | DPA1*01:03 | 1 | DPB1*01:01 |
| 2 | DPA1*01:12 | 2 | DPB1*02:01 |
| 3 | DPA1*02:01 | 3 | DPB1*03:01 |
| 4 | DPA1*02:02 | 4 | DPB1*04:01 |
| 5 | DPA1*02:06 | 5 | DPB1*04:02 |
| 6 | DPA1*02:07 | 6 | DPB1*05:01 |
| 7 | DPA1*03:01 | 7 | DPB1*06:01 |
| 8 | DPA1*04:01 | 8 | DPB1*11:01 |
| | | 9 | DPB1*13:01 |
| | | 10 | DPB1*15:01 |
| | | 11 | DPB1*17:01 |
| | | 12 | DPB1*19:01 |
| | | 13 | DPB1*21:01 |
| | | 14 | DPB1*28:01 |
| | | 15 | DPB1*34:01 |
| | | 16 | DPB1*38:01 |
| | | 17 | DPB1*40:01 |
| | | 18 | DPB1*41:01 |
| | | 19 | DPB1*55:01 |
| | | 20 | DPB1*85:01 |
| | | 21 | DPB1*281:01 |
| | | 22 | DPB1*415:01 |

TABLE 11C
List of DP antigens selected for feasibility epitope panel
production. 45 Ag are selected. 9 Ag are already included
on One Lambda LABScreen ™ CII SAB panels (in bold)
and 8 Ag only in Werfen LSA ™ NEXA2 SAB panel (underlined).

| Count | DPA | DPB |
|---|---|---|
| 1 | DPA1*01:03 | DPB1*01:01 |
| 2 | DPA1*01:03 | DPB1*02:01 |
| 3 | DPA1*01:03 | DPB1*03:01 |
| 4 | DPA1*01:03 | DPB1*04:01 |
| 5 | DPA1*01:03 | DPB1*04:02 |
| 6 | DPA1*01:03 | DPB1*05:01 |
| 7 | DPA1*01:03 | DPB1*06:01 |
| 8 | DPA1*01:03 | DPB1*13:01 |
| 9 | DPA1*01:03 | DPB1*15:01 |
| 10 | DPA1*01:03 | DPB1*17:01 |
| 11 | DPA1*01:03 | DPB1*21:01 |
| 12 | DPA1*01:03 | DPB1*34:01 |
| 13 | DPA1*01:03 | DPB1*38:01 |
| 14 | DPA1*01:03 | DPB1*40:01 |
| 15 | DPA1*01:03 | DPB1*41:01 |
| 16 | DPA1*01:03 | DPB1*85:01 |
| 17 | DPA1*01:03 | DPB1*281:01 |
| 18 | DPA1*01:03 | DPB1*415:01 |
| 19 | DPA1*01:12 | DPB1*01:01 |
| 20 | DPA1*01:12 | DPB1*03:01 |
| 21 | DPA1*01:12 | DPB1*04:01 |
| 22 | DPA1*01:12 | DPB1*17:01 |
| 23 | DPA1*02:01 | DPB1*03:01 |
| 24 | DPA1*02:01 | DPB1*04:01 |
| 25 | DPA1*02:01 | DPB1*11:01 |
| 26 | DPA1*02:01 | DPB1*15:01 |
| 27 | DPA1*02:02 | DPB1*01:01 |
| 28 | DPA1*02:02 | DPB1*03:01 |
| 29 | DPA1*02:02 | DPB1*04:01 |
| 30 | DPA1*02:02 | DPB1*17:01 |
| 31 | DPA1*02:06 | DPB1*05:01 |
| 32 | DPA1*02:07 | DPB1*19:01 |
| 33 | DPA1*03:01 | DPB1*01:01 |
| 34 | DPA1*03:01 | DPB1*03:01 |
| 35 | DPA1*03:01 | DPB1*04:01 |
| 36 | DPA1*03:01 | DPB1*17:01 |
| 37 | DPA1*03:01 | DPB1*55:01 |
| 38 | DPA1*04:01 | DPB1*01:01 |
| 39 | DPA1*04:01 | DPB1*03:01 |
| 40 | DPA1*04:01 | DPB1*04:01 |
| 41 | DPA1*04:01 | DPB1*05:01 |
| 42 | DPA1*04:01 | DPB1*17:01 |
| 43 | DPA1*04:01 | DPB1*21:01 |
| 44 | DPA1*04:01 | DPB1*28:01 |
| 45 | DPA1*04:01 | DPB1*40:01 |

A DP epitope panel can be produced for the panel disclosed in Table 11C, according to the methods disclosed in Examples 1-6.

### Design of HLA-DP Engineered Variants

The total as well as prioritized variant positions are listed in Table 12A. Prioritized variant positions are selected based on their proposed Ab accessible positions and/or positions likely impact peptide selection for presentation. Such positions are more likely either directly in contact of an Ab or directly influence the presentation of a peptide which is in contact with an Ab. Embedded residues could in contact with multiple neighboring residues and critical for structure integrity, therefore deprioritized for EV generation.

TABLE 12A
List of exemplary HLA-DP variant positions
among common alleles suitable for EV generation.
HLA-DP Variant Positions

| | |
|---|---|
| DPA1 | DPB1 |

| Total | Prioritized | Total | Prioritized |
|---|---|---|---|
| 11 | | 8 | |
| 18 | | 9 | |
| 28 | 28 | 11 | 11 |
| 31 | 31 | 17 | |
| 43 | 43 | 33 | 33 |
| 50 | 50 | 35 | |
| 51 | 51 | 36 | |
| 66 | 66 | 55 | 55 |
| 72 | 72 | 56 | |
| 73 | 73 | 57 | 57 |
| 83 | 83 | 65 | 65 |
| 91 | | 69 | 69 |
| 96 | | 72 | 72 |
| 111 | 111 | 76 | 76 |
| 127 | 127 | 84 | 84 |
| 160 | 160 | 85 | 85 |
| 190 | | 86 | 86 |
| | | 87 | 87 |
| | | 90 | 90 |
| | | 91 | 91 |
| | | 96 | 96 |
| | | 162 | 162 |
| | | 170 | 170 |
| | | 178 | 178 |
| | | 188 | |
| | | 194 | |

Even with prioritized positions, there were still numerous choices (1,000s-10,000s). Therefore, the wildtype alleles are also prioritized based on their frequency in the targeted population(s). Only common alleles with a total frequency ≥1% are listed as exemplary LV that can be generated for ER and/or AR verification (Tables 12B and 12C).

Tables 12B and 12C. List of exemplary designed HLA-DP engineered variants suitable for ER and/or AR verification. For DPA1 (Table 12B) and DPB1 (Table 12C) alleles, only representative alleles with total frequency ≥1% (CIWD 3.0) are listed as examples for EV designs. Some EV designs are underlined because they are changes among small residues (amino acids A, G and S) that may not result in an conspicuous impact.

TABLE 12B
Exemplary HLA-DPA1 EVs.
DPA1 Prioritized EV Design

| Change to existing e p | Chan |
|---|---|
| DPA1*01:01_E28D | DPA1*01:01_E28A |
| DPA1*01:01_M31Q | DPA1*01:01_M31A |
| DPA1*01:01_W43C | DPA1*01:01_W43A |
| DPA1*01:01_Q50R | DPA1*01:01_Q50A |
| DPA1*01:01_A51T | |
| DPA1*01:01_L66S | DPA1*01:01_L66A |
| DPA1*01:01_T72I | DPA1*01:01_T724 |
| DPA1*01:01_L73A | |
| DPA1*01:01_T83A | |
| DPA1*01:01_K111R | DPA1*01:01_K1114 |
| DPA1*01:01_L127P | DPA1*01:01_L127A |
| DPA1*01:01_F160V | DPA1*01:01_F160A |
| DPA1*02:01_E28D | DPA1*02:01_E28A |
| DPA1*02:01_Q31M | DPA1*02:01_Q31A |
| DPA1*02:01_W43C | DPA1*02:01_W43A |
| DPA1*02:01_R50Q | DPA1*02:01_R50A |
| DPA1*02:01_A51T | |
| DPA1*02:01_L66S | DPA1*02:01_L66A |
| DPA1*02:01_T72I | DPA1*02:01_T72A |
| DPA1*02:01_L73A | |
| DPA1*02:01_A83T | |
| DPA1*02:01_R111K | DPA1*02:01_R111A |
| DPA1*02:01_P127L | DPA1*02:01_P127A |
| DPA1*02:01_V160F | DPA1_02:01_V160A |
| DPA1*03:01_E28D | DPA1*03:01_E28A |
| DPA1*03:01_M31Q | DPA1*03:01_M31A |
| DPA1*03:01_W43C | DPA1_03:01_W43A |
| DPA1*03:01_Q508 | DPA1*03:01_Q50A |
| DPA1*03:01_A51T | |
| DPA1*03:01_S66L | DPA1*03:01_S66A |
| DPA1*03:01_T72I | DPA1*03:01_T72A |
| DPA1*03:01_L73A | |
| DPA1*03:01_T83A | |
| DPA1*03:01_K111R | DPA1*03:01_K111A |
| DPA1*03:01_L127P | DPA1*03:01_L127A |
| DPA1*03:01_F160V | DPA1*03:01_F160A |
| DPA1*04:01_D28E | DPA1*04:01_D28A |
| DPA1*04:01_M31Q | DPA1*04:01_M31A |
| DPA1*04:01_W43C | DPA1*04:01_W43A |
| DPA1*04:01_R50Q | DPA1*04:01_R50A |
| DPA1*04:01_A51T | |
| DPA1*04:01_L66S | DPA1*04:01_L66A |
| DPA1*04:01_I72T | DPA1*04:01_I72A |
| DPA1*04:01_A73L | |
| DPA1*04:01_A83T | |
| DPA1*04:01_K111R | DPA1*04:01_K111A |
| DPA1*04:01_P127L | DPA1*04:01_P127A |
| DPA1*04:01_V160F | DPA1*04:01_V160A |

TABLE 12C
Exemplary HLA-DPB1 EVs.
DPB1 Prioritized EV Design

| Change to existing e p | Change to alanine |
|---|---|
| DPB1*01:01_G11L | DPB1*01:01_G11A |
| DPB1*01:01_E33Q | DPB1*01:01_E33A |
| DPB1*01:01_A55E | |
| DPB1*01:01_E57D | DPB1*01:01_E57A |
| DPB1*01:01_I65L | DPB1*01:01_I65A |
| DPB1*01:01_K69E | DPB1*01:01_K69A |
| DPB1*01:01_V72L | DPB1*01:01_V72A |
| DPB1*01:01_V76M | DPB1*01:01_V76A |
| DPB1*01:01_D84V | DPB1*01:01_D84A |
| DPB1*01:01_E85G | DPB1*01:01_E85A |
| DPB1*01:01_A86P | |
| DPB1*01:01_V87M | DPB1*01:01_V87A |
| DPB1*01:01_Q90K | DPB1*01:01_Q90A |
| DPB1*01:01_R91H | DPB1*01:01_R91A |
| DPB1*01:01_K96R | DPB1*01:01_K96A |
| DPB1*01:01_T162N | DPB1*01:01_T162A |
| DPB1*01:01_I170T | DPB1*01:01_I170A |
| DPB1*01:01_L178M | DPB1*01:01_L178A |
| DPB1*02:01_G11L | DPB1*02:01_G11A |
| DPB1*02:01_E33Q | DPB1*02:01_E33A |
| DPB1*02:01_D55E | DPB1*02:01_D55A |
| DPB1*02:01_E57D | DPB1*02:01_E57A |
| DPB1*02:01_I65L | DPB1*02:01_I65A |
| DPB1*02:01_E69K | DPB1*02:01_E69A |
| DPB1*02:01_V72L | DPB1*02:01_V72A |
| DPB1*02:01_V76M | DPB1*02:01_V76A |
| DPB1*02:01_G84V | DPB1*02:01_G84A |
| DPB1*02:01_G85E | DPB1*02:01_G85A |
| DPB1*02:01_P86A | |
| DPB1*02:01_M87V | DPB1*02:01_M87A |
| DPB1*02:01_Q90K | DPB1*02:01_Q90A |
| DPB1*02:01_R91H | DPB1*02:01_R91A |
| DPB1*02:01_R96R | DPB1*02:01_R96A |
| DPB1*02:01_T162N | DPB1*02:01_T162A |
| DPB1*02:01_T170I | DPB1*02:01_T170A |
| DPB1*02:01_L178M | DPB1*02:01_L178A |
| DPB1*03:01_L11G | DPB1*03:01_L11A |
| DPB1*03:01_E33Q | DPB1*03:01_E33A |
| DPB1*03:01_D55E | DPB1*03:01_D55A |
| DPB1*03:01_D57E | DPB1*03:01_D57A |
| DPB1*03:01_L65I | DPB1*03:01_L65A |
| DPB1*03:01_K69E | DPB1*03:01_K69A |
| DPB1*03:01_V72L | DPB1*03:01_V72A |
| DPB1*03:01_V76M | DPB1*03:01_V76A |
| DPB1*03:01_D84G | DPB1*03:01_D84A |
| DPB1*03:01_E85G | DPB1*03:01_E85A |
| DPB1*03:01_A86P | |
| DPB1*03:01_V87M | DPB1*03:01_V87A |
| DPB1*03:01_Q90K | DPB1*03:01_Q90A |
| DPB1*03:01_R91H | DPB1*03:01_R91A |
| DPB1*03:01_K96R | DPB1*03:01_K96A |
| DPB1*03:01_T162N | DPB1*03:01_T162A |
| DPB1*03:01_I170T | DPB1*03:01_I170A |
| DPB1*03:01_L178M | DPB1*03:01_L178A |
| DPB1*04:01_G11L | DPB1*04:01_G11A |
| DPB1*04:01_E33Q | DPB1*04:01_E33A |
| DPB1*04:01_A55D | |
| DPB1*04:01_E57D | DPB1*04:01_E57A |
| DPB1*04:01_L65I | DPB1*04:01_L65A |
| DPB1*04:01_K69E | DPB1*04:01_K69A |
| DPB1*04:01_V72L | DPB1*04:01_V72A |
| DPB1*04:01_M76V | DPB1*04:01_M76A |
| DPB1*04:01_G84V | DPB1*04:01_G84A |
| DPB1*04:01_G85E | DPB1*04:01_G85A |
| DPB1*04:01_P86A | |
| DPB1*04:01_M87V | DPB1*04:01_M87A |
| DPB1*04:01_Q90K | DPB1*04:01_Q90A |
| DPB1*04:01_R91H | DPB1*04:01_R91A |
| DPB1*04:01_R96K | DPB1*04:01_R96A |
| DPB1*04:01_T162N | DPB1*04:01_T162A |
| DPB1*04:01_T170I | DPB1*04:01_T170A |
| DPB1*04:01_L178M | DPB1*04:01_L178A |
| DPB1*05:01_G11L | DPB1*05:01_G11A |
| DPB1*05:01_E33Q | DPB1*05:01_E33A |
| DPB1*05:01_E55D | DPB1*05:01_E55A |
| DPB1*05:01_E57D | DPB1*05:01_E57A |
| DPB1*05:01_I65L | DPB1*05:01_I65A |
| DPB1*05:01_K69E | DPB1*05:01_K69A |
| DPB1*05:01_V72L | DPB1*05:01_V72A |
| DPB1*05:01_M76V | DPB1*05:01_M76A |
| DPB1*05:01_D84V | DPB1*05:01_G84A |
| DPB1*05:01_E85G | DPB1*05:01_G85A |
| DPB1*05:01_A86P | |
| DPB1*05:01_V87M | DPB1*05:01_V87A |
| DPB1*05:01_Q90K | DPB1*05:01_Q90A |
| DPB1*05:01_R91H | DPB1*05:01_R91A |
| DPB1*05:01_K96R | DPB1*05:01_K96A |
| DPB1*05:01_T162N | DPB1*05:01_T162A |
| DPB1*05:01_I170T | DPB1*05:01_I170A |
| DPB1*05:01_L178M | DPB1*05:01_L178A |
| DPB1*17:01_L11G | DPB1*17:01_L11A |
| DPB1*17:01_E33Q | DPB1*17:01_E33A |
| DPB1*17:01_D55E | DPB1*17:01_D55A |
| DPB1*17:01_D57E | DPB1*17:01_D57A |
| DPB1*17:01_I65L | DPB1*17:01_I65A |
| DPB1*17:01_E69K | DPB1*17:01_E69A |
| DPB1*17:01_V72L | DPB1*17:01_V72A |
| DPB1*17:01_M76V | DPB1*17:01_M76A |
| DPB1*17:01_D84V | DPB1*17:01_D84A |
| DPB1*17:01_E85G | DPB1*17:01_E85A |
| DPB1*17:01_A86P | |
| DPB1*17:01_V87M | DPB1*17:01_V87A |
| DPB1*17:01_Q90K | DPB1*17:01_Q90A |
| DPB1*17:01_R91H | DPB1*17:01_R91A |
| DPB1*17:01_R96K | DPB1*17:01_R96A |
| DPB1*17:01_T162N | DPB1*17:01_T162A |
| DPB1*17:01_T170I | DPB1*17:01_T170A |
| DPB1*17:01_L178M | DPB1*17:01_L178A |

### Example 8: Design of HLA-DR Epitope Panel and Engineered Variants

### Design of HLA-DR Epitope Panel

As described in Example 1, Common DRB1 alleles listed in CIWD 3.0 were included for initial coverage. As to DRB3, DRB4 and DRB5 (DRB345) alleles, the highest frequency assignments only reach WD status and many of them are of much lower frequency than DRB1 Common alleles. For this reason, a cut-off frequency is applied to DRB345 and only alleles of frequencies above the cut-off are included for coverage consideration. The resulting minimal number of HLA-DR alleles using AA3D panel design are listed in Table 13.

TABLE 13
List of DR antigens selected for feasibility epitope panel
production. 46 Ag were selected. 26 Ag are already included
on One Lambda LABScreen ™ CII SAB panels (in bold)
and 3 Ag only in Werfen LSA ™ NEXA2 SAB panel (underlined).

| Count | DRB1 | Count | DRB3/4/5 |
|---|---|---|---|
| 1 | DRB1*01:03 | 1 | DRB3*01:01 |
| 2 | DRB1*03:02 | 2 | DRB3*02:01 |
| 3 | DRB1*03:04 | 3 | DRB3*03:01 |
| 4 | DRB1*03:15 | 4 | DRB4*01:01 |
| 5 | DRB1*04:01 | 5 | DRB4*01:02 |
| 6 | DRB1*04:02 | 6 | DRB5*01:01 |
| 7 | DRB1*04:03 | 7 | DRB5*01:03 |
| 8 | DRB1*04:04 | 8 | DRB5*02:02 |
| 9 | DRB1*04:06 | | |
| 10 | DRB1*07:01 | | |
| 11 | DRB1*07:03 | | |
| 12 | DRB1*08:07 | | |
| 13 | DRB1*08:11 | | |
| 14 | DRB1*09:01 | | |
| 15 | DRB1*10:01 | | |
| 16 | DRB1*11:03 | | |
| 17 | DRB1*11:08 | | |
| 18 | DRB1*11:12 | | |
| 19 | DRB1*11:15 | | |
| 20 | DRB1*11:66 | | |
| 21 | DRB1*11:129 | | |
| 22 | DRB1*12:01 | | |
| 23 | DRB1*13:03 | | |
| 24 | DRB1*13:15 | | |
| 25 | DRB1*13:16 | | |
| 26 | DRB1*13:59 | | |
| 27 | DRB1*14:01 | | |
| 28 | DRB1*14:02 | | |
| 29 | DRB1*14:05 | | |
| 30 | DRB1*14:08 | | |
| 31 | DRB1*14:10 | | |
| 32 | DRB1*14:11 | | |
| 33 | DRB1*14:24 | | |
| 34 | DRB1*15:03 | | |
| 35 | DRB1*15:04 | | |
| 36 | DRB1*15:06 | | |
| 37 | DRB1*16:01 | | |
| 38 | DRB1*16:02 | | |

A DR epitope panel can be produced for the panel disclosed in Table 13, according to the methods disclosed in Examples 1-6.

### Design of HLA-DR Engineered Variants

Total and prioritized variant positions among DRB1Common alleles or DRB3/4/5 (CIWD 3.0) alleles are listed in Table 14A. Prioritized variant positions cover proposed Ab accessible positions and positions likely impact peptide selection for presentation.

TABLE 14A
List of HLA-DR variant positions among common
alleles suitable for EV generation.
HLA-DR Variant Positions

| DRB1 | | DR3/4/5 | | DRB1 | | DR3/4/5 | |
|---|---|---|---|---|---|---|---|
| Total | Prioritized | Total | Prioritized | Total | Prioritized | Total | Prioritized |
| 4 | 4 | 4 | 4 | 71 | | 71 | |
| 9 | 9 | 6 | | 73 | 73 | 73 | 73 |
| 10 | | 9 | 9 | 74 | 74 | 74 | 74 |
| 11 | | 10 | | 77 | 77 | 76 | 76 |
| 12 | | 11 | 11 | 78 | | 77 | 77 |
| 13 | | 12 | | 85 | 85 | 78 | |
| 14 | | 13 | | 86 | | 81 | 81 |
| 16 | 16 | 18 | 18 | 96 | 96 | 85 | 85 |
| 25 | 25 | 25 | 25 | 98 | 98 | 86 | |
| 26 | | 26 | | 104 | 104 | 96 | 96 |
| 27 | | 28 | | 112 | 112 | 98 | 98 |
| 28 | | 29 | | 120 | | 104 | 104 |
| 29 | | 30 | | 133 | 133 | 105 | 105 |
| 30 | | 31 | 31 | 140 | 140 | 108 | 108 |
| 31 | 31 | 32 | 32 | 142 | 142 | 120 | |
| 32 | 32 | 34 | | 149 | | 135 | 135 |
| 33 | | 37 | | 166 | 166 | 140 | 140 |
| 37 | | 38 | | 179 | 179 | 149 | |
| 38 | | 40 | | 180 | 180 | 157 | |
| 40 | | 41 | | 181 | 181 | 164 | 164 |
| 47 | | 44 | 44 | 189 | 189 | 170 | |
| 50 | 50 | 47 | | | | 180 | 180 |
| 51 | 51 | 48 | 48 | | | 181 | 181 |
| 57 | | 55 | 55 | | | 183 | |
| 58 | 58 | 57 | | | | 187 | 187 |
| 60 | 60 | 60 | | | | 189 | 189 |
| 67 | 67 | 67 | 67 | | | 191 | 191 |
| 70 | 70 | 70 | 70 | | | | |

For DRB1 alleles, only representative alleles with total frequency ≥5% (CIWD 3.0), and for DRB3/4/5 alleles, ≥1% are listed as examples for EV designs (Table 14B). Some EV designs are underlined because they are changes among small residues (amino acids A, G and S) that may not result in conspicuous impact.

Tables 14B and 14C. List of designed HLA-DR engineered variants suitable for ER and/or AR verification.

TABLE 14B
Exemplary HLA-DRB1 EVs.
DRB1 Prioritized EV Design

| Change to existing e p | Change to alanine |
|---|---|
| DRB1*01:01_R4Q | DRB1*01:01_R4A |
| DRB1*01:01_W9E | DRB1*01:01_W9A |
| DRB1*01:01_H16Y | DRB1*01:01_H16A |
| DRB1*01:01_R25Q | DRB1*01:01_R25A |
| DRB1*01:01_I31F | DRB1*01:01_I31A |
| DRB1*01:01_Y32H | DRB1*01:01_Y32A |
| DRB1*01:01_V50A | |
| DRB1*01:01_T51M | DRB1*01:01_T51A |
| DRB1*01:01_A58E | |
| DRB1*01:01_Y60S | DRB1*01:01_Y60A |
| DRB1*01:01_L67F | DRB1*01:01_L67A |
| DRB1*01:01_Q70D | DRB1*01:01_Q70A |
| DRB1*01:01_A73G | |
| DRB1*01:01_A74R | |
| DRB1*01:01_T77N | DRB1*01:01_T77A |
| DRB1*01:01_V85A | |
| DRB1*01:01_E96H | DRB1*01:01_E96A |
| DRB1*01:01_K98E | DRB1*01:01_K98A |
| DRB1*01:01_S104A | |
| DRB1*01:01_H112Y | DRB1*01:01_H112A |
| DRB1*01:01_R133L | DRB1*01:01_R133A |
| DRB1*01:01_A140T | |
| DRB1*01:01_V142M | DRB1*01:01_V142A |
| DRB1*01:01_R166Q | DRB1*01:01_R166A |
| DRB1*01:01_S179N | DRB1*01:01_S179A |
| DRB1*01:01_V180L | DRB1*01:01_V180A |
| DRB1*01:01_T181M | DRB1*01:01_T181A |
| DRB1*01:01_R189S | DRB1*01:01_R189A |
| DRB1*03:01_R4Q | DRB1*03:01_R4A |
| DRB1*03:01_E9W | DRB1*03:01_E9A |
| DRB1*03:01_H16Y | DRB1*03:01_H16A |
| DRB1*03:01_R25Q | DRB1*03:01_R25A |
| DRB1*03:01_I31F | DRB1*03:01_I31A |
| DRB1*03:01_H32Y | DRB1*03:01_H32A |
| DRB1*03:01_V50A | |
| DRB1*03:01_T51M | DRB1*03:01_T51A |
| DRB1*03:01_A58E | |
| DRB1*03:01_Y60S | DRB1*03:01_Y60A |
| DRB1*03:01_L67F | DRB1*03:01_L67A |
| DRB1*03:01_Q70D | DRB1*03:01_Q70A |
| DRB1*03:01_G73A | |
| DRB1*03:01_R74Q | DRB1*03:01_R74A |
| DRB1*03:01_N77T | DRB1*03:01_N77A |
| DRB1*03:01_V85A | |
| DRB1*03:01_H96E | DRB1*03:01_H96A |
| DRB1*03:01_K98E | DRB1*03:01_K98A |
| DRB1*03:01_S104A | |
| DRB1*03:01_H112Y | DRB1*03:01_H112A |
| DRB1*03:01_R133L | DRB1*03:01_R133A |
| DRB1*03:01_T140A | |
| DRB1*03:01_V142M | DRB1*03:01_V142A |
| DRB1*03:01_R166Q | DRB1*03:01_R166A |
| DRB1*03:01_S179N | DRB1*03:01_S179A |
| DRB1*03:01_V180L | DRB1*03:01_V180A |
| DRB1*03:01_T181M | DRB1*03:01_T181A |
| DRB1*03:01_R189S | DRB1*03:01_R189A |
| DRB1*04:01_R4Q | DRB1*04:01_R4A |
| DRB1*04:01_E9W | DRB1*04:01_E9A |
| DRB1*04:01_H16Y | DRB1*04:01_H16A |
| DRB1*04:01_R25Q | DRB1*04:01_R25A |
| DRB1*04:01_F31I | DRB1*04:01_F31A |
| DRB1*04:01_Y32H | DRB1*04:01_Y32A |
| DRB1*04:01_V50A | |
| DRB1*04:01_T51M | DRB1*04:01_T51A |
| DRB1*04:01_A58E | |
| DRB1*04:01_Y60S | DRB1*04:01_Y60A |
| DRB1*04:01_L67F | DRB1*04:01_L67A |
| DRB1*04:01_Q70D | DRB1*04:01_Q70A |
| DRB1*04:01_A73G | |
| DRB1*04:01_A74E | |
| DRB1*04:01_T77N | DRB1*04:01_T77A |
| DRB1*04:01_V85A | |
| DRB1*04:01_Y96H | DRB1*04:01_Y96A |
| DRB1*04:01_E98K | DRB1*04:01_E98A |
| DRB1*04:01_A104S | |
| DRB1*04:01_H112Y | DRB1*04:01_H112A |
| DRB1*04:01_R133L | DRB1*04:01_R133A |
| DRB1*04:01_T140A | |
| DRB1*04:01_V142M | DRB1*04:01_V142A |
| DRB1*04:01_R166Q | DRB1*04:01_R166A |
| DRB1*04:01_S179N | DRB1*04:01_S179A |
| DRB1*04:01_L180V | DRB1*04:01_L180A |
| DRB1*04:01_T181M | DRB1*04:01_T181A |
| DRB1*04:01_R189S | DRB1*04:01_R189A |
| DRB1*07:01_Q4A | DRB1*07:01_Q4A |
| DRB1*07:01_W9E | DRB1*07:01_W9A |
| DRB1*07:01_H16Y | DRB1*07:01_H16A |
| DRB1*07:01_Q25R | DRB1*07:01_Q25A |
| DRB1*07:01_F31I | DRB1*07:01_F31A |
| DRB1*07:01_Y32H | DRB1*07:01_Y32A |
| DRB1*07:01_V50A | |
| DRB1*07:01_T51M | DRB1*07:01_T51A |
| DRB1*07:01_A58E | |
| DRB1*07:01_S60Y | DRB1*07:01_S60A |
| DRB1*07:01_I67F | DRB1*07:01_I67A |
| DRB1*07:01_D70R | DRB1*07:01_D70A |
| DRB1*07:01_G73A | |
| DRB1*07:01_Q74L | DRB1*07:01_Q74A |
| DRB1*07:01_T77N | DRB1*07:01_T77A |
| DRB1*07:01_V85A | |
| DRB1*07:01_H96Q | DRB1*07:01_H96A |
| DRB1*07:01_E98K | DRB1*07:01_E98A |
| DRB1*07:01_A104S | |
| DRB1*07:01_H112Y | DRB1*07:01_H112A |
| DRB1*07:01_R133L | DRB1*07:01_R133A |
| DRB1*07:01_A140T | |
| DRB1*07:01_V142M | DRB1*07:01_V142A |
| DRB1*07:01_R166Q | DRB1*07:01_R166A |
| DRB1*07:01_S179N | DRB1*07:01_S179A |
| DRB1*07:01_V180L | DRB1*07:01_V180A |
| DRB1*07:01_M181T | DRB1*07:01_M181A |
| DRB1*07:01_R189S | DRB1*07:01_R189A |
| DRB1*11:01_R4Q | DRB1*11:01_R4A |
| DRB1*11:01_E9W | DRB1*11:01_E9A |
| DRB1*11:01_H16Y | DRB1*11:01_H16A |
| DRB1*11:01_R25Q | DRB1*11:01_R25A |
| DRB1*11:01_F31I | DRB1*11:01_F31A |
| DRB1*11:01_Y32H | DRB1*11:01_Y32A |
| DRB1*11:01_V50A | |
| DRB1*11:01_T51M | DRB1*11:01_T51A |
| DRB1*11:01_E58A | |
| DRB1*11:01_Y60H | DRB1*11:01_Y60A |
| DRB1*11:01_F67I | DRB1*11:01_F67A |
| DRB1*11:01_D70R | DRB1*11:01_D70A |
| DRB1*11:01_A73G | |
| DRB1*11:01_A74E | |
| DRB1*11:01_T77N | DRB1*11:01_T77A |
| DRB1*11:01_V85A | |
| DRB1*11:01_H96Q | DRB1*11:01_H96A |
| DRB1*11:01_K98E | DRB1*11:01_K98A |
| DRB1*11:01_S104A | |
| DRB1*11:01_H112Y | DRB1*11:01_H112A |
| DRB1*11:01_R133L | DRB1*11:01_R133A |
| DRB1*11:01_T140A | |
| DRB1*11:01_V142M | DRB1*11:01_V142A |
| DRB1*11:01_R166Q | DRB1*11:01_R166A |
| DRB1*11:01_S179N | DRB1*11:01_S179A |
| DRB1*11:01_V180L | DRB1*11:01_V180A |
| DRB1*11:01_T181M | DRB1*11:01_T181A |
| DRB1*11:01_R189S | DRB1*11:01_R189A |
| DRB1*13:01_R4Q | DRB1*13:01_R4A |
| DRB1*13:01_E9W | DRB1*13:01_E9A |
| DRB1*13:01_H16Y | DRB1*13:01_H16A |
| DRB1*13:01_R25Q | DRB1*13:01_R25A |
| DRB1*13:01_F31I | DRB1*13:01_F31A |
| DRB1*13:01_H32Y | DRB1*13:01_H32A |
| DRB1*13:01_V50A | |
| DRB1*13:01_T51M | DRB1*13:01_T51A |
| DRB1*13:01_A58E | |
| DRB1*13:01_Y60H | DRB1*13:01_Y60A |
| DRB1*13:01_I67F | DRB1*13:01_I67A |
| DRB1*13:01_D70R | DRB1*13:01_D70A |
| DRB1*13:01_A73G | |
| DRB1*13:01_A74E | |
| DRB1*13:01_T77N | DRB1*13:01_T77A |
| DRB1*13:01_V85A | |
| DRB1*13:01_H96Q | DRB1*13:01_H96A |
| DRB1*13:01_K98E | DRB1*13:01_K98A |
| DRB1*13:01_S104A | |
| DRB1*13:01_H112Y | DRB1*13:01_H112A |
| DRB1*13:01_R133L | DRB1*13:01_R133A |
| DRB1*13:01_T140A | |
| DRB1*13:01_V142M | DRB1*13:01_V142A |
| DRB1*13:01_R166Q | DRB1*13:01_R166A |
| DRB1*13:01_S179N | DRB1*13:01_S179A |
| DRB1*13:01_V180L | DRB1*13:01_V180A |
| DRB1*13:01_T181M | DRB1*13:01_T181A |
| DRB1*13:01_R189S | DRB1*13:01_R189A |

TABLE 14C
Exemplary HLA-DRB3/4/5 EVs.
DRB3/4/5 Prioritized EV Design

| Change to existing e p | Change to alanine |
|---|---|
| DRB3*01:01_R4Q | DRB3*01:01_R4A |
| DRB3*01:01_E9Q | DRB3*01:01_E9A |
| DRB3*01:01_R11L | DRB3*01:01_R11A |
| DRB3*01:01_F18L | DRB3*01:01_F18A |
| DRB3*01:01_R25W | DRB3*01:01_R25A |
| DRB3*01:01_F31I | DRB3*01:01_F31A |
| DRB3*01:01_H32Y | DRB3*01:01_H32A |
| DRB3*01:01_V44L | DRB3*01:01_V44A |
| DRB3*01:01_R48Q | DRB3*01:01_R48A |
| DRB3*01:01_R55L | DRB3*01:01_R55A |
| DRB3*01:01_L67F | DRB3*01:01_L67A |
| DRB3*01:01_Q70R | DRB3*01:01_Q70A |
| DRB3*01:01_G73A | |
| DRB3*01:01_R74Q | DRB3*01:01_R74A |
| DRB3*01:01_D76G | DRB3*01:01_D76A |
| DRB3*01:01_N77T | DRB3*01:01_N77A |
| DRB3*01:01_H81Y | DRB3*01:01_H81A |
| DRB3*01:01_V85A | |
| DRB3*01:01_H96Q | DRB3*01:01_H96A |
| DRB3*01:01_Q96K | DRB3*01:01_Q96A |
| DRB3*01:01_A104S | |
| DRB3*01:01_K105S | DRB3*01:01_K105A |
| DRB3*01:01_P108T | DRB3*01:01_P108A |
| DRB3*01:01_G135S | DRB3*01:01_G135A |
| DRB3*01:01_A140T | |
| DRB3*01:01_V164F | DRB3*01:01_V164A |
| DRB3*01:01_V180M | DRB3*01:01_V180A |
| DRB3*01:01_T181M | DRB3*01:01_T181A |
| DRB3*01:01_E187Q | DRB3*01:01_E187A |
| DRB3*01:01_R189S | DRB3*01:01_R189A |
| DRB3*01:01_R191Q | DRB3*01:01_R191A |
| DRB3*02:02_R4Q | DRB3*02:02_R4A |
| DRB3*02:02_E9Q | DRB3*02:02_E9A |
| DRB3*02:02_L11R | DRB3*02:02_L11A |
| DRB3*02:02_F18L | DRB3*02:02_F18A |
| DRB3*02:02_R25W | DRB3*02:02_R25A |
| DRB3*02:02_F31I | DRB3*02:02_F31A |
| DRB3*02:02_H32Y | DRB3*02:02_H32A |
| DRB3*02:02_V44L | DRB3*02:02_V44A |
| DRB3*02:02_R48Q | DRB3*02:02_R48A |
| DRB3*02:02_R55L | DRB3*02:02_R55A |
| DRB3*02:02_L67F | DRB3*02:02_L67A |
| DRB3*02:02_Q70R | DRB3*02:02_Q70A |
| DRB3*02:02_G73A | |
| DRB3*02:02_Q74R | DRB3*02:02_Q74A |
| DRB3*02:02_D76G | DRB3*02:02_D76A |
| DRB3*02:02_N77T | DRB3*02:02_N77A |
| DRB3*02:02_H81Y | DRB3*02:02_H81A |
| DRB3*02:02_V85A | |
| DRB3*02:02_H96Q | DRB3*02:02_H96A |
| DRB3*02:02_Q96K | DRB3*02:02_Q96A |
| DRB3*02:02_A104S | |
| DRB3*02:02_K105S | DRB3*02:02_K105S |
| DRB3*02:02_P108T | DRB3*02:02_P108S |
| DRB3*02:02_G135S | DRB3*02:02_G135A |
| DRB3*02:02_A140T | |
| DRB3*02:02_V164F | DRB3*02:02_V164A |
| DRB3*02:02_V180M | DRB3*02:02_V180A |
| DRB3*02:02_T181M | DRB3*02:02_T181A |
| DRB3*02:02_E187Q | DRB3*02:02_E187A |
| DRB3*02:02_S189R | DRB3*02:02_S189A |
| DRB3*02:02_R191Q | DRB3*02:02_R191A |
| DRB3*03:01_R4Q | DRB3*03:01_R4A |
| DRB3*03:01_E9Q | DRB3*03:01_E9A |
| DRB3*03:01_L11R | DRB3*03:01_L11A |
| DRB3*03:01_F18L | DRB3*03:01_F18A |
| DRB3*03:01_R25W | DRB3*03:01_R25A |
| DRB3*03:01_F31I | DRB3*03:01_F31A |
| DRB3*03:01_H32Y | DRB3*03:01_H32A |
| DRB3*03:01_V44L | DRB3*03:01_V44A |
| DRB3*03:01_R48Q | DRB3*03:01_R48A |
| DRB3*03:01_R55L | DRB3*03:01_R55A |
| DRB3*03:01_L67F | DRB3*03:01_L67A |
| DRB3*03:01_Q70R | DRB3*03:01_Q70A |
| DRB3*03:01_G73A | |
| DRB3*03:01_Q74R | DRB3*03:01_Q74A |
| DRB3*03:01_D76G | DRB3*03:01_D76A |
| DRB3*03:01_N77T | DRB3*03:01_N77A |
| DRB3*03:01_H81Y | DRB3*03:01_H81A |
| DRB3*03:01_V85A | |
| DRB3*03:01_H96Q | DRB3*03:01_H96A |
| DRB3*03:01_Q96K | DRB3*03:01_Q96A |
| DRB3*03:01_A104S | |
| DRB3*03:01_K105R | DRB3*03:01_K105A |
| DRB3*03:01_P108T | DRB3*03:01_P108A |
| DRB3*03:01_G135S | DRB3*03:01_G135A |
| DRB3*03:01_T140A | |
| DRB3*03:01_V164F | DRB3*03:01_V164A |
| DRB3*03:01_V180M | DRB3*03:01_V180A |
| DRB3*03:01_T181M | DRB3*03:01_T181A |
| DRB3*03:01_E187Q | DRB3*03:01_E187A |
| DRB3*03:01_R189S | DRB3*03:01_R189A |
| DRB3*03:01_R191Q | DRB3*03:01_R191A |
| DRB4*01:01_Q4R | DRB4*01:01_Q4A |
| DRB4*01:01_E9Q | DRB4*01:01_E9A |
| DRB4*01:01_A11D | |
| DRB4*01:01_L18F | DRB4*01:01_L18A |
| DRB4*01:01_W25R | DRB4*01:01_W25A |
| DRB4*01:01_I31F | DRB4*01:01_I31A |
| DRB4*01:01_Y32H | DRB4*01:01_Y32A |
| DRB4*01:01_L44V | DRB4*01:01_L44A |
| DRB4*01:01_Q48R | DRB4*01:01_Q48A |
| DRB4*01:01_R55L | DRB4*01:01_R55A |
| DRB4*01:01_F67L | DRB4*01:01_F67A |
| DRB4*01:01_R70D | DRB4*01:01_R70A |
| DRB4*01:01_A73G | |
| DRB4*01:01_E74L | DRB4*01:01_E74A |
| DRB4*01:01_D76G | DRB4*01:01_D76A |
| DRB4*01:01_T77N | DRB4*01:01_T77A |
| DRB4*01:01_Y81H | DRB4*01:01_Y81A |
| DRB4*01:01_V85A | |
| DRB4*01:01_Q96H | DRB4*01:01_Q96A |
| DRB4*01:01_K96Q | DRB4*01:01_K96A |
| DRB4*01:01_S104A | |
| DRB4*01:01_K105R | DRB4*01:01_K105A |
| DRB4*01:01_P108T | DRB4*01:01_P108A |
| DRB4*01:01_S135G | DRB4*01:01_S135A |
| DRB4*01:01_A140T | |
| DRB4*01:01_V164F | DRB4*01:01_V164A |
| DRB4*01:01_M180V | DRB4*01:01_M180A |
| DRB4*01:01_M181T | DRB4*01:01_M181A |
| DRB4*01:01_Q187E | DRB4*01:01_Q187A |
| DRB4*01:01_S189R | DRB4*01:01_S189A |
| DRB4*01:01_R191Q | DRB4*01:01_R191A |
| DRB5*01:01_R4Q | DRB5*01:01_R4A |
| DRB5*01:01_Q9E | DRB5*01:01_Q9A |
| DRB5*01:01_D11L | DRB5*01:01_D11A |
| DRB5*01:01_F18L | DRB5*01:01_F18A |
| DRB5*01:01_R25W | DRB5*01:01_R25A |
| DRB5*01:01_I31F | DRB5*01:01_I31A |
| DRB5*01:01_Y32H | DRB5*01:01_Y32A |
| DRB5*01:01_V44L | DRB5*01:01_V44A |
| DRB5*01:01_R48Q | DRB5*01:01_R48A |
| DRB5*01:01_R55L | DRB5*01:01_R55A |
| DRB5*01:01_F67L | DRB5*01:01_F67A |
| DRB5*01:01_D70R | DRB5*01:01_D70A |
| DRB5*01:01_A73G | |
| DRB5*01:01_A74L | |
| DRB5*01:01_D76G | DRB5*01:01_D76A |
| DRB5*01:01_T77N | DRB5*01:01_T77A |
| DRB5*01:01_H81Y | DRB5*01:01_H81A |
| DRB5*01:01_V85A | |
| DRB5*01:01_E96Q | DRB5*01:01_E96A |
| DRB5*01:01_K96Q | DRB5*01:01_K96A |
| DRB5*01:01_A104S | |
| DRB5*01:01_R105K | DRB5*01:01_R105A |
| DRB5*01:01_T108P | DRB5*01:01_T108A |
| DRB5*01:01_S135G | DRB5*01:01_S135A |
| DRB5*01:01_A140T | |
| DRB5*01:01_V164F | DRB5*01:01_V164A |
| DRB5*01:01_V180M | DRB5*01:01_V180A |
| DRB5*01:01_T181M | DRB5*01:01_T181A |
| DRB5*01:01_E187Q | DRB5*01:01_E187A |
| DRB5*01:01_R189S | DRB5*01:01_R189A |
| DRB5*01:01_Q191R | DRB5*01:01_Q191A |
| DRB5*02:02_R4Q | DRB5*02:02_R4A |
| DRB5*02:02_Q9E | DRB5*02:02_Q9A |
| DRB5*02:02_D11L | DRB5*02:02_D11A |
| DRB5*02:02_F18L | DRB5*02:02_F18A |
| DRB5*02:02_R25W | DRB5*02:02_R25A |
| DRB5*02:02_I31F | DRB5*02:02_I31A |
| DRB5*02:02_Y32H | DRB5*02:02_Y32A |
| DRB5*02:02_V44L | DRB5*02:02_V44A |
| DRB5*02:02_R48Q | DRB5*02:02_R48A |
| DRB5*02:02_R55L | DRB5*02:02_R55A |
| DRB5*02:02_I67L | DRB5*02:02_I67A |
| DRB5*02:02_Q70D | DRB5*02:02_Q70A |
| DRB5*02:02_A73G | |
| DRB5*02:02_A74L | |
| DRB5*02:02_D76G | DRB5*02:02_D76A |
| DRB5*02:02_T77N | DRB5*02:02_T77A |
| DRB5*02:02_H81Y | DRB5*02:02_H81A |
| DRB5*02:02_A85V | |
| DRB5*02:02_E96Q | DRB5*02:02_E96A |
| DRB5*02:02_K96Q | DRB5*02:02_K96A |
| DRB5*02:02_A104S | |
| DRB5*02:02_R105K | DRB5*02:02_R105A |
| DRB5*02:02_T108P | DRB5*02:02_T108A |
| DRB5*02:02_G135S | DRB5*02:02_G135A |
| DRB5*02:02_A140T | |
| DRB5*02:02_V164F | DRB5*02:02_V164A |
| DRB5*02:02_V180M | DRB5*02:02_V180A |
| DRB5*02:02_T181M | DRB5*02:02_T181A |
| DRB5*02:02_E187Q | DRB5*02:02_E187A |
| DRB5*02:02_R189S | DRB5*02:02_R189A |
| DRB5*02:02_Q191R | DRB5*02:02_Q191A |

### Example 9: Design of HLA Class I Epitope Panel and Engineered Variants

### Design of HLA Class I Epitope Panel

Similar as described in the design of HLA-DR panel, HLA-CI Common alleles were retrieved from CIWD 3.0 and analyzed with AA3D for panel design. Because HLA-A, HLA-B and HLA-C Ag share a very similar sequence and structural framework, all CI Ag sequences are commonly compared as a group. The resulting panel design by AA3D is listed in Table 15A and Table 15B.

TABLE 15A
List of HLA CI antigens selected for feasibility epitope
panel based on combined analysis of all 3 CI loci. 151
Ag are selected. 56 Ag are already included on One Lambda
LABScreen ™ CII SAB panels (in bold) and 2 Ag
only in Werfen LSA ™ Class I SAB panel (underlined).

| Count | A | Count | B | Count | C |
|---|---|---|---|---|---|
| 1 | A*01:02 | 1 | B*07:04 | 1 | C*01:03 |
| 2 | A*01:09 | 2 | B*07:10 | 2 | C*01:08 |
| 3 | A*02:02 | 3 | B*08:01 | 3 | C*02:02 |
| 4 | A*02:03 | 4 | B*13:02 | 4 | C*02:14 |
| 5 | A*02:04 | 5 | B*14:02 | 5 | C*03:02 |
| 6 | A*02:05 | 6 | B*15:02 | 6 | C*03:06 |
| 7 | A*02:06 | 7 | B*15:08 | 7 | C*03:88 |
| 8 | A*02:07 | 8 | B*15:11 | 8 | C*04:19 |
| 9 | A*02:09 | 9 | B*15:12 | 9 | C*04:27 |
| 10 | A*02:10 | 10 | B*15:16 | 10 | C*05:01 |
| 11 | A*02:17 | 11 | B*15:21 | 11 | C*06:27 |
| 12 | A*02:30 | 12 | B*15:27 | 12 | C*07:02 |
| 13 | A*02:33 | 13 | B*15:29 | 13 | C*07:04 |
| 14 | A*02:35 | 14 | B*15:30 | 14 | C*07:05 |
| 15 | A*02:60 | 15 | B*15:32 | 15 | C*07:29 |
| 16 | A*02:64 | 16 | B*15:35 | 16 | C*07:35 |
| 17 | A*02:85 | 17 | B*15:75 | 17 | C*08:06 |
| 18 | A*03:01 | 18 | B*18:01 | 18 | C*12:12 |
| 19 | A*03:02 | 19 | B*18:02 | 19 | C*14:03 |
| 20 | A*11:02 | 20 | B*18:05 | 20 | C*15:13 |
| 21 | A*23:15 | 21 | B*27:03 | 21 | C*16:01 |
| 22 | A*24:07 | 22 | B*27:06 | 22 | C*16:04 |
| 23 | A*24:14 | 23 | B*27:07 | 23 | C*17:01 |
| 24 | A*24:17 | 24 | B*27:09 | 24 | C*18:01 |
| 25 | A*24:22 | 25 | B*27:12 | | |
| 26 | A*24:23 | 26 | B*27:14 | | |
| 27 | A*24:25 | 27 | B*35:02 | | |
| 28 | A*24:53 | 28 | B*35:03 | | |
| 29 | A*24:95 | 29 | B*35:16 | | |
| 30 | A*26:02 | 30 | B*35:17 | | |
| 31 | A*26:03 | 31 | B*35:23 | | |
| 32 | A*26:08 | 32 | B*35:48 | | |
| 33 | A*26:17 | 33 | B*37:01 | | |
| 34 | A*29:01 | 34 | B*38:09 | | |
| 35 | A*30:01 | 35 | B*39:06 | | |
| 36 | A*30:04 | 36 | B*39:08 | | |
| 37 | A*30:10 | 37 | B*39:09 | | |
| 38 | A*31:02 | 38 | B*39:12 | | |
| 39 | A*31:04 | 39 | B*39:20 | | |
| 40 | A*31:09 | 40 | B*39:24 | | |
| 41 | A*31:12 | 41 | B*39:31 | | |
| 42 | A*31:15 | 42 | B*40:01 | | |
| 43 | A*31:16 | 43 | B*40:04 | | |
| 44 | A*32:01 | 44 | B*40:06 | | |
| 45 | A*33:05 | 45 | B*40:08 | | |
| 46 | A*33:26 | 46 | B*40:13 | | |
| 47 | A*34:05 | 47 | B*40:23 | | |
| 48 | A*68:02 | 48 | B*40:27 | | |
| 49 | A*68:05 | 49 | B*40:90 | | |
| 50 | A*68:07 | 50 | B*42:02 | | |
| 51 | A*68:12 | 51 | B*44:04 | | |
| 52 | A*68:17 | 52 | B*44:15 | | |
| 53 | A*68:24 | 53 | B*44:29 | | |
| 54 | A*69:01 | 54 | B*46:01 | | |
| 55 | A*74:03 | 55 | B*47:01 | | |
| 56 | A*74:09 | 56 | B*47:03 | | |
| 57 | A*74:11 | 57 | B*48:01 | | |
| 58 | A*80:01 | 58 | B*48:02 | | |
| | | 59 | B*51:14 | | |
| | | 60 | B*51:22 | | |
| | | 61 | B*52:04 | | |
| | | 62 | B*54:01 | | |
| | | 63 | B*55:01 | | |
| | | 64 | B*57:01 | | |
| | | 65 | B*57:03 | | |
| | | 66 | B*57:04 | | |
| | | 67 | B*58:02 | | |
| | | 68 | B*73:01 | | |
| | | 69 | B*82:01 | | |

Alternatively, HLA-A, HLA-B and HLA-C Ag can be analyzed separately for each locus. The resulting panel design is listed in Table 15B.

TABLE 15B
List of HLA CI antigens selected for feasibility epitope
panel based on individual analysis of each 3 CI locus.
164 Ag are selected. 56 Ag are already included on One
Lambda LABScreen ™ CII SAB panels (in bold)
and 2 Ag only in Werfen LSA ™ Class I SAB panel (underlined).

| Count | A | Count | B | Count | C |
|---|---|---|---|---|---|
| 1 | A*01:02 | 1 | B*07:04 | 1 | C*01:03 |
| 2 | A*01:06 | 2 | B*07:10 | 2 | C*01:08 |
| 3 | A*01:09 | 3 | B*07:14 | 3 | C*02:02 |
| 4 | A*02:02 | 4 | B*08:01 | 4 | C*02:14 |
| 5 | A*02:03 | 5 | B*13:02 | 5 | C*03:02 |
| 6 | A*02:04 | 6 | B*14:02 | 6 | C*03:05 |
| 7 | A*02:05 | 7 | B*15:02 | 7 | C*03:06 |
| 8 | A*02:07 | 8 | B*15:06 | 8 | C*03:88 |
| 9 | A*02:09 | 9 | B*15:08 | 9 | C*04:03 |
| 10 | A*02:10 | 10 | B*15:11 | 10 | C*04:19 |
| 11 | A*02:16 | 11 | B*15:12 | 11 | C*04:27 |
| 12 | A*02:17 | 12 | B*15:16 | 12 | C*05:01 |
| 13 | A*02:20 | 13 | B*15:17 | 13 | C*06:09 |
| 14 | A*02:30 | 14 | B*15:21 | 14 | C*06:27 |
| 15 | A*02:33 | 15 | B*15:27 | 15 | C*07:04 |
| 16 | A*02:35 | 16 | B*15:29 | 16 | C*07:05 |
| 17 | A*02:60 | 17 | B*15:30 | 17 | C*07:26 |
| 18 | A*02:64 | 18 | B*15:32 | 18 | C*07:29 |
| 19 | A*02:85 | 19 | B*15:33 | 19 | C*07:35 |
| 20 | A*03:01 | 20 | B*15:35 | 20 | C*08:06 |
| 21 | A*03:02 | 21 | B*15:75 | 21 | C*12:12 |
| 22 | A*11:02 | 22 | B*18:02 | 22 | C*14:03 |
| 23 | A*11:03 | 23 | B*18:05 | 23 | C*15:05 |
| 24 | A*23:15 | 24 | B*27:02 | 24 | C*15:13 |
| 25 | A*24:07 | 25 | B*27:03 | 25 | C*16:01 |
| 26 | A*24:14 | 26 | B*27:06 | 26 | C*16:04 |
| 27 | A*24:17 | 27 | B*27:07 | 27 | B*17:01 |
| 28 | A*24:22 | 28 | B*27:09 | 28 | C*18:01 |
| 29 | A*24:23 | 29 | B*27:12 | | |
| 30 | A*24:25 | 30 | B*27:14 | | |
| 31 | A*24:53 | 31 | B*35:02 | | |
| 32 | A*24:95 | 32 | B*35:03 | | |
| 33 | A*26:02 | 33 | B*35:16 | | |
| 34 | A*26:03 | 34 | B*35:17 | | |
| 35 | A*26:08 | 35 | B*35:23 | | |
| 36 | A*26:17 | 36 | B*35:48 | | |
| 37 | A*29:01 | 37 | B*37:01 | | |
| 38 | A*29:10 | 38 | B*38:09 | | |
| 39 | A*30:01 | 39 | B*39:06 | | |
| 40 | A*30:04 | 40 | B*39:08 | | |
| 41 | A*30:10 | 41 | B*39:09 | | |
| 42 | A*31:02 | 42 | B*39:12 | | |
| 43 | A*31:04 | 43 | B*39:20 | | |
| 44 | A*31:09 | 44 | B*39:24 | | |
| 45 | A*31:12 | 45 | B*39:31 | | |
| 46 | A*31:15 | 46 | B*40:01 | | |
| 47 | A*31:16 | 47 | B*40:04 | | |
| 48 | A*32:01 | 48 | B*40:06 | | |
| 49 | A*33:05 | 49 | B*40:08 | | |
| 50 | A*33:26 | 50 | B*40:13 | | |
| 51 | A*34:05 | 51 | B*40:23 | | |
| 52 | A*36:03 | 52 | B*40:27 | | |
| 53 | A*68:02 | 53 | B*40:90 | | |
| 54 | A*68:05 | 54 | B*42:02 | | |
| 55 | A*68:07 | 55 | B*44:04 | | |
| 56 | A*68:12 | 56 | B*44:15 | | |
| 57 | A*68:13 | 57 | B*44:29 | | |
| 58 | A*68:17 | 58 | B*46:01 | | |
| 59 | A*68:24 | 59 | B*47:01 | | |
| 60 | A*69:01 | 60 | B*47:03 | | |
| 61 | A*74:03 | 61 | B*48:02 | | |
| 62 | A*74:09 | 62 | B*48:07 | | |
| 63 | A*74:11 | 63 | B*51:14 | | |
| 64 | A*80:01 | 64 | B*51:22 | | |
| | | 65 | B*52:04 | | |
| | | 66 | B*54:01 | | |
| | | 67 | B*57:01 | | |
| | | 68 | B*57:03 | | |
| | | 69 | B*57:04 | | |
| | | 70 | B*58:02 | | |
| | | 71 | B*73:01 | | |
| | | 72 | B*82:01 | | |

It is apparent that a significantly smaller number of total HLA CI Ag are selected (151 vs 164) if all HLA-A, HLA-B and HLA-C Common alleles are compiled together for panel design by AA3D instead of analyzing individual locus separately. To further minimize the Ag listed in Table 15B, they can be theoretically subject to another round of combined analysis to eliminate cross-loci redundancy. The resulting panel is listed in Table 15C.

TABLE 15C
List of HLA CI antigens selected for feasibility
epitope panel based on combined analysis of all
Ag listed on Table 15B. 147 Ag are selected.

| Count | A | Count | B | Count | C |
|---|---|---|---|---|---|
| 1 | A*01:02 | 1 | B*07:04 | 1 | C*01:03 |
| 2 | A*01:09 | 2 | B*07:10 | 2 | C*01:08 |
| 3 | A*02:02 | 3 | B*08:01 | 3 | C*02:02 |
| 4 | A*02:03 | 4 | B*13:02 | 4 | C*02:14 |
| 5 | A*02:04 | 5 | B*14:02 | 5 | C*03:02 |
| 6 | A*02:05 | 6 | B*15:02 | 6 | C*03:06 |
| 7 | A*02:07 | 7 | B*15:08 | 7 | C*03:88 |
| 8 | A*02:09 | 8 | B*15:11 | 8 | C*04:19 |
| 9 | A*02:10 | 9 | B*15:12 | 9 | C*04:27 |
| 10 | A*02:17 | 10 | B*15:16 | 10 | C*05:01 |
| 11 | A*02:30 | 11 | B*15:21 | 11 | C*06:27 |
| 12 | A*02:33 | 12 | B*15:27 | 12 | C*07:04 |
| 13 | A*02:35 | 13 | B*15:29 | 13 | C*07:05 |
| 14 | A*02:60 | 14 | B*15:30 | 14 | C*07:29 |
| 15 | A*02:64 | 15 | B*15:32 | 15 | C*07:35 |
| 16 | A*02:85 | 16 | B*15:35 | 16 | C*08:06 |
| 17 | A*03:01 | 17 | B*15:75 | 17 | C*12:12 |
| 18 | A*03:02 | 18 | B*18:02 | 18 | C*14:03 |
| 19 | A*11:02 | 19 | B*18:05 | 19 | C*15:13 |
| 20 | A*23:15 | 20 | B*27:03 | 20 | C*16:01 |
| 21 | A*24:07 | 21 | B*27:06 | 21 | C*16:04 |
| 22 | A*24:14 | 22 | B*27:07 | 22 | C*17:01 |
| 23 | A*24:17 | 23 | B*27:09 | 23 | C*18:01 |
| 24 | A*24:22 | 24 | B*27:12 | | |
| 25 | A*24:23 | 25 | B*27:14 | | |
| 26 | A*24:25 | 26 | B*35:02 | | |
| 27 | A*24:53 | 27 | B*35:03 | | |
| 28 | A*24:95 | 28 | B*35:16 | | |
| 29 | A*26:02 | 29 | B*35:23 | | |
| 30 | A*26:03 | 30 | B*35:48 | | |
| 31 | A*26:08 | 31 | B*37:01 | | |
| 32 | A*26:17 | 32 | B*38:09 | | |
| 33 | A*29:01 | 33 | B*39:06 | | |
| 34 | A*30:01 | 34 | B*39:08 | | |
| 35 | A*30:04 | 35 | B*39:09 | | |
| 36 | A*30:10 | 36 | B*39:12 | | |
| 37 | A*31:02 | 37 | B*39:20 | | |
| 38 | A*31:04 | 38 | B*39:24 | | |
| 39 | A*31:09 | 39 | B*39:31 | | |
| 40 | A*31:12 | 40 | B*40:01 | | |
| 41 | A*31:15 | 41 | B*40:04 | | |
| 42 | A*31:16 | 42 | B*40:06 | | |
| 43 | A*32:01 | 43 | B*40:08 | | |
| 44 | A*33:05 | 44 | B*40:13 | | |
| 45 | A*33:26 | 45 | B*40:23 | | |
| 46 | A*34:05 | 46 | B*40:27 | | |
| 47 | A*68:02 | 47 | B*40:90 | | |
| 48 | A*68:05 | 48 | B*42:02 | | |
| 49 | A*68:07 | 49 | B*44:04 | | |
| 50 | A*68:12 | 50 | B*44:15 | | |
| 51 | A*68:17 | 51 | B*44:29 | | |
| 52 | A*68:24 | 52 | B*46:01 | | |
| 53 | A*69:01 | 53 | B*47:01 | | |
| 54 | A*74:03 | 54 | B*47:03 | | |
| 55 | A*74:09 | 55 | B*48:02 | | |
| 56 | A*74:11 | 56 | B*48:07 | | |
| 57 | A*80:01 | 57 | B*51:14 | | |
| | | 58 | B*51:22 | | |
| | | 59 | B*54:01 | | |
| | | 60 | B*52:04 | | |
| | | 61 | B*54:01 | | |
| | | 62 | B*57:01 | | |
| | | 63 | B*57:03 | | |
| | | 64 | B*57:04 | | |
| | | 65 | B*58:02 | | |
| | | 66 | B*73:01 | | |
| | | 67 | B*82:01 | | |

Some differences are noted between the list (Table 15A) directly compiled from combined HLA CI analysis and the list (Table 15C) based on individual locus analysis first before combined analysis. Although the second approach leads to a lower number of Ag (147) compared with the first approach (151 Ag), it is still to be determined which approach provides a better coverage.

A HLA CI epitope panel can be produced for the panels disclosed in Table 15A, 15B or 15C, according to the methods disclosed in Examples 1-6.

### Design of HLA Class I Engineered Variants

Total and prioritized variant positions among A, B or C Common alleles (CIWD 3.0) alleles are listed in Table 16A. Prioritized variant positions cover proposed Ab accessible positions and positions likely impact peptide selection for presentation.

TABLE 16A
List of HLA Class I variant positions among common alleles
of each A, B or C locus suitable for EV generation.
HLA Class I Variant Positions

| A | | B | | C | | A | | B | | C | |
|---|---|---|---|---|---|---|---|---|---|---|---|
| Total | Prioritized | Total | Prioritized | Total | Prioritized | Total | Prioritized | Total | Prioritized | Total | Prioritized |
| 3 | | 4 | | 1 | 1 | 107 | | 138 | 138 | 219 | 219 |
| 6 | | 9 | | 6 | | 109 | 109 | 143 | 143 | 229 | 229 |
| 7 | 7 | 11 | | 9 | 9 | 114 | 114 | 145 | 145 | 248 | 248 |
| 9 | 9 | 12 | | 11 | | 116 | | 147 | 147 | 253 | 253 |
| 12 | | 24 | | 14 | 14 | 125 | | 152 | 152 | 261 | |
| 14 | | 30 | | 16 | 16 | 127 | 127 | 156 | 156 | 267 | 267 |
| 17 | 17 | 32 | | 21 | | 141 | 141 | 158 | 158 | 270 | 270 |
| 19 | 19 | 41 | 41 | 24 | | 142 | 142 | 159 | 159 | 273 | 273 |
| 30 | 30 | 43 | 43 | 35 | | 144 | 144 | 162 | 162 | 275 | 275 |
| 31 | 31 | 45 | | 49 | | 145 | 145 | 163 | 163 | | |
| 33 | 33 | 46 | 46 | 66 | 66 | 149 | 149 | 166 | 166 | | |
| 35 | 35 | 52 | | 73 | 73 | 150 | 150 | 167 | 167 | | |
| 43 | 43 | 58 | | 76 | 76 | 151 | 151 | 171 | 171 | | |
| 44 | 44 | 62 | 62 | 77 | 77 | 152 | 152 | 177 | 177 | | |
| 47 | 47 | 63 | 63 | 80 | 80 | 156 | 156 | 178 | 178 | | |
| 50 | 50 | 65 | 65 | 90 | 90 | 158 | 158 | 180 | 180 | | |
| 54 | 54 | 66 | 66 | 91 | 91 | 161 | 161 | 194 | 194 | | |
| 56 | 56 | 67 | | 94 | | 163 | 163 | 199 | | | |
| 62 | 62 | 68 | 68 | 96 | | 166 | 166 | 211 | | | |
| 63 | 63 | 70 | 70 | 97 | 97 | 167 | 167 | 239 | | | |
| 65 | 65 | 71 | | 99 | 99 | 171 | 171 | 245 | | | |
| 66 | 66 | 74 | | 103 | | 177 | 177 | 253 | 253 | | |
| 67 | 67 | 76 | 76 | 106 | 106 | 184 | 184 | 267 | 267 | | |
| 68 | 68 | 77 | 77 | 113 | | 186 | 186 | 268 | 268 | | |
| 69 | 69 | 80 | 80 | 114 | | 193 | 193 | 270 | 270 | | |
| 70 | 70 | 81 | 81 | 116 | | 194 | 194 | 275 | 275 | | |
| 73 | 73 | 82 | 82 | 138 | 138 | 207 | | | | | |
| 74 | 74 | 83 | 83 | 143 | 143 | 236 | | | | | |
| 76 | 76 | 90 | 90 | 147 | 147 | 245 | | | | | |
| 77 | 77 | 94 | | 151 | 151 | 246 | | | | | |
| 79 | 79 | 95 | | 152 | 152 | 253 | 253 | | | | |
| 80 | 80 | 97 | 97 | 156 | 156 | 255 | 255 | | | | |
| 81 | 81 | 98 | | 160 | | 268 | 268 | | | | |
| 82 | 82 | 99 | 99 | 163 | 163 | 276 | 276 | | | | |
| 83 | 83 | 103 | | 170 | 170 | 245 | | | | | |
| 90 | 90 | 109 | 109 | 173 | 173 | 246 | | | | | |
| 92 | | 113 | | 175 | | 253 | | | | | |
| 95 | 95 | 114 | | 177 | 177 | 255 | | | | | |
| 97 | 97 | 116 | | 184 | 184 | 268 | | | | | |
| 99 | 99 | 127 | 127 | 193 | 193 | 276 | | | | | |
| 102 | | 131 | 131 | 194 | 194 | 280 | | | | | |
| 105 | 105 | 137 | 137 | 211 | | | | | | | |

For HLA Class I alleles, only representative alleles with total frequency ≥5% (CIWD 3.0) are listed as examples for EV designs (FIG. 16B). Some EV designs are underlined because they are changes among small residues (amino acids A, G and S) that may not result in a conspicuous impact.

Tables 16B, 16C, and 16D. List of designed HLA Class I engineered variants suitable for ER and/or AR verification.

TABLE 16B
Exemplary HLA-A EVs.
HLA-A Prioritized EV Design

| Change to existing e p | Change to alanine |
|---|---|
| A*01:01_Y7C | A*01:01_Y7A |
| A*01:01_F9S | A*01:01_F9A |
| A*01:01_R17S | A*01:01_R17A |
| A*01:01_E19K | A*01:01_E19A |
| A*01:01_Q43R | A*01:01_Q43A |
| A*01:01_K44R | A*01:01_K44A |
| A*01:01_P47S | A*01:01_P47A |
| A*01:01_P50L | A*01:01_P50A |
| A*01:01_Q54R | A*01:01_Q54A |
| A*01:01_G56R | A*01:01_G56A |
| A*01:01_Q62G | A*01:01_Q62A |
| A*01:01_E63N | A*01:01_E63A |
| A*01:01_R65G | A*01:01_R65A |
| A*01:01_N66K | A*01:01_N66A |
| A*01:01_M67V | A*01:01_M67A |
| A*01:01_K68R | A*01:01_K68A |
| A*01:01_A69G | |
| A*01:01_H70Q | A*01:01_H70A |
| A*01:01_T73I | A*01:01_T73A |
| A*01:01_D74H | A*01:01_D74A |
| A*01:01_A75V | |
| A*01:01_N77D | A*01:01_N77A |
| A*01:01_G79R | A*01:01_G79A |
| A*01:01_T80I | A*01:01_T80A |
| A*01:01_L81A | |
| A*01:01_R82L | A*01:01_R82A |
| A*01:01_G83R | A*01:01_G83A |
| A*01:01_D90A | |
| A*01:01_I95V | A*01:01_I95A |
| A*02:01_Y7C | A*02:01_Y7A |
| A*02:01_F9S | A*02:01_F9A |
| A*02:01_R17S | A*02:01_R17A |
| A*02:01_E19K | A*02:01_E19A |
| A*02:01_Q43R | A*02:01_Q43A |
| A*02:01_R44K | A*02:01_R44A |
| A*02:01_P47S | A*02:01_P47A |
| A*02:01_P50L | A*02:01_P50A |
| A*02:01_Q54R | A*02:01_Q54A |
| A*02:01_G56R | A*02:01_G56A |
| A*02:01_G62Q | A*02:01_G62A |
| A*02:01_E63N | A*02:01_E63A |
| A*02:01_R65G | A*02:01_R65A |
| A*02:01_K66N | A*02:01_K66A |
| A*02:01_V67M | A*02:01_V67A |
| A*02:01_K68R | A*02:01_K68A |
| A*02:01_A69G | |
| A*02:01_H70Q | A*02:01_H70A |
| A*02:01_T73I | A*02:01_T73A |
| A*02:01_H74D | A*02:01_H74A |
| A*02:01_V75A | |
| A*02:01_D77N | A*02:01_D77A |
| A*02:01_G79R | A*02:01_G79A |
| A*02:01_T80I | A*02:01_T80A |
| A*02:01_L81A | |
| A*02:01_R82L | A*02:01_R82A |
| A*02:01_G83R | A*02:01_G83A |
| A*02:01_A90D | |
| A*02:01_V95I | A*02:01_V95A |
| A*03:01_Y7C | A*03:01_Y7A |
| A*03:01_Y9S | A*03:01_Y9A |
| A*03:01_R17S | A*03:01_R17A |
| A*03:01_E19K | A*03:01_E19A |
| A*03:01_Q43R | A*03:01_Q43A |
| A*03:01_R44K | A*03:01_R44A |
| A*03:01_P47S | A*03:01_P47A |
| A*03:01_P50L | A*03:01_P50A |
| A*03:01_Q54R | A*03:01_Q54A |
| A*03:01_G56R | A*03:01_G56A |
| A*03:01_Q62E | A*03:01_Q62A |
| A*03:01_E63N | A*03:01_E63A |
| A*03:01_R65G | A*03:01_R65A |
| A*03:01_N66K | A*03:01_N66A |
| A*03:01_V67M | A*03:01_V67A |
| A*03:01_K68R | A*03:01_K68A |
| A*03:01_A69G | |
| A*03:01_Q70H | A*03:01_Q70A |
| A*03:01_T73I | A*03:01_T73A |
| A*03:01_D74H | A*03:01_D74A |
| A*03:01_V76E | A*03:01_V76A |
| A*03:01_D77N | A*03:01_D77A |
| A*03:01_G79R | A*03:01_G79A |
| A*03:01_T80I | A*03:01_T80A |
| A*03:01_L81A | |
| A*03:01_R82L | A*03:01_R82A |
| A*03:01_G83R | A*03:01_G83A |
| A*03:01_A90D | |
| A*03:01_I95L | A*03:01_I95A |
| A*11:01_Y7C | A*11:01_Y7A |
| A*11:01_F9S | A*11:01_F9A |
| A*11:01_R17S | A*11:01_R17A |
| A*11:01_E19K | A*11:01_E19A |
| A*11:01_Q43R | A*11:01_Q43A |
| A*11:01_R44K | A*11:01_R44A |
| A*11:01_P47S | A*11:01_P47A |
| A*11:01_P50L | A*11:01_P50A |
| A*11:01_Q54R | A*11:01_Q54A |
| A*11:01_G56R | A*11:01_G56A |
| A*11:01_Q62E | A*11:01_Q62A |
| A*11:01_E63N | A*11:01_E63A |
| A*11:01_R65G | A*11:01_R65A |
| A*11:01_N66K | A*11:01_N66A |
| A*11:01_V67M | A*11:01_V67A |
| A*11:01_K68R | A*11:01_K68A |
| A*11:01_A69G | |
| A*11:01_Q70H | A*11:01_Q70A |
| A*11:01_T73I | A*11:01_T73A |
| A*11:01_D74H | A*11:01_D74A |
| A*11:01_V76E | A*11:01_V76A |
| A*11:01_D77N | A*11:01_D77A |
| A*11:01_G79R | A*11:01_G79A |
| A*11:01_T80I | A*11:01_T80A |
| A*11:01_L81A | |
| A*11:01_R82L | A*11:01_R82A |
| A*11:01_G83R | A*11:01_G83A |
| A*11:01_A90D | |
| A*11:01_I95L | A*11:01_I95A |
| A*24:02_Y7C | A*24:02_Y7A |
| A*24:02_S9F | A*24:02_S9A |
| A*24:02_R17S | A*24:02_R17A |
| A*24:02_E19K | A*24:02_E19A |
| A*24:02_Q43R | A*24:02_Q43A |
| A*24:02_R44K | A*24:02_R44A |
| A*24:02_P47S | A*24:02_P47A |
| A*24:02_P50L | A*24:02_P50A |
| A*24:02_Q54R | A*24:02_Q54A |
| A*24:02_G56R | A*24:02_G56A |
| A*24:02_E62R | A*24:02_E62A |
| A*24:02_E63N | A*24:02_E63A |
| A*24:02_G65R | A*24:02_G65A |
| A*24:02_K66N | A*24:02_K66A |
| A*24:02_V67M | A*24:02_V67A |
| A*24:02_K68R | A*24:02_K68A |
| A*24:02_A69G | |
| A*24:02_H70Q | A*24:02_H70A |
| A*24:02_T73I | A*24:02_T73A |
| A*24:02_D74H | A*24:02_D74A |
| A*24:02_E76V | A*24:02_E76A |
| A*24:02_N77S | A*24:02_N77A |
| A*24:02_R79G | A*24:02_R79A |
| A*24:02_I80T | A*24:02_I80A |
| A*24:02_L81A | |
| A*24:02_L82R | A*24:02_L82A |
| A*24:02_R83G | A*24:02_R83A |
| A*24:02_A90D | |
| A*24:02_L95V | A*24:02_L95A |

| | |
|---|---|
| A*01:01_I97M | A*01:01_I97A |
| A*01:01_Y99F | A*01:01_Y99A |
| A*01:01_P105S | A*01:01_P105A |
| A*01:01_F109L | A*01:01_F109A |
| A*01:01_R114H | A*01:01_R114A |
| A*01:01_N127K | A*01:01_N127A |
| A*01:01_Q141E | A*01:01_Q141A |
| A*01:01_I142T | A*01:01_I142A |
| A*01:01_K144Q | A*01:01_K144A |
| A*01:01_R145H | A*01:01_R145A |
| A*01:01_A149T | |
| A*01:01_V150A | |
| A*01:01_H151R | A*01:01_H151A |
| A*01:01_A152V | |
| A*01:01_R156L | A*01:01_R156A |
| A*01:01_V158A | |
| A*01:01_E161D | A*01:01_E161A |
| A*01:01_R163T | A*01:01_R163A |
| A*01:01_D166E | A*01:01_D166A |
| A*01:01_G167W | A*01:01_G167A |
| A*01:01_Y171H | A*01:01_Y171A |
| A*01:01_E177K | A*01:01_E177A |
| A*01:01_P184A | |
| A*01:01_K186R | A*01:01_K186A |
| A*01:01_P193A | |
| A*01:01_I194V | A*01:01_I194A |
| A*01:01_E253Q | A*01:01_E253A |
| A*01:01_Q255K | A*01:01_Q255A |
| A*01:01_K268E | A*01:01_K268A |
| A*01:01_L276P | A*01:01_L276A |
| A*02:01_R97M | A*02:01_R97A |
| A*02:01_Y99F | A*02:01_Y99A |
| A*02:01_S105P | A*02:01_S105A |
| A*02:01_F109L | A*02:01_F109A |
| A*02:01_H114R | A*02:01_H114A |
| A*02:01_K127N | A*02:01_K127A |
| A*02:01_Q141E | A*02:01_Q141A |
| A*02:01_T142I | A*02:01_T142A |
| A*02:01_K144Q | A*02:01_K144A |
| A*02:01_H145R | A*02:01_H145A |
| A*02:01_A149T | |
| A*02:01_A150V | |
| A*02:01_H151R | A*02:01_H151A |
| A*02:01_V152E | A*02:01_V152A |
| A*02:01_L156W | A*02:01_L156A |
| A*02:01_A158V | |
| A*02:01_E161D | A*02:01_E161A |
| A*02:01_T163E | A*02:01_T163A |
| A*02:01_E166D | A*02:01_E166A |
| A*02:01_W167G | A*02:01_W167A |
| A*02:01_Y171H | A*02:01_Y171A |
| A*02:01_E177K | A*02:01_E177A |
| A*02:01_A184P | |
| A*02:01_K186R | A*02:01_K186A |
| A*02:01_A193P | |
| A*02:01_V194I | A*02:01_V194A |
| A*02:01_Q253E | A*02:01_Q253A |
| A*02:01_Q255K | A*02:01_Q255A |
| A*02:01_K268E | A*02:01_K268A |
| A*02:01_P276L | A*02:01_P276A |
| A*03:01_I97M | A*03:01_I97A |
| A*03:01_Y99F | A*03:01_Y99A |
| A*03:01_S105P | A*03:01_S105A |
| A*03:01_F109L | A*03:01_F109A |
| A*03:01_R114H | A*03:01_R114A |
| A*03:01_N127K | A*03:01_N127A |
| A*03:01_Q141E | A*03:01_Q141A |
| A*03:01_I142T | A*03:01_I142A |
| A*03:01_K144Q | A*03:01_K144A |
| A*03:01_R145H | A*03:01_R145A |
| A*03:01_A149T | |
| A*03:01_A150V | |
| A*03:01_H151R | A*03:01_H151A |
| A*03:01_E152V | A*03:01_E152A |
| A*03:01_L156W | A*03:01_L156A |
| A*03:01_A158V | |
| A*03:01_D161E | A*03:01_D161A |
| A*03:01_T163E | A*03:01_T163A |
| A*03:01_E166D | A*03:01_E166A |
| A*03:01_W167G | A*03:01_W167A |
| A*03:01_Y171H | A*03:01_Y171A |
| A*03:01_E177K | A*03:01_E177A |
| A*03:01_P184A | |
| A*03:01_K186R | A*03:01_K186A |
| A*03:01_P193A | |
| A*03:01_I194V | A*03:01_I194A |
| A*03:01_E253Q | A*03:01_E253A |
| A*03:01_Q255K | A*03:01_Q255A |
| A*03:01_K268E | A*03:01_K268A |
| A*03:01_L276P | A*03:01_L276A |
| A*11:01_I97M | A*11:01_I97A |
| A*11:01_Y99F | A*11:01_Y99A |
| A*11:01_P105S | A*11:01_P105A |
| A*11:01_F109L | A*11:01_F109A |
| A*11:01_R114H | A*11:01_R114A |
| A*11:01_N127K | A*11:01_N127A |
| A*11:01_Q141E | A*11:01_Q141A |
| A*11:01_I142T | A*11:01_I142A |
| A*11:01_K144Q | A*11:01_K144A |
| A*11:01_R145H | A*11:01_R145A |
| A*11:01_A149T | |
| A*11:01_A150V | |
| A*11:01_H151R | A*11:01_H151A |
| A*11:01_A152E | |
| A*11:01_Q156L | A*11:01_Q156A |
| A*11:01_A158V | |
| A*11:01_E161D | A*11:01_E161A |
| A*11:01_R163T | A*11:01_R163A |
| A*11:01_E166D | A*11:01_E166A |
| A*11:01_W167G | A*11:01_W167A |
| A*11:01_Y171H | A*11:01_Y171A |
| A*11:01_E177K | A*11:01_E177A |
| A*11:01_P184A | |
| A*11:01_K186R | A*11:01_K186A |
| A*11:01_P193A | |
| A*11:01_I194V | A*11:01_I194A |
| A*11:01_E253Q | A*11:01_E253A |
| A*11:01_Q255K | A*11:01_Q255A |
| A*11:01_K268E | A*11:01_K268A |
| A*11:01_L276P | A*11:01_L276A |
| A*24:02_M97R | A*24:02_M97A |
| A*24:02_F99Y | A*24:02_F99A |
| A*24:02_S105P | A*24:02_S105A |
| A*24:02_F109L | A*24:02_F109A |
| A*24:02_H114R | A*24:02_H114A |
| A*24:02_K127N | A*24:02_K127A |
| A*24:02_Q141E | A*24:02_Q141A |
| A*24:02_I142T | A*24:02_I142A |
| A*24:02_K144Q | A*24:02_K144A |
| A*24:02_R145H | A*24:02_R145A |
| A*24:02_A149T | |
| A*24:02_A150V | |
| A*24:02_H151R | A*24:02_H151A |
| A*24:02_V152E | A*24:02_V152A |
| A*24:02_Q156W | A*24:02_Q156A |
| A*24:02_A158V | |
| A*24:02_E161D | A*24:02_E161A |
| A*24:02_T163R | A*24:02_T163A |
| A*24:02_D166E | A*24:02_D166A |
| A*24:02_G167W | A*24:02_G167A |
| A*24:02_Y171H | A*24:02_Y171A |
| A*24:02_E177K | A*24:02_E177A |
| A*24:02_P184A | |
| A*24:02_K186R | A*24:02_K186A |
| A*24:02_P193A | |
| A*24:02_I194V | A*24:02_I194A |
| A*24:02_E253Q | A*24:02_E253A |
| A*24:02_Q255K | A*24:02_Q255A |
| A*24:02_K268E | A*24:02_K268A |
| A*24:02_P276L | A*24:02_P276A |

TABLE 16C
Exemplary HLA-B EVs
HLA-B Prioritized EV Design

| Change to existing e p | Change to alanine |
|---|---|
| B*07:02_A41T | |
| B*07:02_P43Q | B*07:02_P43A |
| B*07:02_E46A | |
| B*07:02_R62G | B*07:02_R62A |
| B*07:02_N63E | B*07:02_N63A |
| B*07:02_Q65R | B*07:02_Q65A |
| B*07:02_I66N | B*07:02_I66A |
| B*07:02_A69T | |
| B*07:02_Q70N | B*07:02_Q70A |
| B*07:02_E76V | B*07:02_E76A |
| B*07:02_S77N | B*07:02_S77A |
| B*07:02_N80T | B*07:02_N80A |
| B*07:02_L81A | |
| B*07:02_R82L | B*07:02_R82A |
| B*07:02_G83R | B*07:02_G83A |
| B*07:02_A90D | |
| B*07:02_S97R | B*07:02_S97A |
| B*07:02_Y99F | B*07:02_Y99A |
| B*07:02_L109F | B*07:02_L109A |
| B*07:02_N127K | B*07:02_N127A |
| B*07:02_R131S | B*07:02_R131A |
| B*07:02_D137E | B*07:02_D137A |
| B*07:02_T138K | B*07:02_T138A |
| B*07:02_T143S | B*07:02_T143A |
| B*07:02_R14 [symbol] L | B*07:02_R14 [symbol] A |
| B*07:02_W147L | B*07:02_W147A |
| B*07:02_E152V | B*07:02_E152A |
| B*07:02_R156D | B*07:02_R156A |
| B*07:02_A158T | |
| B*07:02_Y159H | B*07:02_Y159A |
| B*07:02_G162D | B*07:02_G162A |
| B*07:02_E163T | B*07:02_E163A |
| B*07:02_E166D | B*07:02_E166A |
| B*07:02_W167G | B*07:02_W167A |
| B*07:02_Y171H | B*07:02_Y171A |
| B*07:02_D177E | B*07:02_D177A |
| B*07:02_K178T | B*07:02_K178A |
| B*07:02_E180Q | B*07:02_E180A |
| B*07:02_I194V | B*07:02_I194A |
| B*07:02_E253Q | B*07:02_E253A |
| B*07:02_P267Q | B*07:02_P267A |
| B*07:02_K268E | B*07:02_K268A |
| B*07:02_L270C | B*07:02_L270A |
| B*07:02_E275K | B*07:02_E275A |
| B*08:01_A41T | |
| B*08:01_P43Q | B*08:01_P43A |
| B*08:01_E46A | |
| B*08:01_R62G | B*08:01_R62A |
| B*08:01_N63E | B*08:01_N63A |
| B*08:01_Q65R | B*08:01_Q65A |
| B*08:01_I66N | B*08:01_I66A |
| B*08:01_T69A | |
| B*08:01_N70S | B*08:01_N70A |
| B*08:01_E76V | B*08:01_E76A |
| B*08:01_S77N | B*08:01_S77A |
| B*08:01_N80T | B*08:01_N80A |
| B*08:01_L81A | |
| B*08:01_R82L | B*08:01_R82A |
| B*08:01_G83R | B*08:01_G83A |
| B*08:01_A90D | |
| B*08:01_S97R | B*08:01_S97A |
| B*08:01_Y99F | B*08:01_Y99A |
| B*08:01_L109F | B*08:01_L109A |
| B*08:01_N127K | B*08:01_N127A |
| B*08:01_R131S | B*08:01_R131A |
| B*08:01_D137E | B*08:01_D137A |
| B*08:01_T138K | B*08:01_T138A |
| B*08:01_T143S | B*08:01_T143A |
| B*08:01_R14 [symbol] L | B*08:01_R14 [symbol] A |
| B*08:01_W147L | B*08:01_W147A |
| B*08:01_V152E | B*08:01_V152A |
| B*08:01_D156L | B*08:01_D156A |
| B*08:01_A158T | |
| B*08:01_Y159H | B*08:01_Y159A |
| B*08:01_G162D | B*08:01_G162A |
| B*08:01_T163E | B*08:01_T1 [symbol] 3A |
| B*08:01_E166D | B*08:01_E166A |
| B*08:01_W167G | B*08:01_W167A |
| B*08:01_Y171H | B*08:01_Y171A |
| B*08:01_D177E | B*08:01_D177A |
| B*08:01_T178K | B*08:01_T178A |
| B*08:01_E180Q | B*08:01_E180A |
| B*08:01_I194V | B*08:01_I194A |
| B*08:01_E253Q | B*08:01_E253A |
| B*08:01_P267Q | B*08:01_P267A |
| B*08:01_K268E | B*08:01_K268A |
| B*08:01_L270C | B*08:01_L270A |
| B*08:01_E275K | B*08:01_E275A |
| B*15:01_A41T | |
| B*15:01_P43Q | B*15:01_P43A |
| B*15:01_A46E | |
| B*15:01_R62G | B*15:01_R62A |
| B*15:01_E63N | B*15:01_E63A |
| B*15:01_Q65R | B*15:01_Q65A |
| B*15:01_I66N | B*15:01_I66A |
| B*15:01_T69A | |
| B*15:01_N70S | B*15:01_N70A |
| B*15:01_E76V | B*15:01_E76A |
| B*15:01_S77N | B*15:01_S77A |
| B*15:01_N80T | B*15:01_N80A |
| B*15:01_L81A | |
| B*15:01_R82L | B*15:01_R82A |
| B*15:01_G83R | B*15:01_G83A |
| B*15:01_A90D | |
| B*15:01_R97T | B*15:01_R97A |
| B*15:01_Y99F | B*15:01_Y99A |
| B*15:01_L109F | B*15:01_L109A |
| B*15:01_N127K | B*15:01_N127A |
| B*15:01_S131R | B*15:01_S131A |
| B*15:01_D137E | B*15:01_D137A |
| B*15:01_T138K | B*15:01_T138A |
| B*15:01_T143S | B*15:01_T143A |
| B*15:01_R14 [symbol] L | B*15:01_R14 [symbol] A |
| B*15:01_W147L | B*15:01_W147A |
| B*15:01_E152V | B*15:01_E152A |
| B*15:01_W156L | B*15:01_W156A |
| B*15:01_A158T | |
| B*15:01_Y159H | B*15:01_Y159A |
| B*15:01_G162D | B*15:01_G162A |
| B*15:01_L163E | B*15:01_L163A |
| B*15:01_E166D | B*15:01_E166A |
| B*15:01_W167G | B*15:01_W167A |
| B*15:01_Y171H | B*15:01_Y171A |
| B*15:01_E177D | B*15:01_E177A |
| B*15:01_T178K | B*15:01_T178A |
| B*15:01_Q180E | B*15:01_Q180A |
| B*15:01_I194V | B*15:01_I194A |
| B*15:01_E253Q | B*15:01_E253A |
| B*15:01_P267Q | B*15:01_P267A |
| B*15:01_K268E | B*15:01_K268A |
| B*15:01_L270C | B*15:01_L270A |
| B*15:01_E275K | B*15:01_E275A |
| B*35:01_A41T | |
| B*35:01_P43Q | B*35:01_P43A |
| B*35:01_E46A | |
| B*35:01_R62G | B*35:01_R62A |
| B*35:01_N63E | B*35:01_N63A |
| B*35:01_Q65R | B*35:01_Q65A |
| B*35:01_I66N | B*35:01_I66A |
| B*35:01_T69A | |
| B*35:01_N70S | B*35:01_N70A |
| B*35:01_E76V | B*35:01_E76A |
| B*35:01_S77N | B*35:01_S77A |
| B*35:01_N80T | B*35:01_N80A |
| B*35:01_L81A | |
| B*35:01_R82L | B*35:01_R82A |
| B*35:01_G83R | B*35:01_G83A |
| B*35:01_A90D | |
| B*35:01_R97S | B*35:01_R97A |
| B*35:01_Y99F | B*35:01_Y99A |
| B*35:01_L109F | B*35:01_L109A |
| B*35:01_N127K | B*35:01_N127A |
| B*35:01_S131R | B*35:01_S131A |
| B*35:01_D137E | B*35:01_D137A |
| B*35:01_T138K | B*35:01_T138A |
| B*35:01_T143S | B*35:01_T143A |
| B*35:01_R14 [symbol] L | B*35:01_R14 [symbol] A |
| B*35:01_W147L | B*35:01_W147A |
| B*35:01_V152E | B*35:01_V152A |
| B*35:01_L156R | B*35:01_L156A |
| B*35:01_A158T | |
| B*35:01_Y159H | B*35:01_Y159A |
| B*35:01_G162D | B*35:01_G162A |
| B*35:01_L163E | B*35:01_L163A |
| B*35:01_E166D | B*35:01_E166A |
| B*35:01_W167G | B*35:01_W167A |
| B*35:01_Y171H | B*35:01_Y171A |
| B*35:01_E177D | B*35:01_E177A |
| B*35:01_T178K | B*35:01_T178A |
| B*35:01_Q180E | B*35:01_Q180A |
| B*35:01_V194I | B*35:01_V194A |
| B*35:01_E253Q | B*35:01_E253A |
| B*35:01_P267Q | B*35:01_P267A |
| B*35:01_K268E | B*35:01_K268A |
| B*35:01_L270C | B*35:01_L270A |
| B*35:01_E275K | B*35:01_E275A |
| B*44:02_T41A | |
| B*44:02_P43Q | B*44:02_P43A |
| B*44:02_E46A | |
| B*44:02_R62G | B*44:02_R62A |
| B*44:02_E63N | B*44:02_E63A |
| B*44:02_Q65R | B*44:02_Q65A |
| B*44:02_I66N | B*44:02_I66A |
| B*44:02_T69A | |
| B*44:02_N70S | B*44:02_N70A |
| B*44:02_E76V | B*44:02_E76A |
| B*44:02_N77S | B*44:02_N77A |
| B*44:02_T80N | B*44:02_T80A |
| B*44:02_A81L | |
| B*44:02_L82R | B*44:02_L82A |
| B*44:02_R83G | B*44:02_R83A |
| B*44:02_A90D | |
| B*44:02_R97S | B*44:02_R97A |
| B*44:02_Y99F | B*44:02_Y99A |
| B*44:02_L109F | B*44:02_L109A |
| B*44:02_N127K | B*44:02_N127A |
| B*44:02_S131R | B*44:02_S131A |
| B*44:02_D137E | B*44:02_D137A |
| B*44:02_T138K | B*44:02_T138A |
| B*44:02_T143S | B*44:02_T143A |
| B*44:02_R14 [symbol] L | B*44:02_R14 [symbol] A |
| B*44:02_W147L | B*44:02_W147A |
| B*44:02_V152E | B*44:02_V152A |
| B*44:02_D156L | B*44:02_D156A |
| B*44:02_A158T | |
| B*44:02_Y159H | B*44:02_Y159A |
| B*44:02_G162D | B*44:02_G162A |
| B*44:02_L163E | B*44:02_L163A |
| B*44:02_E166D | B*44:02_E166A |
| B*44:02_S167W | B*44:02_S167A |
| B*44:02_Y171H | B*44:02_Y171A |
| B*44:02_E177D | B*44:02_E177A |
| B*44:02_T178K | B*44:02_T178A |
| B*44:02_Q180E | B*44:02_Q180A |
| B*44:02_I194V | B*44:02_I194A |
| B*44:02_E253Q | B*44:02_E253A |
| B*44:02_P267Q | B*44:02_P267A |
| B*44:02_K268E | B*44:02_K268A |
| B*44:02_L270C | B*44:02_L270A |
| B*44:02_E275K | B*44:02_E275A |
| B*51:01_A41T | |
| B*51:01_P43Q | B*51:01_P43A |
| B*51:01_E46A | |
| B*51:01_R62G | B*51:01_R62A |
| B*51:01_N63E | B*51:01_N63A |
| B*51:01_Q65R | B*51:01_Q65A |
| B*51:01_I66N | B*51:01_I66A |
| B*51:01_T69A | |
| B*51:01_N70S | B*51:01_N70A |
| B*51:01_E76V | B*51:01_E76A |
| B*51:01_N77S | B*51:01_N77A |
| B*51:01_I80N | B*51:01_I80A |
| B*51:01_A81L | |
| B*51:01_L82R | B*51:01_L82A |
| B*51:01_R83G | B*51:01_R83A |
| B*51:01_A90D | |
| B*51:01_T97R | B*51:01_T97A |
| B*51:01_Y99F | B*51:01_Y99A |
| B*51:01_L109F | B*51:01_L109A |
| B*51:01_N127K | B*51:01_N127A |
| B*51:01_S131R | B*51:01_S131A |
| B*51:01_D137E | B*51:01_D137A |
| B*51:01_T138K | B*51:01_T138A |
| B*51:01_T143S | B*51:01_T143A |
| B*51:01_R14 [symbol] L | B*51:01_R14 [symbol] A |
| B*51:01_W147L | B*51:01_W147A |
| B*51:01_E152V | B*51:01_E152A |
| B*51:01_L156R | B*51:01_L156A |
| B*51:01_A158T | |
| B*51:01_Y159H | B*51:01_Y159A |
| B*51:01_G162D | B*51:01_G162A |
| B*51:01_L163E | B*51:01_L163A |
| B*51:01_E166D | B*51:01_E166A |
| B*51:01_W167S | B*51:01_W167A |
| B*51:01_H171Y | B*51:01_H171A |
| B*51:01_E177D | B*51:01_E177A |
| B*51:01_T178K | B*51:01_T178A |
| B*51:01_Q180E | B*51:01_Q180A |
| B*51:01_V194I | B*51:01_V194A |
| B*51:01_E253Q | B*51:01_E253A |
| B*51:01_P267Q | B*51:01_P267A |
| B*51:01_K268E | B*51:01_K268A |
| B*51:01_L270C | B*51:01_L270A |
| B*51:01_E275K | B*51:01_E275A |

[symbol]

indicates data missing or illegible when filed

TABLE 16D
Exemplary HLA-C EVs.
HLA-C Prioritized EV Design

| Change to existing e p | Change to alanine |
|---|---|
| C*03:04_G1C | C*03:04_G1A |
| C*03:04_Y9S | C*03:04_Y9A |
| C*03:04_R14W | C*03:04_R14A |
| C*03:04_G16S | C*03:04_G16A |
| C*03:04_K66N | C*03:04_K66A |
| C*03:04_T73A | |
| C*03:04_V76E | C*03:04_V76A |
| C*03:04_S77N | C*03:04_S77A |
| C*03:04_N80K | C*03:04_N80A |
| C*03:04_A90D | |
| C*03:04_G91R | C*03:04_G91A |
| C*03:04_R97S | C*03:04_R97A |
| C*03:04_Y99F | C*03:04_Y99A |
| C*03:04_R108H | C*03:04_R108A |
| C*03:04_T138K | C*03:04_T138A |
| C*03:04_T143S | C*03:04_T143A |
| C*03:04_W147L | C*03:04_W147A |
| C*03:04_R151C | C*03:04_R151A |
| C*03:04_E152A | |
| C*03:04_L156R | C*03:04_L156A |
| C*03:04_L163T | C*03:04_L163A |
| C*03:04_R170G | C*03:04_R170A |
| C*03:04_K173E | C*03:04_K173A |
| C*03:04_E177K | C*03:04_E177A |
| C*03:04_H184P | C*03:04_H184A |
| C*03:04_P193L | C*03:04_P193A |
| C*03:04_V194L | C*03:04_V194A |
| C*03:04_W219R | C*03:04_W219A |
| C*03:04_E229Q | C*03:04_E229A |
| C*03:04_V248M | C*03:04_V248A |
| C*03:04_E253Q | C*03:04_E253A |
| C*03:04_P267Q | C*03:04_P267A |
| C*03:04_L270C | C*03:04_L270A |
| C*03:04_R273S | C*03:04_R273A |
| C*03:04_E275K | C*03:04_E275A |
| C*04:01_G1C | C*04:01_G1A |
| C*04:01_S9Y | C*04:01_S9A |
| C*04:01_W14R | C*04:01_W14A |
| C*04:01_G16S | C*04:01_G16A |
| C*04:01_K66N | C*04:01_K66A |
| C*04:01_A73T | |
| C*04:01_V76E | C*04:01_V76A |
| C*04:01_N77S | C*04:01_N77A |
| C*04:01_K80N | C*04:01_K80A |
| C*04:01_D90A | |
| C*04:01_G91R | C*04:01_G91A |
| C*04:01_R97S | C*04:01_R97A |
| C*04:01_F99Y | C*04:01_F99A |
| C*04:01_R108H | C*04:01_R108A |
| C*04:01_T138K | C*04:01_T138A |
| C*04:01_T143S | C*04:01_T143A |
| C*04:01_W147L | C*04:01_W147A |
| C*04:01_R151C | C*04:01_R151A |
| C*04:01_E152A | |
| C*04:01_R156L | C*04:01_R156A |
| C*04:01_T163L | C*04:01_T163A |
| C*04:01_R170G | C*04:01_R170A |
| C*04:01_E173K | C*04:01_E173A |
| C*04:01_E177K | C*04:01_E177A |
| C*04:01_H184P | C*04:01_H184A |
| C*04:01_P193L | C*04:01_P193A |
| C*04:01_V194L | C*04:01_V194A |
| C*04:01_W219R | C*04:01_W219A |
| C*04:01_E229Q | C*04:01_E229A |
| C*04:01_V248M | C*04:01_V248A |
| C*04:01_E253Q | C*04:01_E253A |
| C*04:01_P267Q | C*04:01_P267A |
| C*04:01_L270C | C*04:01_L270A |
| C*04:01_R273S | C*04:01_R273A |
| C*04:01_E275K | C*04:01_E275A |
| C*05:01_C1C | C*05:01_C1A |
| C*05:01_Y9D | C*05:01_Y9A |
| C*05:01_R14W | C*05:01_R14A |
| C*05:01_G16S | C*05:01_G16A |
| C*05:01_K66N | C*05:01_K66A |
| C*05:01_T73A | |
| C*05:01_V76E | C*05:01_V76A |
| C*05:01_N77S | C*05:01_N77A |
| C*05:01_K80N | C*05:01_K80A |
| C*05:01_A90D | |
| C*05:01_G91R | C*05:01_G91A |
| C*05:01_R97S | C*05:01_R97A |
| C*05:01_Y99S | C*05:01_Y99A |
| C*05:01_R108H | C*05:01_R108A |
| C*05:01_K138T | C*05:01_K138A |
| C*05:01_T143S | C*05:01_T143A |
| C*05:01_W147L | C*05:01_W147A |
| C*05:01_R151C | C*05:01_R151A |
| C*05:01_E152A | |
| C*05:01_R156L | C*05:01_R156A |
| C*05:01_T163L | C*05:01_T163A |
| C*05:01_R170G | C*05:01_R170A |
| C*05:01_E173K | C*05:01_E173A |
| C*05:01_K177E | C*05:01_K177A |
| C*05:01_H184P | C*05:01_H184A |
| C*05:01_P193L | C*05:01_P193A |
| C*05:01_V194L | C*05:01_V194A |
| C*05:01_R219W | C*05:01_R219A |
| C*05:01_E229Q | C*05:01_E229A |
| C*05:01_V248M | C*05:01_V248A |
| C*05:01_E253Q | C*05:01_E253A |
| C*05:01_P267Q | C*05:01_P267A |
| C*05:01_L270C | C*05:01_L270A |
| C*05:01_R273S | C*05:01_R273A |
| C*05:01_G275E | C*05:01_G275A |
| C*06:02_C1G | C*06:02_C1A |
| C*06:02_Y9D | C*06:02_Y9A |
| C*06:02_R14W | C*06:02_R14A |
| C*06:02_G16S | C*06:02_G16A |
| C*06:02_K66N | C*06:02_K66A |
| C*06:02_T73A | |
| C*06:02_V76E | C*06:02_V76A |
| C*06:02_N77S | C*06:02_N77A |
| C*06:02_K80N | C*06:02_K80A |
| C*06:02_A90D | |
| C*06:02_G91R | C*06:02_G91A |
| C*06:02_R97S | C*06:02_R97A |
| C*06:02_Y99S | C*06:02_Y99A |
| C*06:02_R108H | C*06:02_R108A |
| C*06:02_K138T | C*06:02_K138A |
| C*06:02_T143S | C*06:02_T143A |
| C*06:02_W147L | C*06:02_W147A |
| C*06:02_R151C | C*06:02_R151A |
| C*06:02_E152A | |
| C*06:02_R156L | C*06:02_R156A |
| C*06:02_T163L | C*06:02_T163A |
| C*06:02_R170G | C*06:02_R170A |
| C*06:02_E173K | C*06:02_E173A |
| C*06:02_K177E | C*06:02_K177A |
| C*06:02_H184P | C*06:02_H184A |
| C*06:02_P193L | C*06:02_P193A |
| C*06:02_V194L | C*06:02_V194A |
| C*06:02_R219W | C*06:02_R219A |
| C*06:02_E229Q | C*06:02_E229A |
| C*06:02_V248M | C*06:02_V248A |
| C*06:02_E253Q | C*06:02_E253A |
| C*06:02_P267Q | C*06:02_P267A |
| C*06:02_L270C | C*06:02_L270A |
| C*06:02_R273S | C*06:02_R273A |
| C*06:02_G275E | C*06:02_G275A |
| C*07:01_C1G | C*07:01_C1A |
| C*07:01_D9Y | C*07:01_D9A |
| C*07:01_R14W | C*07:01_R14A |
| C*07:01_G16S | C*07:01_G16A |
| C*07:01_N66K | C*07:01_N66A |
| C*07:01_A73T | |
| C*07:01_V76E | C*07:01_V76A |
| C*07:01_S77N | C*07:01_S77A |
| C*07:01_N80K | C*07:01_N80A |
| C*07:01_D90A | |
| C*07:01_G91R | C*07:01_G91A |
| C*07:01_R97S | C*07:01_R97A |
| C*07:01_Y99S | C*07:01_Y99A |
| C*07:01_R108H | C*07:01_R108A |
| C*07:01_T138K | C*07:01_T138A |
| C*07:01_T143S | C*07:01_T143A |
| C*07:01_L147W | C*07:01_L147A |
| C*07:01_R151C | C*07:01_R151A |
| C*07:01_A152T | |
| C*07:01_L156D | C*07:01_L156A |
| C*07:01_T163A | C*07:01_T163A |
| C*07:01_R170G | C*07:01_R170A |
| C*07:01_E173K | C*07:01_E173A |
| C*07:01_E177K | C*07:01_E177A |
| C*07:01_P184H | C*07:01_P184A |
| C*07:01_P193L | C*07:01_P193A |
| C*07:01_L194V | C*07:01_L194A |
| C*07:01_R219W | C*07:01_R219A |
| C*07:01_E229Q | C*07:01_E229A |
| C*07:01_V248M | C*07:01_V248A |
| C*07:01_Q253E | C*07:01_Q253A |
| C*07:01_Q267P | C*07:01_Q267A |
| C*07:01_L270C | C*07:01_L270A |
| C*07:01_S273R | C*07:01_S273A |
| C*07:01_E275G | C*07:01_E275A |
| C*12:03_C1G | C*12:03_C1A |
| C*12:03_Y9S | C*12:03_Y9A |
| C*12:03_R14W | C*12:03_R14A |
| C*12:03_G16S | C*12:03_G16A |
| C*12:03_K66N | C*12:03_K66A |
| C*12:03_A73T | |
| C*12:03_V76E | C*12:03_V76A |
| C*12:03_S77N | C*12:03_S77A |
| C*12:03_N80K | C*12:03_N80A |
| C*12:03_D90A | |
| C*12:03_G91R | C*12:03_G91A |
| C*12:03_W97R | C*12:03_W97A |
| C*12:03_Y99F | C*12:03_Y99A |
| C*12:03_R108H | C*12:03_R108A |
| C*12:03_T138K | C*12:03_T138A |
| C*12:03_T143S | C*12:03_T143A |
| C*12:03_W147L | C*12:03_W147A |
| C*12:03_R151C | C*12:03_R151A |
| C*12:03_E152A | |
| C*12:03_W156R | C*12:03_W156A |
| C*12:03_T163A | C*12:03_T163A |
| C*12:03_R170G | C*12:03_R170A |
| C*12:03_E173K | C*12:03_E173A |
| C*12:03_E177K | C*12:03_E177A |
| C*12:03_H184R | C*12:03_H184A |
| C*12:03_P193L | C*12:03_P193A |
| C*12:03_V194L | C*12:03_V194A |
| C*12:03_R219W | C*12:03_R219A |
| C*12:03_E229Q | C*12:03_E229A |
| C*12:03_V248M | C*12:03_V248A |
| C*12:03_E253Q | C*12:03_E253A |
| C*12:03_P267Q | C*12:03_P267A |
| C*12:03_L270C | C*12:03_L270A |
| C*12:03_R273S | C*12:03_R273A |
| C*12:03_E275K | C*12:03_E275A |

### Example 10: Further Exemplary Variant Positions

Tables 17A and 17B Est positions in HLA alleles which could be mutated to generate EVs, such as EVs which could be used to confirm predicted ERs and/or to improve serological resolution. EVs with a conservative mutation at one or more positions selected from Tables 17A or 17B are contemplated herein. EVs with a non-conservative mutation at one or more positions selected from Tables 17A or 17B are contemplated herein. EVs with at least 1, 2, 3, 4, 5, 6, 7, 8, 9, 10, 11, 12, 13, 14, 15, or more than 15 mutations at positions selected from Tables 17A or 17B are contemplated herein. EVs with mutations at residues within ≤4 Å of a position selected from Table 17A or 17B are also contemplated herein. In one example, ≤4 Å is measured in the context of an unfolded peptide. In one example, ≤4 Å is measured in the context of a folded HLA protein.

In Tables 17A and 17B, HLA allele HLA-A*01:01 (e.g., SEQ ID NO: 1) serves as a reference for the numbering of HLA locus A; HLA allele HLA-B*07:02 (e.g., SEQ ID NO: 2) serves as a reference for the numbering of HLA locus B; HLA allele HLA-C*01:02 (e.g., SEQ ID NO: 3) serves as a reference for the numbering of HLA locus C; HLA allele HLA-DPA1*01:03 (e.g., SEQ ID NO: 4) serves as a reference for the numbering of HLA locus DPA1; HLA allele HLA-DPB1*01:01 (e.g., SEQ ID NO: 5) serves as a reference for the numbering of HLA locus DPB1; HLA allele HLA-DQA1*01:01 (e.g., SEQ ID NO: 6) serves as a reference for the numbering of HLA locus DQA1; HLA allele HLA-DQB1*02:01 (e.g., SEQ ID NO: 7) serves as a reference for the numbering of HLA locus DQB1; HLA allele HLA-DRB1*01:01 (e.g., SEQ ID NO: 8) serves as a reference for the numbering of HLA locus DRB1; HLA allele HLA-DRB3*01:01 (e.g., SEQ ID NO: 9) serves as a reference for the numbering of HLA locus DRB3/4/5

TABLE 17A
Positions which could be mutated in HLA alleles to generate EVs

| HLA locus | A | B | C | DPA1 | DPB1 | DQA1 | DQB1 | DRB1 | DRB3/4/5 |
|---|---|---|---|---|---|---|---|---|---|
| Variant | 3 | 4 | 1 | 11 | 8 | 1 | 3 | 4 | 4 |
| positions | 6 | 9 | 6 | 18 | 9 | 2 | 9 | 9 | 6 |
| in | 7 | 11 | 9 | 19 | 11 | 11 | 13 | 10 | 9 |
| common | 9 | 12 | 11 | 28 | 17 | 18 | 14 | 11 | 10 |
| alleles | 12 | 24 | 14 | 31 | 33 | 25 | 23 | 12 | 11 |
| targeted | 14 | 30 | 16 | 43 | 35 | 26 | 26 | 13 | 12 |
| for | 17 | 32 | 21 | 50 | 36 | 34 | 28 | 14 | 13 |
| engineered | 19 | 41 | 24 | 51 | 55 | 40 | 30 | 16 | 18 |
| variant | 30 | 43 | 35 | 66 | 56 | 41 | 37 | 25 | 25 |
| generation | 31 | 45 | 49 | 72 | 57 | 45 | 38 | 26 | 26 |
| | 33 | 46 | 66 | 73 | 65 | 47 | 45 | 27 | 28 |
| | 35 | 52 | 73 | 83 | 69 | 48 | 46 | 28 | 29 |
| | 43 | 59 | 76 | 91 | 72 | 50 | 47 | 29 | 30 |
| | 44 | 62 | 77 | 96 | 76 | 51 | 51 | 30 | 31 |
| | 47 | 63 | 80 | 111 | 84 | 52 | 52 | 31 | 32 |
| | 50 | 65 | 90 | 127 | 85 | 53 | 53 | 32 | 34 |
| | 54 | 66 | 91 | 160 | 86 | 54 | 55 | 33 | 37 |
| | 56 | 67 | 94 | 190 | 87 | 55 | 56 | 37 | 38 |
| | 62 | 69 | 95 | | 90 | 56 | 57 | 38 | 40 |
| | 63 | 70 | 97 | | 91 | 59 | 66 | 40 | 41 |
| | 65 | 71 | 99 | | 96 | 61 | 67 | 47 | 44 |
| | 66 | 74 | 103 | | 162 | 64 | 70 | 50 | 47 |
| | 67 | 76 | 108 | | 170 | 66 | 71 | 51 | 48 |
| | 68 | 77 | 113 | | 178 | 69 | 74 | 57 | 55 |
| | 69 | 80 | 114 | | 188 | 75 | 75 | 58 | 57 |
| | 70 | 81 | 116 | | 194 | 76 | 77 | 60 | 60 |
| | 73 | 82 | 138 | | | 80 | 81 | 67 | 67 |
| | 74 | 83 | 143 | | | 107 | 84 | 70 | 70 |
| | 76 | 90 | 147 | | | 129 | 85 | 71 | 71 |
| | 77 | 94 | 151 | | | 130 | 86 | 73 | 73 |
| | 79 | 95 | 152 | | | 156 | 87 | 74 | 74 |
| | 80 | 97 | 156 | | | 160 | 89 | 77 | 76 |
| | 81 | 98 | 160 | | | 161 | 90 | 78 | 77 |
| | 82 | 99 | 163 | | | 163 | 116 | 85 | 78 |
| | 83 | 103 | 170 | | | 175 | 125 | 86 | 81 |
| | 90 | 109 | 173 | | | 187 | 126 | 96 | 85 |
| | 92 | 113 | 175 | | | 187 | 130 | 98 | 86 |
| | 95 | 114 | 177 | | | | 135 | 104 | 96 |
| | 97 | 116 | 184 | | | | 140 | 112 | 98 |
| | 99 | 127 | 193 | | | | 163 | 120 | 104 |
| | 102 | 131 | 194 | | | | 167 | 133 | 105 |
| | 105 | 137 | 211 | | | | 168 | 140 | 108 |
| | 107 | 138 | 219 | | | | 169 | 142 | 120 |
| | 109 | 143 | 229 | | | | 182 | 149 | 135 |
| | 114 | 145 | 248 | | | | 185 | 166 | 140 |
| | 116 | 147 | 253 | | | | 197 | 179 | 149 |
| | 125 | 152 | 261 | | | | | 180 | 157 |
| | 127 | 156 | 267 | | | | | 181 | 164 |
| | 141 | 158 | 270 | | | | | 189 | 170 |
| | 142 | 159 | 273 | | | | | | 180 |
| | 144 | 162 | 275 | | | | | | 181 |
| | 145 | 163 | 273 | | | | | | 183 |
| | 149 | 166 | 275 | | | | | | 187 |
| | 150 | 167 | | | | | | | 189 |
| | 151 | 171 | | | | | | | 191 |
| | 152 | 177 | | | | | | | |
| | 156 | 178 | | | | | | | |
| | 158 | 180 | | | | | | | |
| | 161 | 194 | | | | | | | |
| | 163 | 199 | | | | | | | |
| | 166 | 211 | | | | | | | |
| | 167 | 239 | | | | | | | |
| | 171 | 245 | | | | | | | |
| | 177 | 253 | | | | | | | |
| | 184 | 267 | | | | | | | |
| | 186 | 268 | | | | | | | |
| | 193 | 270 | | | | | | | |
| | 194 | 275 | | | | | | | |
| | 207 | | | | | | | | |
| | 236 | | | | | | | | |
| | 245 | | | | | | | | |
| | 246 | | | | | | | | |
| | 253 | | | | | | | | |
| | 255 | | | | | | | | |
| | 268 | | | | | | | | |
| | 276 | | | | | | | | |
| | 245 | | | | | | | | |
| | 246 | | | | | | | | |
| | 253 | | | | | | | | |
| | 255 | | | | | | | | |
| | 268 | | | | | | | | |
| | 276 | | | | | | | | |
| | 280 | | | | | | | | |

TABLE 17B
Prioritized Positions which could be mutated in HLA alleles to generate EVs

| HLA locus | A | B | C | DPA1 | DPB1 | DQA1 | DQB1 | DRB1 | DRB3/4/5 |
|---|---|---|---|---|---|---|---|---|---|
| Prioritized | 7 | 41 | 1 | 19 | 11 | 1 | 3 | 4 | 4 |
| variant | 9 | 43 | 9 | 28 | 33 | 2 | 13 | 9 | 9 |
| positions | 17 | 46 | 14 | 31 | 55 | 11 | 23 | 16 | 11 |
| likely | 19 | 62 | 16 | 43 | 57 | 18 | 28 | 25 | 18 |
| surface- | 43 | 63 | 66 | 50 | 65 | 25 | 30 | 31 | 25 |
| exposed | 44 | 65 | 73 | 51 | 69 | 26 | 45 | 32 | 31 |
| and/or | 47 | 66 | 76 | 66 | 72 | 34 | 46 | 50 | 32 |
| participating | 50 | 69 | 77 | 72 | 76 | 40 | 51 | 51 | 44 |
| in | 54 | 70 | 80 | 73 | 84 | 41 | 52 | 58 | 48 |
| peptide | 56 | 76 | 90 | 83 | 85 | 47 | 53 | 60 | 55 |
| binding in | 62 | 77 | 91 | 111 | 86 | 50 | 55 | 67 | 67 |
| common | 63 | 80 | 97 | 127 | 87 | 52 | 56 | 70 | 70 |
| alleles | 65 | 81 | 99 | 160 | 90 | 53 | 57 | 73 | 73 |
| targeted | 66 | 82 | 108 | | 91 | 54 | 66 | 74 | 74 |
| for | 67 | 83 | 138 | | 96 | 55 | 67 | 77 | 76 |
| engineered | 68 | 90 | 143 | | 162 | 56 | 70 | 85 | 77 |
| variant | 69 | 97 | 147 | | 170 | 59 | 71 | 96 | 81 |
| generation | 70 | 99 | 151 | | 178 | 61 | 74 | 98 | 85 |
| | 73 | 109 | 152 | | | 64 | 77 | 104 | 96 |
| | 74 | 127 | 156 | | | 69 | 81 | 112 | 98 |
| | 76 | 131 | 163 | | | 75 | 84 | 133 | 104 |
| | 77 | 137 | 170 | | | 76 | 85 | 140 | 105 |
| | 79 | 138 | 173 | | | 80 | 87 | 142 | 108 |
| | 80 | 143 | 177 | | | 129 | 126 | 166 | 135 |
| | 81 | 145 | 184 | | | 130 | 130 | 179 | 140 |
| | 82 | 147 | 193 | | | 160 | 135 | 180 | 164 |
| | 83 | 152 | 194 | | | 161 | 140 | 181 | 180 |
| | 90 | 156 | 219 | | | 163 | 163 | 189 | 181 |
| | 95 | 158 | 229 | | | 175 | 167 | | 187 |
| | 97 | 159 | 248 | | | | 168 | | 189 |
| | 99 | 162 | 253 | | | | 169 | | 191 |
| | 105 | 163 | 267 | | | | 182 | | |
| | 109 | 166 | 270 | | | | 185 | | |
| | 114 | 167 | 273 | | | | | | |
| | 127 | 171 | 275 | | | | | | |
| | 141 | 177 | | | | | | | |
| | 142 | 178 | | | | | | | |
| | 144 | 180 | | | | | | | |
| | 145 | 194 | | | | | | | |
| | 149 | 253 | | | | | | | |
| | 150 | 267 | | | | | | | |
| | 151 | 268 | | | | | | | |
| | 152 | 270 | | | | | | | |
| | 156 | 275 | | | | | | | |
| | 158 | | | | | | | | |
| | 161 | | | | | | | | |
| | 163 | | | | | | | | |
| | 166 | | | | | | | | |
| | 167 | | | | | | | | |
| | 171 | | | | | | | | |
| | 177 | | | | | | | | |
| | 184 | | | | | | | | |
| | 186 | | | | | | | | |
| | 193 | | | | | | | | |
| | 194 | | | | | | | | |
| | 253 | | | | | | | | |
| | 255 | | | | | | | | |
| | 268 | | | | | | | | |
| | 276 | | | | | | | | |

### Example 11: Overview

The following overview summarizes general concepts of eps/ep patterns, panel design, and EVs described herein.

Conserved positions refer to positions of 100% conserved amino acid residues in the extracellular domain among Common alleles (≥1 in 10,000 in a population according to CIWD 3.0) in each locus such as HLA-A, HLA-B, HLA-C, HLA-DRB1, HLA-DQA1, HLA-DQB1, HLA-DPA1 or HLA-DPB1. HLA-DRB3, HLA-DRB4 and HLA-DRB5 loci can be combined for sequence analysis because each person can carry 0, 1 or 2 haplotypes of HLA-DRB3, HLA-DRB4 and/or HLA-DRB5. There are not many HLA-DRB3/4/5 alleles, and only Well Documented (≥5 occurrences in a population, CIWD 3.0) assignment is given.

Besides individual eps (a single amino acid residue at a single position), each allele can also be viewed as made of overlapping eps or ep patterns based on user defined distance, covering all residues of the extracellular domain. The longer the distance, whether linear or 3-dimensional, the higher degree of variety can be found among alleles, and therefore the longer list of alleles to cover all eps or ep patterns present is a targeted population(s).

For panel design, ≤5 Å (minimal distance between any 2 residues) is used for defining ep patterns of each allele in the targeted population. Each ep pattern encompasses immediately adjacent clusters (each amino acid is ˜3.5 Å apart based on alpha-carbon distance) and allows room for accommodating modeling variations.

In some aspects, ≤5 Å (minimal distance between any 2 residues) is chosen to define an ep or ep pattern for panel design because by increasing the distance, more alleles need to be included to cover such wider variations. However, based on the DQ panel example described herein, a panel does not need to go extra length to cover a majority of the frequently encountered specificity profiles.

The eps or ep patterns consider all residues in the extracellular domain of an HLA allele. No matter what the targeted alleles on a list are, the panel design algorithm can identify unique eps or ep patterns among these alleles and select the allele of the highest frequency if multiple alleles carry the same ep or ep pattern. By design, this approach will identify and include all variant positions contributing to the eps and ep patterns.

A panel designed this way contains all eps and ep patterns (≤5 Å) at least once, and for every ep or ep pattern present in 1 allele, it is missing in at least another one or more alleles on the panel. With a combination of positive and negative Ag signals (specificity profile), essential residues/region (ER) of an epitope can be predicted for a monoclonal Ab.

To verify ER and/or reduce ER prediction ambiguities, engineered variants (EV) are created. These EV can also serve a role in identifying ancillary residues/region (AR). The general approach is to mutate the predicted ER residue(s) at variant position(s) to observe their effects on binding. Sometimes neighboring residues (at variant or conserved positions) are also mutated to gain additional information of the full epitope.

This route of EV design can be limited by the availability of monoclonal antibody (mAb) and mAb-like serum samples due to the requirement of an experimental specificity profile and ER prediction. To overcome this limitation, the present disclosure provides methods to preemptively target with prioritizations all variant positions in HLA-A, HLA-B, HLA-C, HLA-DRB1, HLA-DQA1, HLA-DQB1, HLA-DPA1, HLA-DPB1, or HLA-DRB3/4/5 in the extracellular domain for EV designs.

Preferably, these variant positions are Ab accessible and/or involved in peptide presentation, so they have a more direct impact on interaction with the paratope on an Ab. Positions embedded within an HLA molecule may play a critical role in keeping the structural integrity, and are less likely to directly contribute to paratope recognition; therefore, they are deprioritized for EV design. Specific positions of the total and prioritized positions are provided herein.

Neighboring conserved positions (≤5 Å) to the prioritized variant positions can also be subject to EV design because conserved residues could also be part of an ER. Again, these conserved positions can also be prioritized if they are Ab accessible and/or involved in peptide presentation.

After these positions are prioritized for EV designs, which wildtype (WT) alleles are based on to generate these EV are also considered. Conceptually, any WT allele can be based on to generate a population of EV, especially if the WT is positive in detecting Ab binding. However, for practical purposes, common alleles are the focus and can represent the less frequent alleles. In this disclosure, EV designs for each locus or combined loci (HLA-A, HLA-B, HLA-C, HLA-DRB1, HLA-DQA1, HLA-DQB1, HLA-DPA1, HLA-DPB1, or HLA-DRB3/4/5) are listed as examples.

Even with an arbitrary cut-off at ≥5% or ≥1% total frequency, thousands of EV examples are identified. If all Common alleles are considered, the list could be in 10,000s-100,000s range. Therefore, there is a need to further apply panel design algorithm on a list of not only WT common alleles but also prioritized EV designs to improve the utility of the panel.

After the prioritized positions and WT alleles are identified for EV designs, another layer of consideration is on what amino acid(s) the WT residue should change into. Theoretically, any of the remaining 19 non-self native amino acids could provide information. In practice, the positive WT residue is usually changed to another WT residue known at the position especially if the 2^{nd} WT is non-binding. In the absence of experimental data, an amino acid is typically changed to another amino acid of different structure and chemical properties affecting hydrogen bonds, salt bridges, and hydrophobic interactions. Cysteine (Cys) and proline (Pro) are generally avoided unless they are known to be tolerated in certain positions because Cys could form disulfide bond and Pro introduces conformational rigidity that could impact the global conformation of an HLA.

In other examples, instead of considering naturally occurring eps for residue replacement, alanine (Ala) is chosen because it serves the purpose of changing local shape and chemical properties in most cases while maintaining global conformation. Ala is the choice especially when an EV involves changing of a conserved position where no other native residue is known at the position.

In some cases, interchange between small residues such as Ala, glycine (Gly) and serine (Ser) may not be efficient in impacting binding. Similar situations could happen between residues of similar shape and/or chemical properties. However, Ab are known to be capable of differentiating even single amino acid difference, so, even minor changes could result in apparent binding differences.

Although the lists of EV designs disclosed herein are for single changes, double or multiple mutations can be combined in an EV to achieve a synergistic effect.

In summary, an EV is defined as an engineered variant that does not currently exist in IPD-IMGT/HLA database.

### Example 12: Aspects

The following Aspects are illustrative only and do not limit the scope of the present disclosure or the appended claims.

The aspects discussed below may be used to improve pretransplant matching for organ allocation and posttransplant monitoring of antibodies (Ab) produced against human leukocyte antigens (HLA) through essential residue/region (ER) mapping on a multiplex assay platform.
1. Method for designing and/or producing an epitope panel constituting a minimal number of HLA single antigens (SA) that provide coverage of all variant positions and all residues at these positions individually or in combination for a targeted population,
   A. where each residue at each variant position (ep) has the potential to become an ER of an epitope to a monoclonal Ab (mAb),
   B. where each unique combination ep pattern is composed of multiple residues. In some examples, the EV includes or excludes mutations in eps or ep patterns selected according to one or more of the following criteria:
      eps or ep patterns unique among each locus of HLA-A, HLA-B, HLA-C, or combined HLA class I (HLA-A/B/C) loci;
      eps or ep patterns unique among each locus of HLA-DRB1, HLA-DQA1, HLA-DQB1, HLA-DPA1 and HLA-DPB1 or combined HLA-DRB3/4/5 loci;
      eps or ep patterns unique among each group of HLA-DQ or HLA-DP antigens where HLA-DQA1 forms a heterodimer with HLA-DQB1 and HLA-DPA1 with HLA-DPB1;
      eps or ep patterns conserved in >1 alleles among each locus of HLA-A, HLA-B, HLA-C, or combined HLA class I (HLA-A/B/C) loci;
      eps or ep patterns conserved in >1 alleles among each locus of HLA-DRB1, HLA-DQA1, HLA-DQB1, HLA-DPA1, HLA-DPB1, or combined HLA-DRB3/4/5 loci;
      eps or ep patterns conserved in >1 antigen among each group of HLA-DQ or HLA-DP antigens where HLA-DQA1 forms a heterodimer with HLA-DQB1 and HLA-DPA1 with HLA-DPB1;
      eps or ep patterns conserved in at least 0.1%, at least 1%, at least 2%, at least 3%, at least 4%, at least 5%, at least 10%, at least 20%, at least 30%, at least 40%, at least 50%, at least 60%, at least 70%, at least 80%, at least 90%, at least 95%, at least 96%, at least 97%, at least 98%, at least 99%, at least 99.9%, or 100% conserved among Common alleles of each of the HLA antigen group such as HLA-A, HLA-B, HLA-C, HLA class I (HLA-A/B/C), HLA-DR, HLA-DQ, HLA-DP, and Well Documented HLA-DRB3/4/5;
      eps or ep patterns targeted for EV design in which changes are tolerated structurally;
      eps or ep patterns identified as a variant position after aligning at least 2, at least 3, at least 4, at least 5, at least 6, at least 7, at least 8, at least 9, at least 10, at least 15, at least 20, at least 30, at least 40, at least 50, at least 60, at least 70, at least 80, at least 90, at least 100, or more than 100 HLA alleles;
      eps or ep patterns identified as providing support for the structural integrity of a control HLA, such as a WT HLA, are avoided for EV considerations. Embedded and conserved positions in contact with multiple neighboring residues have a higher chance of affecting global conformations;
      eps or ep patterns not affecting the global conformations of a control HLA, such as a WT HLA, and in close proximity to a position identified as a variant position by aligning one or more HLA alleles;
      eps or ep patterns which are linearly close to one another, such as within 15, 14, 13, 12, 11, 10, 9, 8, 7, 6, 5, 4, 3, 2 or 1 residue of one another, as measured in the context of an unfolded peptide;
      eps or ep patterns which are topologically close to one another, such as within 15, 14, 13, 12, 11, 10, 9, 8, 7, 6, 5.5, 5, 4.5, 4, 3.5, 3, 2.5, 2, 1.5, 1, or 0.5 Å of one another, such as when measured in the context of a folded HLA protein or when measured in the context of an assembled HLA heterodimer;
      eps or ep patterns which are linearly close to a known critical residue, such as within 15, 14, 13, 12, 11, 10, 9, 8, 7, 6, 5, 4, 3, 2, or 1 residues of one another, as measured in the context of an unfolded peptide;
      eps or ep patterns which are topologically close to a known critical residue, such as within 15, 14, 13, 12, 11, 10, 9, 8, 7, 6, 5.5, 5, 4.5, 4, 3.5, 3, 2.5, 2, 1.5, 1, or 0.5 Å of one another, such as when measured in the context of a folded HLA protein or when measured in the context of an assembled HLA heterodimer;
      eps or ep patterns which are topologically distant from another candidate ER site such as over 20 Å from one another and/or on the opposite sides of the HLA molecule that exceeding the coverage of the same epitope, as when measured in the context of a folded HLA protein or when measured in the context of an assembled HLA heterodimer;
      eps or ep patterns predicted or confirmed to be surface exposed of a control HLA, such as a WT HLA;
      eps or ep patterns predicted or confirmed to be embedded of a control HLA, such as a WT HLA;
      eps or ep patterns which are considered solvent exposed based on molecular modeling, which in one example includes pHLA3D (phla3d.com.br),
   C. where the targeted population is user defined that could cover a specific geological region and/or ancestral descendants, a combination of several such populations, or a world-wide population. The targeted population preferably covers but is not limited to the common alleles frequently associated with the population. The HLA antigen (Ag or allele compositions of the targeted population continues to evolve as more typing information becomes available,
   D. where the number of SA on a panel can be simultaneously assayed using a multiplex platform to provide a specificity profile sufficient for predicting potential ep(s) or ep pattern(s) implicated as an ER for a mAb or polyclonal Ab (pAb) sample,
      a. the multiplex capacity could be to tens, hundreds, or thousands of HLA SA based on existing and future multiplex platforms;
      b. on the other hand, because the method claimed in designing such an epitope panel can reduce the number of SA needed for mapping ER of an epitope, multiplex capacity for more than xMAP 3D (500 beads) platform is not anticipated. Nonetheless, a multiplex platform that can perform assays simultaneously with all epitope panels combined is beneficial,
   E. where the Ag on the panel can be produced in any form from any expression system,
      a. where preferably a full-length Ag is produced from a human expressing system to best match the native HLA conformations,
         i. where the host cell is derived from human B cell lineage without endogenous Class I and/or Class II HLA expression, or
         ii. an engineered human cell line without endogenous Class I and/or Class II HLA expression.
      b. where a soluble version of HLA can be secreted from a human host cell described above or a non-human expression system such as Chinese hamster ovarian (CHO) cell and insect cell systems commonly used for recombinant expression.
   F. where a mammalian expression vector is used to preferably co-express a bicistronic message containing the 2 coding sequences forming the alpha and beta subunits;
      a. however, the 2 subunits can also be expressed as 2 monocistronic messages from the same vector or 2 separate vectors, and
      b. the same Ag can also be expressed from a non-human expression system although not desirable due to different post-translational modifications.
2. An imputation logic or software that not only designs such an epitope panel based on a targeted population but also analyzes the specificity profile of each mAb or pAb sample to identify potential ep(s) or ep pattern(s) implicated as the ER of an epitope(s). This logic has the options of performing:
   a. single locus specific analysis so signals to other loci likely from a different Ab can be excluded, and
   b. multi loci analysis so signals from inter-locus cross-reactivities are not missed out either.
3. Use of engineered variants (EV) with altered ep(s) and/or ep pattern(s) to confirm the ER predicted through epitope analysis based on the specificity profile(s) provided by the epitope panel(s) described herein;
   A. certain ep(s) and ep pattern(s) have been mapped to be ER of epitopes to certain mAb, and therefore it is likely that the same ep or ep pattern can be recognized by another mAb generated from producers sensitized by mismatched immunizers,
   B. pre-inclusion of such engineered variants on the same epitope panel or a separate panel will move the status of a “predicted” ER to a “verified” one that deserves more clinical attention, and
   C. such an EV may exist in the world-wide population but so long as it has not been recorded in IMGT database, it is considered novel. In fact, a separate EV database, within IMGT or not, is being used to capture the fast-accumulating EV for mapping ER of epitope recognized by mAb.
4. The Ag compositions of the HLA-DQ panel described in the examples include the minimal numbers of HLA-DQ alpha-beta, HLA-DP, HLA-DR, and HLA-Class I paired Ag.
   A. Because it is feasible to substitute the ep or ep pattern coverage of a certain allele with another one or more than one allele, the Ag composition of the epitope panel can be modified to fit the same purpose. In aspects, an epitope panel is composed of a set of Ag with ≥20, 30, 40, 50, 60, 70, 80 or 90% of the Ag of an epitope panel design described here.
   B. Pending on the multiplex platform, customer preference, and manufacturer operations, the epitope panels can be produced, assayed, and analyzed separately or in combination.
      a. In aspects, an epitope panel is generally HLA Class I or HLA Class II specific and it can be further divided into locus specific panels.
      b. In aspects, an epitope panel can be combined with another epitope panel(s) to make a larger epitope panel to cover wider eps and ep patterns among and across loci. For example, combing DR, DQ, and/or DP panels.

Various alterations and/or modifications of the features illustrated herein, and additional applications of the principles illustrated herein, which would occur to one skilled in the relevant art and having possession of this disclosure, can be made to the illustrated aspects without departing from the spirit and scope of the disclosure as defined by the claims, and are to be considered within the scope of this disclosure. Thus, while various aspects and aspects have been disclosed herein, other aspects are contemplated. While a number of methods and components similar or equivalent to those described herein can be used to practice aspects of the present disclosure, only certain components and methods are described herein.

It will also be appreciated that assemblies, systems, processes, panels, compositions and/or products according to certain aspects of the present disclosure may include, incorporate, or otherwise include properties features (e.g., components, members, elements, parts, and/or portions) described in other aspects disclosed and/or described herein. Accordingly, the various features of certain aspects can be compatible with, combined with, included in, and/or incorporated into other aspects of the present disclosure. Thus, disclosure of certain features relative to a specific aspect of the present disclosure should not be construed as limiting application or inclusion of said features to the specific aspect. Rather, it will be appreciated that other aspects can also include said features without necessarily departing from the scope of the present disclosure.

Moreover, unless a feature is described as requiring another feature in combination therewith, any feature herein may be combined with any other feature of a same or different aspect disclosed herein. Furthermore, various well-known aspects of illustrative systems, processes, panels, compositions, products, and the like are not described herein in particular detail in order to avoid obscuring aspects of the example aspects. Such aspects are, however, also contemplated herein.

The present disclosure may be embodied in other specific forms without departing from its spirit or essential characteristics. The described aspects are to be considered in all respects only as illustrative and not restrictive. The scope of the disclosure is, therefore, indicated by the appended claims rather than by the foregoing description. While certain aspects and details have been included herein and in the attached disclosure for purposes of illustrating aspects of the present disclosure, it will be apparent to those skilled in the art that various changes in the methods, compositions, panels, products, devices, and apparatus disclosed herein may be made without departing from the scope of the disclosure, which is defined in the appended claims. All changes which come within the meaning and range of equivalency of the claims are to be embraced within their scope.

What is claimed is:

1. An engineered variant of a human leukocyte antigen (HLA), wherein the variant comprises an extracellular domain having an altered essential residue or region (ER);
wherein the altered ER is capable of contacting an antibody (Ab) complementarity determining region (CDR) and/or a CDR adjacent framework region (FR), and
wherein the altered ER includes at least 1, 2, 3, 4, 5, 6 or more amino acids.

2. The engineered variant of claim 1, wherein the altered ER comprises at least 2 or 3 amino acids within about 3.0-15.0 Å in distance from each other and/or at least 2 or 3 amino acids that are within 1-15 residues of each other.

3. The engineered variant of claim 1, wherein the altered ER further comprises one or more of:
at least one amino acid that is not conserved in all alleles at the HLA locus in a population;
at least one amino acid that is predicted to be surface or solvent exposed; and
at least one amino acid that is capable of participating in peptide binding in a common HLA allele in a population.

4. A composition comprising:
a substrate comprising an immobilized antigen comprising the engineered variant of a HLA of claim 1 or extracellular domain thereof.

5. The composition of claim 4, further comprising a plurality of substrates, each substrate comprising an immobilized antigen; and wherein:
each substrate comprises a different immobilized antigen;
at least one of the immobilized antigens comprises a second engineered variant of a HLA or extracellular domain thereof; or
at least one of the immobilized antigens comprises one or more naturally occurring HLA or extracellular domain thereof.

6. The composition of claim 4, wherein the substrate is a bead.

7. A method of generating a human leukocyte antigen (HLA) panel comprising:
a) selecting a population of HLA alleles;
b) identifying a putative essential residue or region (ER) contained in an extracellular domain of amino acid sequences of the population of HLA alleles;
c) based on b), selecting a minimal set of amino acid sequences from the population of HLA alleles capable of forming the putative ER;
d) determining the putative ER to be an HLA epitope; and
e) based on d), selecting antigens to include in an HLA panel.

8. The method of claim 7, wherein:
the putative ER is capable of contacting an antibody (Ab) complementarity determining region (CDR) and/or a CDR adjacent framework region (FR), and wherein the putative ER includes at least 1, 2, 3, 4, 5, 6 or more amino acid residues;
amino acid residues of the putative ER are topologically close to one another;
amino acid residues of the putative ER are not a conserved residue;
the putative ER is predicted or confirmed to be surface exposed in a control HLA allele;
amino acid residues of the putative ER participate in peptide binding in a control HLA allele; or
amino acid residues of the putative ER are predicted or confirmed to participate in peptide binding in at least 70%, at least 75%, at least 80%, at least 85%, at least 90%, at least 95%, at least 96%, at least 97%, at least 98%, at least 99%, at least 99.9%, or 100% of the alleles at the locus with a frequency of ≥1 in 10,000 in a population.

9. The method of claim 7, further comprising generating an engineered variant of HLA, wherein the variant comprises the putative ER in which at least 1, 2, 3, 4, 5, 6 or more amino acid residues of the putative ER are altered.

10. The method of claim 8, wherein the amino acid residues of the putative ER are not the conserved residue, wherein the conserved residue is a residue which is present in at least 70%, at least 75%, at least 80%, at least 85%, at least 90%, at least 95%, at least 96%, at least 97%, at least 98%, at least 99%, at least 99.9%, or 100% of the alleles at the locus with a frequency of ≥1 in 10,000 in a population.

11. The method of claim 7, wherein the putative ER is topologically distant from a second ER which is altered.

12. A method of generating an engineered variant (EV) of a human leukocyte antigen (HLA), comprising:
obtaining a compilation of HLA allele expressed amino acid sequences;
identifying an essential residue or region (ER) contained in an extracellular domain from the expressed amino acid sequences; and
generating an EV having an altered ER, wherein the altered ER has at least 1, 2, 3, 4, 5, 6 or more amino acid residues which are altered as compared to the identified ER, thereby generating the EV.

13. The method of claim 12, wherein the altered ER is capable of contacting an antibody (Ab) complementarity determining region (CDR) and/or a CDR adjacent framework region (FR), and wherein the altered ER includes at least 1, 2, 3, 4, 5, 6 or more amino acids.

14. The method of claim 12, wherein:
amino acid residues of the identified ER are topologically close to one another;
amino acid residues of the identified ER are not a conserved residue;
the identified ER is predicted or confirmed to be surface exposed in a control HLA allele;
the identified ER is predicted or confirmed to be surface exposed in at least 70%, at least 75%, at least 80%, at least 85%, at least 90%, at least 95%, at least 96%, at least 97%, at least 98%, at least 99%, at least 99.9%, or 100% of the alleles at the locus with a frequency of >1 in 10,000 in a population;
amino acid residues of the identified ER participate in peptide binding in a control HLA allele; or
amino acid residues of the identified ER are predicted or confirmed to participate in peptide binding in at least 70%, at least 75%, at least 80%, at least 85%, at least 90%, at least 95%, at least 96%, at least 97%, at least 98%, at least 99%, at least 99.9%, or 100% of the alleles at the locus with a frequency of ≥1 in 10,000 in a population.

15. The method of claim 14, wherein the amino acid residues of the identified ER are not the conserved residue, wherein the conserved residue is present in at least 70%, at least 75%, at least 80%, at least 85%, at least 90%, at least 95%, at least 96%, at least 97%, at least 98%, at least 99%, at least 99.9%, or 100% of the alleles at the locus with a frequency of ≥1 in 10,000 in a population.

16. The method of claim 14, wherein the identified ER is topologically distant from a second ER which is altered.

17. An engineered variant (EV) of a human leukocyte antigen (HLA) generated using the method of claim 12.

18. A human leukocyte antigen (HLA) panel generated using the method of claim 7.

19. A computing device comprising:
a processor; and
a non-transitory computer readable storage medium storing instructions executable by the processor which when executed, cause the computing device to perform one or more steps of the method of claim 7.

20. The computing device of claim 19, further comprising a machine learning module operable to perform computer processes using a machine learning process.


SEQUENCE LISTING (9 sequences)

SEQ ID NO: 1 - protein, 286 aa, synthetic construct
GSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVG

SEQ ID NO: 2 - protein, 286 aa, synthetic construct
GSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVG

SEQ ID NO: 3 - protein, 286 aa, synthetic construct
CSHSMKYFFTSVSRPGRGEPRFISVGYVDDTQFVRFDSDAASPRGEPRAPWVEQEGPEYWDRETQKYKRQAQTDRVSLRNLRGYYNQSEAGSHTLQWMCGCDLGPDGRLLRGYDQYAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVMVPSGEEQRYTCHVQHEGLPEPLTLRWEPSSQPTIPIVG

SEQ ID NO: 4 - protein, 192 aa, synthetic construct
IKADHVSTYAAFVQTHRPTGEFMFEFDEDEMFYVDLDKKETVWHLEEFGQAFSFEAQGGLANIAILNNNLNTLIQRSNHTQATNDPPEVTVFPKEPVELGQPNTLICHIDKFFPPVLNVTWLCNGELVTEGVAESLFLPRTDYSFHKFHYLTFVPSAEDFYDCRVEHWGLDQPLLKHWEAQEPIQMPETTET

SEQ ID NO: 5 - protein, 196 aa, synthetic construct
RATPENYVYQGRQECYAFNGTQRFLERYIYNREEYARFDSDVGEFRAVTELGRPAAEYWNSQKDILEEKRAVPDRVCRHNYELDEAVTLQRRVQPKVNVSPSKKGPLQHHNLLVCHVTDFYPGSIQVRWFLNGQEETAGVVSTNLIRNGDWTFQILVMLEMTPQQGDVYICQVEHTSLDSPVTVEWKAQSDSAQSK

SEQ ID NO: 6 - protein, 195 aa, synthetic construct
EDIVADHVASCGVNLYQFYGPSGQYTHEFDGDEEFYVDLERKETAWRWPEFSKFGGFDPQGALRNMAVAKHNLNIMIKRYNSTAATNEVPEVTVFSKSPVTLGQPNTLICLVDNIFPPVVNITWLSNGQSVTEGVSETSFLSKSDHSFFKISYLTFLPSADEIYDCKVEHWGLDQPLLKHWEPEIPAPMSELTET

SEQ ID NO: 7 - protein, 198 aa, synthetic construct
RDSPEDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNDQEETAGVVSTPLIRNGDWTFQILVMLEMTPQRGDVYTCHVEHPSLQSPITVEWRAQSESAQSK

SEQ ID NO: 8 - protein, 198 aa, synthetic construct
GDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSK

SEQ ID NO: 9 - protein, 198 aa, synthetic construct; identical to SEQ ID NO: 8